(19) United States
(12) Patent Application Publication
(10) Pub. No.: US 2023/0159538 A1
(43) Pub. Date: May 25, 2023

(54) PYRROLIDINE-PYRAZOLES AS PYRUVATE KINASE ACTIVATORS

(71) Applicant: Global Blood Therapeutics, Inc., South San Francisco, CA (US)

(72) Inventors: Ming Yu, South San Francisco, CA (US); Brian Edwin Cathers, South San Francisco, CA (US); Zhe Li, South San Francisco, CA (US); Brian Walter Metcalf, South San Francisco, CA (US); Lina Q. Setti, South San Francisco, CA (US); Chul Yu, South San Francisco, CA (US); Manuel Zancanella, South San Francisco, CA (US)

(21) Appl. No.: 17/916,530

(22) PCT Filed: Mar. 31, 2021
(86) PCT No.: PCT/US2021/025252

§ 371 Date: Sep. 30, 2022

Related U.S. Application Data
(60) Provisional application No. 63/003,688, filed on Apr. 1, 2020.

(51) Int. Cl. C07D 487/04; C07D 519/00

(52) U.S. Cl. CPC C07D 487/04; C07D 519/00

(57) ABSTRACT

The subject matter described herein is directed to pyruvate kinase activating compounds of Formula I and pharmaceutical salts thereof, methods of preparing the compounds, pharmaceutical compositions comprising the compounds and methods of administering the compounds for the treatment of diseases associated with PKR and/or PKM2, such as pyruvate kinase deficiency, sickle cell disease, and beta-thalassemia.

### CROSS REFERENCE TO RELATED APPLICATIONS

This application claims the benefit of and priority to U.S. Provisional Application No. 63/003,688, filed on Apr. 1, 2020, which is hereby incorporated by reference in its entirety.

### FIELD

The subject matter described herein is directed to pyruvate kinase activating compounds, methods of making the compounds, pharmaceutical compositions, and their use in the treatment of diseases associated with PKR and/or PKM2.

### BACKGROUND

Pyruvate kinase (PK) is an essential component of cellular metabolism, converting ADP and phosphoenolpyruvate (PEP) to pyruvate in the final step of glycolysis. There are four unique isoforms of pyruvate kinase that vary in concentration by different tissue types (Dayton et al., EMBO Rep. 2016 17(12):1721-1730); Israelsen and Vander Heiden, Semin. Cell Dev. Biol. 2015 July; 43:43-51; Mazurek, Int. J. Biochem. Cell Biol. 2011 July; 43(7):969-80). Each isomer is responsible for catalyzing the production of pyruvate and ATP, while being regulated in a manner respective to each tissue type.

Pyruvate kinase in the liver (PKL) and pyruvate kinase from erythrocytes/red blood cells (PKR) are tetrameric enzymes that depend on an endogenous activator called fructose-1,6-bisphosphate (FBP) for activation (Koler and Vanbellinghen, Adv. Enzyme Regul. 1968 6:127-42; Taylor and Bailey, Biochem J. 1967 967 February; 102(2):32C-33C). The PKM1 isoform is found in the brain, heart, and skeletal muscle where it functions as a stable and constitutively active tetrameric protein. PKM1 therefore does not require FBP for activation. The PKM2 isomer is expressed in most tissue types, including cancers, developing embryos, and all proliferative tissues. Similar to PKR and PKL, PKM2 requires FBP for allosteric activation via stabilization of the enzyme in a tetrameric and most active form (Cardenas and Dyson, J. Exp. Zool. 1978 June; 204(3):361-7; Imamura and Tanaka, J. Biochem. 1972 June; 71(6): 1043-51; Strandholm et al., Arch. Biochem. Biophys. 1976 March; 173(1):125-31).

Mature red blood cells (RBCs) rely on glycolysis for energy production. All tumor cells exclusively express the PKM2 isoform, suggesting that PKM2 would be a good target for cancer therapy. PKM2 is also expressed in adipose tissue and activated T-cells. Thus, controlling the regulation of PKM2 activity may be effective for treatment of obesity and diabetes in addition to cancer.

Genetic mutations in all glycolytic enzymes result in hemolytic anemia (Van Wijk and Van Solinge, Blood 2005 106 (13): 4034-4042.). Mutations in PKL and PKR that result in a loss of function are known to cause PK deficiency (PKD) and the clinical manifestation of these mutations appear confined to RBCs.

There are greater than 200 known and reported mutations associated with PKD reported worldwide (Zanella et al., J. Haematol. 2005 July; 130(1):11-25). Some mutations directly disrupt catalytic activity of the PK enzyme while other mutations disrupt the interactions between monomers that stabilize the active tetrameric enzyme. The mutation of Arginine residue 510 to Glutamine is one of the most common mutations found in North American and European patients, ˜40% of patients, and is known to disrupt stability of the PKR tetramer (Kedar et al., Clin. Genet. 2009 February; 75(2):157-62; Wang et al., Blood 2001 Nov. 15; 98(10):3113-20).

Patients with PKD suffer from chronic hemolytic anemia in addition to multiple co-morbidities. Blood transfusions and splenectomy are common treatments and it has been suggested that gene therapies could be used for treatment of PKD in the near future (Garcia-Gomez et al., Molecular Therapy 2016 Aug. 1; 24(7); Grace et al., Am. J. Hematol. 2015 September; 90(9):825-30).

The number of PKD patients worldwide is unknown; however, the prevalence in the general Caucasian population is estimated to be around 1:20,000 people with 51 cases per million people in North America (Beutler and Gelbart, Blood 2000 Jun. 1; 95(11):3585-8).

There are no approved drugs for the treatment of PKD. Clinically, hereditary PKR deficiency disorder manifests as a non-spherocytic hemolytic anemia. The clinical severity of this disorder ranges from no observable symptoms in fully-compensated hemolysis to potentially fatal severe anemia requiring chronic transfusions and/or splenectomy at early development or during physiological stress. For some of the most severe cases, while extremely rare population-wise with estimated frequency of 1 in 20,000 patients, there is no disease modifying treatment besides transfusions. These hereditary non-spherocytic hemolytic anemia patients present a clear unmet medical need. RBCs from patients with either sickle cell anemia or with beta-thalassemia suffer from increased ATP demand to maintain overall RBC health. The activation of PKR in both sickle cell disease patients and beta-thalassemia patients could lead to improved cell fitness and survival.

What is therefore needed and not effectively addressed by the art are compounds that act as pyruvate kinase activators that have desired efficacy and therapeutic potential. This problem as well as others stemming from pyruvate kinase deficiency are addressed by the subject matter described herein.

### BRIEF SUMMARY

In certain embodiments, the subject matter described herein is directed to a compound of Formula I or a pharmaceutically acceptable salt thereof.

In certain embodiments, the subject matter described herein is directed to a pharmaceutical composition comprising a compound of Formula I or a pharmaceutically acceptable salt thereof.

In certain embodiments, the subject matter described herein is directed to a method of treating a disease or disorder associated with modulation of pyruvate kinase (PKR) and/or PKM2 in a subject, comprising administering to the subject an effective amount of a compound of Formula I, a pharmaceutically acceptable salt thereof, or a pharmaceutical composition comprising a compound of Formula I.

In certain embodiments, the subject matter described herein is directed to a method of activating PKR and/or PKM2 in a subject, comprising administering to the subject an effective amount of a compound of Formula I, a pharmaceutically acceptable salt thereof, or a pharmaceutical composition comprising a compound of Formula I.

In certain embodiments, the subject matter described herein is directed to a method of treating a subject afflicted with a disease associated with decreased activity of PKR and/or PKM2, comprising administering to the subject an effective amount of a compound of Formula I, a pharmaceutically acceptable salt thereof, or a pharmaceutical composition comprising a compound of Formula I.

Other embodiments are also described.

### DETAILED DESCRIPTION

Described herein are pyruvate kinase activators of Formula I, methods of making the compounds, pharmaceutical compositions comprising the compounds, and their use in the treatment of diseases associated with decreased pyruvate kinase activity.

PKR activating compounds could be used to treat patients with beta-thalassemia and sickle cell anemia (Alli et al., Hematology 2008 December; 13(6):369-72; Kung et al., Blood 2017 Sep. 14; 130(11):1347-135). As shown in a mouse model of beta-thalassemia, the PKR activator in clinical trials, AG-348, increased PK activity and ATP levels, as well as improved RBC parameters. Similar results were obtained from treating human beta-thalassemia RBCs ex vivo (Kuo et al., Mitapivat (AG-348), an oral PK-R activator, in adults with non-transfusion-dependent thalassemia: A phase 2, open-label, multicenter study in progress; 61^{st} Am. Soc. Hematol. Annual Meeting, December 2019).

The compounds of Formula I described herein are useful in the treatment of diseases or disorders associated with pyruvate kinase function. As demonstrated by the biochemical assays described herein, the compounds of Formula I activate PKR and/or PKM2. In certain embodiments, the compounds described herein are more effective at activating PKR and/or PKM2 than AG-348. The compounds of Formula I are useful in the treatment of diseases including, but not limited to, pyruvate kinase deficiency and sickle cell disease, such as sickle cell anemia, and beta-thalassemia. Also, the compounds are methods described herein are useful in treating cancer.

Pyruvate kinase activators are needed that also possess additional beneficial properties such as improved solubility, stability, and/or potency. An advantage of the pyruvate kinase activator compounds of Formula I described herein is their preparation in sufficient yields by the synthetic routes disclosed herein.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. Unless otherwise defined, all technical and scientific terms used herein have the same meaning as commonly understood by one of ordinary skill in this field. All publications, patent applications, patents, and other references mentioned herein are incorporated by reference in their entirety. In the event that one or more of the incorporated literature, patents, and similar materials differs from or contradicts this application, including but not limited to defined terms, term usage, described techniques, or the like, this application controls.

### I. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through or perpendicular across the end of a line in a structure indicates a specified point of attachment of a group. Unless chemically or structurally required, no directionality or stereochemistry is indicated or implied by the order in which a chemical group is written or named.

The prefix “Cᵤ-Cᵥ” indicates that the following group has from u to v carbon atoms. For example, “C₁-C₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount±50%. In certain other embodiments, the term “about” includes the indicated amount ±20%. In certain other embodiments, the term “about” includes the indicated amount ±10%. In other embodiments, the term “about” includes the indicated amount±5%. In certain other embodiments, the term “about” includes the indicated amount±1%. In certain other embodiments, the term “about” includes the indicated amount±0.5% and in certain other embodiments, 0.1%. Such variations are appropriate to perform the disclosed methods or employ the disclosed compositions. Also, to the term “about x” includes description of “x”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁-C₂₀ alkyl), 1 to 12 carbon atoms (i.e., C₁-C₁₂ alkyl), 1 to 8 carbon atoms (i.e., C₁-C₈ alkyl), 1 to 6 carbon atoms (i.e., C₁-C₆ alkyl), 1 to 4 carbon atoms (i.e., C₁-C₄ alkyl), or 1 to 3 carbon atoms (i.e., C₁-C₃ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

The term “alkylene” by itself or as part of another substituent means a divalent radical derived from an alkane, such as, methylene —CH₂—, ethylene —CH₂CH₂—, and the like. As an example, a “hydroxy-methylene” refers to HO—CH₂-*, where * is the attachment point to the molecule.

Unless indicated explicitly otherwise, where combinations of groups are referred to herein as one moiety, e.g., arylalkyl or aralkyl, the last mentioned group contains the atom by which the moiety is attached to the rest of the molecule.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy and 1,2-dimethylbutoxy. Additional examples are C₁-C₃ alkoxy and C₁-C₆ alkoxy, which refer to “C₁-C₃ alkyl-O” or “C₁-C₆ alkyl-O,” respectively.

“Amino” refers to the group —NR^{y}R^{z} wherein R^{y} and R^{z} are independently hydrogen, alkyl, cycloalkyl, heterocyclyl, aryl, or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆-C₂₀ aryl), 6 to 12 carbon ring atoms (i.e., C₆-C₁₂ aryl), or 6 to 10 carbon ring atoms (i.e., C₆-C₁₀ aryl). Examples of aryl groups include, e.g., phenyl, naphthyl, fluorenyl and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Carboxyl ester” or “ester” refer to both —OC(O)R^{x} and —C(O)OR^{x}, wherein R^{x} is alkyl, cycloalkyl, heterocyclyl, aryl, or heteroaryl; each of which may be optionally substituted, as defined herein.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃-C₂₀ cycloalkyl), 3 to 12 ring carbon atoms (i.e., C₃-C₁₂ cycloalkyl), 3 to 10 ring carbon atoms (i.e., C₃-C₁₀ cycloalkyl), 3 to 8 ring carbon atoms (i.e., C₃-C₈ cycloalkyl), 3 to 7 ring carbon atoms (i.e., C₃-C₇ cycloalkyl), or 3 to 6 ring carbon atoms (i.e., C₃-C₆ cycloalkyl). Monocyclic groups include, for example, cyclopropyl, cyclobutyl, cyclopentyl, cyclohexyl, cycloheptyl and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl and the like. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Halogen” or “halo” refers to atoms occupying group VIIA of the periodic table, such as fluoro, chloro, bromo or iodo.

“Haloalkyl” refers to an unbranched or branched alkyl group as defined above, wherein one or more (e.g., 1 to 6, or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl and the like. Furthermore, “C₁-C₃ haloalkoxy” refers to an alkyl group of 1 to 3 carbons wherein at least one hydrogen atom is replaced by a halogen. Additionally, “C₁-C₆haloalkoxy” refers to an alkyl group of 1 to 6 carbons wherein at least one hydrogen atom is replaced by a halogen.

“Haloalkoxy” refers to an alkoxy group as defined above, wherein one or more (e.g., 1 to 6, or 1 to 3) hydrogen atoms are replaced by a halogen. As used herein, “C₁-C₃ haloalkoxy” refers to a C₁-C₃ alkyl-O-group wherein at least one of the hydrogen atoms of the carbon chain is replaced by a halogen.

“Hydroxyalkyl” or “hydroxyalkylene” and the like refers to an alkyl or alkylene group as defined above, wherein one or more (e.g., 1 to 6, or 1 to 3) hydrogen atoms are replaced by a hydroxy group. By way of example, the term “hydroxy-C₁-C₃ alkyl” or “hydroxy-C₁-C₃ alkylene” refers to a one to three carbon alkyl chain where one or more hydrogens on any carbon is replaced by a hydroxy group, in particular, one hydrogen on one carbon of the chain is replaced by a hydroxy group.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁-C₂₀ heteroaryl), 3 to 12 ring carbon atoms (i.e., C₃-C₁₂ heteroaryl), or 3 to 8 carbon ring atoms (i.e., C₃-C₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 9-10 membered ring systems, 6-10 membered ring systems, 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro, and may comprise one or more (e.g., 1 to 3) oxo (═O) or N-oxide (—O⁻) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂-C₂₀ heterocyclyl), 2 to 12 ring carbon atoms (i.e., C₂-C₁₂ heterocyclyl), 2 to 10 ring carbon atoms (i.e., C₂-C₁₀ heterocyclyl), 2 to 8 ring carbon atoms (i.e., C₂-C₈ heterocyclyl), 3 to 12 ring carbon atoms (i.e., C₃-C₁₂ heterocyclyl), 3 to 8 ring carbon atoms (i.e., C₃-C₈ heterocyclyl), or 3 to 6 ring carbon atoms (i.e., C₃-C₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. When the heterocyclyl ring contains 4- or 6-ring atoms, it is also referred to herein as a 4- or 6-membered heterocyclyl. When the heterocyclyl ring contains 5- to 7-ring atoms, it is also referred to herein as a 5- to 7-membered heterocyclyl. When the heterocyclyl ring contains 5- to 10-ring atoms, it is also referred to herein as a 5- to 10-membered heterocyclyl. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), tetrahydropyranyl, thiomorpholinyl, thiamorpholinyl, 1-oxo-thiomorpholinyl and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as 2-oxa-7-azaspiro[3.5]nonanyl, 2-oxa-6-azaspiro[3.4]octanyl and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl and isoindolinyl, where the heterocyclyl can be bound via either ring of the fused system.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5, 1 to 4, or 1 to 3) hydrogen atoms on the designated atom or group may or may not be replaced by a moiety other than hydrogen.

The term “substituted” used herein means any of the above groups (i.e., alkyl, alkylene, alkoxy, haloalkyl, haloalkoxy, cycloalkyl, aryl, heterocyclyl, and/or heteroaryl) wherein at least one (e.g., 1 to 5, 1 to 4, or 1 to 3) hydrogen atom is replaced by a bond to a non-hydrogen atom such as, but not limited to alkyl, alkoxy, amino, aryl, aralkyl, carboxyl, carboxyl ester, cyano, cycloalkyl, halo, haloalkyl, haloalkoxy, hydroxyalkyl, heteroaryl, heterocyclyl, —NHNH₂, hydroxy, oxo, nitro, —S(O)OH, —S(O)₂OH, N-oxide or —Si(R^{y})₃, wherein each R^{y} is independently hydrogen, alkyl, alkenyl, alkynyl, heteroalkyl, cycloalkyl, aryl, heteroaryl or heterocyclyl.

In certain embodiments, “substituted” includes any of the above alkyl, alkenyl, alkynyl, cycloalkyl, heterocyclyl, aryl or heteroaryl groups in which one or more (e.g., 1 to 5, 1 to 4, or 1 to 3) hydrogen atoms are independently replaced with deuterium, halo, cyano, nitro, oxo, alkyl, haloalkyl, cycloalkyl, heterocyclyl, aryl, heteroaryl, —NR^{g}R^{h}, —NR^{g}C(═O)R^{h}, —NR^{g}C(═O)NR^{g}R^{h}, —NR^{g}C(═O)OR^{h}, —NR^{g}S(═O)₁₋₂R^{h}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —OR^{g}, —SR^{g}, —S(═O)R^{g}, —S(═O)₂R^{g}, —OS(═O)₁₋₂R^{g}, —S(═O)₁₋₂OR^{g}, —NR^{g}S(═O)₁₋₂NR^{g}R^{h}, ═NSO₂R^{g}, ═NOR^{g}, —S(═O)₁₋₂NR^{g}R^{h}, —SF₅, —SCF₃ or —OCF₃. In certain embodiments, “substituted” also means any of the above groups in which one or more (e.g.,1 to 5, 1 to 4, or 1 to 3) hydrogen atoms are replaced with —C(═O)R^{g}, —C(═)OR^{g}, —C(═O)NR^{g}R^{h}, —CH₂SO₂R^{g}, or —CH₂SO₂NR^{g}R^{h}. In the foregoing, R^{g} and R^{h} are the same or different and independently hydrogen, alkyl, alkoxy, aryl, cycloalkyl, haloalkyl, heterocyclyl, and/or heteroaryl. In certain embodiments, “substituted” also means any of the above groups in which one or more (e.g., 1 to 5, 1 to 4, or 1 to 3) hydrogen atoms are replaced by a bond to an amino, cyano, hydroxyl, nitro, oxo, halo, alkyl, alkoxy, alkylamino, aryl, cycloalkyl, haloalkyl, heterocyclyl, N-heterocyclyl, heteroaryl, or two of R^{g} and R^{h} and Rⁱ are taken together with the atoms to which they are attached to form a heterocyclyl ring optionally substituted with oxo, halo or alkyl optionally substituted with oxo, halo, amino, hydroxyl, or alkoxy.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein.

In certain embodiments, as used herein, the phrase “one or more” refers to one to five. In certain embodiments, as used herein, the phrase “one or more” refers to one to four. In certain embodiments, as used herein, the phrase “one or more” refers to one to three.

Any compound or structure given herein, is intended to represent unlabeled forms as well as isotopically labeled forms (isotopologues) of the compounds. These forms of compounds may also be referred to as and include “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium. Further, in some embodiments, the corresponding deuterated analog is provided.

In many cases, the compounds of this disclosure are capable of forming acid and/or base salts by virtue of the presence of amino and/or carboxyl groups or groups similar thereto.

Provided also are a pharmaceutically acceptable salt, isotopically enriched analog, deuterated analog, isomer (such as a stereoisomer), mixture of isomers (such as a mixture of stereoisomers), prodrug, and metabolite of the compounds described herein.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, aluminum, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃) or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine and the like.

The term “hydrate” refers to the complex formed by the combining of a compound described herein and water.

A “solvate” refers to an association or complex of one or more solvent molecules and a compound of the disclosure. Examples of solvents that form solvates include, but are not limited to, water, isopropanol, ethanol, methanol, dimethylsulfoxide, ethylacetate, acetic acid and ethanolamine.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds of the invention, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present invention contemplates various stereoisomers and mixtures thereof and includes “enantiomers,” which refers to two stereoisomers whose molecules are nonsuperimposeable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Prodrugs” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate and benzoate derivatives), amides, guanidines, carbamates (e.g., N,N-dimethylaminocarbonyl) of hydroxy functional groups in compounds described herein and the like. Preparation, selection and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

The term, “metabolite,” as used herein refers to a resulting product formed when a compound disclosed herein is metabolized. As used herein, the term “metabolized” refers to the sum of processes (including but not limited to hydrolysis reactions and reactions catalyzed by enzymes) by which a particular substance, such as a compound disclosed herein, is changed by an organism. For example, an aldehyde moiety (—C(O)H) of the compounds of the invention may be reduced in vivo to a —CH₂OH moiety.

As used herein, the term “activator” refers to a compound of Formula I or a pharmaceutically acceptable salt thereof that increases the activity of pyruvate kinase R (PKR) or pyruvate kinase M2 (PKM2), unless specified otherwise. By “activate” herein is meant to increase the activity of PKR or PKM2 activity to a level that is greater than the basal levels of activity for PKR or PKM2 in the presence of the compound. In some embodiments, the term “activate” means an increase in the activity of PKR or PKM2 of at least about 5%, at least about 10%, at least about 20%, at least about 25%, at least about 50%, at least about 60%, at least about 70%, at least about 80%, at least about 90%, or at least about 95%. In other embodiments, activate means an increase in PKR or PKM2 activity of about 5% to about 25%, about 25% to about 50%, about 50% to about 75%, or about 75% to 100%. In some embodiments, activate means an increase in PKR or PKM2 activity of about 95% to 100%, e.g., an increase in activity of 95%, 96%, 97%, 98%, 99%, or 100%. Such increases can be measured using a variety of techniques that would be recognizable by one of skill in the art, including in vitro assays.

As used herein, the term “pyruvate kinase activator” and the like refers to a compound that activates, increases, or modulates one or more of the biological activities of pyruvate kinase. The activity could increase, for example, at least about 5%, 10%, 15%, 20%, 25%, 30%, 35%, 40%, 45%, 50%, 55%, 60%, 65%, 70%, 75%, 80%, 85%, 95% or 100% of the activity of pyruvate kinase compared to an appropriate control. The increase can be a statistically significant increase.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a pyrukate kinase deficiency (PKD). The therapeutically effective amount may vary depending on the subject, and disease or condition being treated, the weight and age of the subject, the severity of the disease or condition, and the manner of administering, which can readily be determined by one of ordinary skill in the art.

Additional definitions may also be provided below as appropriate.

### II. Compounds

In certain embodiments, the subject matter described herein is directed to compounds of Formula I, and pharmaceutically acceptable salts thereof:

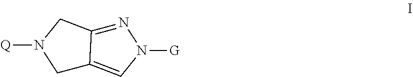

wherein,
G is

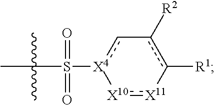

wherein,
   [symbol] represents a single or double bond;
   X⁴ is selected from the group consisting of N, C and CH;
   X¹⁰ and X¹¹ are each independently selected from the group consisting of N, CH₂, and CH, provided that only one of X¹⁰ and X¹¹ can be N;
   R¹ is selected from the group consisting of hydrogen, C₁-C₃ alkyl, C₁-C₃ alkoxy, halogen, C₁-C₃ haloalkoxy, amino, —O—R^{aa}, —(C₁-C₃ alkoxy-)R^{aa}, C₃-C₇ cycloalkyl, and hydroxy;
   R^{aa} is 4- to 7-membered heterocyclyl or C₃-C₇ cycloalkyl;
   R² is amino, C₁-C₃ alkoxy, halogen, C₁-C₃ haloalkoxy, or hydrogen, or, together with R¹ and the carbon to which each of R² and R¹ is attached, forms a 5- or 6-membered heteroaryl or 5- to 7-membered heterocyclyl containing one or two heteroatoms;
      wherein said heteroaryl or heterocyclyl formed by R¹ and R² is optionally substituted with one or two substituents, each independently selected from the group consisting of C₁-C₃ alkyl, C₁-C₃ haloalkyl, C₁-C₃ alkoxy, halogen, and C₁-C₃ haloalkoxy;
   provided that R¹ and R² cannot both be amino or hydrogen;
Q is:
   i. —C(O)—C(J¹)(J²)(J³)
      wherein,
   J¹ is hydrogen or optionally substituted C₁-C₃ alkyl; and
   J² and J³ are each independently selected from the group consisting of C₁-C₃ alkyl, hydroxy, C₁-C₃ alkoxy, hydroxy-C₁-C₃ alkyl, NR^{1a}R^{1b}, —(C₁-C₃ alkyl)-NR^{1a}R^{1b}, C₆-C₁₀ aryl, 5- to 10-membered heteroaryl, C₃-C₁₀ cycloalkyl, and 4- to 10-membered heterocyclyl;
   R^{1a} and R^{1b} are each independently hydrogen, hydroxy-C₁-C₃ alkyl, —C(O)H, or C₁-C₃ alkyl; and
wherein said aryl, heteroaryl, or heterocyclyl of J² and J³ is optionally substituted one or two times, in each instance independently with C₁-C₃ alkyl, C₁-C₃ haloalkyl, hydroxy, NR^{2a}R^{2b}, halogen, C₁-C₃ alkoxy, or C₁-C₃ haloalkoxy;
   wherein R^{2a} and R^{2b} are each independently hydrogen or C₁-C₃ alkyl; or,
   J¹ and J² together with the carbon atom to which they are each attached form a 4- to 7-membered heterocyclyl or C₃-C₇ cycloalkyl;
   ii. —C(O)O—R, wherein R is hydrogen or C₁-C₆ alkyl; or,
   iii. —S(O)₂-M
      wherein M is selected from the group consisting of C₆-C₁₀ aryl, 5- to 10-membered heteroaryl, and 5- to 10-membered heterocyclyl, wherein said aryl, heteroaryl, or heterocyclyl is optionally substituted one or two times, in each instance independently with C₁-C₃ alkyl, C₁-C₃ haloalkyl, NR^{3a}R^{3b}, —C(O)R^{2c}, hydroxy, halogen, C₁-C₃ alkoxy, or C₁-C₃ haloalkoxy;
      wherein R^{3a} and R^{3b} are each independently hydrogen or C₁-C₃ alkyl; and R^{2C} is selected from the group consisting of hydrogen, hydroxy, and C₁-C₃ alkyl;
         provided that the compound is not:

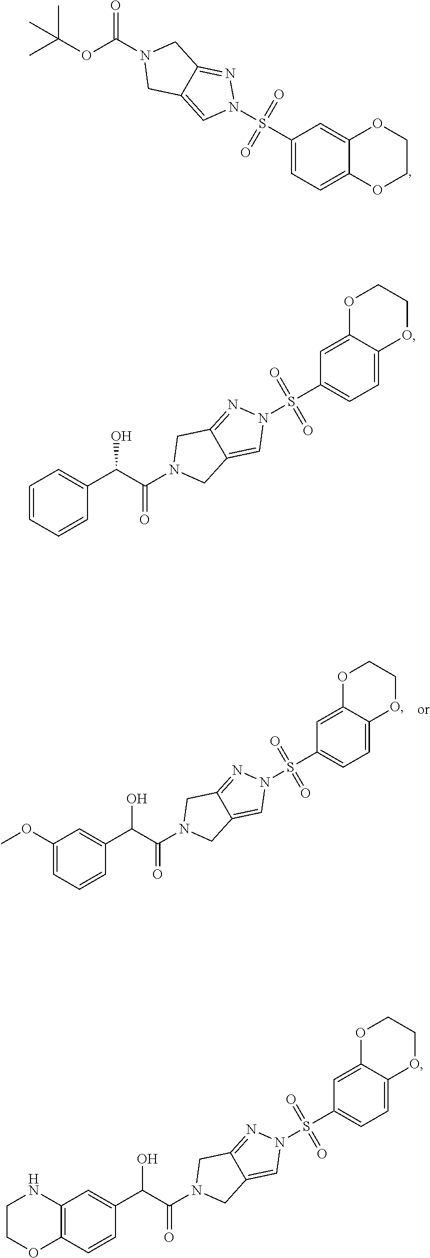 [SMILES: CC(C)(C)OC(=O)N1Cc2cn(S(=O)(=O)c3ccc4c(c3)OCCO4)nc2C1 ; O=C([C@@H](O)c1ccccc1)N1Cc2cn(S(=O)(=O)c3ccc4c(c3)OCCO4)nc2C1 ; COc1cccc(C(O)C(=O)N2Cc3cn(S(=O)(=O)c4ccc5c(c4)OCCO5)nc3C2)c1 ; O=C(C(O)c1ccc2c(c1)NCCO2)N1Cc2cn(S(=O)(=O)c3ccc4c(c3)OCCO4)nc2C1]

or salts thereof.

Useful compounds include all those having variables as described above.

In certain embodiments, compounds include those where [symbol] , in each instance, represents a single or double bond. In certain embodiments, compounds include an aromatic ring where [symbol] , in each instance, is a double bond.

In certain embodiments, compounds include those where R¹ is —O—(CH₂)ᵤ—R^{aa}, wherein u is an integer from 0 to 3 and R^{aa} is 4- to 7-membered heterocyclyl or C₃-C₇ cycloalkyl. In certain embodiments, compounds include those where u is 0.

In certain embodiments, compounds include those where X⁴ is N. In certain embodiments, compounds include those where X⁴ is C. In certain embodiments, compounds include those where X⁴ is CH.

In certain embodiments, compounds include those where X¹⁰ is N. In certain embodiments, compounds include those where X¹⁰ is CH₂. In certain embodiments, compounds include those where X¹⁰ is CH.

In certain embodiments, compounds include those where X¹¹ is N. In certain embodiments, compounds include those where X¹¹ is CH₂. In certain embodiments, compounds include those where X¹¹ is CH.

In certain embodiments, compounds include those where wherein R¹ is selected from the group consisting of hydrogen, C₁-C₃ alkyl, C₁-C₃ alkoxy, halogen, C₁-C₃ haloalkoxy, amino, —O—R^{aa}, —(C₁-C₃ alkoxy)-R^{aa}, C₃-C₇ cycloalkyl, and hydroxy;
R^{aa} is 4- to 7-membered heterocyclyl or C₃-C₇ cycloalkyl; and
R² is amino, C₁-C₃ alkoxy, halogen, C₁-C₃ haloalkoxy, or hydrogen, provided that R¹ and R² cannot both be amino or hydrogen.

In certain embodiments, R² and R¹, together with the carbon atom to which each is attached, do not form a 5- or 6-membered heteroaryl or 5- to 7-membered heterocyclyl.

In certain embodiments, compounds include those where R¹ is C₁-C₃ alkyl, C₁-C₃ alkoxy, halogen, C₁-C₃ haloalkoxy, amino, or hydroxy. In certain embodiments, compounds include those where R¹ is C₁-C₃ haloalkoxy. In certain embodiments, compounds include those where R¹ is —OCHF₂.

In certain embodiments, compounds include those where R² is amino or hydrogen, provided that R¹ and R² cannot both be amino. In certain embodiments, compounds include those where R² is hydrogen.

In certain embodiments, compounds include those where R¹ is C₁-C₃ haloalkoxy, alkoxy, C₃-C₅ cycloalkyl, or —O—R^{aa}; wherein,
R^{aa} is 4- to 6-membered heterocyclyl or C₃-C₅ cycloalkyl; and
R² is hydrogen.

In certain embodiments, compounds include those where R¹ is —OCH₃, —OCH₂F, —OCHF₂, —OCHF₃, cyclopropyl,

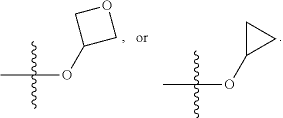 [SMILES: *OC1COC1 ; *OC1CC1]

In certain embodiments, compounds include those where R² is C₁-C₃ haloalkoxy and R¹ is hydrogen. In certain embodiments, compounds include those where R² is —OCHF₂.

In certain embodiments, compounds include those where R² together with R¹ and the carbon to which each of R² and R¹ is attached form a 5- or 6-membered heteroaryl or 5- or 6-membered heterocyclyl. In certain embodiments, compounds include those where R² together with R¹ and the carbon to which each of R² and R¹ is attached form a thiazolyl ring. In certain embodiments, compounds include those where R² together with R¹ and the carbon to which each of R² and R¹ is attached form a morpholinyl, dioxanyl, or dioxolyl ring.

In certain embodiments, compounds include those where G is:

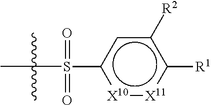

wherein X¹⁰ and X¹¹ are each independently CH or N, provided that only one of X¹⁰ and X¹¹ can be N.

In certain embodiments, compounds include those where G is:

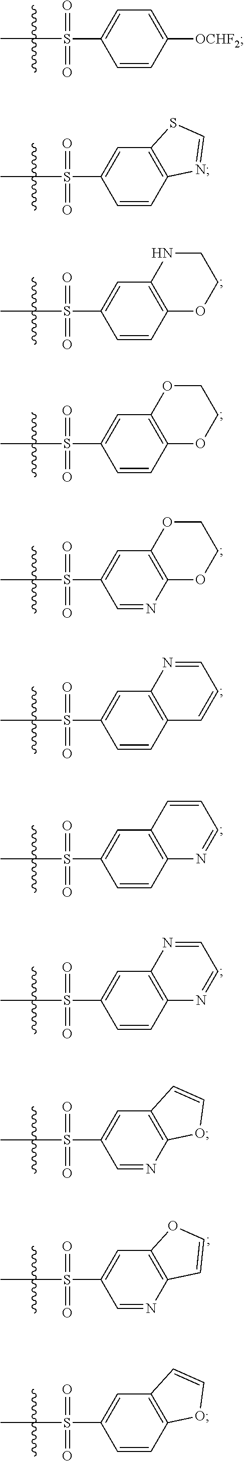

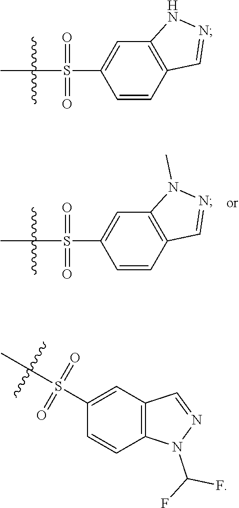 [SMILES: *S(=O)(=O)c1ccc2cn[nH]c2c1 ; *S(=O)(=O)c1ccc2cnn(C)c2c1 ; *S(=O)(=O)c1ccc2c(cnn2C(F)F)c1]

In certain embodiments, compounds include those where Q is

Q is —C(O)—C(J¹)(J²)(J³) wherein,
J¹ is hydrogen or optionally substituted C₁-C₃ alkyl; and
J² and J³ are each independently selected from the group consisting of C₁-C₃ alkyl, hydroxy, alkoxy, hydroxy-C₁-C₃ alkyl, —(CH₂)ₓNR^{1a}R^{1b}, C₆-C₁₀ aryl, 5- to 10-membered heteroaryl, 3- to 10-membered cycloalkyl, and 4- to 10-membered heterocyclyl;
x is 0, 1, or 2;
R^{1a} and R^{1b} are each independently hydrogen, hydroxy-C₁-C₃ alkyl, —C(O)H, or C₁-C₃ alkyl; and

wherein said aryl, heteroaryl, or heterocyclyl of J² and J³ is optionally substituted one or two times, in each instance independently with C₁-C₃ alkyl, C₁-C₃ haloalkyl, hydroxy, NR^{2a}R^{2b}, halogen, alkoxy, or haloalkoxy;
wherein R^{2a} and R^{2b} are each independently hydrogen or C₁-C₃ alkyl; or,
J¹ and J² together with the carbon atom to which they are each attached form a 4- to 7-membered heterocyclyl or 3- to 7-membered cycloalkyl.

In certain embodiments, compounds include those where Q is —C(O)—C(J¹)(J²)(J³), wherein:
J¹ is hydrogen or C₁-C₃ alkyl;
J² is selected from the group consisting of hydroxy, C₁-C₃ alkyl, —(CH₂)ₓNH₂, —(CH₂)ₓN(H)(hydroxy-C₁-C₃ alkyl), —(CH₂)ₓN(H)(C(O)H), 4- to 6-membered heterocyclyl, and hydroxy-C₁-C₃ alkyl;
   wherein x is 0 or 1; and
J³ is C₁-C₃ alkyl or optionally substituted phenyl or 6- to 10-membered heteroaryl;
or,
J¹ and J², together with the carbon atom to which they are each attached, form a 5- or 6-membered heterocyclyl.

In certain embodiments, compounds include those where Q is —C(O)—C(J¹)(J²)(J³), wherein:
J¹ is hydrogen or C₁-C₃ alkyl;
J² is selected from the group consisting of hydroxy, C₁-C₃ alkyl, —NHR^{1b}, —(C₁-C₃ alkyl)-NHR^{1b}, C₁-C₃ alkoxy, 4- to 6-membered heterocyclyl, and hydroxy-C₁-C₃ alkyl;
R^{1b} is hydrogen, hydroxy-C₁-C₃ alkyl, —C(O)H, or C₁-C₃ alkyl; and
J³ is optionally substituted phenyl or 6- to 10-membered heteroaryl;
or,
J¹ and J², together with the carbon atom to which they are each attached, form a 4- to 6-membered heterocyclyl.

In certain embodiments, compounds include those where J³ is phenyl or pyridinyl optionally substituted with halo, methyl, C₁-C₃ haloalkyl, or C₁-C₃ haloalkoxy. In certain embodiments, compounds include those where J³ is phenyl or pyridinyl optionally substituted with chloro, fluoro, methyl, —CH₂F, —CHF₂, —CF₃, —OCH₂F, —OCHF₂, or —OCF₃.

In certain embodiments, compounds include those where J¹ and J², together with the carbon atom to which they are each attached, form a tetrahydrofuranyl, oxetanyl, pyrrolidinyl, or morpholinyl ring.

In certain embodiments, compounds include those where Q is

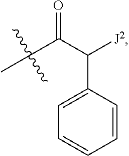

wherein J² is selected from the group consisting of —NH₂, hydroxy, hydroxy-methylene, and —CH(OH)CH₃.

In certain embodiments, compounds include those where Q is

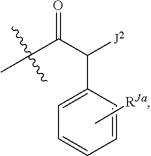

wherein J² is selected from the group consisting of —NH₂, —CH₂NH₂, —NHCH₂CH₂OH, —NHC(O)H, hydroxy, —C(CH₃)₂OH, —CH₂OH, —OCH₃,

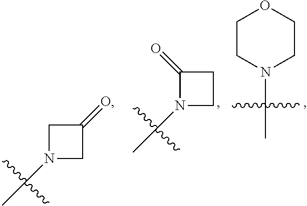

### and —CH(OH)CH₃; and

R^{Ja} is hydrogen, chloro, fluoro, —CF₃, —OCF₃, or methyl.

In certain embodiments, compounds include those where Q is

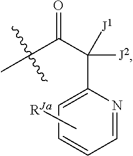

wherein J¹ is methyl or hydrogen and J² is —CH₂OH, or hydroxy; and R^{Ja} is hydrogen, methyl, fluoro, or chloro.

In certain embodiments, compounds include those where Q is

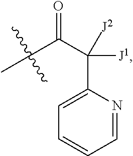

wherein J¹ is methyl and J² is hydroxy-methylene; or

J¹ and J² together with the carbon atom to which they are each attached form a tetrahydrofuranyl ring.

In certain embodiments, compounds include those where J¹ and J², together with the carbon atom to which they are each attached, form a 4- to 7-membered heterocyclyl.

In certain embodiments, compounds include those where J¹ and J², together with the carbon atom to which they are each attached, form a tetrahydrofuranyl, oxetanyl, pyrrolidinyl, or morpholinyl ring.

In certain embodiments, compounds include those where Q is —S(O)₂-M, where M is phenyl, optionally substituted once with NR^{3a}R^{3b} or haloalkoxy; and wherein R^{3a} and R^{3b} are each hydrogen. In certain embodiments, compounds include those where Q is —S(O)₂-M, where M is optionally substituted 5- to 10-membered heterocyclyl or 5- to 10-membered heteroaryl. In certain embodiments, compounds include those where Q is —S(O)₂-M, where M is 9- or 10-membered bicyclic heterocyclyl or 9- or 10-membered bicyclic heteroaryl, optionally substituted once with NR^{3a}R^{3b}, haloalkoxy, or —C(O)R^{2c}; where R^{3a} and R^{3b} are each hydrogen; and R^{2c} is C₁-C₃ alkyl. In certain embodiments, compounds include those where Q is —S(O)₂-M, where M is:

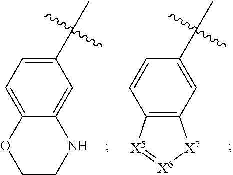

wherein

X⁷ is O, S or NH,

X⁶ is N or CH, and

X⁵ is N or CR²⁰,
wherein R²⁰ is selected from the group consisting of hydrogen, C₁-C₃ alkyl, and hydroxy;

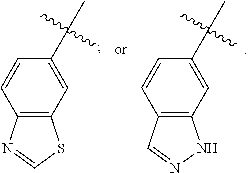 [SMILES: *c1ccc2ncsc2c1 ; *c1ccc2cn[nH]c2c1]

The subject matter described herein includes the following compounds in Table 1, or pharmaceutically acceptable salts thereof:

TABLE 1

| | | | Mass |
|---|---|---|---|
| Cmpd | | | Found |
| No. | Structure ^{a} | IUPAC Name ^{a} | (M + 1) |
| 1 | 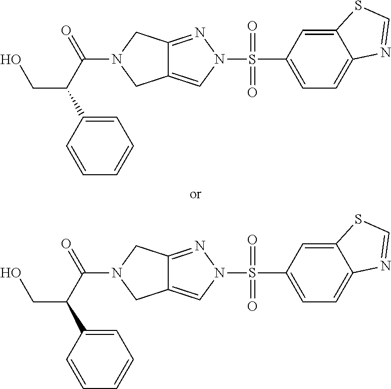 [SMILES: O=C([C@@H](CO)c1ccccc1)N1Cc2cn(S(=O)(=O)c3ccc4ncsc4c3)nc2C1 ; O=C([C@H](CO)c1ccccc1)N1Cc2cn(S(=O)(=O)c3ccc4ncsc4c3)nc2C1] | (2R)-1-[2-(1,3-benzothiazole-6- sulfonyl)-2H,4H,5H,6H- pyrrolo[3,4-c]pyrazol-5-yl]-3- hydroxy-2-phenylpropan-1-one or (2S)-1-[2-(1,3-benzothiazole-6- sulfonyl)-2H,4H,5H,6H- pyrrolo[3,4-c]pyrazol-5-yl]-3- hydroxy-2-phenylpropan-1-one | 455.0 |
| 2 | 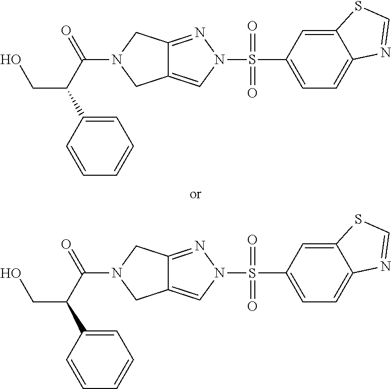 [SMILES: O=C([C@@H](CO)c1ccccc1)N1Cc2cn(S(=O)(=O)c3ccc4ncsc4c3)nc2C1 ; O=C([C@H](CO)c1ccccc1)N1Cc2cn(S(=O)(=O)c3ccc4ncsc4c3)nc2C1] | (2R)-1-[2-(1,3-benzothiazole-6- sulfonyl)-2H,4H,5H,6H- pyrrolo[3,4-c]pyrazol-5-yl]-3- hydroxy-2-phenylpropan-1-one or (2S)-1-[2-(1,3-benzothiazole-6- sulfonyl)-2H,4H,5H,6H- pyrrolo[3,4-c]pyrazol-5-yl]-3- hydroxy-2-phenylpropan-1-one | 455.0 |
| 3 | 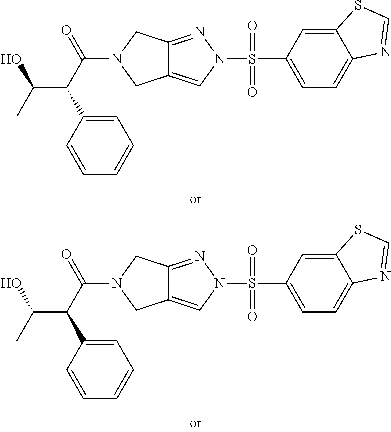 [SMILES: C[C@@H](O)[C@H](C(=O)N1Cc2cn(S(=O)(=O)c3ccc4ncsc4c3)nc2C1)c1ccccc1 ; C[C@H](O)[C@@H](C(=O)N1Cc2cn(S(=O)(=O)c3ccc4ncsc4c3)nc2C1)c1ccccc1] | (2R,3R)-1-[2-(1,3- benzothiazole-6-sulfonyl)- 2H,4H,5H,6H-pyrrolo[3,4- c]pyrazol-5-yl]-3-hydroxy-2- phenylbutan-1-one or (2S,3S)-1-[2-(1,3- benzothiazole-6-sulfonyl)- 2H,4H,5H,6H-pyrrolo[3,4- c]pyrazol-5-yl]-3-hydroxy-2- phenylbutan-1-one or (2R,3S)-1-[2-(1,3- benzothiazole-6-sulfonyl)- 2H,4H,5H,6H-pyrrolo[3,4- c]pyrazol-5-yl]-3-hydroxy-2- phenylbutan-1-one or (2S,3R)-1-[2-(1,3- benzothiazole-6-sulfonyl)- 2H,4H,5H,6H-pyrrolo[3,4- c]pyrazol-5-yl]-3-hydroxy-2- phenylbutan-1-one | 469.1 |
| | 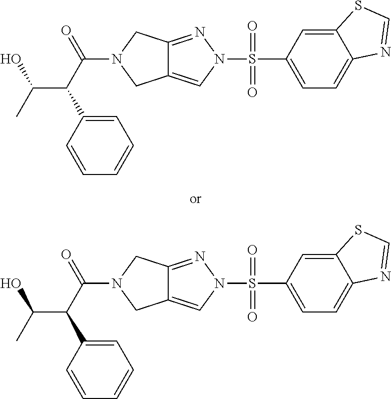 [SMILES: C[C@H](O)[C@H](C(=O)N1Cc2cn(S(=O)(=O)c3ccc4ncsc4c3)nc2C1)c1ccccc1 ; C[C@@H](O)[C@@H](C(=O)N1Cc2cn(S(=O)(=O)c3ccc4ncsc4c3)nc2C1)c1ccccc1] | | |
| 4 | 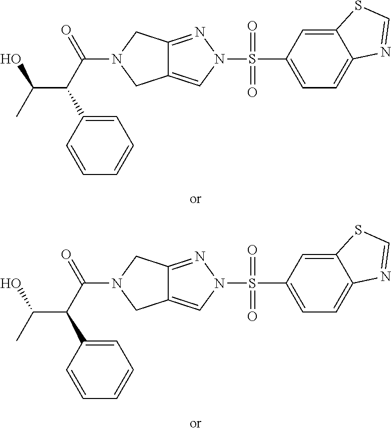 [SMILES: C[C@@H](O)[C@H](C(=O)N1Cc2cn(S(=O)(=O)c3ccc4ncsc4c3)nc2C1)c1ccccc1 ; C[C@H](O)[C@@H](C(=O)N1Cc2cn(S(=O)(=O)c3ccc4ncsc4c3)nc2C1)c1ccccc1] | (2R,3R)-1-[2-(1,3- benzothiazole-6-sulfonyl)- 2H,4H,5H,6H-pyrrolo[3,4- c]pyrazol-5-yl]-3-hydroxy-2- phenylbutan-1-one or (2S,3S)-1-[2-(1,3- benzothiazole-6-sulfonyl)- 2H,4H,5H,6H-pyrrolo[3,4- c]pyrazol-5-yl]-3-hydroxy-2- phenylbutan-1-one or (2R,3S)-1-[2-(1,3- benzothiazole-6-sulfonyl)- 2H,4H,5H,6H-pyrrolo[3,4- c]pyrazol-5-yl]-3-hydroxy-2- phenylbutan-1-one or (2S,3R)-1-[2-(1,3- benzothiazole-6-sulfonyl)- 2H,4H,5H,6H-pyrrolo[3,4- c]pyrazol-5-yl]-3-hydroxy-2- phenylbutan-1-one | 469.1 |
| | 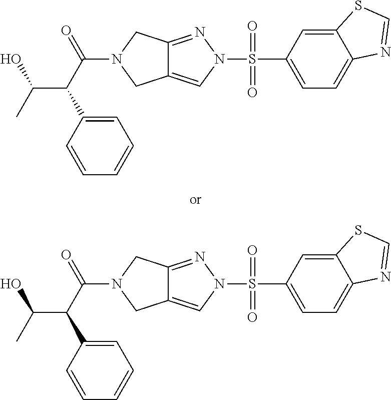 [SMILES: C[C@H](O)[C@H](C(=O)N1Cc2cn(S(=O)(=O)c3ccc4ncsc4c3)nc2C1)c1ccccc1 ; C[C@@H](O)[C@@H](C(=O)N1Cc2cn(S(=O)(=O)c3ccc4ncsc4c3)nc2C1)c1ccccc1] | | |
| 5 | 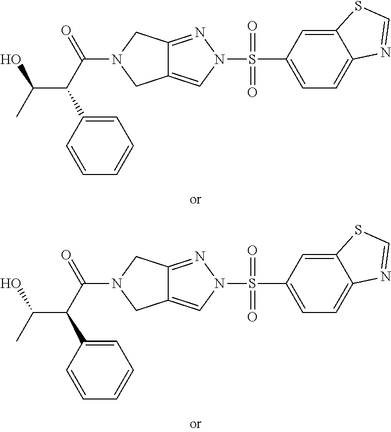 [SMILES: C[C@@H](O)[C@H](C(=O)N1Cc2cn(S(=O)(=O)c3ccc4ncsc4c3)nc2C1)c1ccccc1 ; C[C@H](O)[C@@H](C(=O)N1Cc2cn(S(=O)(=O)c3ccc4ncsc4c3)nc2C1)c1ccccc1] | (2R,3R)-1-[2-(1,3- benzothiazole-6-sulfonyl)- 2H,4H,5H,6H-pyrrolo[3,4- c]pyrazol-5-yl]-3-hydroxy-2- phenylbutan-1-one or (2S,3S)-1-[2-(1,3- benzothiazole-6-sulfonyl)- 2H,4H,5H,6H-pyrrolo[3,4- c]pyrazol-5-yl]-3-hydroxy-2- phenylbutan-1-one or (2R,3S)-1-[2-(1,3- benzothiazole-6-sulfonyl)- 2H,4H,5H,6H-pyrrolo[3,4- c]pyrazol-5-yl]-3-hydroxy-2- phenylbutan-1-one or (2S,3R)-1-[2-(1,3- benzothiazole-6-sulfonyl)- 2H,4H,5H,6H-pyrrolo[3,4- c]pyrazol-5-yl]-3-hydroxy-2- phenylbutan-1-one | 469.1 |
| | 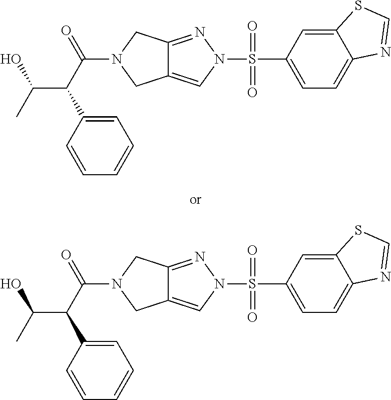 [SMILES: C[C@H](O)[C@H](C(=O)N1Cc2cn(S(=O)(=O)c3ccc4ncsc4c3)nc2C1)c1ccccc1 ; C[C@@H](O)[C@@H](C(=O)N1Cc2cn(S(=O)(=O)c3ccc4ncsc4c3)nc2C1)c1ccccc1] | | |
| 6 | 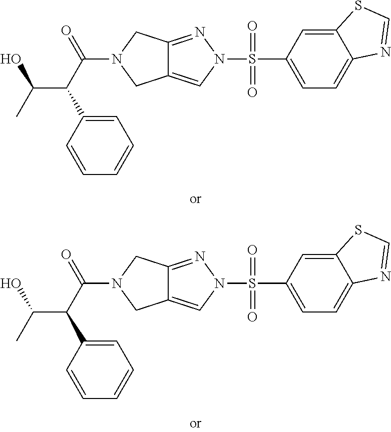 [SMILES: C[C@@H](O)[C@H](C(=O)N1Cc2cn(S(=O)(=O)c3ccc4ncsc4c3)nc2C1)c1ccccc1 ; C[C@H](O)[C@@H](C(=O)N1Cc2cn(S(=O)(=O)c3ccc4ncsc4c3)nc2C1)c1ccccc1] | (2R,3R)-1-[2-(1,3- benzothiazole-6-sulfonyl)- 2H,4H,5H,6H-pyrrolo[3,4- c]pyrazol-5-yl]-3-hydroxy-2- phenylbutan-1-one or (2S,3S)-1-[2-(1,3- benzothiazole-6-sulfonyl)- 2H,4H,5H,6H-pyrrolo[3,4- c]pyrazol-5-yl]-3-hydroxy-2- phenylbutan-1-one or (2R,3S)-1-[2-(1,3- benzothiazole-6-sulfonyl)- 2H,4H,5H,6H-pyrrolo[3,4- c]pyrazol-5-yl]-3-hydroxy-2- phenylbutan-1-one or (2S,3R)-1-[2-(1,3- benzothiazole-6-sulfonyl)- 2H,4H,5H,6H-pyrrolo[3,4- c]pyrazol-5-yl]-3-hydroxy-2- phenylbutan-1-one | 469.1 |
| | 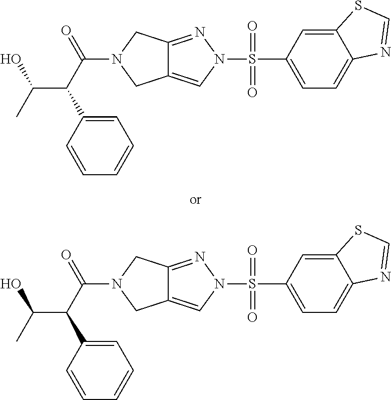 [SMILES: C[C@H](O)[C@H](C(=O)N1Cc2cn(S(=O)(=O)c3ccc4ncsc4c3)nc2C1)c1ccccc1 ; C[C@@H](O)[C@@H](C(=O)N1Cc2cn(S(=O)(=O)c3ccc4ncsc4c3)nc2C1)c1ccccc1] | | |
| 7 | 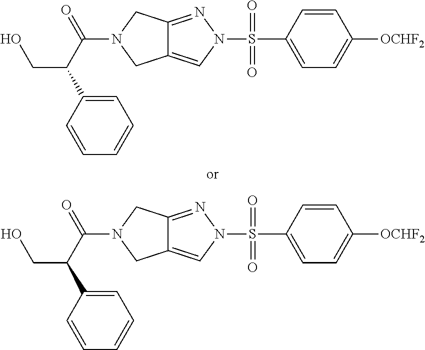 [SMILES: O=C([C@@H](CO)c1ccccc1)N1Cc2cn(S(=O)(=O)c3ccc(OC(F)F)cc3)nc2C1 ; O=C([C@H](CO)c1ccccc1)N1Cc2cn(S(=O)(=O)c3ccc(OC(F)F)cc3)nc2C1] | (2R)-1-{2-[4- (difluoromethoxy)benzenesulfonyl]- 2H,4H,5H,6H-pyrrolo[3,4- c]pyrazol-5-yl}-3-hydroxy-2- phenylpropan-1-one or (2S)-1-{2-[4- (difluoromethoxy)benzenesulfonyl]- 2H,4H,5H,6H-pyrrolo[3,4- c]pyrazol-5-yl}-3-hydroxy-2- phenylpropan-1-one | 464.0 |
| 8 | 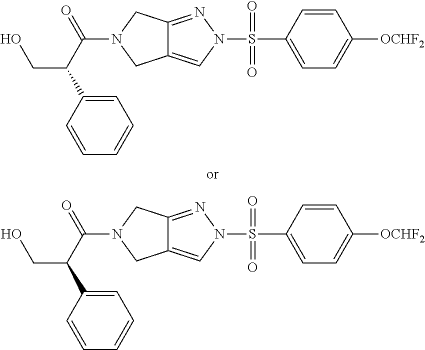 [SMILES: O=C([C@@H](CO)c1ccccc1)N1Cc2cn(S(=O)(=O)c3ccc(OC(F)F)cc3)nc2C1 ; O=C([C@H](CO)c1ccccc1)N1Cc2cn(S(=O)(=O)c3ccc(OC(F)F)cc3)nc2C1] | (2R)-1-{2-[4- (difluoromethoxy)benzenesulfonyl]- 2H,4H,5H,6H-pyrrolo[3,4- c]pyrazol-5-yl}-3-hydroxy-2- phenylpropan-1-one or (2S)-1-{2-[4- (difluoromethoxy)benzenesulfonyl]- 2H,4H,5H,6H-pyrrolo[3,4- c]pyrazol-5-yl}-3-hydroxy-2- phenylpropan-1-one | 464.0. |
| 9 | 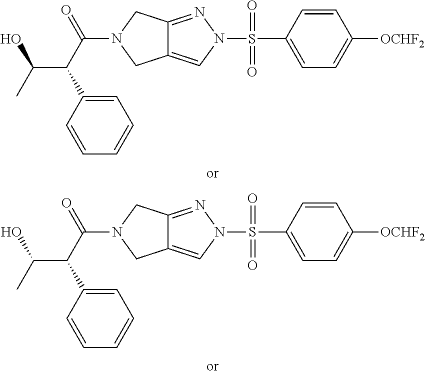 [SMILES: C[C@@H](O)[C@H](C(=O)N1Cc2cn(S(=O)(=O)c3ccc(OC(F)F)cc3)nc2C1)c1ccccc1 ; C[C@H](O)[C@H](C(=O)N1Cc2cn(S(=O)(=O)c3ccc(OC(F)F)cc3)nc2C1)c1ccccc1] | (2R,3R)-1-{2-[4- (difluoromethoxy)benzenesulfonyl]- 2H,4H,5H,6H-pyrrolo[3,4- c]pyrazol-5-yl}-3-hydroxy-2- phenylbutan-1-one or (2R,3S)-1-{2-[4- (difluoromethoxy)benzenesulfonyl]- 2H,4H,5H,6H-pyrrolo[3,4- c]pyrazol-5-yl}-3-hydroxy-2- phenylbutan-1-one or (2S,3S)-1-{2-[4- (difluoromethoxy)benzenesulfonyl]- 2H,4H,5H,6H-pyrrolo[3,4- c]pyrazol-5-yl}-3-hydroxy-2- phenylbutan-1-one or (2S,3R)-1-{2-[4- (difluoromethoxy)benzenesulfonyl]- 2H,4H,5H,6H-pyrrolo[3,4- c]pyrazol-5-yl}-3-hydroxy-2- phenylbutan-1-one | 478.2 |
| | 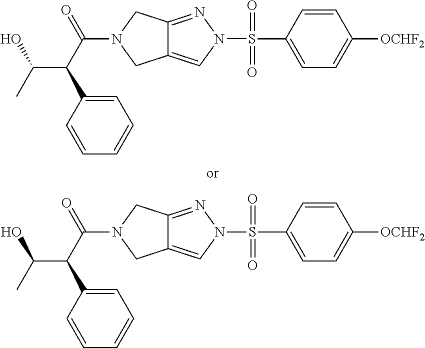 [SMILES: C[C@H](O)[C@@H](C(=O)N1Cc2cn(S(=O)(=O)c3ccc(OC(F)F)cc3)nc2C1)c1ccccc1 ; C[C@@H](O)[C@@H](C(=O)N1Cc2cn(S(=O)(=O)c3ccc(OC(F)F)cc3)nc2C1)c1ccccc1] | | |
| 10 | 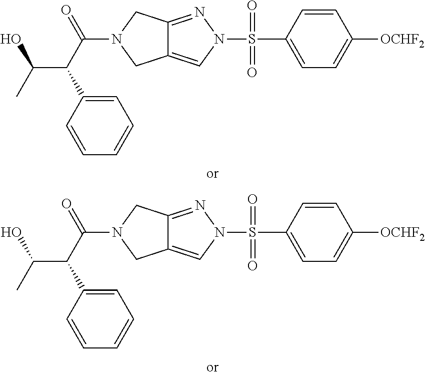 [SMILES: C[C@@H](O)[C@H](C(=O)N1Cc2cn(S(=O)(=O)c3ccc(OC(F)F)cc3)nc2C1)c1ccccc1 ; C[C@H](O)[C@H](C(=O)N1Cc2cn(S(=O)(=O)c3ccc(OC(F)F)cc3)nc2C1)c1ccccc1] | (2R,3R)-1-{2-[4- (difluoromethoxy)benzenesulfonyl]- 2H,4H,5H,6H-pyrrolo[3,4- c]pyrazol-5-yl}-3-hydroxy-2- phenylbutan-1-one or (2R,3S)-1-{2-[4- (difluoromethoxy)benzenesulfonyl]- 2H,4H,5H,6H-pyrrolo[3,4- c]pyrazol-5-yl}-3-hydroxy-2- phenylbutan-1-one or (2S,3S)-1-{2-[4- (difluoromethoxy)benzenesulfonyl]- 2H,4H,5H,6H-pyrrolo[3,4- c]pyrazol-5-yl}-3-hydroxy-2- phenylbutan-1-one or (2S,3R)-1-{2-[4- (difluoromethoxy)benzenesulfonyl]- 2H,4H,5H,6H-pyrrolo[3,4- c]pyrazol-5-yl}-3-hydroxy-2- phenylbutan-1-one | 478.2 |
| | 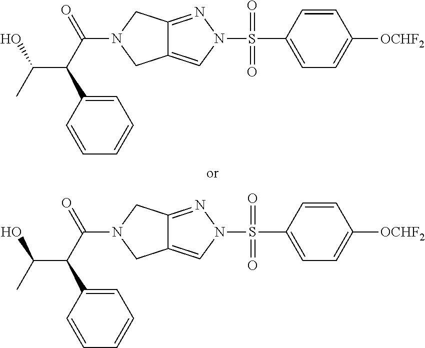 [SMILES: C[C@H](O)[C@@H](C(=O)N1Cc2cn(S(=O)(=O)c3ccc(OC(F)F)cc3)nc2C1)c1ccccc1 ; C[C@@H](O)[C@@H](C(=O)N1Cc2cn(S(=O)(=O)c3ccc(OC(F)F)cc3)nc2C1)c1ccccc1] | | |
| 11 | 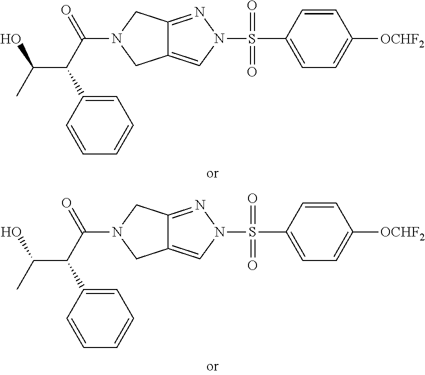 [SMILES: C[C@@H](O)[C@H](C(=O)N1Cc2cn(S(=O)(=O)c3ccc(OC(F)F)cc3)nc2C1)c1ccccc1 ; C[C@H](O)[C@H](C(=O)N1Cc2cn(S(=O)(=O)c3ccc(OC(F)F)cc3)nc2C1)c1ccccc1] | (2R,3R)-1-{2-[4- (difluoromethoxy)benzenesulfonyl]- 2H,4H,5H,6H-pyrrolo[3,4- c]pyrazol-5-yl}-3-hydroxy-2- phenylbutan-1-one or (2R,3S)-1-{2-[4- (difluoromethoxy)benzenesulfonyl]- 2H,4H,5H,6H-pyrrolo[3,4- c]pyrazol-5-yl}-3-hydroxy-2- phenylbutan-1-one or (2S,3S)-1-{2-[4- (difluoromethoxy)benzenesulfonyl]- 2H,4H,5H,6H-pyrrolo[3,4- c]pyrazol-5-yl}-3-hydroxy-2- phenylbutan-1-one or (2S,3R)-1-{2-[4- (difluoromethoxy)benzenesulfonyl]- 2H,4H,5H,6H-pyrrolo[3,4- c]pyrazol-5-yl}-3-hydroxy-2- phenylbutan-1-one | 478.2 |
| | 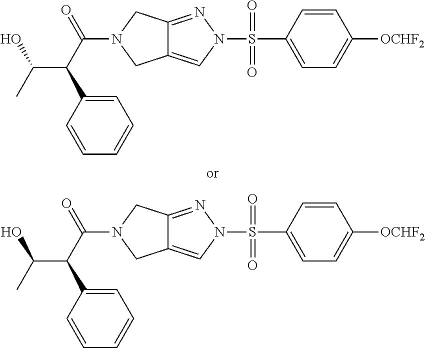 [SMILES: C[C@H](O)[C@@H](C(=O)N1Cc2cn(S(=O)(=O)c3ccc(OC(F)F)cc3)nc2C1)c1ccccc1 ; C[C@@H](O)[C@@H](C(=O)N1Cc2cn(S(=O)(=O)c3ccc(OC(F)F)cc3)nc2C1)c1ccccc1] | | |
| 12 | 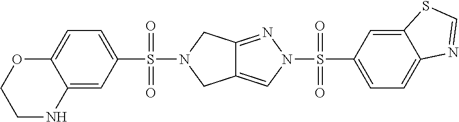 [SMILES: O=S(=O)(c1ccc2c(c1)NCCO2)N1Cc2cn(S(=O)(=O)c3ccc4ncsc4c3)nc2C1] | 6-{[2-(1,3-benzothiazole-6- sulfonyl)-2H,4H,5H,6H- pyrrolo[3,4-c]pyrazol-5- yl]sulfonyl}-3,4-dihydro-2H- 1,4-benzoxazine | 503.8 |
| 13 | 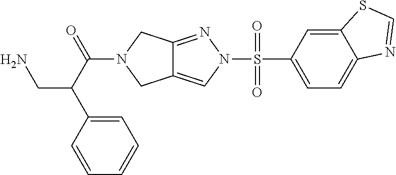 [SMILES: NCC(C(=O)N1Cc2cn(S(=O)(=O)c3ccc4ncsc4c3)nc2C1)c1ccccc1] | 3-amino-1-[2-(1,3- benzothiazole-6-sulfonyl)- 2H,4H,5H,6H-pyrrolo[3,4- c]pyrazol-5-yl]-2- phenylpropan-1-one | 454.3 |
| 14 | 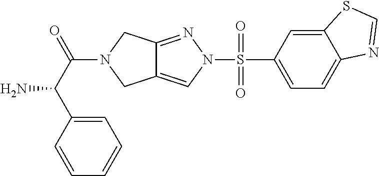 [SMILES: N[C@H](C(=O)N1Cc2cn(S(=O)(=O)c3ccc4ncsc4c3)nc2C1)c1ccccc1] | (2S)-2-amino-1-[2-(1,3- benzothiazole-6-sulfonyl)- 2H,4H,5H,6H-pyrrolo[3,4- c]pyrazol-5-yl]-2-phenylethan- 1-one | 440.3 |
| 15 | 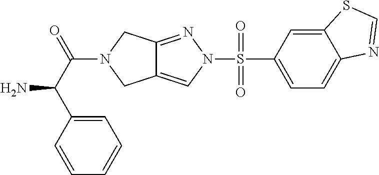 [SMILES: N[C@@H](C(=O)N1Cc2cn(S(=O)(=O)c3ccc4ncsc4c3)nc2C1)c1ccccc1] | (2R)-2-amino-1-[2-(1,3- benzothiazole-6-sulfonyl)- 2H,4H,5H,6H-pyrrolo[3,4- c]pyrazol-5-yl]-2-phenylethan- 1-one | 540.2 |
| 16 | 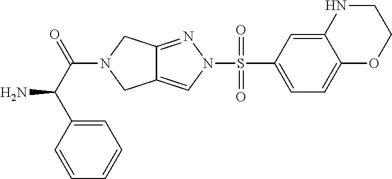 [SMILES: N[C@@H](C(=O)N1Cc2cn(S(=O)(=O)c3ccc4c(c3)NCCO4)nc2C1)c1ccccc1] | (2R)-2-amino-1-[2-(3,4- dihydro-2H-1,4-benzoxazine-6- sulfonyl)-2H,4H,5H,6H- pyrrolo[3,4-c]pyrazol-5-yl]-2- phenylethan-1-one | 440.4 |
| 17 | 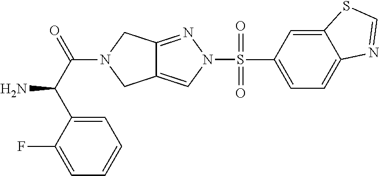 [SMILES: N[C@@H](C(=O)N1Cc2cn(S(=O)(=O)c3ccc4ncsc4c3)nc2C1)c1ccccc1F] | (2R)-2-amino-1-[2-(1,3- benzothiazole-6-sulfonyl)- 2H,4H,5H,6H-pyrrolo[3,4- c]pyrazol-5-yl]-2-(2- fluorophenyl)ethan-1-one | 440.4 |
| 18 | 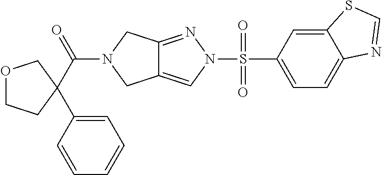 [SMILES: O=C(N1Cc2cn(S(=O)(=O)c3ccc4ncsc4c3)nc2C1)C1(c2ccccc2)CCOC1] | 6-{[5-(3-phenyloxolane-3- carbonyl)-2H,4H,5H,6H- pyrrolo[3,4-c]pyrazol-2- yl]sulfonyl}-1,3-benzothiazole | 481.3 |
| 19 | 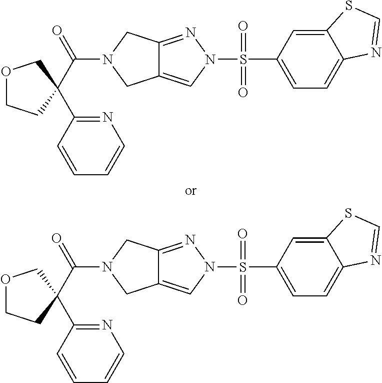 [SMILES: O=C(N1Cc2cn(S(=O)(=O)c3ccc4ncsc4c3)nc2C1)[C@]1(c2ccccn2)CCOC1 ; "or" ; O=C(N1Cc2cn(S(=O)(=O)c3ccc4ncsc4c3)nc2C1)[C@@]1(c2ccccn2)CCOC1] | 6-({5-[(3S)-3-(pyridin-2- yl)oxolane-3-carbonyl]- 2H,4H,5H,6H-pyrrolo[3,4- c]pyrazol-2-yl}sulfonyl)-1,3- benzothiazole or 6-({5-[(3R)-3-(pyridin-2- yl)oxolane-3-carbonyl]- 2H,4H,5H,6H-pyrrolo[3,4- c]pyrazol-2-yl}sulfonyl)-1,3- benzothiazole | 482.1 |
| 20 | 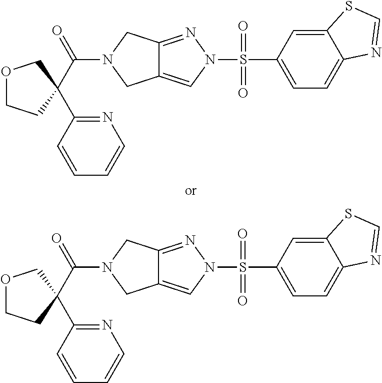 [SMILES: O=C(N1Cc2cn(S(=O)(=O)c3ccc4ncsc4c3)nc2C1)[C@]1(c2ccccn2)CCOC1 ; "or" ; O=C(N1Cc2cn(S(=O)(=O)c3ccc4ncsc4c3)nc2C1)[C@@]1(c2ccccn2)CCOC1] | 6-({5-[(3S)-3-(pyridin-2- yl)oxolane-3-carbonyl]- 2H,4H,5H,6H-pyrrolo[3,4- c]pyrazol-2-yl}sulfonyl)-1,3- benzothiazole or 6-({5-[(3R)-3-(pyridin-2- yl)oxolane-3-carbonyl]- 2H,4H,5H,6H-pyrrolo[3,4- c]pyrazol-2-yl}sulfonyl)-1,3- benzothiazole | 482.1 |
| 21 | 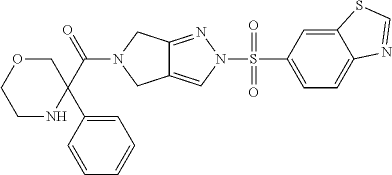 [SMILES: O=C(N1Cc2cn(S(=O)(=O)c3ccc4ncsc4c3)nc2C1)C1(c2ccccc2)COCCN1] | 6-{[5-(3-phenylmorpholine-3- carbonyl)-2H,4H,5H,6H- pyrrolo[3,4-c]pyrazol-2- yl]sulfonyl}-1,3-benzothiazole | 496.1 |
| 22 | 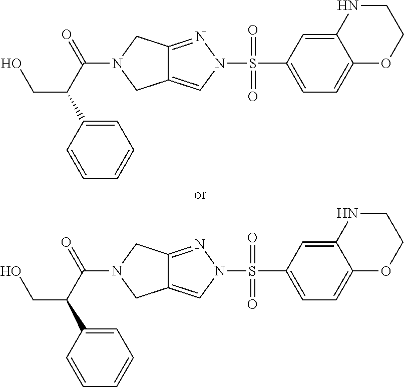 [SMILES: O=C([C@@H](CO)c1ccccc1)N1Cc2cn(S(=O)(=O)c3ccc4c(c3)NCCO4)nc2C1 ; "or" ; O=C([C@H](CO)c1ccccc1)N1Cc2cn(S(=O)(=O)c3ccc4c(c3)NCCO4)nc2C1] | (2R)-1-[2-(3,4-dihydro-2H-1,4- benzoxazine-6-sulfonyl)- 2H,4H,5H,6H-pyrrolo[3,4- c]pyrazol-5-yl]-3-hydroxy-2- phenylpropan-1-one or (2S)-1-[2-(3,4-dihydro-2H-1,4- benzoxazine-6-sulfonyl)- 2H,4H,5H,6H-pyrrolo[3,4- c]pyrazol-5-yl]-3-hydroxy-2- phenylpropan-1-one | 455.4 |
| 23 | 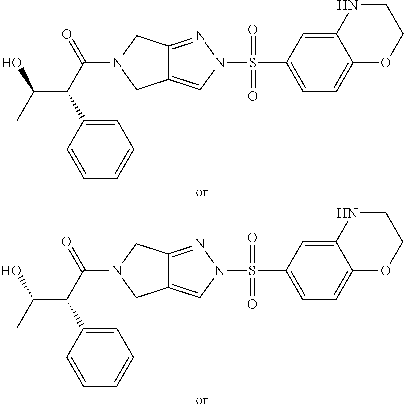 [SMILES: C[C@@H](O)[C@H](C(=O)N1Cc2cn(S(=O)(=O)c3ccc4c(c3)NCCO4)nc2C1)c1ccccc1 ; "or" ; C[C@H](O)[C@H](C(=O)N1Cc2cn(S(=O)(=O)c3ccc4c(c3)NCCO4)nc2C1)c1ccccc1 ; "or"] | (2R,3R)-1-[2-(3,4-dihydro-2H- 1,4-benzoxazine-6-sulfonyl)- 2H,4H,5H,6H-pyrrolo[3,4- c]pyrazol-5-yl]-3-hydroxy-2- phenylbutan-1-one or (2R,3S)-1-[2-(3,4-dihydro-2H- 1,4-benzoxazine-6-sulfonyl)- 2H,4H,5H,6H-pyrrolo[3,4- c]pyrazol-5-yl]-3-hydroxy-2- phenylbutan-1-one or (2S,3S)-1-[2-(3,4-dihydro-2H- 1,4-benzoxazine-6-sulfonyl)- 2H,4H,5H,6H-pyrrolo[3,4- c]pyrazol-5-yl]-3-hydroxy-2- phenylbutan-1-one or (2S,3R)-1-[2-(3,4-dihydro-2H- 1,4-benzoxazine-6-sulfonyl)- 2H,4H,5H,6H-pyrrolo[3,4- c]pyrazol-5-yl]-3-hydroxy-2- phenylbutan-1-one | 469.4 |
| | 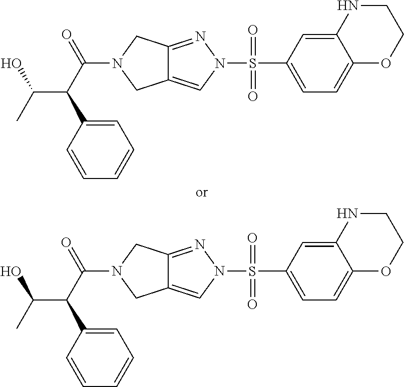 [SMILES: C[C@H](O)[C@@H](C(=O)N1Cc2cn(S(=O)(=O)c3ccc4c(c3)NCCO4)nc2C1)c1ccccc1 ; "or" ; C[C@@H](O)[C@@H](C(=O)N1Cc2cn(S(=O)(=O)c3ccc4c(c3)NCCO4)nc2C1)c1ccccc1] | | |
| 24 | 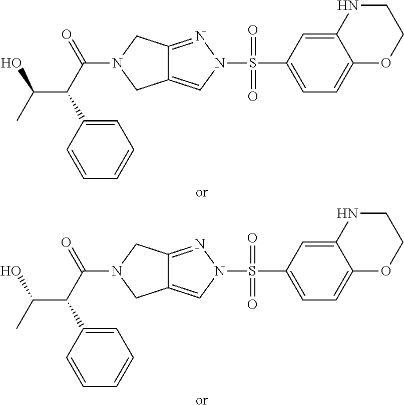 [SMILES: C[C@@H](O)[C@H](C(=O)N1Cc2cn(S(=O)(=O)c3ccc4c(c3)NCCO4)nc2C1)c1ccccc1 ; "or" ; C[C@H](O)[C@H](C(=O)N1Cc2cn(S(=O)(=O)c3ccc4c(c3)NCCO4)nc2C1)c1ccccc1 ; "or"] | (2R,3R)-1-[2-(3,4-dihydro-2H- 1,4-benzoxazine-6-sulfonyl)- 2H,4H,5H,6H-pyrrolo[3,4- c]pyrazol-5-yl]-3-hydroxy-2- phenylbutan-1-one or (2R,3S)-1-[2-(3,4-dihydro-2H- 1,4-benzoxazine-6-sulfonyl)- 2H,4H,5H,6H-pyrrolo[3,4- c]pyrazol-5-yl]-3-hydroxy-2- phenylbutan-1-one or (2S,3S)-1-[2-(3,4-dihydro-2H- 1,4-benzoxazine-6-sulfonyl)- 2H,4H,5H,6H-pyrrolo[3,4- c]pyrazol-5-yl]-3-hydroxy-2- phenylbutan-1-one or (2S,3R)-1-[2-(3,4-dihydro-2H- 1,4-benzoxazine-6-sulfonyl)- 2H,4H,5H,6H-pyrrolo[3,4- c]pyrazol-5-yl]-3-hydroxy-2- phenylbutan-1-one | 469.2 |
| | 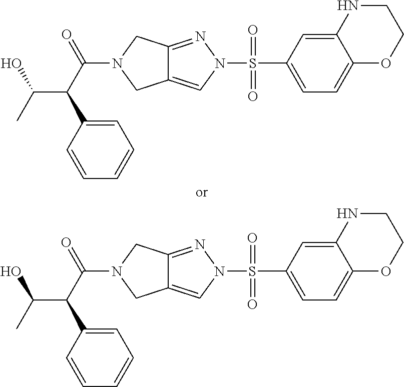 [SMILES: C[C@H](O)[C@@H](C(=O)N1Cc2cn(S(=O)(=O)c3ccc4c(c3)NCCO4)nc2C1)c1ccccc1 ; "or" ; C[C@@H](O)[C@@H](C(=O)N1Cc2cn(S(=O)(=O)c3ccc4c(c3)NCCO4)nc2C1)c1ccccc1] | | |
| 25 | 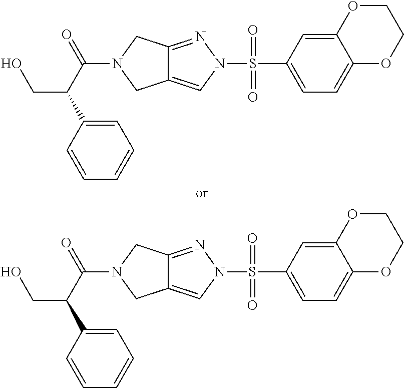 [SMILES: O=C([C@@H](CO)c1ccccc1)N1Cc2cn(S(=O)(=O)c3ccc4c(c3)OCCO4)nc2C1 ; "or" ; O=C([C@H](CO)c1ccccc1)N1Cc2cn(S(=O)(=O)c3ccc4c(c3)OCCO4)nc2C1] | (2R)-1-[2-(2,3-dihydro-1,4- benzodioxine-6-sulfonyl)- 2H,4H,5H,6H-pyrrolo[3,4- c]pyrazol-5-yl]-3-hydroxy-2- phenylpropan-1-one or (2S)-1-[2-(2,3-dihydro-1,4- benzodioxine-6-sulfonyl)- 2H,4H,5H,6H-pyrrolo[3,4- c]pyrazol-5-yl]-3-hydroxy-2- phenylpropan-1-one | 456.1 |
| 26 | 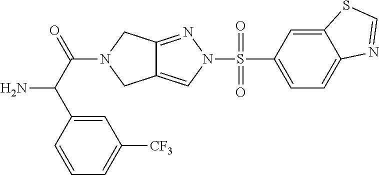 [SMILES: NC(C(=O)N1Cc2cn(S(=O)(=O)c3ccc4ncsc4c3)nc2C1)c1cccc(C(F)(F)F)c1] | 2-amino-1-[2-(1,3- benzothiazole-6-sulfonyl)- 2H,4H,5H,6H-pyrrolo[3,4- c]pyrazol-5-yl]-2-[3- (trifluoromethyl)phenyl]ethan- 1-one (Racemic) | 508.0 |
| 27 | 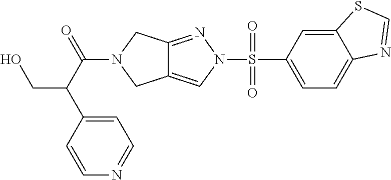 [SMILES: O=C(C(CO)c1ccncc1)N1Cc2cn(S(=O)(=O)c3ccc4ncsc4c3)nc2C1] | 1-[2-(1,3-benzothiazole-6- sulfonyl)-2H,4H,5H,6H- pyrrolo[3,4-c]pyrazol-5-yl]-3- hydroxy-2-(pyridin-4- yl)propan-1-one (Racemic) | 456.0 |
| 28 | 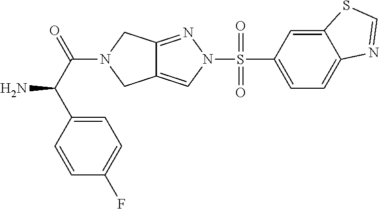 [SMILES: N[C@@H](C(=O)N1Cc2cn(S(=O)(=O)c3ccc4ncsc4c3)nc2C1)c1ccc(F)cc1] | (2R)-2-amino-1-[2-(1,3- benzothiazole-6-sulfonyl)- 2H,4H,5H,6H-pyrrolo[3,4- c]pyrazol-5-yl]-2-(4- fluorophenyl)ethan-1-one | 458.1 |
| 29 | 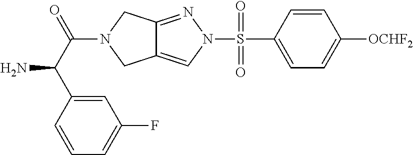 [SMILES: N[C@@H](C(=O)N1Cc2cn(S(=O)(=O)c3ccc(OC(F)F)cc3)nc2C1)c1cccc(F)c1] | (2R)-2-amino-1-{2-[4- (difluoromethoxy)benzenesulfonyl]- 2H,4H,5H,6H-pyrrolo[3,4- c]pyrazol-5-yl}-2-(3- fluorophenyl)ethan-1-one | 467 |
| 30 | 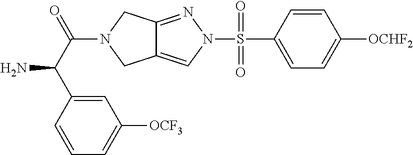 [SMILES: N[C@@H](C(=O)N1Cc2cn(S(=O)(=O)c3ccc(OC(F)F)cc3)nc2C1)c1cccc(OC(F)(F)F)c1] | (2R)-2-amino-1-{2-[4- (difluoromethoxy)benzenesulfonyl]- 2H,4H,5H,6H-pyrrolo[3,4- c]pyrazol-5-yl}-2-[3- (trifluoromethoxy)phenyl]ethan- 1-one | 533.0 |
| 31 | 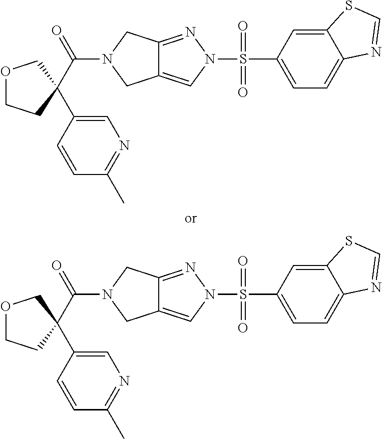 [SMILES: Cc1ccc([C@]2(C(=O)N3Cc4cn(S(=O)(=O)c5ccc6ncsc6c5)nc4C3)CCOC2)cn1 ; "or" ; Cc1ccc([C@@]2(C(=O)N3Cc4cn(S(=O)(=O)c5ccc6ncsc6c5)nc4C3)CCOC2)cn1] | 6-({5-[(3R)-3-(6- methylpyridin-3-yl)oxolane-3- carbonyl]-2H,4H,5H,6H- pyrrolo[3,4-c]pyrazol-2- yl}sulfonyl)-1,3-benzothiazole or 6-({5-[(3S)-3-(6-methylpyridin- 3-yl)oxolane-3-carbonyl]- 2H,4H,5H,6H-pyrrolo[3,4- c]pyrazol-2-yl}sulfonyl)-1,3- benzothiazole | 469.0 |
| 32 | 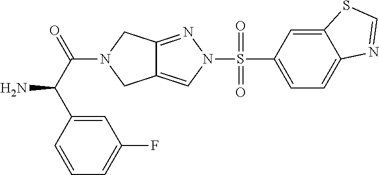 [SMILES: N[C@@H](C(=O)N1Cc2cn(S(=O)(=O)c3ccc4ncsc4c3)nc2C1)c1cccc(F)c1] | (2R)-2-amino-1-[2-(1,3- benzothiazole-6-sulfonyl)- 2H,4H,5H,6H-pyrrolo[3,4- c]pyrazol-5-yl]-2-(3- fluorophenyl)ethan-1-one | 458.6 |
| 33 | 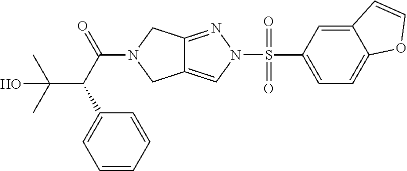 [SMILES: CC(C)(O)[C@H](C(=O)N1Cc2cn(S(=O)(=O)c3ccc4occc4c3)nc2C1)c1ccccc1] | (2R)-1-[2-(1-benzofuran-5- sulfonyl)-2H,4H,5H,6H- pyrrolo[3,4-c]pyrazol-5-yl]-3- hydroxy-3-methyl-2- phenylbutan-1-one | 466.0 |
| 34 | 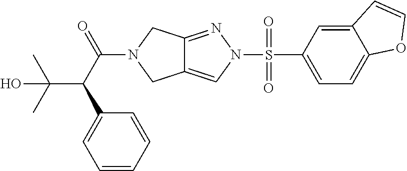 [SMILES: CC(C)(O)[C@@H](C(=O)N1Cc2cn(S(=O)(=O)c3ccc4occc4c3)nc2C1)c1ccccc1] | (2S)-1-[2-(1-benzofuran-5- sulfonyl)-2H,4H,5H,6H- pyrrolo[3,4-c]pyrazol-5-yl]-3- hydroxy-3-methyl-2- phenylbutan-1-one | 466.0 |
| 35 | 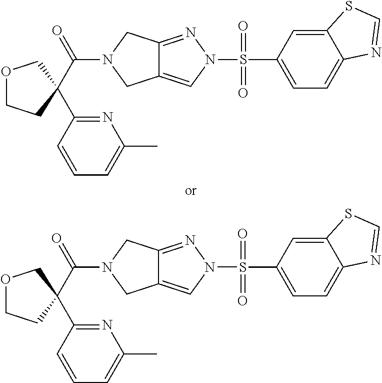 [SMILES: Cc1cccc([C@]2(C(=O)N3Cc4cn(S(=O)(=O)c5ccc6ncsc6c5)nc4C3)CCOC2)n1 ; "or" ; Cc1cccc([C@@]2(C(=O)N3Cc4cn(S(=O)(=O)c5ccc6ncsc6c5)nc4C3)CCOC2)n1] | 6-({5-[(3R)-3-(6- methylpyridin-2-yl)oxolane-3- carbonyl]-2H,4H,5H,6H- pyrrolo[3,4-c]pyrazol-2- yl}sulfonyl)-1,3-benzothiazole or 6-({5-[(3S)-3-(6-methylpyridin- 2-yl)oxolane-3-carbonyl]- 2H,4H,5H,6H-pyrrolo[3,4- c]pyrazol-2-yl}sulfonyl)-1,3- benzothiazole | 496.0 |
| 36 | 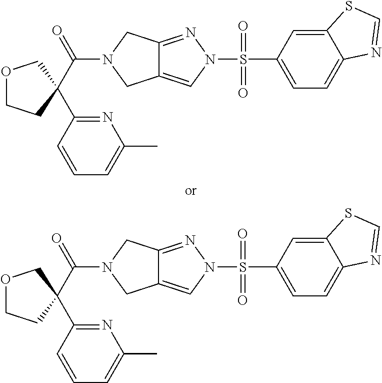 [SMILES: Cc1cccc([C@]2(C(=O)N3Cc4cn(S(=O)(=O)c5ccc6ncsc6c5)nc4C3)CCOC2)n1 ; "or" ; Cc1cccc([C@@]2(C(=O)N3Cc4cn(S(=O)(=O)c5ccc6ncsc6c5)nc4C3)CCOC2)n1] | 6-({5-[(3R)-3-(6- methylpyridin-2-yl)oxolane-3- carbonyl]-2H,4H,5H,6H- pyrrolo[3,4-c]pyrazol-2- yl}sulfonyl)-1,3-benzothiazole or 6-({5-[(3S)-3-(6-methylpyridin- 2-yl)oxolane-3-carbonyl]- 2H,4H,5H,6H-pyrrolo[3,4- c]pyrazol-2-yl}sulfonyl)-1,3- benzothiazole | 496.0 |
| 37 | 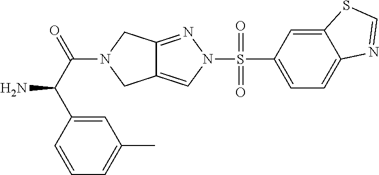 [SMILES: Cc1cccc([C@@H](N)C(=O)N2Cc3cn(S(=O)(=O)c4ccc5ncsc5c4)nc3C2)c1] | (2R)-2-amino-1-[2-(1,3- benzothiazole-6-sulfonyl)- 2H,4H,5H,6H-pyrrolo[3,4- c]pyrazol-5-yl]-2-(3- methylphenyl)ethan-1-one | 454.0 |
| 38 | 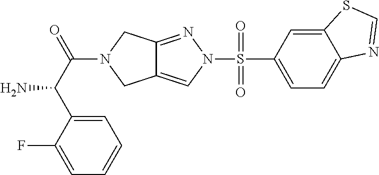 [SMILES: N[C@H](C(=O)N1Cc2cn(S(=O)(=O)c3ccc4ncsc4c3)nc2C1)c1ccccc1F] | (2S)-2-amino-1-[2-(1,3- benzothiazole-6-sulfonyl)- 2H,4H,5H,6H-pyrrolo[3,4- c]pyrazol-5-yl]-2-(2- fluorophenyl)ethan-1-one | 458.1 |
| 39 | 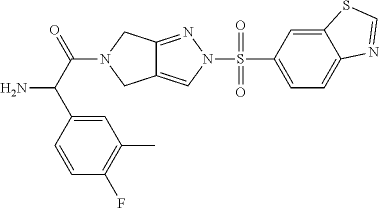 [SMILES: Cc1cc(C(N)C(=O)N2Cc3cn(S(=O)(=O)c4ccc5ncsc5c4)nc3C2)ccc1F] | 2-amino-1-[2-(1,3- benzothiazole-6-sulfonyl)- 2H,4H,5H,6H-pyrrolo[3,4- c]pyrazol-5-yl]-2-(4-fluoro-3- methylphenyl)ethan-1-one (Racemic) | 472.2 |
| 40 | 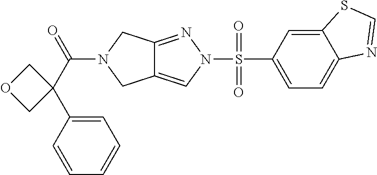 [SMILES: O=C(N1Cc2cn(S(=O)(=O)c3ccc4ncsc4c3)nc2C1)C1(c2ccccc2)COC1] | 6-{[5-(3-phenyloxetane-3- carbonyl)-2H,4H,5H,6H- pyrrolo[3,4-c]pyrazol-2- yl]sulfonyl}-1,3-benzothiazole | 467.2 |
| 42 | 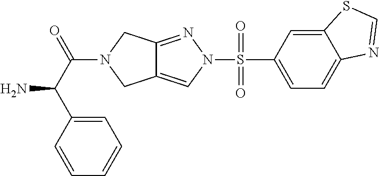 [SMILES: N[C@@H](C(=O)N1Cc2cn(S(=O)(=O)c3ccc4ncsc4c3)nc2C1)c1ccccc1] | (2R)-1-[2-(1,3-benzothiazole-6- sulfonyl)-2H,4H,5H,6H- pyrrolo[3,4-c]pyrazol-5-yl]-2- hydroxy-2-phenylethan-1-one | 441.2 |
| 43 | 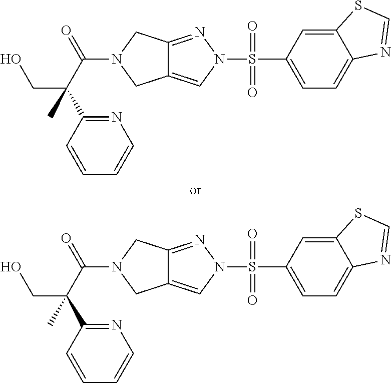 [SMILES: C[C@@](CO)(C(=O)N1Cc2cn(S(=O)(=O)c3ccc4ncsc4c3)nc2C1)c1ccccn1 ; "or" ; C[C@](CO)(C(=O)N1Cc2cn(S(=O)(=O)c3ccc4ncsc4c3)nc2C1)c1ccccn1] | (2R)-1-[2-(1,3-benzothiazole-6- sulfonyl)-2H,4H,5H,6H- pyrrolo[3,4-c]pyrazol-5-yl]-3- hydroxy-2-methyl-2-(pyridin-2- yl)propan-1-one or (2S)-1-2-(1,3- benzothiazole-6-sulfonyl)- 2H,4H,5H,6H-pyrrolo[3,4- c]pyrazol-5-yl]-3-hydroxy-2- methyl-2-(pyridin-2- yl)propan-1-one | 470.1 |
| 44 | 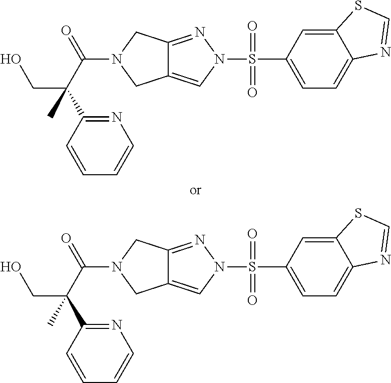 [SMILES: C[C@@](CO)(C(=O)N1Cc2cn(S(=O)(=O)c3ccc4ncsc4c3)nc2C1)c1ccccn1 ; "or" ; C[C@](CO)(C(=O)N1Cc2cn(S(=O)(=O)c3ccc4ncsc4c3)nc2C1)c1ccccn1] | (2R)-1-[2-(1,3-benzothiazole-6- sulfonyl)-2H,4H,5H,6H- pyrrolo[3,4-c]pyrazol-5-yl]-3- hydroxy-2-methyl-2-(pyridin-2- yl)propan-1-one or (2S)-1-[2-(1,3- benzothiazole-6-sulfonyl)- 2H,4H,5H,6H-pyrrolo[3,4- c]pyrazol-5-yl]-3-hydroxy-2- methyl-2-(pyridin-2- yl)propan-1-one | 470.1 |
| 45 | 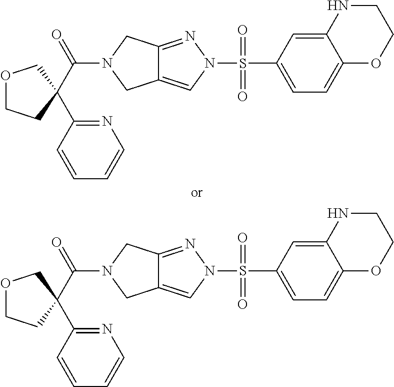 [SMILES: O=C(N1Cc2cn(S(=O)(=O)c3ccc4c(c3)NCCO4)nc2C1)[C@@]1(c2ccccn2)CCOC1 ; "or" ; O=C(N1Cc2cn(S(=O)(=O)c3ccc4c(c3)NCCO4)nc2C1)[C@]1(c2ccccn2)CCOC1] | 6-({5-[(3R)-3-(pyridin-2- yl)oxolane-3-carbonyl]- 2H,4H,5H,6H-pyrrolo[3,4- c]pyrazol-2-yl}sulfonyl)-3,4- dihydro-2H-1,4-benzoxazine or 6-({5-[(3S)-3-(pyridin-2- yl)oxolane-3-carbonyl]- 2H,4H,5H,6H-pyrrolo[3,4- c]pyrazol-2-yl}sulfonyl)-3,4- dihydro-2H-1,4-benzoxazine | 482 |
| 46 | 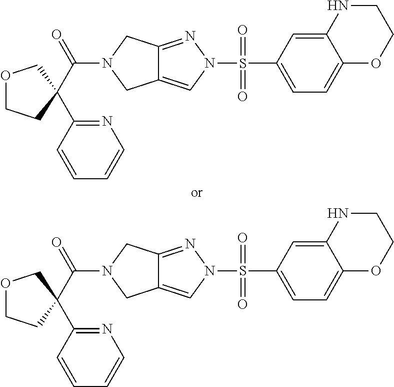 [SMILES: O=C(N1Cc2cn(S(=O)(=O)c3ccc4c(c3)NCCO4)nc2C1)[C@@]1(c2ccccn2)CCOC1 ; "or" ; O=C(N1Cc2cn(S(=O)(=O)c3ccc4c(c3)NCCO4)nc2C1)[C@]1(c2ccccn2)CCOC1] | 6-({5-[(3R)-3-(pyridin-2- yl)oxolane-3-carbonyl]- 2H,4H,5H,6H-pyrrolo[3,4- c]pyrazol-2-yl}sulfonyl)-3,4- dihydro-2H-1,4-benzoxazine or 6-({5-[(3S)-3-(pyridin-2- yl)oxolane-3-carbonyl]- 2H,4H,5H,6H-pyrrolo[3,4- c]pyrazol-2-yl}sulfonyl)-3,4- dihydro-2H-1,4-benzoxazine | 482 |
| 47 | 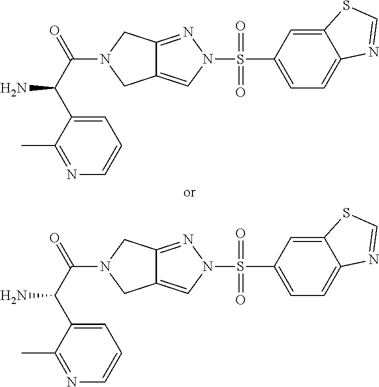 [SMILES: Cc1ncccc1[C@@H](N)C(=O)N1Cc2cn(S(=O)(=O)c3ccc4ncsc4c3)nc2C1 ; "or" ; Cc1ncccc1[C@H](N)C(=O)N1Cc2cn(S(=O)(=O)c3ccc4ncsc4c3)nc2C1] | (2R)-2-amino-1-[2-(1,3- benzothiazole-6-sulfonyl)- 2H,4H,5H,6H-pyrrolo[3,4- c]pyrazol-5-yl]-2-(2- methylpyridin-3-yl)ethan-1-one or (2S)-2-amino-1-[2-(1,3- benzothiazole-6-sulfonyl)- 2H,4H,5H,6H-pyrrolo[3,4- c]pyrazol-5-yl]-2-(2- methylpyridin-3-yl)ethan-1-one | 455 |
| 48 | 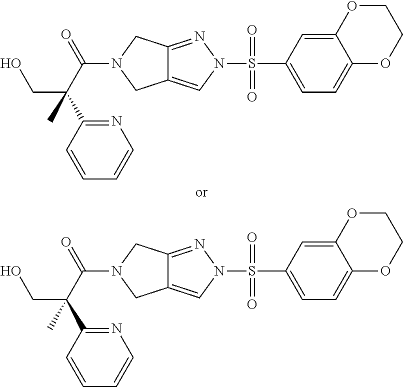 [SMILES: C[C@@](CO)(C(=O)N1Cc2cn(S(=O)(=O)c3ccc4c(c3)OCCO4)nc2C1)c1ccccn1 ; "or" ; C[C@](CO)(C(=O)N1Cc2cn(S(=O)(=O)c3ccc4c(c3)OCCO4)nc2C1)c1ccccn1] | (2R)-1-[2-(2,3-dihydro-1,4- benzodioxine-6-sulfonyl)- 2H,4H,5H,6H-pyrrolo[3,4- c]pyrazol-5-yl]-3-hydroxy-2- methyl-2-(pyridin-2-yl)propan- 1-one or (2S)-1-[2-(2,3-dihydro-1,4- benzodioxine-6-sulfonyl)- 2H,4H,5H,6H-pyrrolo[3,4- c]pyrazol-5-yl]-3-hydroxy-2- methyl-2-(pyridin-2-yl)propan- 1-one | 471.0 |
| 49 | 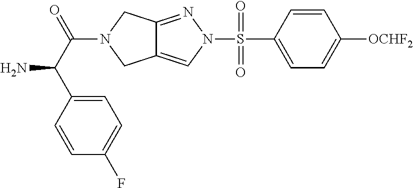 [SMILES: N[C@@H](C(=O)N1Cc2cn(S(=O)(=O)c3ccc(OC(F)F)cc3)nc2C1)c1ccc(F)cc1] | (2R)-2-amino-1-{2-[4- (difluoromethoxy)benzenesulfonyl]- 2H,4H,5H,6H-pyrrolo[3,4- c]pyrazol-5-yl}-2-(4- fluorophenyl)ethan-1-one | 467.1 |
| 50 | 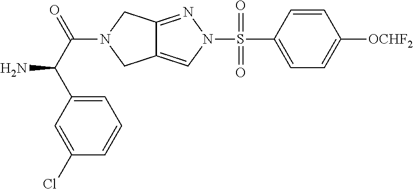 [SMILES: N[C@@H](C(=O)N1Cc2cn(S(=O)(=O)c3ccc(OC(F)F)cc3)nc2C1)c1cccc(Cl)c1] | (2R)-2-amino-2-(3- chlorophenyl)-1-{2-[4- (difluoromethoxy)benzenesulfonyl]- 2H,4H,5H,6H-pyrrolo[3,4- c]pyrazol-5-yl}ethan-1-one | 483 |
| 51 | 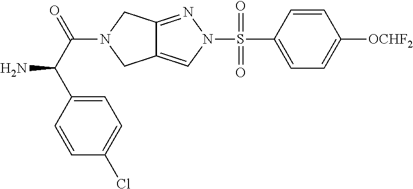 [SMILES: N[C@@H](C(=O)N1Cc2cn(S(=O)(=O)c3ccc(OC(F)F)cc3)nc2C1)c1ccc(Cl)cc1] | (2R)-2-amino-2-(4- chlorophenyl)-1-{2-[4- (difluoromethoxy)benzenesulfonyl]- 2H,4H,5H,6H-pyrrolo[3,4- c]pyrazol-5-yl}ethan-1-one | 483.0 |
| 52 | 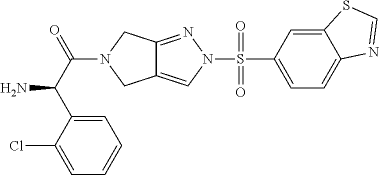 [SMILES: N[C@@H](C(=O)N1Cc2cn(S(=O)(=O)c3ccc4ncsc4c3)nc2C1)c1ccccc1Cl] | (2R)-2-amino-1-[2-(1,3- benzothiazole-6-sulfonyl)- 2H,4H,5H,6H-pyrrolo[3,4- c]pyrazol-5-yl]-2-(2- chlorophenyl)ethan-1-one | 474.1 |
| 53 | 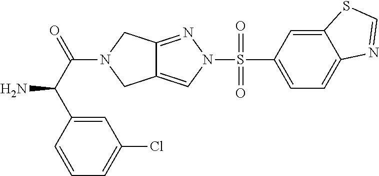 [SMILES: N[C@@H](C(=O)N1Cc2cn(S(=O)(=O)c3ccc4ncsc4c3)nc2C1)c1cccc(Cl)c1] | (2R)-2-amino-1-[2-(1,3- benzothiazole-6-sulfonyl)- 2H,4H,5H,6H-pyrrolo[3,4- c]pyrazol-5-yl]-2-(3- chlorophenyl)ethan-1-one | 474.6 |
| 54 | 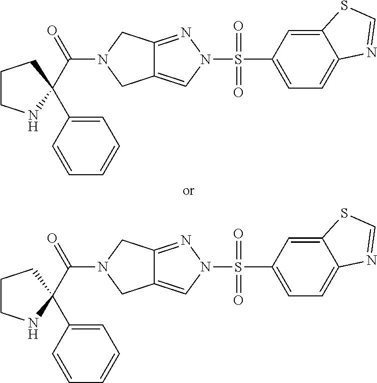 [SMILES: O=C(N1Cc2cn(S(=O)(=O)c3ccc4ncsc4c3)nc2C1)[C@@]1(c2ccccc2)CCCN1 ; "or" ; O=C(N1Cc2cn(S(=O)(=O)c3ccc4ncsc4c3)nc2C1)[C@]1(c2ccccc2)CCCN1] | 6-({5-[(2S)-2- phenylpyrrolidine-2-carbonyl]- 2H,4H,5H,6H-pyrrolo[3,4- c]pyrazol-2-yl}sulfonyl)-1,3- benzothiazole or 6-({5-[(2R)-2- phenylpyrrolidine-2-carbonyl]- 2H,4H,5H,6H-pyrrolo[3,4- c]pyrazol-2-yl}sulfonyl)-1,3- benzothiazole | 480.0 |
| 55 | 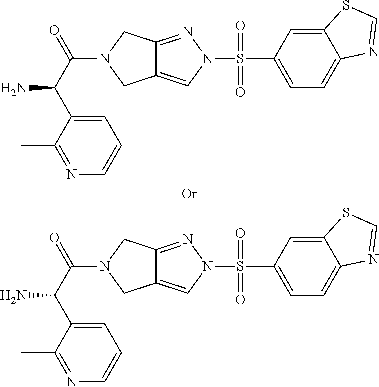 | (2R)-2-amino-1-[2-(1,3- benzothiazole-6-sulfonyl)- 2H,4H,5H,6H-pyrrolo[3,4- c]pyrazol-5-yl]-2-(2- methylpyridin-3-yl)ethan-1-one or (2S)-2-amino-1-[2-(1,3- benzothiazole-6-sulfonyl)- 2H,4H,5H,6H-pyrrolo[3,4- c]pyrazol-5-yl]-2-(2- methylpyridin-3-yl)ethan-1-one | 455.0 |
| 56 | 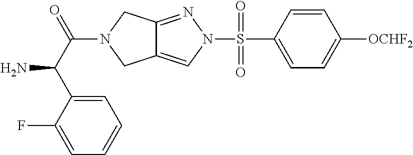 [SMILES: N[C@@H](C(=O)N1Cc2cn(S(=O)(=O)c3ccc(OC(F)F)cc3)nc2C1)c1ccccc1F] | (2R)-2-amino-1-{2-[4- (difluoromethoxy)benzenesulfonyl]- 2H,4H,5H,6H-pyrrolo[3,4- c]pyrazol-5-yl}-2-(2- fluorophenyl)ethan-1-one | 467.1 |
| 57 | 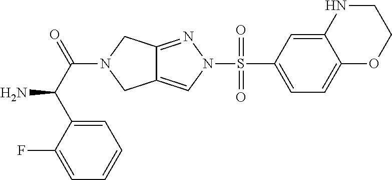 [SMILES: N[C@@H](C(=O)N1Cc2cn(S(=O)(=O)c3ccc4c(c3)NCCO4)nc2C1)c1ccccc1F] | (2R)-2-amino-1-[2-(3,4- dihydro-2H-1,4-benzoxazine-6- sulfonyl)-2H,4H,5H,6H- pyrrolo[3,4-c]pyrazol-5-yl]-2- (2-fluorophenyl)ethan-1-one | 458.2 |
| 58 | 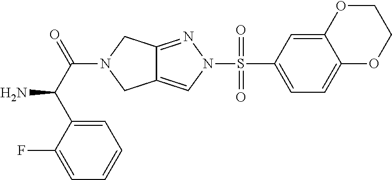 [SMILES: N[C@@H](C(=O)N1Cc2cn(S(=O)(=O)c3ccc4c(c3)OCCO4)nc2C1)c1ccccc1F] | (2R)-2-amino-1-[2-(2,3- dihydro-1,4-benzodioxine-6- sulfonyl)-2H,4H,5H,6H- pyrrolo[3,4-c]pyrazol-5-yl]-2- (2-fluorophenyl)ethan-1-one | 459.1 |
| 59 | 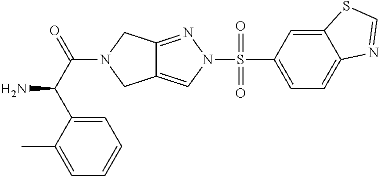 [SMILES: Cc1ccccc1[C@@H](N)C(=O)N1Cc2cn(S(=O)(=O)c3ccc4ncsc4c3)nc2C1] | (2R)-2-amino-1-[2-(1,3- benzothiazole-6-sulfonyl)- 2H,4H,5H,6H-pyrrolo[3,4- c]pyrazol-5-yl]-2-(2- methylphenyl)ethan-1-one | 454.0 |
| 60 | 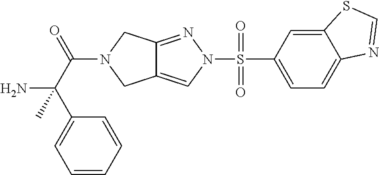 [SMILES: C[C@](N)(C(=O)N1Cc2cn(S(=O)(=O)c3ccc4ncsc4c3)nc2C1)c1ccccc1] | (2R)-2-amino-1-[2-(1,3- benzothiazole-6-sulfonyl)- 2H,4H,5H,6H-pyrrolo[3,4- c]pyrazol-5-yl]-2- phenylpropan-1-one | 454.0 |
| 61 | 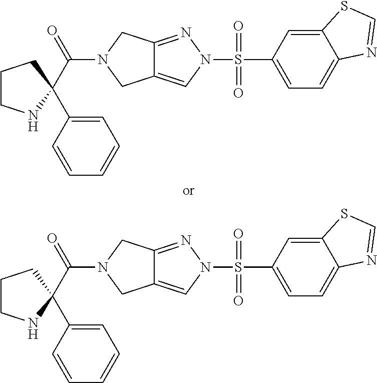 [SMILES: O=C(N1Cc2cn(S(=O)(=O)c3ccc4ncsc4c3)nc2C1)[C@@]1(c2ccccc2)CCCN1 ; "or" ; O=C(N1Cc2cn(S(=O)(=O)c3ccc4ncsc4c3)nc2C1)[C@]1(c2ccccc2)CCCN1] | 6-({5-[(2S)-2- phenylpyrrolidine-2-carbonyl]- 2H,4H,5H,6H-pyrrolo[3,4- c]pyrazol-2-yl}sulfonyl)-1,3- benzothiazole or 6-({5-[(2R)-2- phenylpyrrolidine-2-carbonyl]- 2H,4H,5H,6H-pyrrolo[3,4- c]pyrazol-2-yl}sulfonyl)-1,3- benzothiazole | 480.0 |
| 62 | 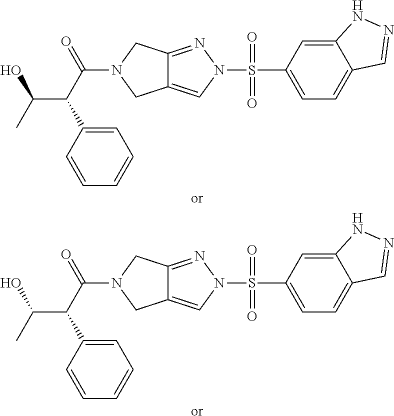 [SMILES: C[C@@H](O)[C@H](C(=O)N1Cc2cn(S(=O)(=O)c3ccc4cn[nH]c4c3)nc2C1)c1ccccc1 ; "or" ; C[C@H](O)[C@H](C(=O)N1Cc2cn(S(=O)(=O)c3ccc4cn[nH]c4c3)nc2C1)c1ccccc1 ; "or"] | (2R,3R)-3-hydroxy-1-[2-(1H- indazole-6-sulfonyl)- 2H,4H,5H,6H-pyrrolo[3,4- c]pyrazol-5-yl]-2-phenylbutan- 1-one or (2R,3S)-3-hydroxy-1-[2-(1H- indazole-6-sulfonyl)- 2H,4H,5H,6H-pyrrolo[3,4- c]pyrazol-5-yl]-2-phenylbutan- 1-one or (2S,3S)-3-hydroxy-1-[2-(1H- indazole-6-sulfonyl)- 2H,4H,5H,6H-pyrrolo[3,4- c]pyrazol-5-yl]-2-phenylbutan- 1-one or (2S,3R)-3-hydroxy-1-[2-(1H- indazole-6-sulfonyl)- 2H,4H,5H,6H-pyrrolo[3,4- c]pyrazol-5-yl]-2-phenylbutan- 1-one | 451.9 |
| | 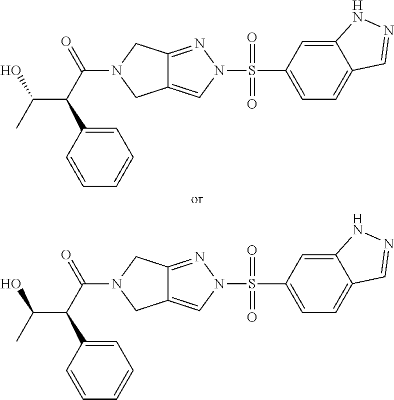 [SMILES: C[C@H](O)[C@@H](C(=O)N1Cc2cn(S(=O)(=O)c3ccc4cn[nH]c4c3)nc2C1)c1ccccc1 ; "or" ; C[C@@H](O)[C@@H](C(=O)N1Cc2cn(S(=O)(=O)c3ccc4cn[nH]c4c3)nc2C1)c1ccccc1] | | |
| 63 | 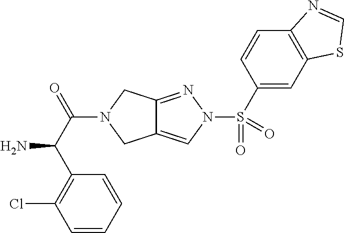 [SMILES: N[C@@H](C(=O)N1Cc2cn(S(=O)(=O)c3ccc4ncsc4c3)nc2C1)c1ccccc1Cl] | (2R)-2-amino-1-[2-(1,3- benzothiazole-6-sulfonyl)- 2H,4H,5H,6H-pyrrolo[3,4- c]pyrazol-5-yl]-2-(2- chlorophenyl)ethan-1-one | |
| 64 | 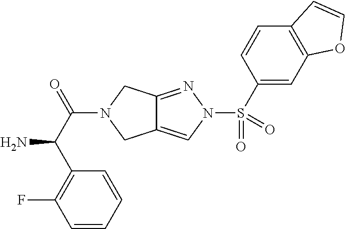 [SMILES: N[C@@H](C(=O)N1Cc2cn(S(=O)(=O)c3ccc4ccoc4c3)nc2C1)c1ccccc1F] | (2R)-2-amino-1-[2-(1- benzofuran-5-sulfonyl)- 2H,4H,5H,6H-pyrrolo[3,4- c]pyrazol-5-yl]-2-(2- fluorophenyl)ethan-1-one | 441 |
| 65 | 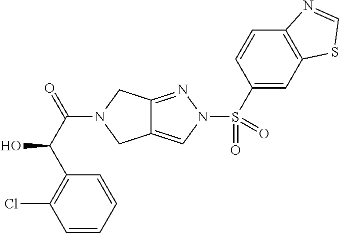 [SMILES: O=C([C@H](O)c1ccccc1Cl)N1Cc2cn(S(=O)(=O)c3ccc4ncsc4c3)nc2C1] | (2R)-1-[2-(1,3-benzothiazole-6- sulfonyl)-2H,4H,5H,6H- pyrrolo[3,4-c]pyrazol-5-yl]-2- (2-chlorophenyl)-2- hydroxyethan-1-one | 475.1 |
| 66 | 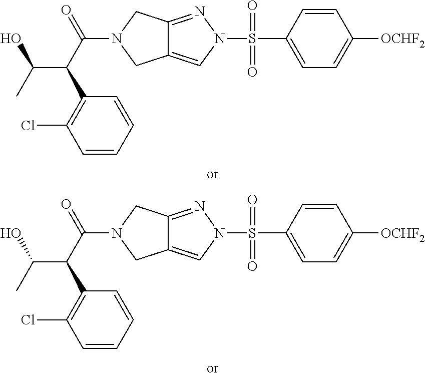 [SMILES: C[C@@H](O)[C@@H](C(=O)N1Cc2cn(S(=O)(=O)c3ccc(OC(F)F)cc3)nc2C1)c1ccccc1Cl ; C[C@H](O)[C@@H](C(=O)N1Cc2cn(S(=O)(=O)c3ccc(OC(F)F)cc3)nc2C1)c1ccccc1Cl] | (2S,3R)-2-(2-chlorophenyl)- 1-{2-[4- (difluoromethoxy)benzenesulfonyl]- 2H,4H,5H,6H- pyrrolo[3,4-c]pyrazol-5-yl}- 3-hydroxybutan-1-one or (2S,3S)-2-(2-chlorophenyl)- 1-{2-[4- (difluoromethoxy)benzenesulfonyl]- 2H,4H,5H,6H- pyrrolo[3,4-c]pyrazol-5-yl}- 3-hydroxybutan-1-one or (2R,3S)-2-(2-chlorophenyl)- 1-{2-[4- (difluoromethoxy)benzenesulfonyl]- 2H,4H,5H,6H- pyrrolo[3,4-c]pyrazol-5-yl}- 3-hydroxybutan-1-one or (2R,3R)-2-(2-chlorophenyl)- 1-{2-[4- (difluoromethoxy)benzenesulfonyl]- 2H,4H,5H,6H- pyrrolo[3,4-c]pyrazol-5-yl}- 3-hydroxybutan-1-one | 512 |
| | 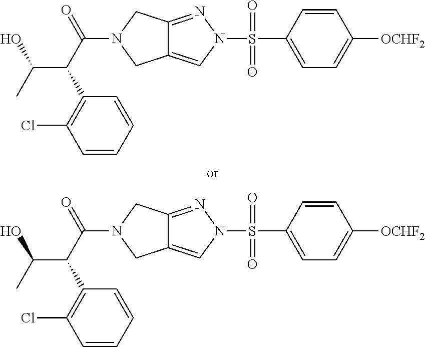 [SMILES: C[C@H](O)[C@H](C(=O)N1Cc2cn(S(=O)(=O)c3ccc(OC(F)F)cc3)nc2C1)c1ccccc1Cl ; C[C@@H](O)[C@H](C(=O)N1Cc2cn(S(=O)(=O)c3ccc(OC(F)F)cc3)nc2C1)c1ccccc1Cl] | | |
| 67 | 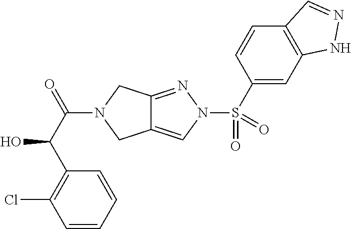 [SMILES: O=C([C@H](O)c1ccccc1Cl)N1Cc2cn(S(=O)(=O)c3ccc4cn[nH]c4c3)nc2C1] | (2R)-2-(2-chlorophenyl)-2- hydroxy-1-[2-(1H-indazole-6- sulfonyl)-2H,4H,5H,6H- pyrrolo[3,4-c]pyrazol-5- yl]ethan-1-one | 458 |
| 68 | 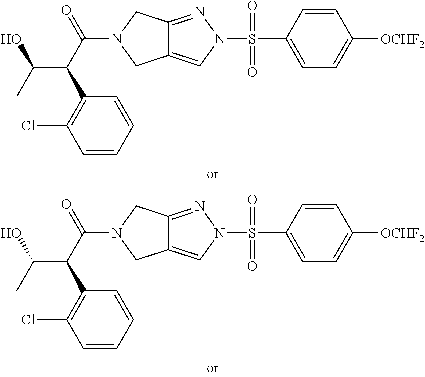 [SMILES: C[C@@H](O)[C@@H](C(=O)N1Cc2cn(S(=O)(=O)c3ccc(OC(F)F)cc3)nc2C1)c1ccccc1Cl ; C[C@H](O)[C@@H](C(=O)N1Cc2cn(S(=O)(=O)c3ccc(OC(F)F)cc3)nc2C1)c1ccccc1Cl] | (2S,3R)-2-(2-chlorophenyl)- 1-{2-[4- (difluoromethoxy)benzenesulfonyl]- 2H,4H,5H,6H- pyrrolo[3,4-c]pyrazol-5-yl}- 3-hydroxybutan-1-one or (2S,3S)-2-(2-chlorophenyl)- 1-{2-[4- (difluoromethoxy)benzenesulfonyl]- 2H,4H,5H,6H- pyrrolo[3,4-c]pyrazol-5-yl}- 3-hydroxybutan-1-one or (2R,3S)-2-(2-chlorophenyl)- 1-{2-[4- (difluoromethoxy)benzenesulfonyl]- 2H,4H,5H,6H- pyrrolo[3,4-c]pyrazol-5-yl}- 3-hydroxybutan-1-one or (2R,3R)-2-(2-chlorophenyl)- 1-{2-[4- (difluoromethoxy)benzenesulfonyl]- 2H,4H,5H,6H- pyrrolo[3,4-c]pyrazol-5-yl}- 3-hydroxybutan-1-one | 512 |
| | 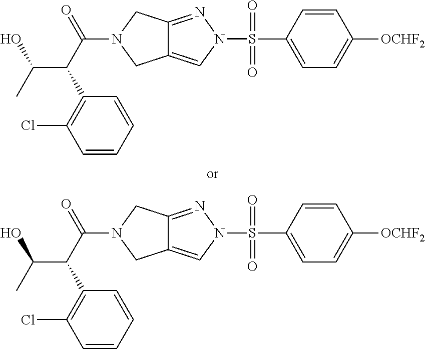 [SMILES: C[C@H](O)[C@H](C(=O)N1Cc2cn(S(=O)(=O)c3ccc(OC(F)F)cc3)nc2C1)c1ccccc1Cl ; C[C@@H](O)[C@H](C(=O)N1Cc2cn(S(=O)(=O)c3ccc(OC(F)F)cc3)nc2C1)c1ccccc1Cl] | | |
| 69 | 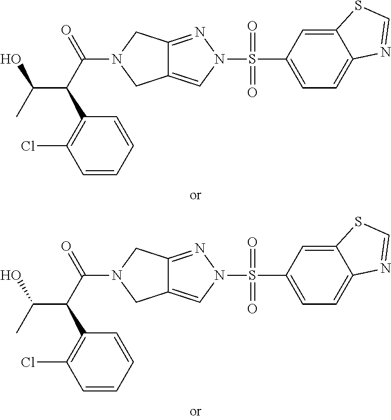 [SMILES: C[C@@H](O)[C@@H](C(=O)N1Cc2cn(S(=O)(=O)c3ccc4ncsc4c3)nc2C1)c1ccccc1Cl ; C[C@H](O)[C@@H](C(=O)N1Cc2cn(S(=O)(=O)c3ccc4ncsc4c3)nc2C1)c1ccccc1Cl] | (2S,3S)-2-(2-chlorophenyl)- 1-{2-[4- (difluoromethoxy)benzenesulfonyl]- 2H,4H,5H,6H- pyrrolo[3,4-c]pyrazol-5-yl}- 3-hydroxybutan-1-one or (2S,3R)-2-(2-chlorophenyl)- 1-{2-[4- (difluoromethoxy)benzenesulfonyl]- 2H,4H,5H,6H- pyrrolo[3,4-c]pyrazol-5-yl}- 3-hydroxybutan-1-one or (2R,3R)-2-(2-chlorophenyl)- 1-{2-[4- (difluoromethoxy)benzenesulfonyl]- 2H,4H,5H,6H- pyrrolo[3,4-c]pyrazol-5-yl}- 3-hydroxybutan-1-one or (2R,3S)-2-(2-chlorophenyl)- 1-{2-[4- (difluoromethoxy)benzenesulfonyl]- 2H,4H,5H,6H- pyrrolo[3,4-c]pyrazol-5-yl}- 3-hydroxybutan-1-one | 503 |
| | 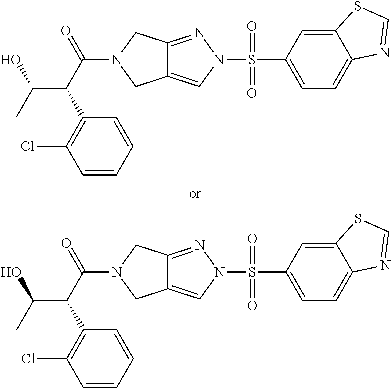 [SMILES: C[C@H](O)[C@H](C(=O)N1Cc2cn(S(=O)(=O)c3ccc4ncsc4c3)nc2C1)c1ccccc1Cl ; C[C@@H](O)[C@H](C(=O)N1Cc2cn(S(=O)(=O)c3ccc4ncsc4c3)nc2C1)c1ccccc1Cl] | | |
| 70 | 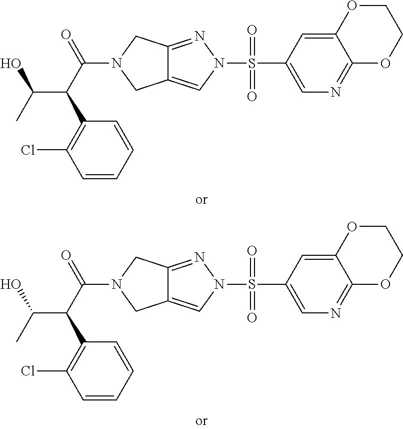 [SMILES: C[C@@H](O)[C@@H](C(=O)N1Cc2cn(S(=O)(=O)c3cnc4c(c3)OCCO4)nc2C1)c1ccccc1Cl ; C[C@H](O)[C@@H](C(=O)N1Cc2cn(S(=O)(=O)c3cnc4c(c3)OCCO4)nc2C1)c1ccccc1Cl] | (2S,3R)-2-(2-chlorophenyl)- 1-(2-[2H,3H- [1,4]dioxino[2,3-b]pyridine- 7-sulfonyl]-4H,6H- pyrrolo[3,4-c]pyrazol-5-yl)- 3-hydroxybutan-1-one or (2R,3R)-2-(2-chlorophenyl)- 1-(2-[2H,3H- [1,4]dioxino[2,3-b]pyridine- 7-sulfonyl]-4H,6H- pyrrolo[3,4-c]pyrazol-5-yl)- 3-hydroxybutan-1-one or (2R,3S)-2-(2-chlorophenyl)- 1-(2-[2H,3H- [1,4]dioxino[2,3-b]pyridine- 7-sulfonyl]-4H,6H- pyrrolo[3,4-c]pyrazol-5-yl)- 3-hydroxybutan-1-one or (2S,3S)-2-(2-chlorophenyl)- 1-(2-[2H,3H- [1,4]dioxino[2,3-b]pyridine- 7-sulfonyl]-4H,6H- pyrrolo[3,4-c]pyrazol-5-yl)- 3-hydroxybutan-1-one | 505 |
| | 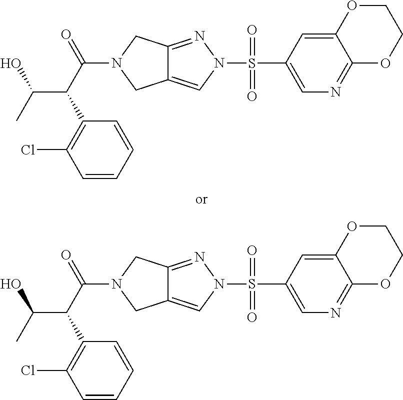 [SMILES: C[C@H](O)[C@H](C(=O)N1Cc2cn(S(=O)(=O)c3cnc4c(c3)OCCO4)nc2C1)c1ccccc1Cl ; C[C@@H](O)[C@H](C(=O)N1Cc2cn(S(=O)(=O)c3cnc4c(c3)OCCO4)nc2C1)c1ccccc1Cl] | | |
| 71 | 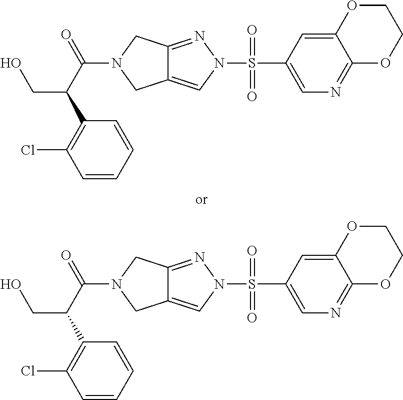 [SMILES: O=C([C@H](CO)c1ccccc1Cl)N1Cc2cn(S(=O)(=O)c3cnc4c(c3)OCCO4)nc2C1 ; O=C([C@@H](CO)c1ccccc1Cl)N1Cc2cn(S(=O)(=O)c3cnc4c(c3)OCCO4)nc2C1] | (2S,3R)-2-(2-chlorophenyl)- 1-(2-[2H,3H- [1,4]dioxino[2,3-b]pyridine- 7-sulfonyl]-4H,6H- pyrrolo[3,4-c]pyrazol-5-yl)- 3-hydroxybutan-1-one or (2R,3R)-2-(2-chlorophenyl)- 1-(2-[2H,3H- [1,4]dioxino[2,3-b]pyridine- 7-sulfonyl]-4H,6H- pyrrolo[3,4-c]pyrazol-5-yl)- 3-hydroxybutan-1-one | 491 |
| 72 | 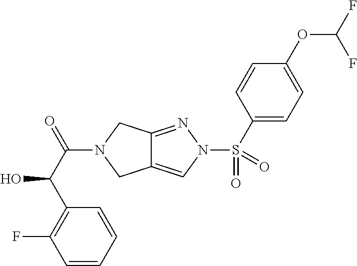 [SMILES: O=C([C@H](O)c1ccccc1F)N1Cc2cn(S(=O)(=O)c3ccc(OC(F)F)cc3)nc2C1] | (2R)-1-{2-[4- (difluoromethoxy)benzenesulfonyl]- 2H,4H,5H,6H-pyrrolo[3,4- c]pyrazol-5-yl}-2-(2- fluorophenyl)-2-hydroxyethan- 1-one | 468 |
| 73 | 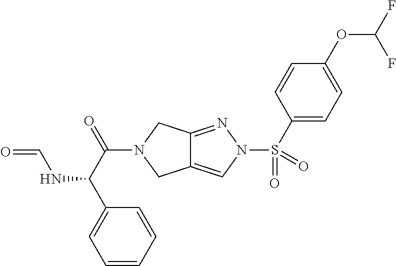 [SMILES: O=CN[C@H](C(=O)N1Cc2cn(S(=O)(=O)c3ccc(OC(F)F)cc3)nc2C1)c1ccccc1] | N-[(1S)-2-{2-[4- (difluoromethoxy)benzenesulfonyl]- 2H,4H,5H,6H-pyrrolo[3,4- c]pyrazol-5-yl}-2-oxo-1- phenylethyl]formamide | 476.99 |
| 74 | 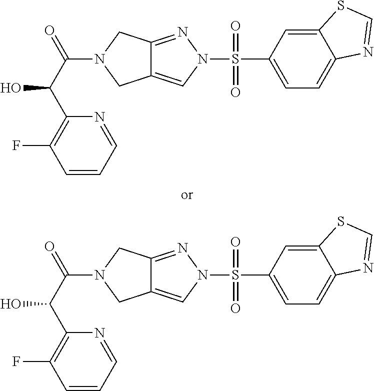 [SMILES: O=C([C@H](O)c1ncccc1F)N1Cc2cn(S(=O)(=O)c3ccc4ncsc4c3)nc2C1 ; O=C([C@@H](O)c1ncccc1F)N1Cc2cn(S(=O)(=O)c3ccc4ncsc4c3)nc2C1] | (2R)-1-2-(1,3- benzothiazole-6-sulfonyl)- 2H,4H,5H,6H-pyrrolo[3,4- c]pyrazol-5-yl]-2-(3- fluoropyridin-2-yl)-2- hydroxyethan-1-one or (2S)-1-2-(1,3- benzothiazole-6-sulfonyl)- 2H,4H,5H,6H-pyrrolo[3,4- c]pyrazol-5-yl]-2-(3- fluoropyridin-2-yl)-2- hydroxyethan-1-one | 460 |
| 75 | 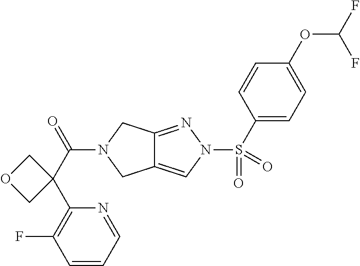 [SMILES: O=C(N1Cc2cn(S(=O)(=O)c3ccc(OC(F)F)cc3)nc2C1)C1(c2ncccc2F)COC1] | 3-chloro-2-(3-{2-[4- (difluoromethoxy)benzenesulfonyl]- 2H,4H,5H,6H-pyrrolo[3,4- c]pyrazole-5-carbonyl}oxetan- 3-yl)pyridine | 511 |
| 76 | 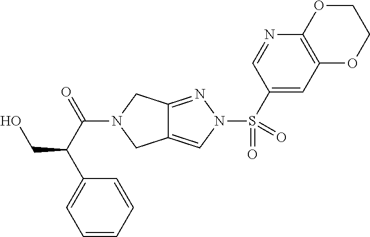 [SMILES: O=C([C@H](CO)c1ccccc1)N1Cc2cn(S(=O)(=O)c3cnc4c(c3)OCCO4)nc2C1] | (2S)-1-(2-{2H,3H- [1,4]dioxino[2,3-b]pyridine-7- sulfonyl}-2H,4H,5H,6H- pyrrolo[3,4-c]pyrazol-5-yl)-3- hydroxy-2-phenylpropan-1-one | 457 |
| 77 | 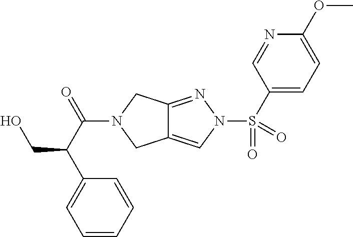 [SMILES: COc1ccc(S(=O)(=O)n2cc3c(n2)CN(C(=O)[C@H](CO)c2ccccc2)C3)cn1] | (2S)-3-hydroxy-1-{2-[(6- methoxypyridin-3-yl)sulfonyl]- 2H,4H,5H,6H-pyrrolo[3,4- c]pyrazol-5-yl}-2- phenylpropan-1-one | 429.8 |
| 78 | 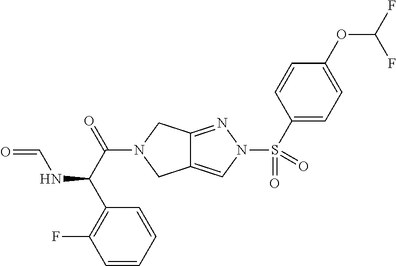 [SMILES: O=CN[C@@H](C(=O)N1Cc2cn(S(=O)(=O)c3ccc(OC(F)F)cc3)nc2C1)c1ccccc1F] | N-[(1R)-2-{2-[4- (difluoromethoxy)benzenesulfonyl]- 2H,4H,5H,6H-pyrrolo[3,4- c]pyrazol-5-yl}-1-(2- fluorophenyl)-2- oxoethyl]formamide | 494.98 |
| 79 | 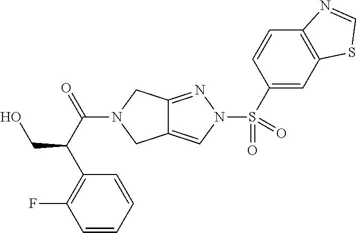 [SMILES: O=C([C@H](CO)c1ccccc1F)N1Cc2cn(S(=O)(=O)c3ccc4ncsc4c3)nc2C1] | (2S)-1-[2-(1,3-benzothiazole-6- sulfonyl)-2H,4H,5H,6H- pyrrolo[3,4-c]pyrazol-5-yl]-2- (2-fluorophenyl)-3- hydroxypropan-1-one | 473 |
| 80 | 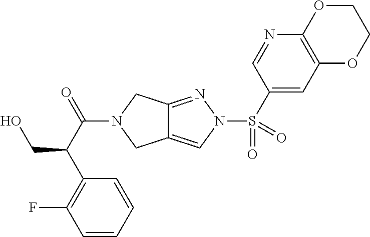 [SMILES: O=C([C@H](CO)c1ccccc1F)N1Cc2cn(S(=O)(=O)c3cnc4c(c3)OCCO4)nc2C1] | (2S)-1-(2-{2H,3H- [1,4]dioxino[2,3-b]pyridine-7- sulfonyl}-2H,4H,5H,6H- pyrrolo[3,4-c]pyrazol-5-yl)-2- (2-fluorophenyl)-3- hydroxypropan-1-one | 475 |
| 81 | 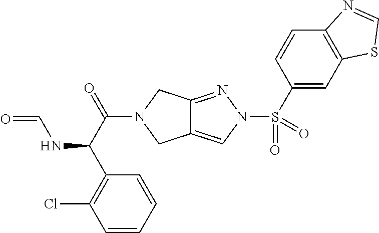 [SMILES: O=CN[C@@H](C(=O)N1Cc2cn(S(=O)(=O)c3ccc4ncsc4c3)nc2C1)c1ccccc1Cl] | N-[(1R)-2-[2-(1,3- benzothiazole-6-sulfonyl)- 2H,4H,5H,6H-pyrrolo[3,4- c]pyrazol-5-yl]-1-(2- chlorophenyl)-2- oxoethyl]formamide | 503.06 |
| 82 | 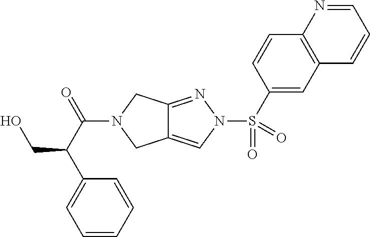 [SMILES: O=C([C@H](CO)c1ccccc1)N1Cc2cn(S(=O)(=O)c3ccc4ncccc4c3)nc2C1] | (2S)-3-hydroxy-2-phenyl-1-[2- (quinoline-6-sulfonyl)- 2H,4H,5H,6H-pyrrolo[3,4- c]pyrazol-5-yl]propan-1-one | 449.1 |
| 83 | 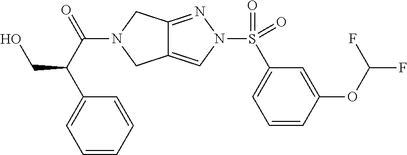 [SMILES: O=C([C@H](CO)c1ccccc1)N1Cc2cn(S(=O)(=O)c3cccc(OC(F)F)c3)nc2C1] | (2S)-1-{2-[3- (difluoromethoxy)benzenesulfonyl]- 2H,4H,5H,6H-pyrrolo[3,4- c]pyrazol-5-yl}-3-hydroxy-2- phenylpropan-1-one | 464.3 |
| 84 | 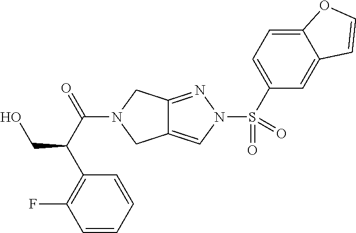 [SMILES: O=C([C@H](CO)c1ccccc1F)N1Cc2cn(S(=O)(=O)c3ccc4occc4c3)nc2C1] | (2S)-1-[2-(1-benzofuran-5- sulfonyl)-2H,4H,5H,6H- pyrrolo[3,4-c]pyrazol-5-yl]-2- (2-fluorophenyl)-3- hydroxypropan-1-one | 456 |
| 85 | 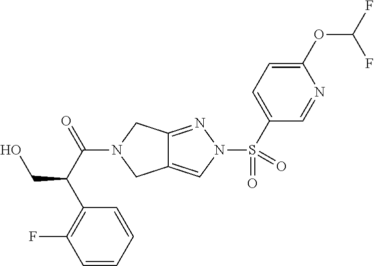 [SMILES: O=C([C@H](CO)c1ccccc1F)N1Cc2cn(S(=O)(=O)c3ccc(OC(F)F)nc3)nc2C1] | (2S)-1-(2-{[6- (difluoromethoxy)pyridin-3- yl]sulfonyl}-2H,4H,5H,6H- pyrrolo[3,4-c]pyrazol-5-yl)-2- (2-fluorophenyl)-3- hydroxypropan-1-one | 483 |
| 86 | 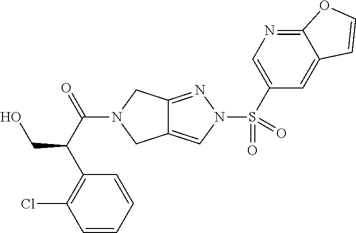 [SMILES: O=C([C@H](CO)c1ccccc1Cl)N1Cc2cn(S(=O)(=O)c3cnc4occc4c3)nc2C1] | (2S)-2-(2-chlorophenyl)-1-(2- {furo[2,3-b]pyridine-5- sulfonyl}-2H,4H,5H,6H- pyrrolo[3,4-c]pyrazol-5-yl)-3- hydroxypropan-1-one | 473 |
| 87 | 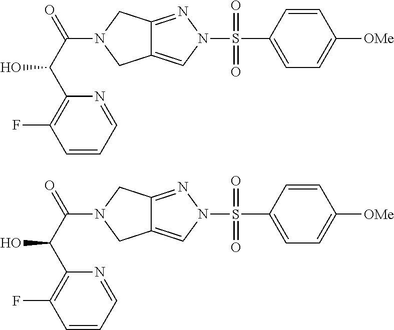 | (2S)-2-(3-fluoropyridin-2-yl)-2- hydroxy-1-[2-(4- methoxybenzenesulfonyl)- 2H,4H,5H,6H-pyrrolo[3,4- c]pyrazol-5-yl]ethan-1-one or (2R)-2-(3-fluoropyridin-2-yl)- 2-hydroxy-1-[2-(4- methoxybenzenesulfonyl)- 2H,4H,5H,6H-pyrrolo[3,4- c]pyrazol-5-yl]ethan-1-one | 433 |
| 88 | 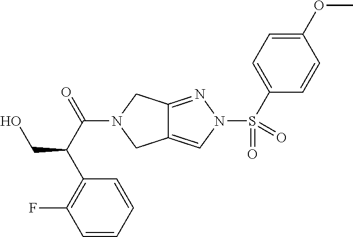 [SMILES: COc1ccc(S(=O)(=O)n2cc3c(n2)CN(C(=O)[C@H](CO)c2ccccc2F)C3)cc1] | (2S)-2-(2-fluorophenyl)-3- hydroxy-1-[2-(4- methoxybenzenesulfonyl)- 2H,4H,5H,6H-pyrrolo[3,4- c]pyrazol-5-yl]propan-1-one | 446 |
| 89 | 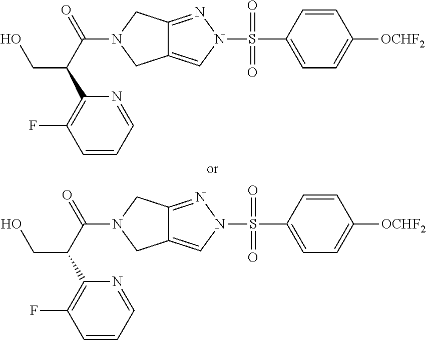 | (2S)-1-{2-[4- (difluoromethoxy)benzenesulfonyl]- 2H,4H,5H,6H- pyrrolo[3,4-c]pyrazol-5-yl}- 2-(3-fluoropyridin-2-yl)-3- hydroxypropan-1-one or (2R)-1-{2-[4- (difluoromethoxy)benzenesulfonyl]- 2H,4H,5H,6H- pyrrolo[3,4-c]pyrazol-5-yl}- 2-(3-fluoropyridin-2-yl)-3- hydroxypropan-1-one | 483 |
| 90 | 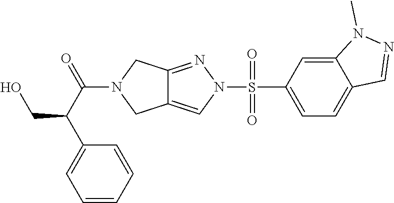 [SMILES: Cn1ncc2ccc(S(=O)(=O)n3cc4c(n3)CN(C(=O)[C@H](CO)c3ccccc3)C4)cc21] | (2S)-3-hydroxy-1-{2-[(1- methyl-1H-indazol-6- yl)sulfonyl]-2H,4H,5H,6H- pyrrolo[3,4-c]pyrazol-5-yl}-2- phenylpropan-1-one | 452.2 |
| 91 | 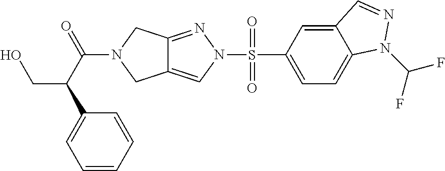 [SMILES: O=C([C@H](CO)c1ccccc1)N1Cc2cn(S(=O)(=O)c3ccc4c(cnn4C(F)F)c3)nc2C1] | (2S)-1-(2-{[1- (difluoromethyl)-1H-indazol- 5-yl]sulfonyl}- 2H,4H,5H,6H-pyrrolo[3,4- c]pyrazol-5-yl)-3-hydroxy-2- phenylpropan-1-one | 488.1 |
| 92 | 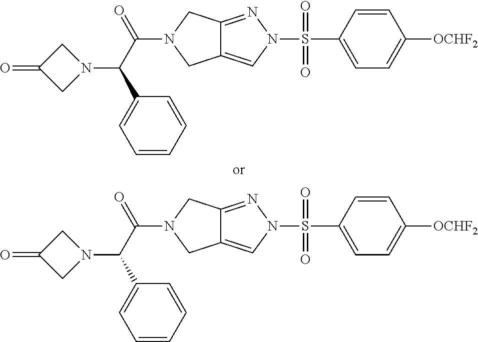 | 1-[(1R)-2-{2-[4- (difluoromethoxy)benzenesulfonyl]- 2H,4H,5H,6H-pyrrolo[3,4- c]pyrazol-5-yl}-2-oxo-1- phenylethyl]azetidin-3-one or 1-[(1S)-2-{2-[4- (difluoromethoxy)benzenesulfonyl]- 2H,4H,5H,6H-pyrrolo[3,4- c]pyrazol-5-yl}-2-oxo-1- phenylethyl]azetidin-3-one | 503 |
| 93 | 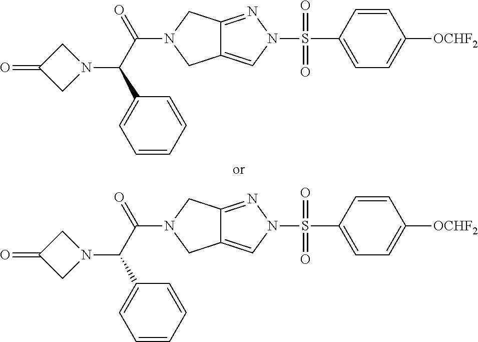 | 1-[(1R)-2-{2-[4- (difluoromethoxy)benzenesulfonyl]- 2H,4H,5H,6H-pyrrolo[3,4- c]pyrazol-5-yl}-2-oxo-1- phenylethyl]azetidin-3-one or 1-[(1S)-2-{2-[4- (difluoromethoxy)benzenesulfonyl]- 2H,4H,5H,6H-pyrrolo[3,4- c]pyrazol-5-yl}-2-oxo-1- phenylethyl]azetidin-3-one | 503 |
| 94 | 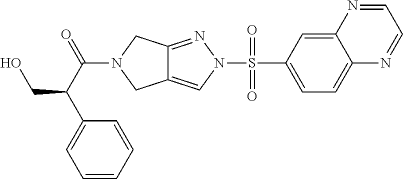 [SMILES: O=C([C@H](CO)c1ccccc1)N1Cc2cn(S(=O)(=O)c3ccc4nccnc4c3)nc2C1] | (2S)-3-hydroxy-2-phenyl-1- [2-(quinoxaline-6-sulfonyl)- 2H,4H,5H,6H-pyrrolo[3,4- c]pyrazol-5-yl]propan-1-one | 450.2 |
| 95 | 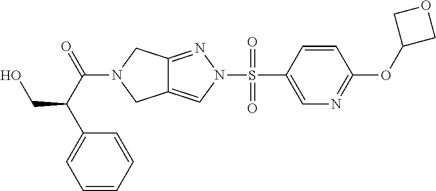 [SMILES: O=C([C@H](CO)c1ccccc1)N1Cc2cn(S(=O)(=O)c3ccc(OC4COC4)nc3)nc2C1] | (2S)-3-hydroxy-1-(2-{[6- (oxetan-3-yloxy)pyridin-3- yl]sulfonyl}-2H,4H,5H,6H- pyrrolo[3,4-c]pyrazol-5-yl)-2- phenylpropan-1-one | 470.99 |
| 96 | 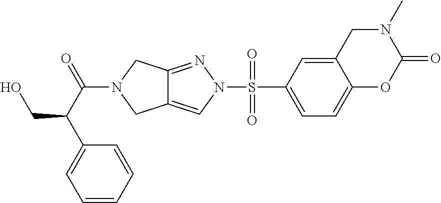 [SMILES: CN1Cc2cc(S(=O)(=O)n3cc4c(n3)CN(C(=O)[C@H](CO)c3ccccc3)C4)ccc2OC1=O] | 6-({5-[(2S)-3-hydroxy-2- phenylpropanoyl]- 2H,4H,5H,6H-pyrrolo[3,4- c]pyrazol-2-yl}sulfonyl)-3- methyl-3,4-dihydro-2H-1,3- benzoxazin-2-one | 483.1 |
| 97 | 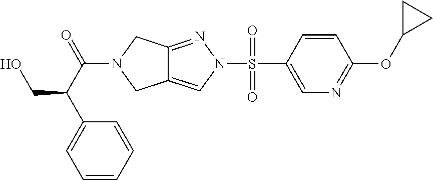 [SMILES: O=C([C@H](CO)c1ccccc1)N1Cc2cn(S(=O)(=O)c3ccc(OC4CC4)nc3)nc2C1] | (2S)-1-{2-[(6- cyclopropoxypyridin-3- yl)sulfonyl]-2H,4H,5H,6H- pyrrolo[3,4-c]pyrazol-5-yl}-3- hydroxy-2-phenylpropan-1-one | 455.06 |
| 98 | 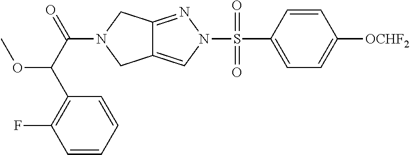 [SMILES: COC(C(=O)N1Cc2cn(S(=O)(=O)c3ccc(OC(F)F)cc3)nc2C1)c1ccccc1F] | 1-{2-[4- (difluoromethoxy)benzenesulfonyl]- 2H,4H,5H,6H-pyrrolo[3,4- c]pyrazol-5-yl}-2-(2- fluorophenyl)-2-methoxyethan- 1-one (Racemic) | 482.2 |
| 99 | 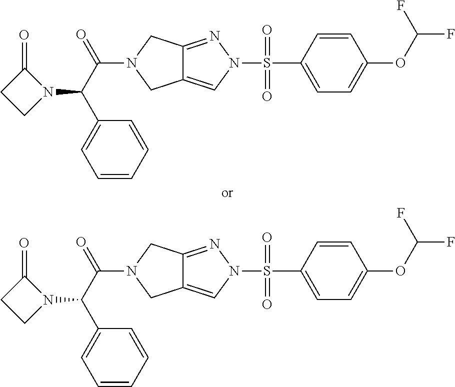 [SMILES: O=C([C@@H](c1ccccc1)N1CCC1=O)N1Cc2cn(S(=O)(=O)c3ccc(OC(F)F)cc3)nc2C1 ; "or" ; O=C([C@H](c1ccccc1)N1CCC1=O)N1Cc2cn(S(=O)(=O)c3ccc(OC(F)F)cc3)nc2C1] | 1-[(1R)-2-{2-[4- (difluoromethoxy)benzenesulfonyl]- 2H,4H,5H,6H-pyrrolo[3,4- c]pyrazol-5-yl}-2-oxo-1- phenylethyl]azetidin-2-one or 1-[(1S)-2-{2-[4- (difluoromethoxy)benzenesulfonyl]- 2H,4H,5H,6H-pyrrolo[3,4- c]pyrazol-5-yl}-2-oxo-1- phenylethyl]azetidin-2-one | 503 |
| 100 | 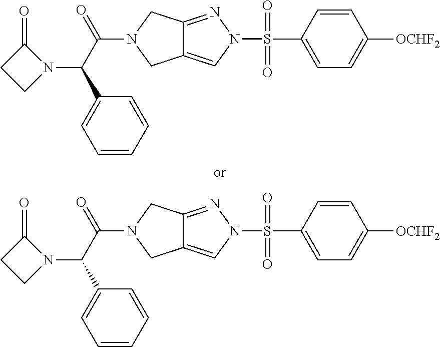 [SMILES: O=C([C@@H](c1ccccc1)N1CCC1=O)N1Cc2cn(S(=O)(=O)c3ccc(OC(F)F)cc3)nc2C1 ; "or" ; O=C([C@H](c1ccccc1)N1CCC1=O)N1Cc2cn(S(=O)(=O)c3ccc(OC(F)F)cc3)nc2C1] | 1-[(1R)-2-{2-[4- (difluoromethoxy)benzenesulfonyl]- 2H,4H,5H,6H-pyrrolo[3,4- c]pyrazol-5-yl}-2-oxo-1- phenylethyl]azetidin-2-one or 1-[(1S)-2-{2-[4- (difluoromethoxy)benzenesulfonyl]- 2H,4H,5H,6H-pyrrolo[3,4- c]pyrazol-5-yl}-2-oxo-1- phenylethyl]azetidin-2-one | 503 |
| 101 | 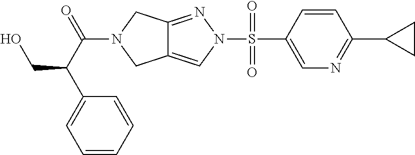 [SMILES: O=C([C@H](CO)c1ccccc1)N1Cc2cn(S(=O)(=O)c3ccc(C4CC4)nc3)nc2C1] | (2S)-1-{2-[(6- cyclopropylpyridin-3- yl)sulfonyl]-2H,4H,5H,6H- pyrrolo[3,4-c]pyrazol-5-yl}-3- hydroxy-2-phenylpropan-1-one | 438.98 |
| 102 | 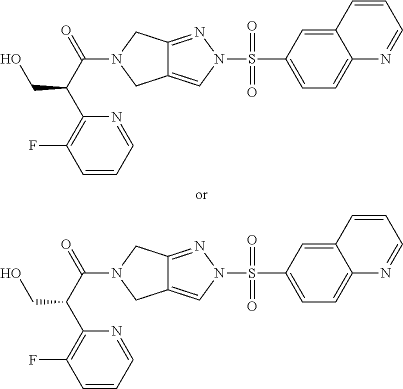 [SMILES: O=C([C@H](CO)c1ncccc1F)N1Cc2cn(S(=O)(=O)c3ccc4ncccc4c3)nc2C1 ; O=C([C@@H](CO)c1ncccc1F)N1Cc2cn(S(=O)(=O)c3ccc4ncccc4c3)nc2C1] | (2S)-2-(3-fluoropyridin-2- yl)-3-hydroxy-1-[2- (quinoline-6-sulfonyl)- 2H,4H,5H,6H-pyrrolo[3,4- c]pyrazol-5-yl]propan-1-one or (2R)-2-(3-fluoropyridin-2- yl)-3-hydroxy-1-[2- (quinoline-6-sulfonyl)- 2H,4H,5H,6H-pyrrolo[3,4- c]pyrazol-5-yl]propan-1-one | 468 |
| 103 | 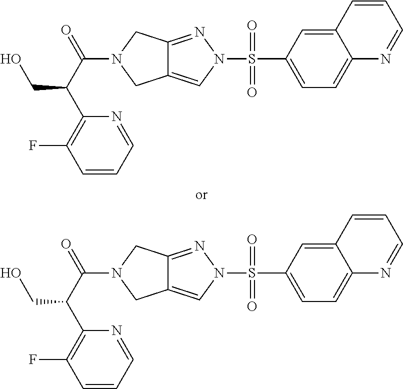 [SMILES: O=C([C@H](CO)c1ncccc1F)N1Cc2cn(S(=O)(=O)c3ccc4ncccc4c3)nc2C1 ; O=C([C@@H](CO)c1ncccc1F)N1Cc2cn(S(=O)(=O)c3ccc4ncccc4c3)nc2C1] | (2S)-2-(3-fluoropyridin-2- yl)-3-hydroxy-1-[2- (quinoline-6-sulfonyl)- 2H,4H,5H,6H-pyrrolo[3,4- c]pyrazol-5-yl]propan-1-one or (2R)-2-(3-fluoropyridin-2- yl)-3-hydroxy-1-[2- (quinoline-6-sulfonyl)- 2H,4H,5H,6H-pyrrolo[3,4- c]pyrazol-5-yl]propan-1-one | 468 |
| 104 | 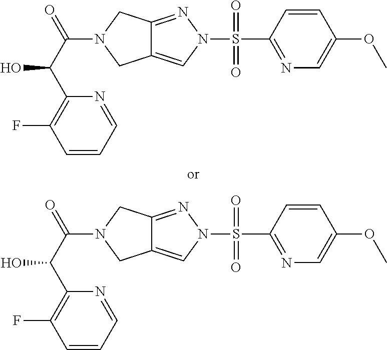 [SMILES: COc1ccc(S(=O)(=O)n2cc3c(n2)CN(C(=O)[C@H](O)c2ncccc2F)C3)nc1 ; COc1ccc(S(=O)(=O)n2cc3c(n2)CN(C(=O)[C@@H](O)c2ncccc2F)C3)nc1] | (2R)-2-(3-fluoropyridin-2-yl)- 2-hydroxy-1-{2-[(5- methoxypyridin-2-yl)sulfonyl]- 2H,4H,5H,6H-pyrrolo[3,4- c]pyrazol-5-yl}ethan-1-one or (2S)-2-(3-fluoropyridin-2-yl)- 2-hydroxy-1-{2-[(5- methoxypyridin-2-yl)sulfonyl]- 2H,4H,5H,6H-pyrrolo[3,4- c]pyrazol-5-yl}ethan-1-one | 433 |
| 105 | 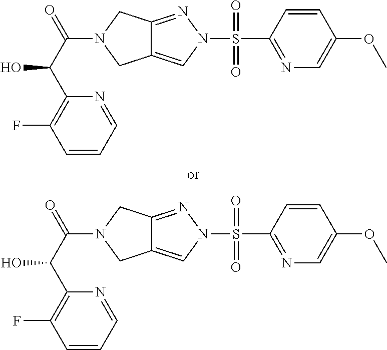 [SMILES: COc1ccc(S(=O)(=O)n2cc3c(n2)CN(C(=O)[C@H](O)c2ncccc2F)C3)nc1 ; COc1ccc(S(=O)(=O)n2cc3c(n2)CN(C(=O)[C@@H](O)c2ncccc2F)C3)nc1] | (2R)-2-(3-fluoropyridin-2-yl)- 2-hydroxy-1-{2-[(5- methoxypyridin-2-yl)sulfonyl]- 2H,4H,5H,6H-pyrrolo[3,4- c]pyrazol-5-yl}ethan-1-one or (2S)-2-(3-fluoropyridin-2-yl)- 2-hydroxy-1-{2-[(5- methoxypyridin-2-yl)sulfonyl]- 2H,4H,5H,6H-pyrrolo[3,4- c]pyrazol-5-yl}ethan-1-one | 433 |
| 106 | 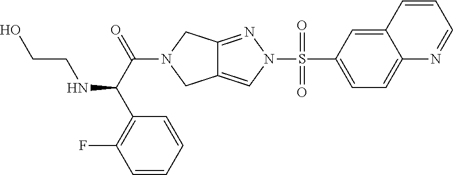 [SMILES: O=C([C@H](NCCO)c1ccccc1F)N1Cc2cn(S(=O)(=O)c3ccc4ncccc4c3)nc2C1] | (2R)-2-(2-fluorophenyl)-2- [(2-hydroxyethyl)amino]-1- [2-(quinoline-6-sulfonyl)- 2H,4H,5H,6H-pyrrolo[3,4- c]pyrazol-5-yl]ethan-1-one | 496 |
| 107 | 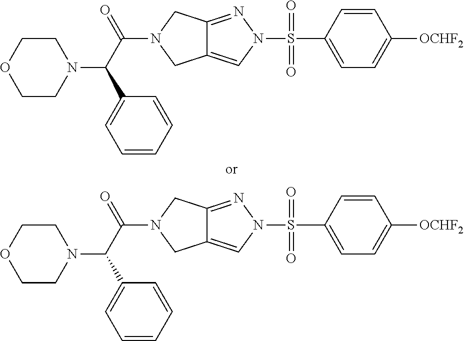 | (2R)-1-{2-[4- (difluoromethoxy)benzenesulfonyl]- 2H,4H,5H,6H-pyrrolo[3,4- c]pyrazol-5-yl}-2-(morpholin- 4-yl)-2-phenylethan-1-one or (2S)-1-{2-[4- (difluoromethoxy)benzenesulfonyl]- 2H,4H,5H,6H-pyrrolo[3,4- c]pyrazol-5-yl}-2-(morpholin- 4-yl)-2-phenylethan-1-one | 519.1 |
| 108 | 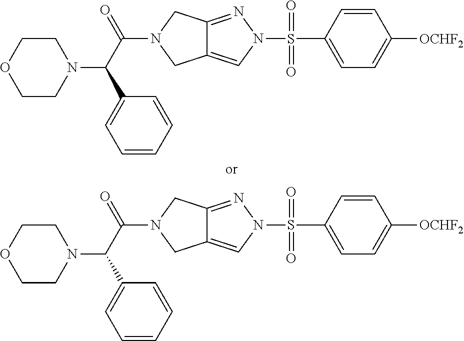 | (2R)-1-{2-[4- (difluoromethoxy)benzenesulfonyl]- 2H,4H,5H,6H-pyrrolo[3,4- c]pyrazol-5-yl}-2-(morpholin- 4-yl)-2-phenylethan-1-one or (2S)-1-{2-[4- (difluoromethoxy)benzenesulfonyl]- 2H,4H,5H,6H-pyrrolo[3,4- c]pyrazol-5-yl}-2-(morpholin- 4-yl)-2-phenylethan-1-one | 519.2 |

^{a} Unless made from an optically pure commercial starting material, absolute stereochemistry was not determined for each individual enantiomer or diastereomer.

### III. Pharmaceutical Compositions and Modes of Administration

Compounds provided herein are usually administered in the form of pharmaceutical compositions. Thus, provided herein are also pharmaceutical compositions that comprise one or more of the compounds described herein or a pharmaceutically acceptable salt, a stereoisomer, or a mixture of stereoisomers thereof and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, a stereoisomer, or a mixture of stereoisomers thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, a stereoisomer, or a mixture of stereoisomers thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, a stereoisomer, or a mixture of stereoisomers thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject. A dose may be administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

### IV. Methods of Treatment

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

In certain embodiments, the subject matter disclosed herein is directed to a method of activating PKR and/or PKM2, including methods of treating a disease or disorder in a subject by administering a therapeutically effective amount of a compound of Formula I, a pharmaceutically acceptable salt thereof, or a pharmaceutical composition comprising a compound of Formula I. In certain embodiments, the disease or disorder is selected from the group consisting of PKD (pyruvate kinase deficiency), SCD (e.g., sickle cell anemia), and thalassemia (e.g., beta-thalassemia).

In certain embodiments, the subject matter disclosed herein is directed to a method of treating a subject afflicted with a disease associated with decreased activity of PKR and/or PKM2, comprising administering to the subject an effective amount of a compound of Formula I, a pharmaceutically acceptable salt thereof, or a pharmaceutical composition comprising a compound of Formula I. In certain embodiments, the disease associated with decreased activity of PKR is selected from the group consisting of hereditary non-spherocytic hemolytic anemia, hemolytic anemia (e.g., chronic hemolytic anemia caused by phosphoglycerate kinase deficiency), hereditary spherocytosis, hereditary elliptocytosis, abetalipoproteinemia (or Bassen-Kornzweig syndrome), paroxysmal nocturnal hemoglobinuria, acquired hemolytic anemia (e.g., congenital anemias (e.g., enzymopathies)), and anemia of chronic diseases.

In certain embodiments, the subject matter described herein is directed to a method of treating a disease or disorder associated with modulation of PKR and/or PKM2 in a subject, comprising administering to the subject an effective amount of a compound of Formula I, a pharmaceutically acceptable salt thereof, or a pharmaceutical composition comprising a compound of Formula I.

In certain embodiments, the subject matter described herein is directed to a method of treating cancer in a subject in need thereof, comprising administering an effective amount of a compound of Formula I, a pharmaceutically acceptable salt thereof, or a pharmaceutical composition comprising a compound of Formula I. In certain embodiments, the cancer is selected from the group consisting of bladder cancer, breast cancer (e.g., ductal carcinoma), cervical cancer (e.g., squamous cell carcinoma), colorectal cancer (e.g., adenocarcinoma), esophageal cancer (e.g., squamous cell carcinoma), gastric cancer (e.g., adenocarcinoma, medulloblastoma, colon cancer, choriocarcinoma, squamous cell carcinoma), head and neck cancer, hematologic cancer (e.g., acute lymphocytic anemia, acute myeloid leukemia, acute lymphoblastic B cell leukemia, anaplastic large cell lymphoma, B-cell lymphoma, Burkitt's lymphoma, chronic lymphocytic leukemia, chronic eosinophillic leukemia/hypereosinophillic syndrome, chronic myeloid leukemia, Hodgkin's lymphoma, mantle cell lymphoma, multiple myeloma, T-cell acute lymphoblastic leukemia), lung cancer (e.g., bronchioloalveolar adenocarcinoma, mesothelioma, mucoepidermoid carcinoma, small-cell lung cancer, non-small cell lung cancer, adenocarcinoma, squamous cell carcinoma), liver cancer (e.g., hepatocellular carcinoma), lymphoma, neurological cancer (e.g., glioblastoma, neuroblastoma, neuroglioma), ovarian (e.g., adenocarcinoma), pancreatic cancer (e.g., ductal carcinoma), prostate cancer (e.g., adenocarcinoma), renal cancer (e.g., renal cell carcinoma, clear cell renal carcinoma), sarcoma (e.g., chondrosarcoma, Ewings sarcoma, fibrosarcoma, multipotential sarcoma, osteosarcoma, rhabdomyosarcoma, synovial sarcoma), skin cancer (e.g., melanoma, epidermoid carcinoma, squamous cell carcinoma), thyroid cancer (e.g., medullary carcinoma), and uterine cancer. In a preferred embodiment, the cancer is lung cancer.

In certain embodiments, compounds described herein, or pharmaceutically acceptable salts thereof, are useful as inhibitors of ubiquitin specific peptidase 9X (USP9X). USP9X inhibiting compounds are useful in the treatment of diseases and disorders associated with modulation of USP9X, such as cancer. See, for example WO/2020/061261 or WO/2021/055668.

In certain embodiments, the methods of administering and treating described herein further comprise co-administration of one or more additional pharmaceutically active compounds.

In a combination therapy, the pharmaceutically active compounds can be administered at the same time, in the same formulation, or at different times. Such combination therapy comprises co-administration of a compound of Formula I or a pharmaceutically acceptable salt thereof with at least one additional pharmaceutically active compound. Combination therapy in a fixed dose combination therapy comprises co-administration of a compound of Formula I or a pharmaceutically acceptable salt thereof with at least one additional pharmaceutically active compound in a fixed-dose formulation. Combination therapy in a free dose combination therapy comprises co-administration of a compound of Formula I or a pharmaceutically acceptable salt thereof and at least one additional pharmaceutically active compound in free doses of the respective compounds, either by simultaneous administration of the individual compounds or by sequential use of the individual compounds over a period of time.

### V. Methods of Preparing Compounds of Formula I and Pharmaceutically Acceptable Salts Thereof

The starting materials and reagents used in preparing the compounds described herein are either available from commercial suppliers such as Sigma-Aldrich Chemical Co., (Milwaukee, Wis.), Bachem (Torrance, Calif.), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Synthetic chemistry transformations and protecting group methodologies (protection and deprotection) useful in synthesizing compounds and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3rd Ed., John Wiley and Sons (1999); and L. Paquette, ed., Encyclopedia of Reagents for Organic Synthesis, John Wiley and Sons (1995) and subsequent editions thereof.

Compounds may be prepared singly or as compound libraries comprising at least 2, for example 5 to 1,000 compounds, or 10 to 100 compounds. Libraries of compounds of Formula I may be prepared by a combinatorial ‘split and mix’ approach or by multiple parallel syntheses using either solution phase or solid phase chemistry, by procedures known to those skilled in the art. Thus, according to a further aspect, there is provided a compound library comprising at least 2 compounds, or pharmaceutically acceptable salts thereof.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

### General Synthetic Schemes

Compounds of Formula I can be prepared by the following general procedure described in Scheme 1 below:

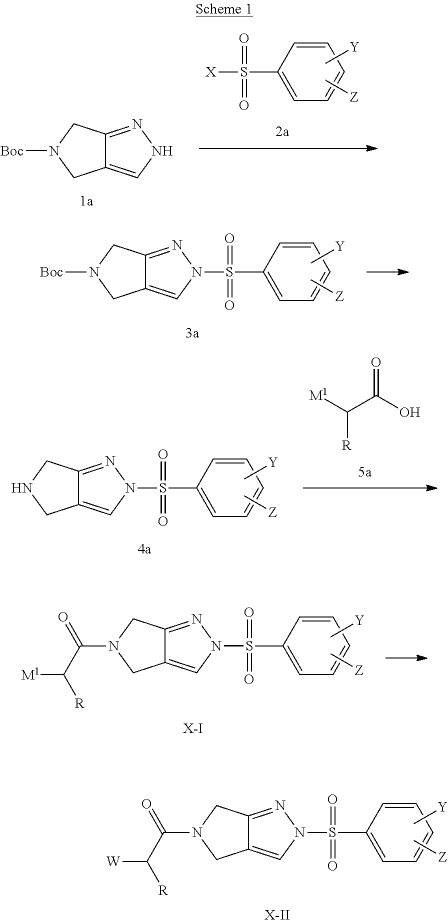

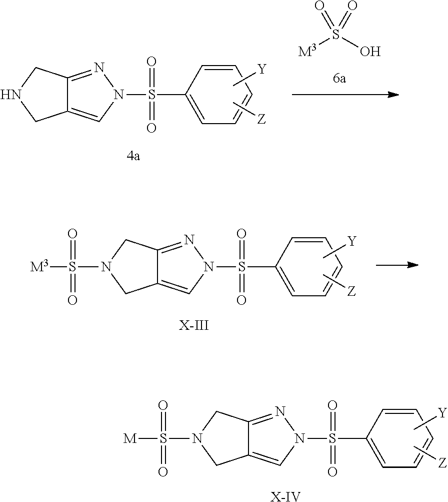

Compounds of Formula 1a (purchased from Combi-Blocks) are reacted with sulfonyl chloride or sulfonyl fluoride (2a, X=Cl or F) in the presence of base (e.g. NaH) to form intermediate compound 3a, which upon further treatment with either HCl or ZnBr₂, provides amino intermediate 4a. Compound 4a is converted into a compound of Formula X-1 by an amide coupling reaction with an appropriate carboxylic acid 5a, in an appropriate solvent such as dimethylformamide (DMF) or 1,4-dioxane at ambient temperature, and facilitated with an appropriate base (e.g. N-methyl morpholine, triethyl amine, diisopropylethyl amine, or pyridine) and an amide coupling reagent, such as 1-[bis(dimethylamino)-methylene]-1H-1,2,3-triazolo[4,5-b]pyridinium 3-oxid hexafluorophosphate (HATU) and hydroxybenzotriazol (HOBt), 1-ethyl-3-(3-dimethyl-aminopropyl)carbodiimide (EDCI) and HOBt, or benzotriazol-1-yl-oxytripyrrolidino-phosphonium hexafluorophosphate (PyBOP). In certain embodiments, compounds of formula 5a can be purchased from commercial resources. In other embodiments, compounds of formula 5a can be prepared from commercially available precursors by methods well known in the art (see representative synthetic examples below for intermediate II-1 to intermediate II-8). In certain embodiments, compounds of formula X-I are further converted to compounds of formula X-II through appropriate chemical manipulations of the M¹ groups (e.g. De-Boc with HCl or ZnBr₂).

Compound 4a can also react with sulfonyl chloride or sulfonyl fluoride (6a, X═Cl or F) in the presence of a base (e.g. pyridine or triethyl amine) to form intermediate compound X-III. In certain embodiments, compounds of formula X-III are further converted to compounds of formula X-IV through appropriate chemical manipulations of the M³ group.

The subject matter described herein includes but is not limited to the following embodiments:

1A. A compound of Formula I:

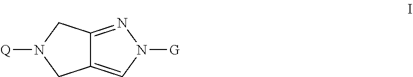

or a pharmaceutically acceptable salt thereof;

wherein,
G is

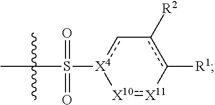

wherein,
   [symbol] represents a single or double bond;
   X⁴ is selected from the group consisting of N, C and CH;
   X¹⁰ and X¹¹ are each independently selected from the group consisting of N, CH₂, and CH, provided that only one of X¹⁰ and X¹¹ can be N;
   R¹ is selected from the group consisting of hydrogen, C₁-C₃ alkyl, C₁-C₃ alkoxy, halogen, C₁-C₃ haloalkoxy, amino, —O—R^{aa}, —(C₁-C₃ alkoxy)-R^{aa}, C₃-C₇ cycloalkyl, and hydroxy;
   R^{aa} is 4- to 7-membered heterocyclyl or C₃-C₇ cycloalkyl;
   R² is amino, C₁-C₃ alkoxy, halogen, C₁-C₃ haloalkoxy, or hydrogen, or, together with R¹ and the carbon to which each of R² and R¹ is attached, forms a 5- or 6-membered heteroaryl or 5- to 7-membered heterocyclyl containing one or two heteroatoms;
      wherein said heteroaryl or heterocyclyl formed by R¹ and R² is optionally substituted with one or two substituents, each independently selected from the group consisting of C₁-C₃ alkyl, C₁-C₃ haloalkyl, C₁-C₃ alkoxy, halogen, and C₁-C₃ haloalkoxy;
   provided that R¹ and R² cannot both be amino or hydrogen;
Q is:
   i. —C(O)—C(J¹)(J²)(J³)
      wherein,
   J¹ is hydrogen or optionally substituted C₁-C₃ alkyl; and
   J² and J³ are each independently selected from the group consisting of C₁-C₃ alkyl, hydroxy, C₁-C₃ alkoxy, hydroxy-C₁-C₃ alkyl, NR^{1a}R^{1b}, —(C₁-C₃ alkyl)-NR^{1a}R^{1b}, C₆-C₁₀ aryl, 5- to 10-membered heteroaryl, C₃-C₁₀ cycloalkyl, and 4- to 10-membered heterocyclyl;
   R^{1a} and R^{1b} are each independently hydrogen, hydroxy-C₁-C₃ alkyl, —C(O)H, or C₁-C₃ alkyl; and
wherein said aryl, heteroaryl, or heterocyclyl of J² and J³ is optionally substituted one or two times, in each instance independently with C₁-C₃ alkyl, C₁-C₃ haloalkyl, hydroxy, NR^{2a}R^{2b}, halogen, C₁-C₃ alkoxy, or C₁-C₃ haloalkoxy;
   wherein R^{2a} and R^{2b} are each independently hydrogen or C₁-C₃ alkyl; or,
   J¹ and J² together with the carbon atom to which they are each attached form a 4- to 7-membered heterocyclyl or C₃-C₇ cycloalkyl;
   ii. —C(O)O—R, wherein R is hydrogen or C₁-C₆ alkyl;

or,
iii. —S(O)₂-M
   wherein M is selected from the group consisting of C₆-C₁₀ aryl, 5- to 10-membered heteroaryl, and 5- to 10-membered heterocyclyl, wherein said aryl, heteroaryl, or heterocyclyl is optionally substituted one or two times, in each instance independently with C₁-C₃ alkyl, C₁-C₃ haloalkyl, NR^{3a}R^{3b}, —C(O)R^{2c}, hydroxy, halogen, C₁-C₃ alkoxy, or C₁-C₃ haloalkoxy;
      wherein R^{3a} and R^{3b} are each independently hydrogen or C₁-C₃ alkyl; and R^{2C} is selected from the group consisting of hydrogen, hydroxy, and C₁-C₃ alkyl;
         provided that the compound is not:

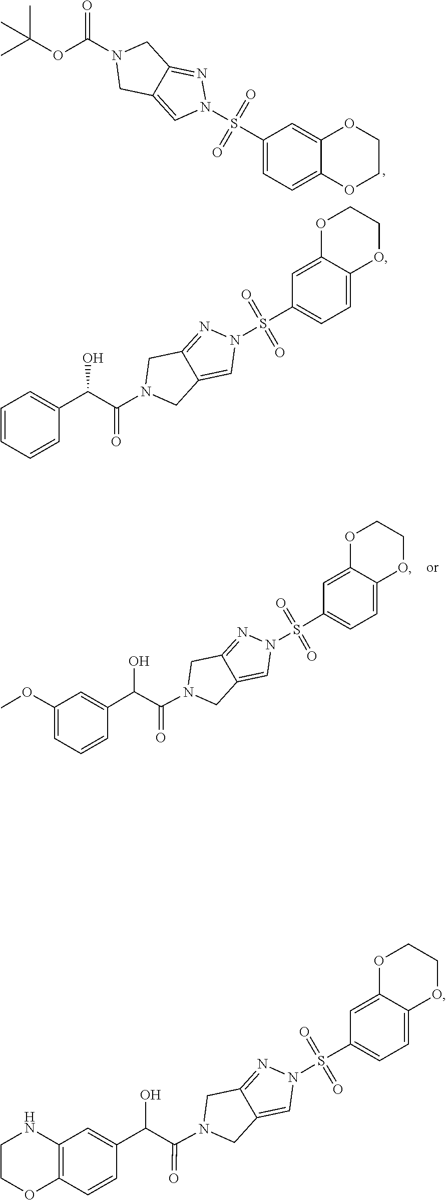

or salts thereof.

1B. A compound of Formula I:

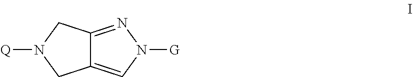

wherein,
G is

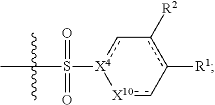

wherein,
   [symbol] represents a single or double bond;
   X⁴ is selected from the group consisting of N, C and CH;
   X¹⁰ is selected from the group consisting of N, CH₂, and CH;
   R¹ is selected from the group consisting of C₁-C₃ alkyl, alkoxy, halogen, haloalkoxy, amino, and hydroxy;
   R² is amino or hydrogen, or, together with R¹ and the carbon to which each of R² and R¹ is attached, forms a 5- or 6-membered heteroaryl or 5- to 7-membered heterocyclyl containing one or two heteroatoms;
   provided that R¹ and R² cannot both be amino;
Q is:
   i. —C(O)—C(J¹)(J²)(J³)
      wherein,
   J¹ is hydrogen or optionally substituted C₁-C₃ alkyl; and
   J² and J³ are each independently selected from the group consisting of C₁-C₃ alkyl, hydroxy, hydroxy-C₁-C₃ alkyl, —(CH₂)ₓNR^{1a}R^{1b}, C₆-C₁₀ aryl, 5- to 10-membered heteroaryl, 3- to 10-membered cycloalkyl, and 5- to 10-membered heterocyclyl;
   x is 0, 1, or 2;
   R^{1a} and R^{1b} are each independently hydrogen or C₁-C₃ alkyl; and wherein said aryl, heteroaryl, or heterocyclyl is optionally substituted one or two times, in each instance independently with C₁-C₃ alkyl, C₁-C₃ haloalkyl, hydroxy, NR^{2a}R^{2b}, halogen, alkoxy, or haloalkoxy;
      wherein R^{2a} and R^{2b} are each independently hydrogen or C₁-C₃ alkyl; or,
   J¹ and J² together with the carbon atom to which they are each attached form a 4- to 7-membered heterocyclyl or 3- to 7-membered cycloalkyl;
   ii. —C(O)O—R, wherein R is hydrogen or C₁-C₆ alkyl;

or,
iii. —S(O)₂-M
   wherein, M is selected from the group consisting of C₆-C₁₀ aryl, 5- to 10-membered heteroaryl, and 5- to 10-membered heterocyclyl, wherein said aryl, heteroaryl, or heterocyclyl is optionally substituted one or two times, in each instance independently with C₁-C₃ alkyl, C₁-C₃ haloalkyl, NR^{3a}R^{3b}, —C(O)R^{2c}, hydroxy, halogen, alkoxy, or haloalkoxy;
      wherein R^{3a} and R^{3b} are each independently hydrogen or C₁-C₃ alkyl; and R^{2C} is selected from the group consisting of hydrogen, hydroxy, and C₁-C₃ alkyl;
         or a pharmaceutically acceptable salt thereof.

2B. The compound of embodiment 1B, wherein G is:

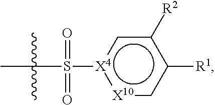

wherein X⁴ is N or C; and
X¹⁰ is N or CH.

3B. The compound of embodiment 1 or 2, wherein,

Q is —C(O)—C(J¹)(J²)(J³), wherein:
J¹ is hydrogen or C₁-C₃ alkyl;
J² is selected from the group consisting of hydroxy, C₁-C₃ alkyl, —(CH₂)ₓNH₂, and hydroxy-C₁-C₃ alkyl;
   wherein x is 0 or 1; and
J³ is C₁-C₃ alkyl or optionally substituted phenyl or 6- to 10-membered heteroaryl;
or,
J¹ and J², together with the carbon atom to which they are each attached, form a 5- or 6-membered heterocyclyl.

4B. The compound of embodiment 3B, wherein J³ is phenyl or pyridinyl optionally substituted with halo, methyl, or haloalkoxy.

5B. The compound of any one of embodiments 1A or 1B-4B, wherein Q is

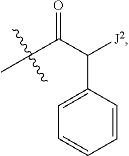

wherein J² is selected from the group consisting of —NH₂, hydroxy, hydroxy-methylene, and —CH(OH)CH₃.

6B. The compound of any one of embodiments 1A or 1B-4B, wherein J¹ and J², together with the carbon atom to which they are each attached, form a tetrahydrofuranyl, oxetanyl, pyrrolidinyl, or morpholinyl ring.

7B. The compound of any one of embodiments 1A or 1B-4B, wherein Q is

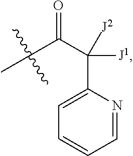

wherein J¹ is methyl and J² is hydroxy-methylene;

or

J¹ and J² together with the carbon atom to which they are each attached form a tetrahydrofuranyl ring.

8B. The compound of any one of embodiments 1A or 1B-2B, wherein Q is —S(O)₂-M,
wherein M is phenyl, optionally substituted once with NR^{3a}R^{3b} or haloalkoxy; and wherein R^{3a} and R^{3b}are each hydrogen.

9B. The compound of any one of embodiments 1A or 1B-2B, wherein Q is —S(O)₂-M,
wherein M is optionally substituted 5- to 10-membered heterocyclyl or 5- to 10-membered heteroaryl.

10B. The compound of embodiment 9B, wherein M is 9- or 10-membered bicyclic heterocyclyl or 9- or 10-membered bicyclic heteroaryl, optionally substituted once with NR^{3a}R^{3b}, haloalkoxy, or —C(O)R^{2c};
wherein R^{3a} and R^{3b} are each hydrogen; and R^{2c} is C₁-C₃ alkyl.

11B. The compound of embodiment 10B, wherein M is:

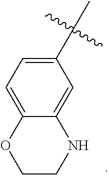 [SMILES: *c1ccc2c(c1)NCCO2]

12B. The compound of embodiment 10B, wherein M is:

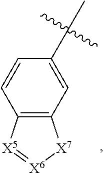

wherein,
   X⁷ is O, S or NH;
   X⁶ is N or CH; and
   X⁵ is N or CR²⁰;
      wherein R²⁰ is selected from the group consisting of hydrogen, C₁-C₃ alkyl, and hydroxy.

13B. The compound of embodiment 12B, wherein M is:

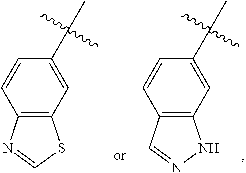 [SMILES: *c1ccc2ncsc2c1 ; *c1ccc2cn[nH]c2c1]

14B. The compound of any one of embodiments 1A or 1B-13B, wherein X⁴ is C.

15B. The compound of any one of embodiments 1A or 1B-14B, wherein X¹⁰ is CH.

16B. The compound of any one of the embodiments 1A, or 1A-15B, wherein R¹ is haloalkoxy.

17B. The compound of embodiment 16B, wherein R¹ is —OCHF₂.

18B. The compound of any one of embodiments 14B-17B, wherein R² is hydrogen.

19B. The compound of any one of embodiments 14B-18B, wherein G is:

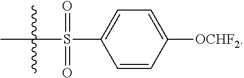 [SMILES: *S(=O)(=O)c1ccc(OC(F)F)cc1]

20B. The compound of embodiment 1A, 1B, or 2B, wherein R² together with R¹ and the carbon to which each of R² and R¹ is attached form a 5- or 6-membered heteroaryl or 5- or 6-membered heterocyclyl.

21B. The compound of embodiment 20B, wherein R² together with R¹ and the carbon to which each of R² and R¹ is attached form a thiazolyl ring.

22B. The compound of embodiment 21B, wherein G is

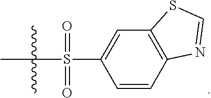 [SMILES: *S(=O)(=O)c1ccc2ncsc2c1]

23B. The compound of embodiment 20B, wherein R² together with R¹ and the carbon to which each of R² and R¹ is attached form a morpholinyl, dioxanyl, or dioxolyl ring.

24B. The compound of embodiment 23B, wherein G is selected from the group consisting of:

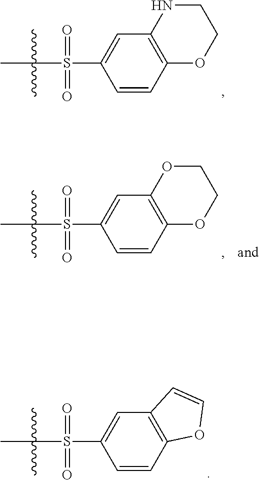 [SMILES: *S(=O)(=O)c1ccc2c(c1)NCCO2 ; *S(=O)(=O)c1ccc2c(c1)OCCO2 ; *S(=O)(=O)c1ccc2occc2c1]

2A. The compound of embodiment 1A, wherein G is:

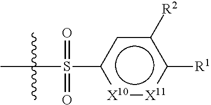

wherein X¹⁰ and X¹¹ are each independently CH or N, provided that only one of X¹⁰ and X¹¹ can be N.

3A. The compound of embodiment 1A or 2A, wherein,

Q is —C(O)—C(J¹)(J²)(J³), wherein:
J¹ is hydrogen or C₁-C₃ alkyl;
J² is selected from the group consisting of hydroxy, C₁-C₃ alkyl, —NHR^{1b}, —(C₁-C₃ alkyl)-NHR^{1b}, C₁-C₃ alkoxy, 4- to 6-membered heterocyclyl, and hydroxy-C₁-C₃ alkyl;
R^{1b} is hydrogen, hydroxy-C₁-C₃ alkyl, —C(O)H, or C₁-C₃ alkyl; and
J³ is optionally substituted phenyl or 6- to 10-membered heteroaryl;
or,
J¹ and J², together with the carbon atom to which they are each attached, form a 4- to 6-membered heterocyclyl.

4A. The compound of embodiment 3A, wherein J³ is phenyl or pyridinyl optionally substituted with halo, methyl, C₁-C₃ haloalkyl, or C₁-C₃ haloalkoxy.

5A. The compound of embodiment 4A, wherein J³ is phenyl or pyridinyl optionally substituted with chloro, fluoro, methyl, —CH₂F, —CHF₂, —CF₃, —OCH₂F, —OCHF₂, or —OCF₃.

6A. The compound of any one of embodiments 1A-5A, wherein Q is

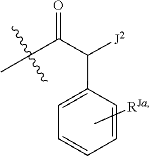

wherein J² is selected from the group consisting of —NH₂, —CH₂NH₂, —NHCH₂CH₂OH, —NHC(O)H, hydroxy, —C(CH₃)₂OH, —CH₂OH, —OCH₃,

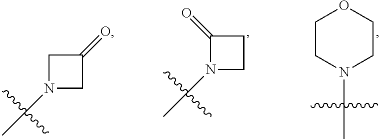 [SMILES: *N1CC(=O)C1 ; *N1CCC1=O ; *N1CCOCC1]

### and —CH(OH)CH₃; and

R^{Ja} is hydrogen, chloro, fluoro, —CF₃, —OCF₃, or methyl.

7A. The compound of any one of embodiments 1A-5A, wherein Q is

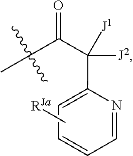

wherein J¹ is methyl or hydrogen and J² is —CH₂OH, or hydroxy; and
R^{Ja} is hydrogen, methyl, fluoro, or chloro.

8A. The compound of embodiment 7A, wherein J¹ is hydrogen.

9A. The compound of embodiment 6A or 7A, wherein R^{Ja} is ortho on the phenyl or pyridinyl ring.

10A. The compound of any one of embodiments 1A-5A, wherein J¹ and J², together with the carbon atom to which they are each attached, form a 4- to 7-membered heterocyclyl.

11A. The compound of embodiment 10A, wherein J¹ and J², together with the carbon atom to which they are each attached, form a tetrahydrofuranyl, oxetanyl, pyrrolidinyl, or morpholinyl ring.

12A. The compound of embodiment 1A or 2A, wherein Q is —S(O)₂-M, wherein M is optionally substituted 5- to 10-membered heterocyclyl or 5- to 10-membered heteroaryl.

13A. The compound of embodiment 12A, wherein M is 9- or 10-membered bicyclic heterocyclyl or 9- or 10-membered bicyclic heteroaryl, optionally substituted once with NR^{3a}R^{3b}, C₁-C₃ haloalkoxy, or —C(O)R^{2c};
wherein R^{3a} and R^{3b} are each hydrogen; and R^{2c} is C₁₋C₃ alkyl.

14A. The compound of embodiment 13A, wherein M is:

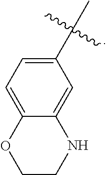 [SMILES: *c1ccc2c(c1)NCCO2]

15A. The compound of embodiment 1A, wherein X⁴ is C.

16A. The compound of any one of embodiments 1A-15A, wherein X¹⁰ is CH.

17A. The compound of any one of embodiments 1A-15A, wherein X¹⁰ is N.

18A. The compound of any one of embodiment 1A-16A, wherein X¹¹ is N.

19A. The compound of any one of embodiments 1A-17A, wherein X¹¹ is CH.

20A. The compound of any one of embodiments 1A-19A, wherein R¹ is selected from the group consisting of hydrogen, C₁-C₃ alkyl, C₁-C₃ alkoxy, halogen, C₁-C₃ haloalkoxy, amino, —O—R^{aa}, —(C₁-C₃ alkoxy)-R^{aa}, C₃-C₇ cycloalkyl, and hydroxy;
R^{aa} is 4- to 7-membered heterocyclyl or C₃-C₇ cycloalkyl; and
R² is amino, C₁-C₃ alkoxy, halogen, C₁-C₃ haloalkoxy, or hydrogen, provided that R¹ and R² cannot both be amino or hydrogen.

21A. The compound of any one of the preceding embodiments, wherein R¹ is C₁-C₃ haloalkoxy, C₁-C₃ alkoxy, C₃-C₅ cycloalkyl, or —O—R^{aa}; wherein,
R^{aa} is 4- to 6-membered heterocyclyl or C₃-C₅ cycloalkyl; and
R² is hydrogen.

22A. The compound of embodiment 21A, wherein R¹ is —OCH₃, —OCH₂F, —OCHF₂, —OCHF₃, cyclopropyl,

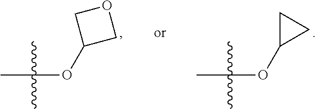 [SMILES: *OC1COC1 ; *OC1CC1]

23A. The compound of embodiment 21A or 22A, wherein R¹ is —OCHF₂.

24A. The compound of any one of embodiments 15A-23A, wherein G is:

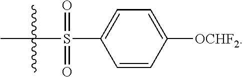 [SMILES: *S(=O)(=O)c1ccc(OC(F)F)cc1]

25A. The compound of any one of embodiments 1A-20A, wherein R² is C₁-C₃ haloalkoxy and R¹ is hydrogen.

26A. The compound of embodiment 25A, wherein R² is —OCHF₂.

27A. The compound of embodiment 1A or 2A, wherein R² together with R¹ and the carbon to which each of R² and R¹ is attached form a 5- or 6-membered heteroaryl or 5- or 6-membered heterocyclyl, wherein said heteroaryl or heterocyclyl is optionally substituted with one substituent selected from the group consisting of C₁-C₃ alkyl and C₁-C₃ haloalkoxy.

28A. The compound of embodiment 27A, wherein R² together with R¹ and the carbon to which each of R² and R¹ is attached form a thiazolyl ring.

29A. The compound of embodiment 28A, wherein G is

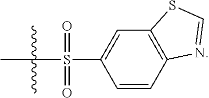 [SMILES: *S(=O)(=O)c1ccc2ncsc2c1]

30A. The compound of embodiment 27A, wherein R² together with R¹ and the carbon to which each of R² and R¹ is attached form a morpholinyl, dioxanyl, pyridinyl, pyrimidinyl, or dioxolyl ring.

31A. The compound of embodiment 30A, wherein G is selected from the group consisting of

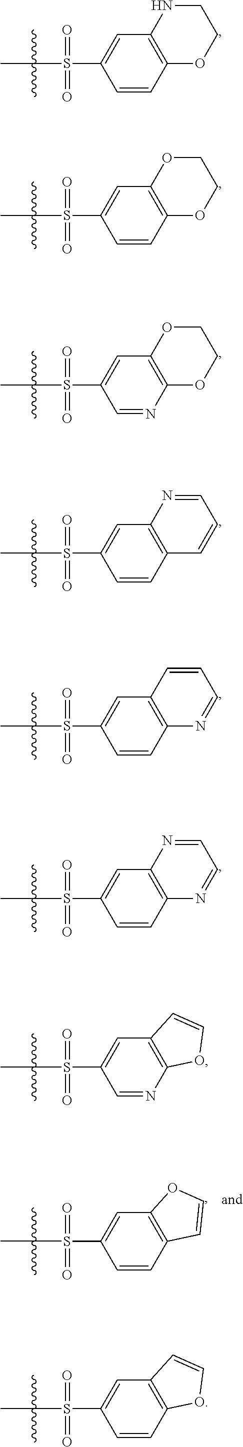

32A. The compound of embodiment 27A, wherein R² together with R¹ and the carbon to which each of R² and R¹ is attached form a pyrazolyl ring, wherein said pyrazolyl is optionally substituted with one substituent selected from the group consisting of methyl, —CH₂F, —CHF₂, and —CF_{3.} 33A. The compound of embodiment 32A, wherein G is selected from the group consisting of

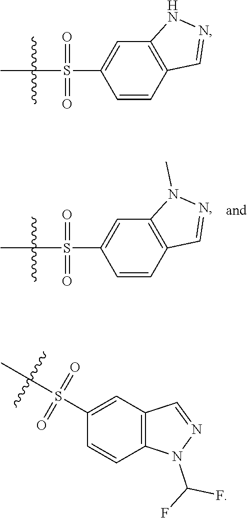 [SMILES: *S(=O)(=O)c1ccc2cn[nH]c2c1 ; *S(=O)(=O)c1ccc2cnn(C)c2c1 ; *S(=O)(=O)c1ccc2c(cnn2C(F)F)c1]

25B. The compound of embodiment 1A or 1B, selected from the compounds of Table 1, or a pharmaceutically acceptable salt thereof.

26B. A pharmaceutical composition comprising a compound of any one of embodiments 1B-25B, 1A-33A, or a pharmaceutically acceptable salt thereof, and a pharmaceutically acceptable excipient.

27B. A method of treating a disease or disorder associated with modulation of pyruvate kinase (PKR) and/or PKM2 in a subject, comprising administering to the subject an effective amount of a compound of any one of embodiments 1B-25B, 1A-33A or the pharmaceutical composition of embodiment 26B.

28B. A method of activating PKR and/or PKM2 in a subject, comprising administering to the subject an effective amount of a compound of any one of embodiments 1B-25B, 1A-33A or the pharmaceutical composition of embodiment 26B.

29B. A method of treating a subject afflicted with a disease associated with decreased activity of PKR and/or PKM2, comprising administering to the subject an effective amount of a compound of any one of embodiments 1B-25B, 1A-33A or the pharmaceutical composition of embodiment 26B.

30B. The method of embodiment 29B, wherein the disease is selected from the group consisting of sickle cell disease, sickle cell anemia, thalassemia, hereditary non-spherocytic hemolytic anemia, hemolytic anemia, hereditary spherocytosis, hereditary elliptocytosis, abetalipoproteinemia, paroxysmal nocturnal hemoglobinuria, acquired hemolytic, and anemia of chronic diseases.

The following examples are offered by way of illustration and not by way of limitation.

### EXAMPLES

### I. Synthetic Examples

Several intermediates used in the synthetic preparations of the compounds described herein are provided below:

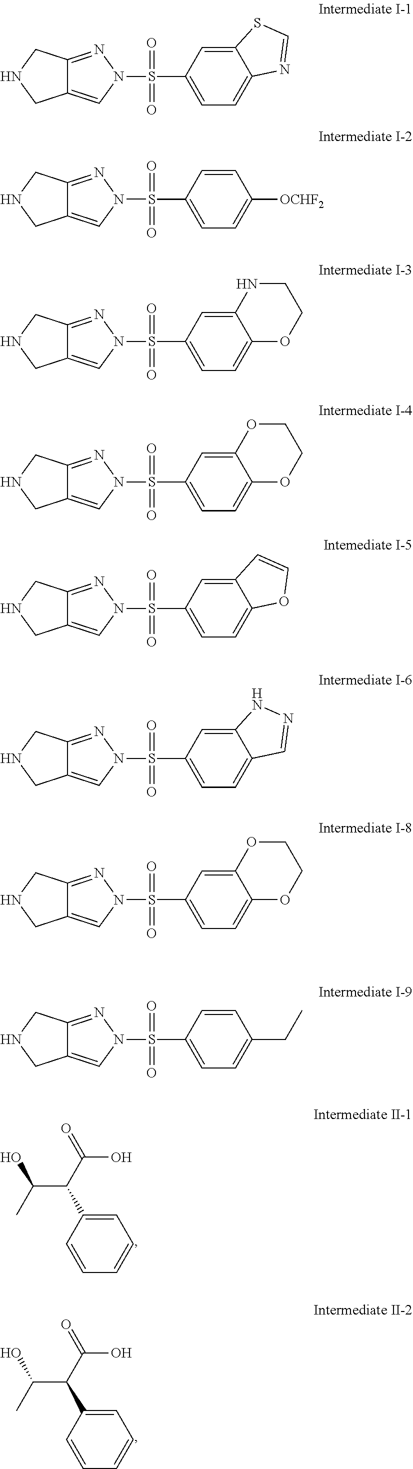

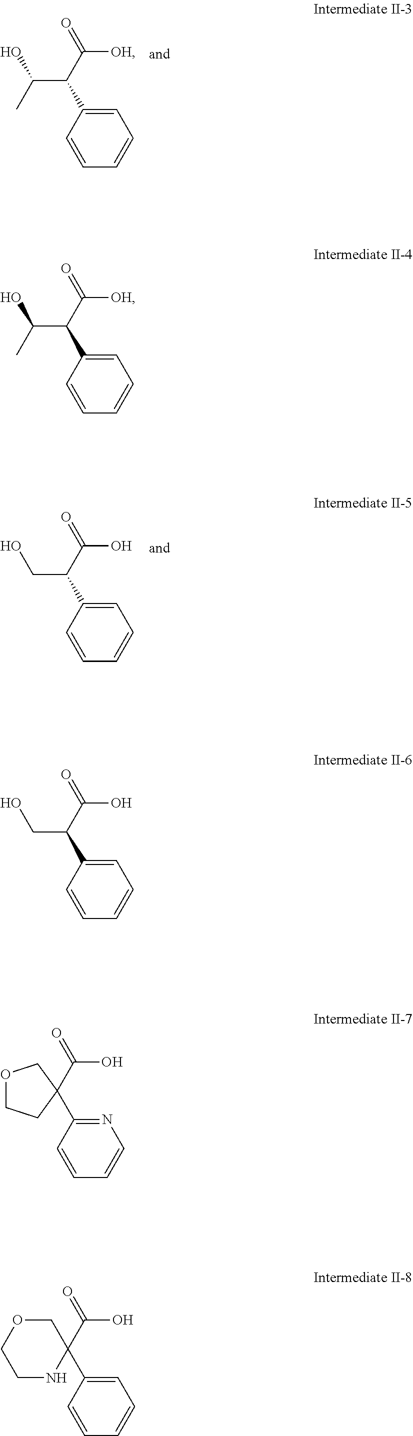

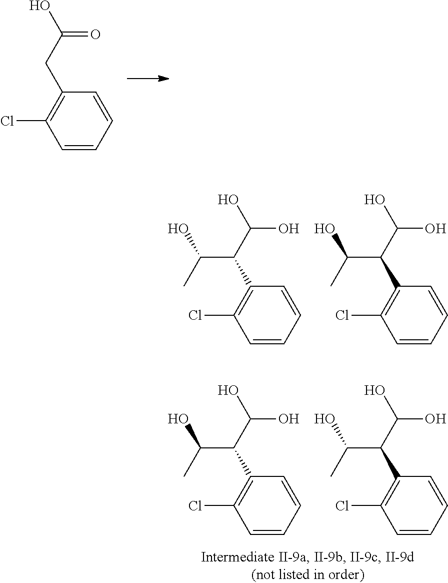

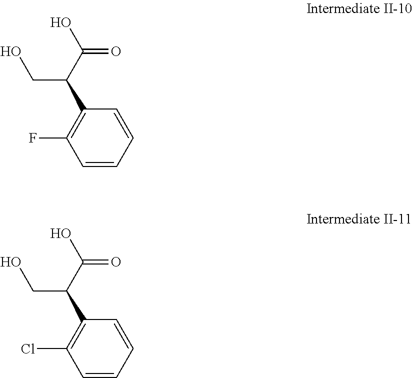 [SMILES: O=C(O)[C@H](CO)c1ccccc1F ; O=C(O)[C@H](CO)c1ccccc1Cl]

### Example 1.1

### Synthesis of 6-{2H,4H,5H,6H-pyrrolo[3,4-c]pyrazole-2-sulfonyl}-1,3-benzothiazole (Intermediate I-1)

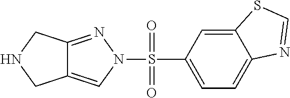 [SMILES: O=S(=O)(c1ccc2ncsc2c1)n1cc2c(n1)CNC2]

Step 1

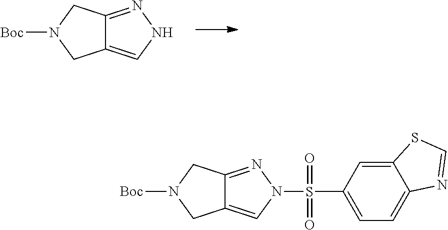 [SMILES: CC(C)(C)OC(=O)N1Cc2c[nH]nc2C1 ; CC(C)(C)OC(=O)N1Cc2cn(S(=O)(=O)c3ccc4ncsc4c3)nc2C1]

To a suspension of sodium hydride (0.70 g; 60%, 17.41 mmol; 1.30 eq.) in dry THF (15 mL) in an ice/water bath under an N₂ atmosphere was added a solution of N-{2H,4H,5H,6H-pyrrolo[3,4-c]pyrazol-5-yl}(tert-butoxy)formamide (3.0 g; 13.40 mmol; 1.00 eq.) in THF (15 mL) over 5 min. The mixture was allowed to stir in the ice bath for 30 min. A solution of 1,3-benzothiazole-6-sulfonyl chloride (3 130.39 mg; 13.40 mmol; 1.00 eq.) in THF (15 mL) was added dropwise over 5 min. The mixture was allowed to stir in the ice bath for 45 min, and at rt for 3 h subsequently. To the resulting mixture under N₂ atmosphere was added aq. NH₄Cl solution (15 mL) and HOAc to adjust the pH to ˜5. The organic layer was separated, and the aqueous layer was back extracted with 10% MeOH/CH₂Cl₂ (15) thrice. The combined organic solutions were washed with water and brine. Removal of the volatiles under reduced pressure provided the desired crude product of tert-butyl 2-(1,3-benzothiazole-6-sulfonyl)-2H,4H,5H,6H-pyrrolo[3,4-c]pyrazole-5-carboxylate as a pale yellow solid (5.1 g, 90%), which was directly used in next step. ¹H NMR (DMSO-d₆) δ: 9.70 (s, 1H), 9.02 (s, 1H), 8.37-8.17 (m, 2H), 8.03 (dt, J=8.7, 1.6 Hz, 1H), 4.30 (dd, J=9.8, 3.8 Hz, 4H), 1.39 (s, 9H); LCMS (ES) [M+1]⁺ m/z 407.3.

Step 2

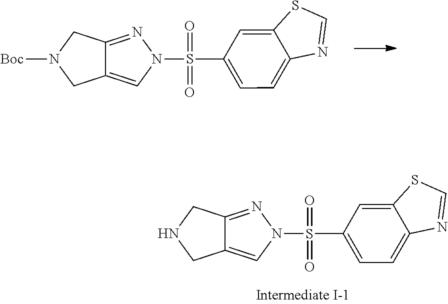 [SMILES: CC(C)(C)OC(=O)N1Cc2cn(S(=O)(=O)c3ccc4ncsc4c3)nc2C1 ; O=S(=O)(c1ccc2ncsc2c1)n1cc2c(n1)CNC2]

To a solution of tert-butyl 2-(1,3-benzothiazole-6-sulfonyl)-2H,4H,5H,6H-pyrrolo[3,4-c]pyrazole-5-carboxylate (4.1 g; 10.0 mol; 1.00 eq.) in CH₂Cl₂ (10 mL) at rt was added a solution of HCl in dioxane (15 mL; 4.00 mol/L; 60 mmol; 6.00 eq.). After stirring at rt for 75 min, the resulting precipitate was filtered, washed with cold CH₂Cl₂ (15) twice, and left on high vacuum overnight to provide the hydrogen chloride salt of the title compound as a white solid (2.86, 93%). LCMS (ES) [M+1]⁺ m/z 307.3; ¹H NMR (DMSO-d₆) δ: 10.22 (s, 2H), 9.72 (s, 1H), 9.07 (d, J=2.0 Hz, 1H), 8.45-8.20 (m, 2H), 8.07 (dd, J=8.7, 2.1 Hz, 1H), 4.27 (d, J=7.1 Hz, 4H).

### Example 1.2

### Synthesis of 2-[4-(difluoromethoxy)benzenesulfonyl]-2H,4H,5H,6H-pyrrolo[3,4-c]pyrazole (Intermediate 1-2)

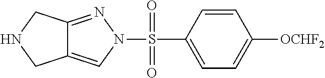 [SMILES: O=S(=O)(c1ccc(OC(F)F)cc1)n1cc2c(n1)CNC2]

The title compound was made from N-{2H,4H,5H,6H-pyrrolo[3,4-c]pyrazol-5-yl}(tert-butoxy)formamide following a procedure similar to that described for the synthesis of Intermediate I-1, except that 4-(difluoromethoxy)benzenesulfonyl chloride was used in the place of 1,3-benzothiazole-6-sulfonyl chloride (step 1). The crude product was purified by silica gel column with 0-10% MeOH/CH₂Cl₂ (0.1% NH₄OH) as the eluent to provide the desired product as a white solid. LCMS (ES) [M+1]⁺ m/z 316.0.

### Example 1.3

### Synthesis of 6-[2H,4H,5H,6H-pyrrolo[3,4-c]pyrazole-2-sulfonyl]-3,4-dihydro-2H-1,4-benzoxazine (Intermediate 1-3)

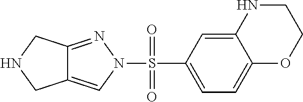 [SMILES: O=S(=O)(c1ccc2c(c1)NCCO2)n1cc2c(n1)CNC2]

Step 1

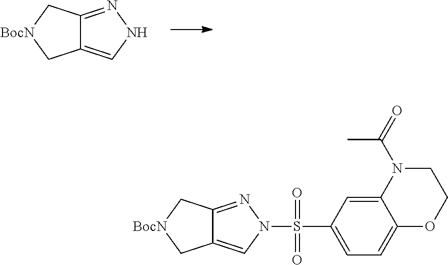 [SMILES: CC(C)(C)OC(=O)N1Cc2c[nH]nc2C1 ; CC(=O)N1CCOc2ccc(S(=O)(=O)n3cc4c(n3)CN(C(=O)OC(C)(C)C)C4)cc21]

To a suspension of sodium hydride (1.05 g; 60%, 26.1 mmol; 1.30 eq.) in dry THF (25 mL) in an ice/water bath under N₂ atmosphere was added a solution of tert-butyl 2H,4H,5H,6H-pyrrolo[3,4-c]pyrazole-5-carboxylate (4.5 g; 20.1 mmol; 1.00 eq.) in THF (23 mL) over 5 min. The mixture was allowed to stir in the ice bath for 30 min. A solution of 4-acetyl-3,4-dihydro-2H-benzo[b][1,4]oxazine-6-sulfonyl chloride (5.54 g; 20.1 mmol; 1.00 eq.) in THF (15 mL) was added dropwise over 8 min. The mixture was allowed to stir in the ice bath for 45 min, and at rt for 15 h. To the resulting mixture under N₂ atmosphere was added aq. NH₄Cl solution (20 mL) and HOAc to adjust the pH to ˜5. The organic layer was separated, and the aqueous layer was back extracted with 10% MeOH/CH₂Cl₂ (10 mL) thrice. The combined organic solutions were washed with water and brine. After removal of the volatiles under reduced pressure, the remaining solid was purified by column chromatography on silica gel eluted with 0-100% solvent A in CH₂Cl₂ (solvent A: 0.1% NH₄OH/10% MeOH/89.9% CH₂Cl₂) to provide desired tert-butyl 2-[(4-acetyl-3,4-dihydro-2H-1,4-benzoxazin-_6-yl)sulfonyl]-2H,4H,5H,6H-pyrrolo[3,4-c]pyrazole-5-carboxylate as a light yellow solid (6.1 g, 68%). LCMS (ES) [M+1]⁺ m/z 449.3;

Step 2

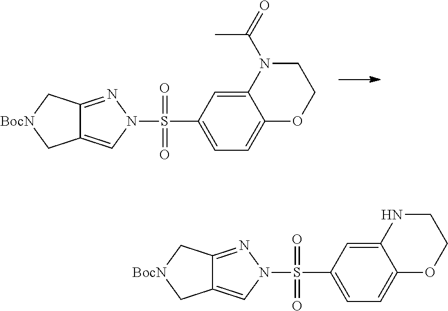 [SMILES: CC(=O)N1CCOc2ccc(S(=O)(=O)n3cc4c(n3)CN(C(=O)OC(C)(C)C)C4)cc21 ; CC(C)(C)OC(=O)N1Cc2cn(S(=O)(=O)c3ccc4c(c3)NCCO4)nc2C1]

To a solution of tert-butyl 2-[(4-acetyl-3,4-dihydro-2H-1,4-benzoxazin-6-yl)sulfonyl]-2H,4H,5H,6H-pyrrolo[3,4-c]pyrazole-5-carboxylate (2.01 g; 4.5 mmol; 1.00 eq.) in THF (20 mL) in an ice bath under N₂ atmosphere was added LiBH₄ (390 mg, 18.0 mmol, 4.00 eq.). This mixture was allowed to warm in the ice bath to rt and allowed to stir at rt for 15 h. The resulting mixture was treated with saturated aq. NH₄Cl (15 mL), and the pH value was adjusted to ˜5 with aq. HCl (1.0 N), and extracted with EtOAc (20 mL) thrice. The combined solutions were washed with water and brine. After removal of the volatiles under reduced pressure, the remaining crude product was purified by column chromatography on silica gel eluted with 0-100% solvent A in CH₂Cl₂ (Solvent A: 0.1% NH₄OH/10% MeOH/89.95 CH₂Cl₂) to provide tert-butyl 2-(3,4-dihydro-2H-1,4-benzoxazine-6-sulfonyl-2H,4H,5H,6H-pyrrolo[3,4-c]pyrazole-5-carboxylate as a white solid (1.66 g, 91%). LCMS (ES) [M+1]⁺ m/z 407.4.

Step 3

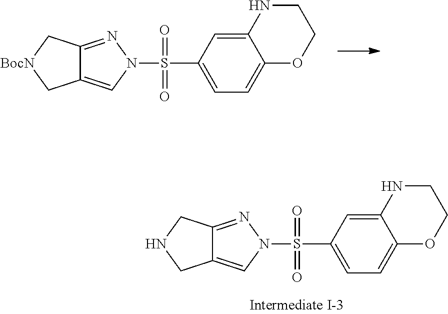 [SMILES: CC(C)(C)OC(=O)N1Cc2cn(S(=O)(=O)c3ccc4c(c3)NCCO4)nc2C1 ; O=S(=O)(c1ccc2c(c1)NCCO2)n1cc2c(n1)CNC2]

To a mixture of tert-butyl 2-(3,4-dihydro-2H-1,4-benzoxazine-6-sulfonyl)-2H,4H,5H,6H-pyrrolo[3,4-c]pyrazole-5-carboxylate (600.00 mg; 1.48 mol) in 1,4-dioxane (4 mL) and 1,2-dichloroethane (4) was added ZnBr₂ (997.25 mg; 4.43 mol; 3.00 eq.). This mixture was allowed to stir at 60° C. until LCMS indicated reaction completion. The resulting mixture was placed into an ice bath to cool to 0° C., treated with water (10 mL) and aqueous NH₄OH (30%, 4 mL), and extracted with 10% MeOH/CH₂Cl₂ thrice. The organic layers were combined and then washed with water and brine. After removal of the volatiles under reduced pressure, the remaining residue was purified by column chromatography on silica gel eluted with 0-100% solvent A in CH₂Cl₂ (solvent A: 0.1% NH₄OH/10% MeOH/89.9% CH₂Cl₂) to provide the title compound as a white solid (380 mg, 84%). LCMS (ES) [M+1]⁺ m/z 307.2.

### Example 1.4

### Synthesis of 2-(2,3-dihydro-1,4-benzodioxine-6-sulfonyl)-2H,4H,5H,6H-pyrrolo[3,4-c]pyrazole (Intermediate I-4)

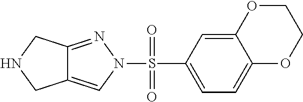 [SMILES: O=S(=O)(c1ccc2c(c1)OCCO2)n1cc2c(n1)CNC2]

Step 1

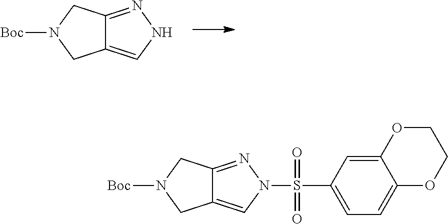 [SMILES: CC(C)(C)OC(=O)N1Cc2c[nH]nc2C1 ; CC(C)(C)OC(=O)N1Cc2cn(S(=O)(=O)c3ccc4c(c3)OCCO4)nc2C1]

To a suspension of sodium hydride (0.80 g; 60%, 20.1 mmol; 1.5 eq.) in dry THF (30 mL) in an ice/water bath under an N₂ atmosphere was added a solution of N-{2H,4H,5H,6H-pyrrolo[3,4-c]pyrazol-5-yl}(tert-butoxy)formamide (3.0 g; 13.40 mmol; 1.00 eq.). The mixture was allowed to stir in the ice bath for 30 min. A solution of 1,3-benzothiazole-6-sulfonyl chloride (3 130.39 mg; 13.40 mmol; 1.00 eq.) in THF (5 mL) was added dropwise over 5 min. The mixture was allowed to stir in the ice bath for 45 min, and at rt for 2 h subsequently. To the resulting mixture under N₂ atmosphere was added aq. NH₄Cl solution (15 mL) and HOAc to adjust the pH to ˜5. The organic layer was separated, and the aqueous layer was back extracted with 10% MeOH/CH₂Cl₂ (15) thrice. The combined organic solutions were washed with water and brine. Removal of the volatiles under reduced pressure provided the desired crude product of tert-butyl 2-(2,3-dihydro-1,4-benzodioxine-6-sulfonyl)-2H,4H,5H,6H-pyrrolo[3,4-c]pyrazole-5-carboxylate as a pale yellow solid (5.2 g, 92%), which was directly used in the next step. LCMS (ES) [M+1]⁺ m/z 408.1

Step 2

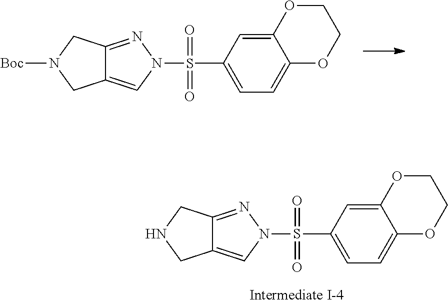 [SMILES: CC(C)(C)OC(=O)N1Cc2cn(S(=O)(=O)c3ccc4c(c3)OCCO4)nc2C1 ; O=S(=O)(c1ccc2c(c1)OCCO2)n1cc2c(n1)CNC2]

The title compound (Intermediate I-4) was made from tert-butyl 2-(2,3-dihydro-1,4-benzodioxine-6-sulfonyl)-2H,4H,5H,6H-pyrrolo[3,4-c]pyrazole-5-carboxylate following a similar procedure as that described in step 3 for the synthesis of 6-{2H,4H,5H,6H-pyrrolo[3,4-c]pyrazole-2-sulfonyl}-3,4-dihydro-2H-1,4-benzoxazine (Intermediate I-3). The crude product was purified by silica gel column with 0-10% MeOH/CH₂Cl₂ (0.1% NH₄OH) as the eluent to provide the title product as a white solid. LCMS (ES) [M+1]⁺ m/z 308.0; ¹H NMR (DMSO-d₆) δ: 8.01 (d, J=1.3 Hz, 1H), 7.40 (dd, J=8.6, 2.3 Hz, 1H), 7.35 (d, J=2.3 Hz, 1H), 7.07 (d, J=8.6 Hz, 1H), 4.40-4.21 (m, 4H), 3.80-3.66 (m, 4H).

### Example 1.5

### Synthesis of 2-(1-benzofuran-5-sulfonyl)-2H,4H,5H,6H-pyrrolo[3,4-c]pyrazole (Intermediate I-5)

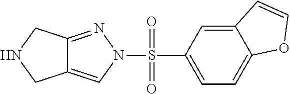 [SMILES: O=S(=O)(c1ccc2occc2c1)n1cc2c(n1)CNC2]

Step 1

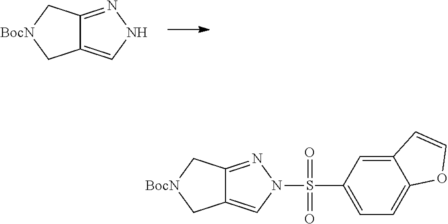 [SMILES: CC(C)(C)OC(=O)N1Cc2c[nH]nc2C1 ; CC(C)(C)OC(=O)N1Cc2cn(S(=O)(=O)c3ccc4occc4c3)nc2C1]

Into a 100-mL 3-necked round-bottom flask, was placed tert-butyl 2H,4H,6H-pyrrolo[3,4-c]pyrazole-5-carboxylate (1.80 g, 8.60 mmol, 1.00 eq.), THF (50.00 mL). This was followed by the addition of NaH (0.41 g, 10.25 mmol, 1.19 eq., 60%) at 0° C. and stirred for 30 min. To this was added 1-benzofuran-5-sulfonyl chloride (1.86 g, 8.60 mmol, 1.00 eq.) at 0° C. and the resulting solution was stirred for 2 h at 0° C. The reaction was then quenched by the addition of 100 mL of water. The resulting solution was extracted with 3×100 mL of ethyl acetate and the organic layers were combined and concentrated under vacuum. The residue was applied onto a silica gel column and eluted with ethyl acetate/petroleum ether (1:5). This resulted in 2.70 g (80.60%) of tert-butyl 2-(1-benzofuran-5-sulfonyl)-4H,6H-pyrrolo[3,4-c]pyrazole-5-carboxylate as a light yellow solid. LCMS (ES, m/z): [M+H]⁺: 390.

Step 2

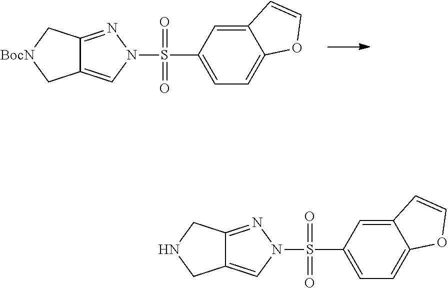 [SMILES: CC(C)(C)OC(=O)N1Cc2cn(S(=O)(=O)c3ccc4occc4c3)nc2C1 ; O=S(=O)(c1ccc2occc2c1)n1cc2c(n1)CNC2]

Into a 100-mL round-bottom flask, was placed tert-butyl 2-(1-benzofuran-5-sulfonyl)-4H,6H-pyrrolo[3,4-c]pyrazole-5-carboxylate (1.00 g, 2.56 mmol, 1.00 eq.), DCM (50.00 mL), and ZnBr2 (1.73 g, 7.68 mmol, 2.99 eq.). The resulting solution was stirred for 12 h at 25° C. Then, it was concentrated under vacuum and the residue was quenched by the addition of 100 mL of water. The resulting solution was extracted with 3×100 mL of ethyl acetate/THF=5:1 and the organic layers were combined, washed with 3×100 ml of brine, dried over anhydrous sodium sulfate, and concentrated under vacuum. This resulted in 700 mg (94.22%) of 2-(1-benzofuran-5-sulfonyl)-4H,5H,6H-pyrrolo[3,4-c]pyrazole (Intermediate-I-5) as a light yellow solid. LCMS (ES, m/z): [M+H]⁺: 290.

### Example 1.6

### Synthesis of 6-{2H,4H,5H,6H-pyrrolo[3,4-c]pyrazole-2-sulfonyl}-1H-indazole (Intermediate I-6)

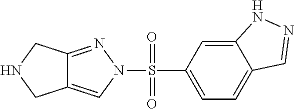 [SMILES: O=S(=O)(c1ccc2cn[nH]c2c1)n1cc2c(n1)CNC2]

Step 1

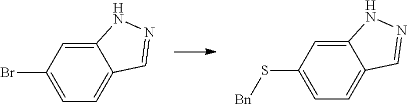 [SMILES: Brc1ccc2cn[nH]c2c1 ; c1ccc(CSc2ccc3cn[nH]c3c2)cc1]

To a solution of 6-bromo-1H-indazole (10.00 g, 50.752 mmol, 1.00 eq.) and benzyl mercaptan (12.61 g, 101.505 mmol, 2 eq.) in dioxane (200.00 mL, 2360.817 mmol, 46.52 eq.) was added DIEA (19.68 g, 152.257 mmol, 3 eq.), xantphos (2.94 g, 5.075 mmol, 0.1 eq.), and Pd₂(dba)₃ (2.32 g, 2.538 mmol, 0.05 eq.). After stirring for 16 h at 100° C. under a nitrogen atmosphere, the resulting mixture was concentrated under reduced pressure. The residue was purified by silica gel column chromatography eluting with PE/THF (1:1) to afford 6-(benzylsulfanyl)-1H-indazole (9.8 g, 80.35%) as a yellow solid. LCMS (ES) [M+1]⁺ m/z: 241

Step 2

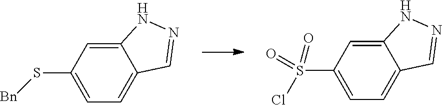 [SMILES: c1ccc(CSc2ccc3cn[nH]c3c2)cc1 ; O=S(=O)(Cl)c1ccc2cn[nH]c2c1]

To a stirred solution of 6-(benzylsulfanyl)-1H-indazole (5 g, 20.806 mmol, 1.00 eq.) in AcOH (45.00 mL, 785.319 mmol, 37.75 eq.) and H₂O (15.00 mL, 832.626 mmol, 40.02 eq.) was added NCS (8.33 g, 0.000 mmol, 3.00 eq.) in at 0° C. under air atmosphere and the resulting mixture was stirred for 2 h at 0° C. under air atmosphere. The reaction mixture was then extracted with EtOAc (3×100 mL). The combined organic layers were washed with water (3×100 mL), dried over anhydrous Na₂SO₄, and concentrated under reduced pressure. The residue was purified by silica gel column chromatography eluting with PE/EtOAc (3:1) to afford 1H-indazole-6-sulfonyl chloride (3.2 g, 71.00%) as a white solid. LCMS (ES) [M+1]⁺ m/z: 217.

Step 3

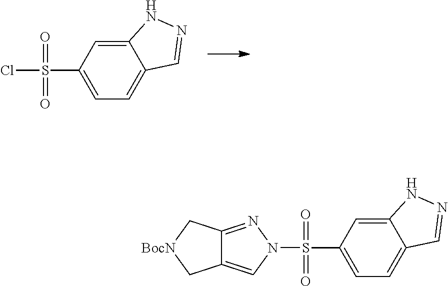 [SMILES: O=S(=O)(Cl)c1ccc2cn[nH]c2c1 ; CC(C)(C)OC(=O)N1Cc2cn(S(=O)(=O)c3ccc4cn[nH]c4c3)nc2C1]

To a solution of tert-butyl 2H,4H,6H-pyrrolo[3,4-c]pyrazole-5-carboxylate (2.66 g, 12.712 mmol, 1.10 eq.) in THF (50.00 mL, 617.150 mmol, 53.48 eq.) was added NaH (0.60 g, 15.002 mmol, 1.30 eq., 60% in oil)) at 0° C. and this mixture was stirred for 30 min. To the above mixture was added 1H-indazole-6-sulfonyl chloride (2.50 g, 11.540 mmol, 1.00 eq.) at 0° C. and the resulting mixture was stirred for an additional 1 h at 0° C. The reaction was then quenched with HOAc (15 mL) at 0° C. and extracted with EtOAc (3×100 mL). The combined organic layers were washed with water (1×100 mL), dried over anhydrous Na₂SO₄, and concentrated under reduced pressure. The residue was purified by silica gel column chromatography eluting with PE/EtOAc (1:1) to afford tert-butyl 2-(1H-indazole-6-sulfonyl)-4H,6H-pyrrolo[3,4-c]pyrazole-5-carboxylate (1.5 g, 33.38%) as a white solid. LCMS (ES) [M+1]⁺ m/z: 390.

Step 4

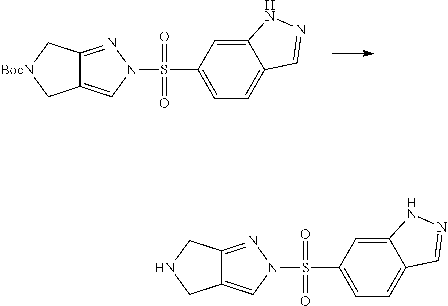 [SMILES: CC(C)(C)OC(=O)N1Cc2cn(S(=O)(=O)c3ccc4cn[nH]c4c3)nc2C1 ; O=S(=O)(c1ccc2cn[nH]c2c1)n1cc2c(n1)CNC2]

To a stirred solution of tert-butyl 2-(1H-indazole-6-sulfonyl)-4H,6H-pyrrolo[3,4-c]pyrazole-5-carboxylate (3.00 g, 7.704 mmol, 1.00 eq.) and 2.6-lutidine (3.30 g, 30.814 mmol, 4 eq.) in DCM (30.00 mL, 471.901 mmol, 61.26 eq.) was added TMSOTf (5.14 g, 23.111 mmol, 3 eq.) dropwise at 0° C. under an air atmosphere. The resulting mixture was stirred for 1 h at 0-25° C. under an air atmosphere. The reaction was then quenched with MeOH at room temperature and the precipitated solids were collected by filtration and washed with MeOH (1×10 mL).The solid was dried to afford the title compound (Intermediate-I-6) as a white solid (1.6 g, 70%). LCMS (ES) [M+1]⁺ m/z: 290.

### Example 1.7

### Synthesis of 2-{2H,3H-[1,4] dioxino[2,3-b]pyridine-7-sulfonyl}-2H,4H,5H,6H-pyrrolo[3,4-c]pyrazole (Intermediate I-8)

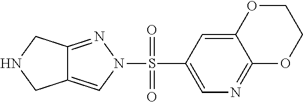 [SMILES: O=S(=O)(c1cnc2c(c1)OCCO2)n1cc2c(n1)CNC2]

Step 1

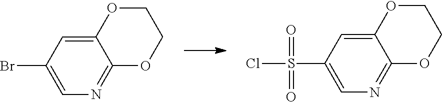 [SMILES: Brc1cnc2c(c1)OCCO2 ; O=S(=O)(Cl)c1cnc2c(c1)OCCO2]

Into a 100-mL 3-necked round-bottom flask purged and maintained with an atmosphere of nitrogen, was placed a solution of n-BuLi in hexane (2.5 M, 2.00 mL, 0.54 eq.) and n-Bu₂Mg in heptanes (1.0 M, 4.80 mL, 0.53 eq.). The resulting solution was stirred for 10 min at room temperature followed by the dropwise addition of a solution of 7-bromo-2,3-dihydro-[1,4]dioxino[2,3-b]pyridine (2.00 g, 9.258 mmol, 1.00 eq.) in THF (16.00 mL) at −10° C., over 10 min. The resulting solution was stirred for 1 h at −10° C., then it was slowly added to a solution of sulfonyl chloride (16 mL) at −10° C. The resulting solution was stirred for 0.5 h at −10° C. The reaction was then quenched by the addition of 50 mL of saturated ammonium chloride solution at 0° C., extracted with 3×50 mL of dichloromethane, and washed with 2×50 ml of brine. The mixture was then dried over anhydrous sodium sulfate and concentrated under vacuum. This resulted in 1.6 g crude of 2,3-dihydro-[1,4]dioxino[2,3-b]pyridine-7-sulfonyl chloride as a light yellow oil. LCMS (ES) [M+1]⁺ m/z: 236.

Step 2

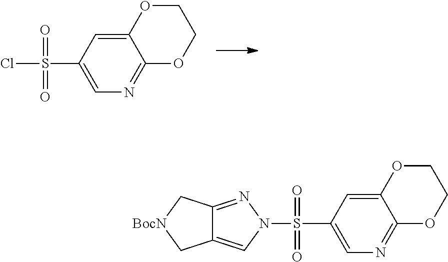 [SMILES: O=S(=O)(Cl)c1cnc2c(c1)OCCO2 ; CC(C)(C)OC(=O)N1Cc2cn(S(=O)(=O)c3cnc4c(c3)OCCO4)nc2C1]

Into a 100-mL 3-necked round-bottom flask purged and maintained with an atmosphere of nitrogen, was placed tert-butyl 2H,4H,6H-pyrrolo[3,4-c]pyrazole-5-carboxylate (2.22 g, 10.609 mmol, 1.00 eq.) in THF (30.00 mL), followed by the addition of NaH (0.55 g, 13.792 mmol, 1.3 eq., 60% in mineral oil) at 0° C. The resulting solution was stirred for 1 h at 0° C. then 2H, 3H-[1,4]dioxino[2,3-b]pyridine-7-sulfonyl chloride (2.50 g, 10.609 mmol, 1.00 eq.) in THF was added at 0° C. The resulting solution was stirred for 2 h at room temperature. The reaction was then quenched by the addition of 80 mL of NH₄Cl (aq), extracted with 2×100 mL of dichloromethane, washed with 2×100 ml of brine, dried over anhydrous sodium sulfate, and concentrated under vacuum. The residue was applied onto a silica gel column and eluted with ethyl acetate/petroleum ether (1:1). This resulted in 2.4 g (55.39%) of tert-butyl 2-((2,3-dihydro-[1,4]dioxino[2,3-b]pyridin-7-yl)sulfonyl)-2,6-dihydropyrrolo[3,4-c]pyrazole-5(4H)-carboxylate as a white solid. LCMS (ES) [M+1]+ m/z: 409.

Step 3

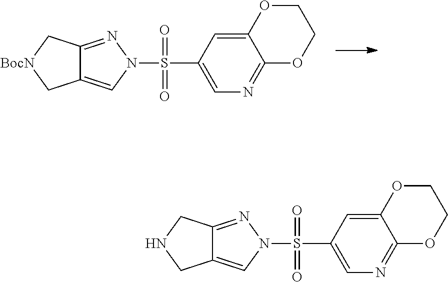 [SMILES: CC(C)(C)OC(=O)N1Cc2cn(S(=O)(=O)c3cnc4c(c3)OCCO4)nc2C1 ; O=S(=O)(c1cnc2c(c1)OCCO2)n1cc2c(n1)CNC2]

Into a 100-mL round-bottom flask, was placed tert-butyl 2-((2,3-dihydro-[1,4]dioxino[2,3-b]pyridin-7-yl)sulfonyl)-2,6-dihydropyrrolo[3,4-c]pyrazole-5(4H)-carboxylate (2.40 g, 5.876 mmol, 1.00 eq.), DCM (30.00 mL), ZnBr2 (13.23 g, 58.743 mmol, 10.00 eq.), and the resulting solution was stirred for 16 h at room temperature. The reaction mixture was then concentrated and the residue was applied onto a silica gel column and eluted with dichloromethane/methanol (10:1). This resulted in 1.6 g (88.32%) of 7-((5,6-dihydropyrrolo[3,4-c]pyrazol-2(4H)-yl)sulfonyl)-2,3-dihydro-[1,4]dioxino[2,3-b]pyridine (Intermediate-I-8) as a white solid. LCMS (ES) [M+1]+ m/z: 309.

### Example 1.8

### Synthesis of 2-(4-methoxybenzenesulfonyl)-2H,4H,5H,6H-pyrrolo[3,4-c]pyrazole (Intermediate I-9)

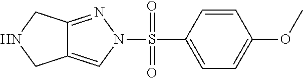 [SMILES: COc1ccc(S(=O)(=O)n2cc3c(n2)CNC3)cc1]

Step 1

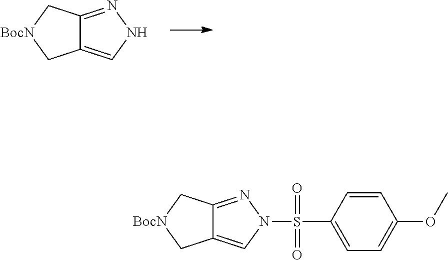 [SMILES: CC(C)(C)OC(=O)N1Cc2c[nH]nc2C1 ; COc1ccc(S(=O)(=O)n2cc3c(n2)CN(C(=O)OC(C)(C)C)C3)cc1]

Into a 250-mL 3-necked round-bottom flask, was placed tert-butyl 2H,4H,6H-pyrrolo[3,4-c]pyrazole-5-carboxylate (2.00 g, 9.60 mmol, 1.00 eq.) in THF (30.00 mL), followed by the addition of NaH (0.30 g, 12.48 mmol, 1.30 eq.) at 0° C. To this was added 4-methoxybenzenesulfonyl chloride (2.18 g, 10.55 mmol, 1.10 eq.) dropwise with stirring at 0° C. and the resulting mixture was stirred for 2 h at room temperature. The reaction was then quenched by the addition of 2 mL of HOAc, diluted with 30 mL of Sat. NH₄Cl, and extracted with 3×30 mL of dichloromethane. The organic layers were combined, dried over anhydrous sodium sulfate, and concentrated. This resulted in 4 g of crude tert-butyl 2-(4-methoxybenzenesulfonyl)-4H,6H-pyrrolo[3,4-c]pyrazole-5-carboxylate as a yellow solid. LCMS (ES) [M+1]⁺ m/z: 380.

Step 2

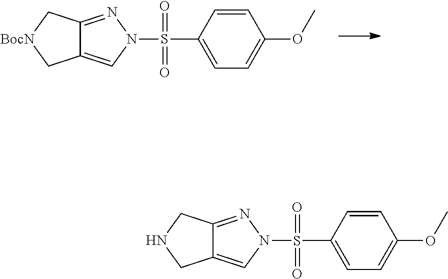 [SMILES: COc1ccc(S(=O)(=O)n2cc3c(n2)CN(C(=O)OC(C)(C)C)C3)cc1 ; COc1ccc(S(=O)(=O)n2cc3c(n2)CNC3)cc1]

Into a 100-mL round-bottom flask, was placed tert-butyl 2-(4-methoxybenzenesulfonyl)-4H,6H-pyrrolo[3,4-c]pyrazole-5-carboxylate (4 g, 10.54 mmol, 1.00 eq.), DCM (20.00 mL) and HCl (gas) in 1,4-dioxane (13.18 mL, 52.72 mmol, 5.00 eq.), and the resulting solution was stirred for 1.5 h at room temperature. The solids were collected by filtration and the pH value of the solution was adjusted to 8 with Sat. NaHCO₃. The resulting solution was extracted with 3×30 mL of dichloromethane and the organic layers were combined, dried over anhydrous sodium sulfate, and concentrated. This resulted in 700 mg (23.77%) of 2-(4-methoxybenzenesulfonyl)-4H,5H,6H-pyrrolo[3,4-c]pyrazole (Intermediate I-9) as an orange solid. LCMS (ES) [M+1]⁺ m/z: 280.

### Example 1.9

### Synthesis of (2R,3R)-3-hydroxy-2-phenylbutanoic Acid, (2s,3s)-3-hydroxy-2-phenylbutanoic Acid, (2R,3S)-3-hydroxy-2-phenylbutanoic Acid, and (2S,3R)-3-hydroxy-2-phenylbutanoic Acid (Intermediates II-1, II-2, II-3, and II-4)

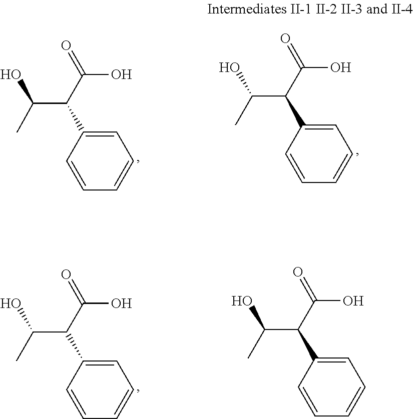

Step 1

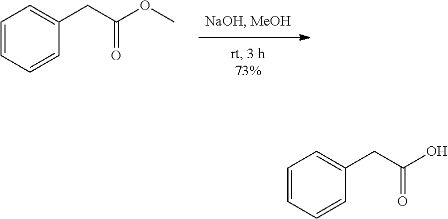

Into a 1-L round-bottom flask, was placed a mixture of methyl phenylacetate (30.0 g, 199 mmol, 1.00 equiv), MeOH (500 mL), water (100 mL), and NaOH (15.9 g, 399 mmol, 2.00 equiv). The resulting mixture was allowed to stir at rt for 3 h and then concentrated under reduced pressure. The resulting solution was diluted with H₂O, and the pH value was adjusted to ˜3 with aq. HCl (2 N). The precipitate was collected by filtration, washed with water, and left on high vacuum over 3 days to provide 2-phenylacetic acid as a white solid (20.0 g, 73.5%). LCMS (ES) [M−1]− m/z 135.1.

Step 2

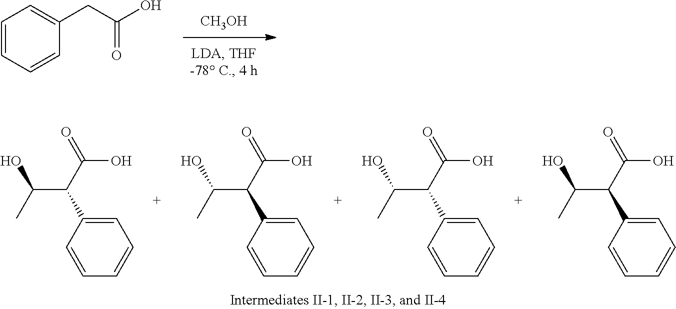

Into a mixture of 2-phenylacetic acid (20.0 g, 146 mmol, 1.00 equiv) in THF (500 mL) at −50° C. was added LDA (221 mL, 2 equiv), followed by acetaldehyde (19.4 g, 440 mmol, 3.00 eq.). The resulting solution was stirred at 50° C. for 1.0 h, warmed to rt, and stirred at rt for an additional 1.0 h. The reaction mixture was then quenched with aq. HCl (200 mL, 2.0 N) and extracted with EtOAc twice (600 mL). The combined organic layers were dried over anhydrous sodium sulfate. After removal of the volatiles under reduced pressure, the remaining residue was subjected to reverse phase preparative MPLC (Prep-C18, 20-45 mM, 120 g, Tianjin Bonna-Agela Technologies; gradient elution of 5% MeCN in water to 33% MeCN in water over a 16 min period, where both solvents contain 0.1% formic acid), to provide two fraction (Fraction I and Fraction II) as follows:

The first fraction (Fraction I, PREP-MPLC RT: 0.83 min), was collected as a colorless oil (4.20 g, 15.9%). LCMS (ES) [M−1]− m/z 179.1;

The second fraction (Fraction II, PREP-MPLC RT: 0.96 min), was collected as a white solid (4.80 g (18.1%). LCMS (ES) [M−1]− m/z 179.1.

The First fraction above (Fraction I, 4.20 g, PREP-MPLC RT: 0.83 min) was subjected to Chiral Prep-HPLC with the following conditions. Column: chiralPAK-AD-3; mobile phase A: n-Hexane (0.1% TFA), mobile phase B: Ethanol; gradient elution of 20% B to 100% B in 10 min. This separation resulted in collections of the following two fractions (fraction II-1, fraction II-2).

Fraction (II-1) was collected as a white solid and assigned as Intermediate II-1 (1.21 g, 28.9%). RT: 6.24 min, ¹H NMR (300 MHz, DMSO-d6, ppm): 7.36-7.21 (m, 5H), 4.15 (dq, J=8.4, 6.1 Hz, 1H), 3.40 (d, J=8.4 Hz, 1H), 1.16 (d, J=6.1 Hz, 3H); LCMS (ES) [M−1]− m/z 179.1.

Fraction (II-2) was collected as a white solid and assigned as Intermediate II-2 (1.20 g, 28.7%). RT: 7.62 min. ¹H NMR (300 MHz, DMSO-d6, ppm): 7.37-7.24 (m, 5H), 4.16 (dq, J=8.3, 6.1 Hz, 1H), 3.40 (d, J=8.4 Hz, 1H), 1.16 (d, J=6.1 Hz, 3H). LCMS (ES) [M−1]− m/z 179.1.

The Second fraction above (Fraction II, 4.80 g, PREP-MPLC RT: 0.96 min) was subjected to Chiral Prep-HPLC with the following conditions. Column: chiralPAK-AD-3; mobile phase A: n-Hexane (0.1% TFA), mobile phase B: Ethanol; gradient elution of 20% B to 100% B in 10 min. This separation resulted in collections of the following two fractions (fraction II-1, fraction II-2):

Fraction (II-1) was collected as a white solid and assigned as Intermediate II-3 (1.3327 g, 27.8%). RT: 5.47 min, ¹H NMR (300 MHz, DMSO-d6, ppm): 12.16 (br, 1H), 7.35-7.23 (m, 5H), 4.93 (br, 1H), 4.17 (dq, J=9.7, 6.2 Hz, 1H), 3.33 (d, J=9.8 Hz, 1H), 0.83 (d, J=6.2 Hz, 4H); LCMS (ES) [M−1]− m/z 179.1.

Fraction (II-2) was collected as a white solid and assigned as Intermediate II-4 (1.2464 g, 26.0%). RT: 7.20 min. ¹H NMR (300 MHz, DMSO-d6, ppm): 12.16 (s, 1H), 7.35-7.23 (m, 5H), 4.92 (br, 1H), 4.17 (dq, J=9.7, 6.2 Hz, 1H), 3.33 (d, J=9.8 Hz, 1H), 0.83 (d, J=6.2 Hz, 3H); LCMS (ES) [M−1]− m/z 179.1.

### Example 1.10

### Synthesis of (R)-3-hydroxy-2-phenylpropanoic Acid and (S)-3-hydroxy-2-phenylpropanoic Acid (Intermediate II-5 and Intermediate II-6)

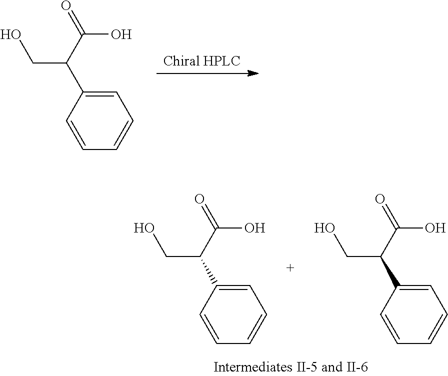 [SMILES: O=C(O)C(CO)c1ccccc1 ; "Chiral HPLC" ; O=C(O)[C@@H](CO)c1ccccc1 ; O=C(O)[C@H](CO)c1ccccc1]

A solution of (±)-3-hydroxy-2-phenylpropanoic acid (3.00 g) in EtOH (20 mL) was subjected to Chiral-Prep-HPLC (Mobile phase A: n-Hexane (0.2% TFA); Mobile phase B: Ethanol; Flow rate: 90 mL/min Column: CHIRALPAK Lux-Amylose-1, 50*250 mm, 10 μm Gradient: 20%-40% B in 17 min; detector UV220 nm) to afford the following two fractions:

The fraction collected at t=2.49 min was assigned as Intermediate II-5 (1.11 g, 37.0%). RT: 2.49 min, ¹H NMR (300 MHz, DMSO-d6) δ 12.36 (br, 1H), 7.38-7.22 (m, 5H), 3.95-3.89 (m, 1H), 3.68-3.52 (m, 2H). LCMS (ES) [M−1]−m/z 165.1.

The fraction collected at t=3.33 min was assigned as Intermediate II-6 (1.11 g, 37.0%). RT: 3.33 min, ¹H NMR (300 MHz, DMSO-d6) δ 12.36 (br, 1H), 7.38-7.22 (m, 5H), 3.96-3.88 (m, 1H), 3.68-3.52 (m, 2H). LCMS (ES) [M−1]− m/z 165.1.

### Example 1.11

### Synthesis of 3-(pyridin-2-yl)oxolane-3-carboxylic Acid (Intermediate II-7)

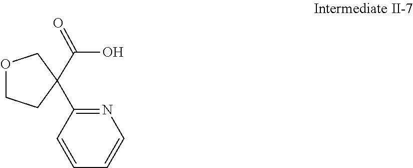 [SMILES: O=C(O)C1(c2ccccn2)CCOC1]

Step 1

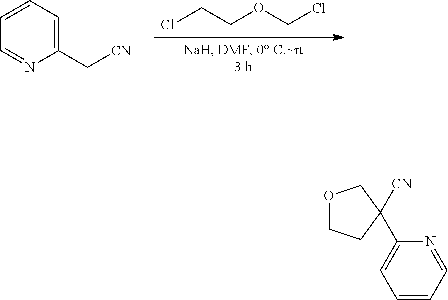

Into a solution of 2-(pyridin-2-yl)acetonitrile (5.0 g, 42.37 mmol, 1.0 equiv) in DMF (70 mL) at 0° C. was added NaH (60% in mineral oil) (1.86 g, 46.61 mmol, 1.1 equiv) in three batches over 10 min. The resulting mixture was allowed to stir in the ice bath for 0.5 h. To the mixture was added 1-chloro-2-(chloromethoxy)ethane (5.42 g, 42.37 mmol, 1.0 equiv) dropwise. After addition, the mixture was allowed to stir at room temperature for 3.0 h, quenched with H₂O (100 mL), and extracted with ethyl acetate (100 mL*2). The combined organic layers were dried over anhydrous sodium sulfate, filtered, and the filtrate was concentrated under reduced pressure. The residue was purified by silica gel column with ethyl acetate/petroleum ether (1:4) to provide 3-(pyridin-2-yl)oxolane-3-carbonitrile as a yellow oil (2.0 g, 76%). LCMS (ESI): [M+H]⁺: 175.

Step 2

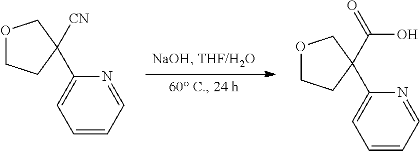 [SMILES: N#CC1(c2ccccn2)CCOC1 ; "60° C., 24 h" ; "NaOH, THF/H2O" ; O=C(O)C1(c2ccccn2)CCOC1]

Into a rt 50-mL round-bottom flask, was placed 3-(pyridin-2-yl)oxolane-3-carbonitrile (1.5 g, 8.62 mmol, 1.0 equiv), THF/H₂O (1:1) (20.0 mL), followed by NaOH (1.0 g, 25.86 mmol, 3.0 equiv). The mixture was stirred for 24 h at 60° C. After removal of THF solvent under reduced pressure, the residue was cooled to 0° C., and the pH value was to adjusted to 5-6 with HCl (3.0 N). After removal of volatiles under reduced pressure, the crude product was purified by Prep-HPLC (conditions: Column, C18-120 g, mobile phase A: H₂O, mobile phase B: CH₃CN, gradient elution of 0-60% B in A over 10 min. Flow rate, 70 mL/min, Detector, 254 nm) to provide a fraction containing the title compound as a white solid (800 mg, purity: 20%), which was used directly without further purification. LCMS (ESI):[M+H]⁺:194.

### Example 1.12

### Synthesis of 3-phenylmorpholine-3-carboxylic Acid (Intermediate II-8)

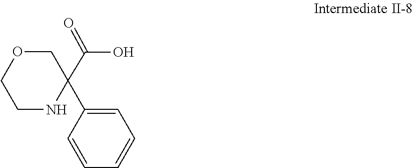 [SMILES: O=C(O)C1(c2ccccc2)COCCN1]

Step 1

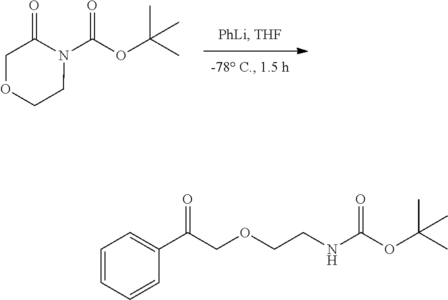 [SMILES: CC(C)(C)OC(=O)N1CCOCC1=O ; CC(C)(C)OC(=O)NCCOCC(=O)c1ccccc1]

Into a −78° C. solution of tert-butyl 3-oxomorpholine-4-carboxylate (8.0 g, 39.75 mmol, 1.00 equiv) in THF (100.0 mL) was added PhLi (21.0 mL, 39.9 mmol, 1.00 equiv) dropwise over 5 min. The resulting solution was stirred for 1.5 h at −78° C. The reaction was then quenched by the addition of 50 mL of NH₄Cl. The resulting solution was extracted with ethyl acetate (3×50 mL). The combined organic layers were dried over anhydrous sodium sulfate and concentrated. The residue was applied onto a silica gel column with ethyl acetate/petroleum ether (1/3). This resulted in tert-butyl N-[2-(2-oxo-2-phenylethoxy)ethyl]carbamate (9.4 g, 84.6%) as a light yellow solid. LCMS (ESI):[M+H]⁺:280.

Step 2

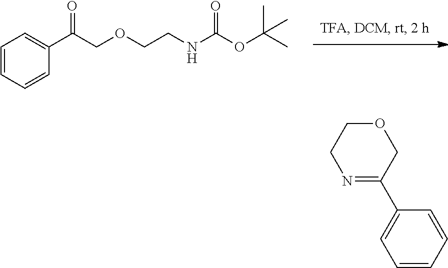 [SMILES: CC(C)(C)OC(=O)NCCOCC(=O)c1ccccc1 ; c1ccc(C2=NCCOC2)cc1]

Into a 250-mL round-bottom flask, was placed tert-butyl N-[2-(2-oxo-2-phenylethoxy)ethyl]carbamate (9.40 g, 33.65 mmol, 1.00 equiv), DCM (90.0 mL), and TFA (30.0 mL). The resulting solution was stirred for 2 hr at room temperature. The resulting mixture was concentrated. This resulted in crude 5-phenyl-3,6-dihydro-2H-oxazine (5.0 g) as a brown oil. LCMS (ESI):[M+H]⁺:162

Step 3

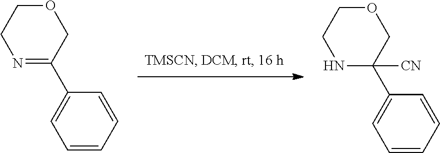 [SMILES: c1ccc(C2=NCCOC2)cc1 ; N#CC1(c2ccccc2)COCCN1]

Into a 100-mL round-bottom flask, was placed 5-phenyl-3,6-dihydro-2H-oxazine (5.00 g, 31.01 mmol, 1.00 eq.), DCM (60.0 mL), and TMSCN (15.39 g, 155.08 mmol, 5.0 equiv). The resulting solution was stirred for 16 hr at room temperature. The resulting mixture was concentrated. The residue was applied onto a silica gel column with dichloromethane/methanol (10/1). This resulted in a crude product of 3-phenylmorpholine-3-carbonitrile (2.5 g) as a brown oil. LCMS (ESI): [M+H]⁺:189

Step 4

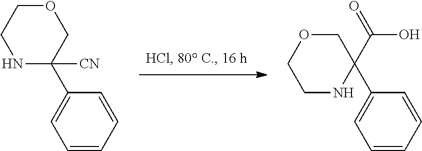 [SMILES: N#CC1(c2ccccc2)COCCN1 ; O=C(O)C1(c2ccccc2)COCCN1]

Into a 100-mL round-bottom flask, was placed 3-phenylmorpholine-3-carbonitrile (2.50 g, 13.28 mmol, 1.00 equiv) and HCl (12 N, 30.0 mL). The resulting solution was stirred for 16 h at 80° C. The resulting solution was diluted with 20 mL of H₂O. The pH value of the solution was adjusted to 7 with aq. NaHCO₃ (10%). The resulting solution was extracted with 50 mL of ethyl acetate and the aqueous layers were combined. The crude product was purified by Flash-Prep-HPLC with the following conditions (IntelFlash-1): Column, C18 silica gel; mobile phase, H₂O/ACN=10/1 increasing to H₂O/ACN=7/1 within 15 min; Detector, UV) to provide the title product of 3-phenylmorpholine-3-carboxylic acid as an off-white solid (1.0 g, 36.3%). LCMS (ESI):[M+H]⁺:208.

### Example 1.13

### Synthesis of (2R,3S)-2-(2-chlorophenyl)-3-hydroxybutanoic Acid, (2S,3R)-2-(2-chlorophenyl)-3-hydroxybutanoic Acid, (2R,3R)-2-(2-chlorophenyl)-3-hydroxybutanoic Acid, and (2S,3S)-2-(2-chlorophenyl)-3-hydroxybutanoic Acid (Intermediate II-9)

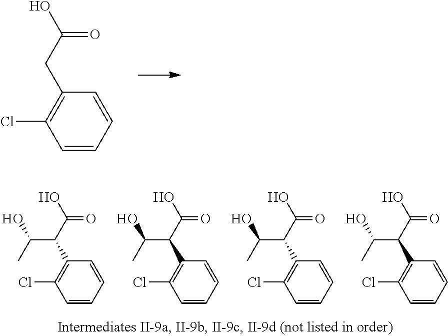

Into a 250-mL 3-necked round-bottom flask purged and maintained with an atmosphere of nitrogen, was placed (2-chlorophenyl)acetic acid (5.00 g, 29.31 mmol, 1.0 eq.) in THF (50 mL) cooled to −78° C., a solution of LDA (4.3 mL, 32.24 mmol, 1.1 eq.) was added dropwise and the reaction was stirred for 2 h at −78° C. A solution of acetaldehyde (2.58 g, 58.62 mmol, 2.0 eq.) was then added dropwise and the resulting solution was stirred for 2 h allowing it to warm from −78° C. to 0° C. The reaction mixture was then quenched with saturated NH₄Cl (20 mL), extracted with EtOAc (50mL×3), dried, and concentrated. The residue was pre-purified by silica gel column chromatography, followed by separation by Pre CHIRAL_HPLC with the following conditions: Column: Lux Amylose-1, 50*250 mm, 10 um; Mobile phase: A=n-Hexanes, B=Ethanol; Flow rate: 90 mL/min; Gradient: 50% B in 50 min; 220 nm. This resulted in the following four products:

The 1^{st} fraction with Retention time=1.894 min in ANAL_SFC: (500 mg, 7.95%); LCMS (ES) [M−1]⁻ m/z: 213. This material was assigned as Intermediate II-9a

The 2^{nd} fraction with Retention time=1.944 min in ANAL_SFC: (500 mg, 7.95%); LCMS (ES) [M−1]⁻ m/z: 213. This material was assigned as Intermediate II-9c

The 3^{rd} fraction with Retention time=2.082 min in ANAL_SFC: (500 mg, 7.95%); LCMS (ES) [M−1]⁻ m/z: 213. This material was assigned as Intermediate II-9b

The 4^{th} fraction with Retention time=2.440 min in ANAL_SFC: (500 mg, 7.95%); LCMS (ES) [M−1]⁻ m/z: 213. This material was assigned as Intermediate II-9d

### Example 1.14

### Synthesis of (S)-2-(2-fluorophenyl)-3-hydroxypropanoic Acid (Intermediate II-10)

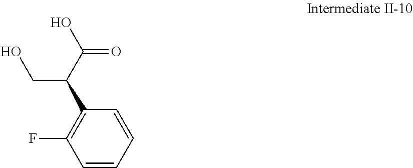 [SMILES: O=C(O)[C@H](CO)c1ccccc1F]

Step 1

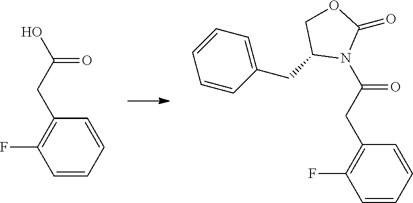 [SMILES: O=C(O)Cc1ccccc1F ; O=C(Cc1ccccc1F)N1C(=O)OC[C@H]1Cc1ccccc1]

Into a 250-mL 3-necked round-bottom flask purged and maintained with an atmosphere of nitrogen, was placed (4R)-4-benzyl-1,3-oxazolidin-2-one (10.00 g, 56.43 mmol, 1.0 eq.) in THF (100 mL). The mixture was cooled to −78° C., and a solution of n-BuLi in hexanes (25.0 mL, 263.27 mmol, 4.6 eq.) was added dropwise. After the reaction was stirred at −78° C. for 1 h, another solution of (2-fluorophenyl)acetyl chloride (10.71 g, 62.07 mmol, 1.1 eq.) in THF (20 mL) was added. The resulting solution was stirred for 2 h while allowing it to warm from −78° C. to 0° C. The reaction was then quenched by the addition of 50 mL of saturated NH₄Cl. The resulting solution was extracted with 3×80 mL of ethyl acetate dried over anhydrous sodium sulfate and concentrated. The residue was applied onto a silica gel column and eluted with ethyl acetate/petroleum ether (1:50 to 1:5). This resulted in 12.5 g (70.69%) of (4R)-4-benzyl-3-[2-(2-fluorophenyl)acetyl]-1,3-oxazolidin-2-one as an off-white solid. LCMS (ES) [M+1]⁺ m/z: 314.

Step 2

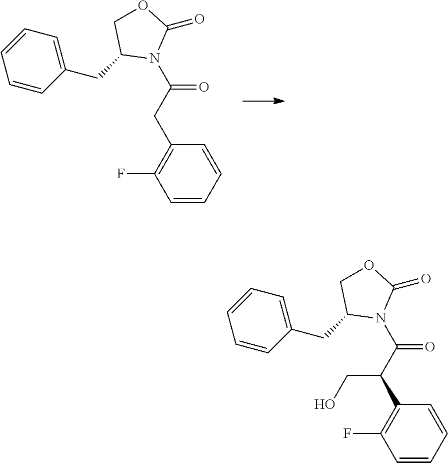 [SMILES: O=C(Cc1ccccc1F)N1C(=O)OC[C@H]1Cc1ccccc1 ; O=C1OC[C@@H](Cc2ccccc2)N1C(=O)[C@H](CO)c1ccccc1F]

Into a 100 mL 3-necked round-bottom flask purged and maintained with an atmosphere of nitrogen, was placed (4R)-4-benzyl-3-[2-(2-fluorophenyl)acetyl]-1,3-oxazolidin-2-one (2.50 g, 7.98 mmol, 1.0 eq.) and DCM (30 mL). After the reaction was cooled to 0° C., a solution of TiCl₄ (1.66 g, 8.78 mmol, 1.1 eq.) in DCM (5 mL) was added dropwise. The reaction was stirred for 5 min, DIEA (1.13 g, 8.78 mmol, 1.1 eq.) was added, and the mixture was stirred at 0° C. for 1 h. Then 1,3,5 trioxane (0.86 g, 9.57 mmol, 1.2 eq.) and TiCl₄ (1.66 g, 8.78 mmol, 1.1 eq.) were added. The resulting solution was stirred for another 2 h at 0° C., then it was quenched with saturated NH₄Cl (10 mL). The mixture was extracted with DCM (50 mL×3) and the combined organic layers were dried and concentrated. The residue was purified by silica gel column chromatography (PE/EA=100:1 to 1:1) to give (4R)-4-benzyl-3-[(2S)-2-(2-fluorophenyl)-3-hydroxypropanoyl]-1,3-oxazolidin-2-one (2 g, 73.00%) as a yellow gum. LCMS (ES) [M+1]⁺ m/z: 344.

Step 3

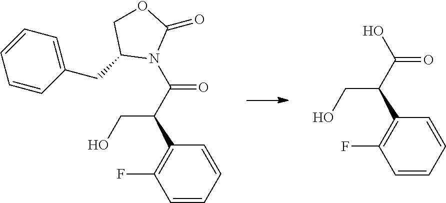 [SMILES: O=C1OC[C@@H](Cc2ccccc2)N1C(=O)[C@H](CO)c1ccccc1F ; O=C(O)[C@H](CO)c1ccccc1F]

Into a 100 mL round-bottom flask, was placed (4R)-4-benzyl-3-[(2S)-2-(2-fluorophenyl)-3-hydroxypropanoyl]-1,3-oxazolidin-2-one (2.00 g, 5.83 mmol, 1.0 eq.) and THF (8 mL) After the reaction was cooled to 0° C., a solution of LiOH.H₂O (0.49 g, 11.65 mmol, 2.0 eq.) in H₂O (2 mL) and H₂O₂ (30%) (0.99 g, 29.12 mmol, 5.0 eq.) were added dropwise. The reaction was stirred at 0° C. for 2 h then it was quenched with saturated Na₂SO₃ (10 mL) and extracted with DCM (50 mL×5). The aqueous mixture was adjusted with HCl (5 N) to pH=3, extracted with 5×30 mL of DCM, dried over anhydrous sodium sulfate and concentrated. The residue was purified by prep. HPLC (C18 silica gel column with Phase A: Water/0.05% TFA, Mobile Phase B: ACN; Flow rate: 1.5 mL/min; Gradient: 5% B to 100% B in 1.2 min, hold 0.6 min). This resulted in (2S)-2-(2-fluorophenyl)-3-hydroxypropanoic acid (0.4 g, 37.29%) as a yellow solid.

CHIRAL_HPLC: Retention time 0.716 min. ¹H-NMR: (300 MHz, DMSO-d₆, ppm): δ 12.5 (br, 1H), 7.42-7.28 (m, 2H), 7.21-7.15 (m, 2H), 4.95 (br, 1H), 3.98-3.90 (m, 2H), 3.69-3.61 (m, 1H); LCMS (ES) [M−1]⁻ m/z: 183.0; Retention time 0.716 min.

### Example 1.15

### Synthesis of (S)-2-(2-fluorophenyl)-3-hydroxypropanoic acid (Intermediate II-11)

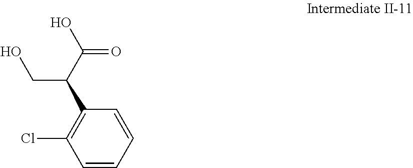 [SMILES: O=C(O)[C@H](CO)c1ccccc1Cl]

Step 1

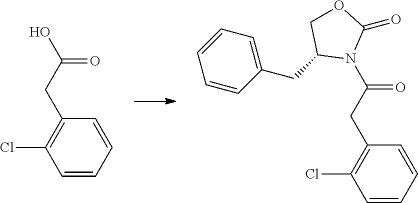 [SMILES: O=C(O)Cc1ccccc1Cl ; O=C(Cc1ccccc1Cl)N1C(=O)OC[C@H]1Cc1ccccc1]

Into a 100-mL 3-necked round-bottom flask purged and maintained with an atmosphere of nitrogen, was placed (4R)-4-benzyl-1,3-oxazolidin-2-one (1.00 g, 5.64 mmol, 1.00 eq.) in THF (15.00 mL) and the mixture was cooled to −78° C. This was followed by the addition of n-butyllithium in hexanes (2.48 mL, 6.20 mmol, 1.10 eq.) dropwise with stirring at −78° C. After 1 h, (2-chlorophenyl)acetyl chloride (1.60 g, 8.46 mmol, 1.50 eq.) was added dropwise with stirring at −78° C. After 1 h, the reaction was quenched by the addition of 3 mL of Sat. NH₄Cl, cooled to room temperature, extracted with 3×20 mL of ethyl acetate, and the organic layers were combined, dried over anhydrous sodium sulfate, and concentrated. The residue was applied onto a silica gel column and eluted with THF/PE (10.6%). This resulted in 1 g (53.73%) of (4R)-4-benzyl-3-[2-(2-chlorophenyl)acetyl]-1,3-oxazolidin-2-one as a yellow oil. LCMS (ES) [M+1]⁺ m/z: 330.

Step 2

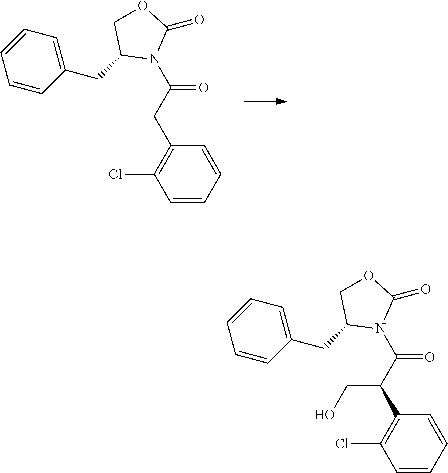 [SMILES: O=C(Cc1ccccc1Cl)N1C(=O)OC[C@H]1Cc1ccccc1 ; O=C1OC[C@@H](Cc2ccccc2)N1C(=O)[C@H](CO)c1ccccc1Cl]

Into a 100-mL 3-necked round-bottom flask, was placed (4R)-4-benzyl-3-[2-(2-chlorophenyl)acetyl]-1,3-oxazolidin-2-one (1.00 g, 3.03 mmol, 1.00 eq.) and DCM (20.00 mL). This was followed by the addition of a solution of TiCl₄ (0.63 g, 3.34 mmol, 1.10 eq.) in DCM (2.00 mL) dropwise with stirring at 0° C. After 5 min, DIEA (0.45 g, 3.48 mmol, 1.15 eq.) was added dropwise with stirring at 0° C. and the resulting solution was stirred for 30 min at 0° C. A solution of trioxane (0.30 g, 3.34 mmol, 1.10 eq.) in DCM (2.00 mL) was then added dropwise with stirring at 0° C. After 5 min, a solution of TiCl₄ (0.63 g, 3.34 mmol, 1.10 eq.) in DCM (2.00 mL) was added dropwise with stirring at 0° C. and the resulting solution was stirred for 1 h at 0° C. The reaction was then quenched by the addition of 5 mL of Sat. NH₄Cl, extracted with 3×20 mL of dichloromethane. The combined organic layers were combined, washed with 20 ml of Sat. NaHCO₃, dried over anhydrous sodium sulfate and concentrated. This resulted in 0.8 g (73.32%) of (4R)-4-benzyl-3-[(2S)-2-(2-chlorophenyl)-3-hydroxypropanoyl]-1,3-oxazolidin-2-one as a yellow oil. LCMS (ES) [M+1]⁺ m/z: 360.

Step 3

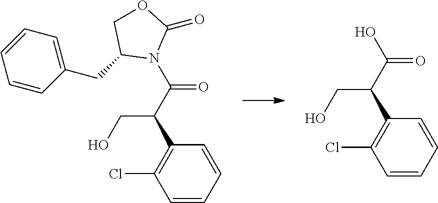 [SMILES: O=C1OC[C@@H](Cc2ccccc2)N1C(=O)[C@H](CO)c1ccccc1Cl ; O=C(O)[C@H](CO)c1ccccc1Cl]

Into a 100-mL 3-necked round-bottom flask, was placed (4R)-4-benzyl-3-[(2S)-2-(2-chlorophenyl)-3-hydroxypropanoyl]-1,3-oxazolidin-2-one (0.80 g, 2.22 mmol, 1.00 eq.) in THF (9.00 mL) and H₂O (2.00 mL). This was followed by the addition of a solution of H₂O₂ (0.38 g, 11.12 mmol, 5.00 eq.) in water (1 mL) dropwise with stirring at 0° C., followed by the addition of a solution of LiOH.H₂O (0.19 g, 4.45 mmol, 2.00 eq.) in water (1 mL) dropwise with stirring at 0° C. The resulting solution was stirred for 2 h at 0° C. then it was quenched by the addition of 3 mL of Sat. NaSO₃. The resulting solution was extracted with 2×10 mL of dichloromethane and the aqueous layers were combined. The pH value of the solution was adjusted to 3 with HCl (1 mol/L) and extracted with 3×20 mL of DCM/MeOH=10/1. The organic layers were combined, dried over anhydrous sodium sulfate, and concentrated. The crude product (0.5 g) was purified by Prep-HPLC with the following conditions: Column, Xbridge Prep C18 OBD Column, 19 cm, 150 mm, 5 um; mobile phase, Water (FA) and CAN (5% Phase B up to 30% in 11 min); Detector, 254. This resulted in 170 mg (38.11%) of (2S)-2-(2-chlorophenyl)-3-hydroxypropanoic acid as an off-white solid. LCMS (ES) [M−1]⁺ m/z: 199.

### Example 1.16

### Synthesis of (2R)-1-[2-(1,3-benzothiazole-6-sulfonyl)-2H,4H,5H,6H-pyrrolo[3,4-c]pyrazol-5-yl]-3-hydroxy-2-phenylpropan-1-one or (2S)-1-[2-(1,3-benzothiazole-6-sulfonyl)-2H,4H,5H,6H-pyrrolo[3,4-c]pyrazol-5-yl]-3-hydroxy-2-phenylpropan-1-one (Compound 1)

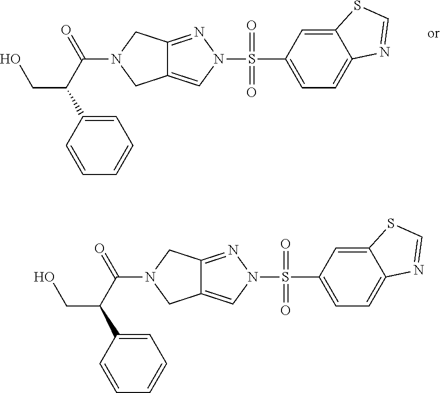 [SMILES: O=C([C@@H](CO)c1ccccc1)N1Cc2cn(S(=O)(=O)c3ccc4ncsc4c3)nc2C1 ; O=C([C@H](CO)c1ccccc1)N1Cc2cn(S(=O)(=O)c3ccc4ncsc4c3)nc2C1]

Into a rt solution of 6-{2H,4H,5H,6H-pyrrolo[3,4-c]pyrazole-2-sulfonyl}-1,3-benzothiazole (Intermediate I-1) (460 mg, 1.5 mmol, 1.0 eq.) and Intermediate II-5 (323 mg, 1.95 mmol, 1.3 eq.) in DMF (5 mL) was added diisopropylethylamine (0.78 mL, 4.5 mmol, 3.00 eq.) followed by HATU (743 mg, 1.95 mmol, 1.3 eq.). The resulting mixture was allowed to stir at rt for 5 h, treated with water (14 mL), and extracted with 10% MeOH/CH₂Cl₂ thrice (6 mL). The organic layers were combined. After removal of the volatiles under reduced pressure, the remaining residue was subjected to reverse phase preparative HPLC (Prep-C18, 20-45M, 120 g, Tianjin Bonna-Agela Technologies; gradient elution of 35% MeCN in water to 60% MeCN in water over a 10 min period, where both solvents contained 0.1% FA) to provide the title product as a white solid. ¹H NMR (300 MHz, DMSO-d₆) δ 9.71 (s, 1H), 9.02 (d, J=2.0 Hz, 1H), 8.34-8.26 (m, 2H), 8.03 (dd, J=8.7, 2.0 Hz, 1H), 7.37-7.17 (m, 5H), 4.90-4.75 (m, 2H), 4.50-4.25 (m, 3H), 4.03-3.92 (m, 2H), 3.48-3.53 (m, 1H). LCMS (ES) [M+1]⁺ m/z 455.0.

### Example 1.17

### Synthesis of (2S)-1-[2-(1,3-benzothiazole-6-sulfonyl)-2H,4H,5H,6H-pyrrolo[3,4-c]pyrazol-5-yl]-3-hydroxy-2-phenylpropan-1-one or (2R)-1-[2-(1,3-benzothiazole-6-sulfonyl)-2H,4H,5H,6H-pyrrolo[3,4-c]pyrazol-5-yl]-3-hydroxy-2-phenylpropan-1-one (Compound 2)

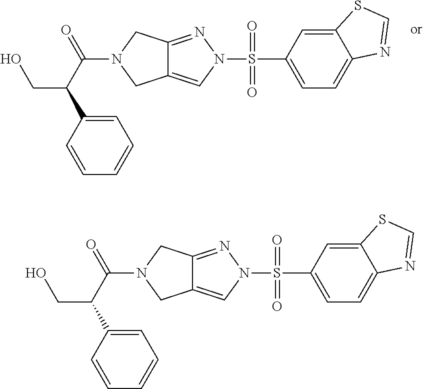 [SMILES: O=C([C@H](CO)c1ccccc1)N1Cc2cn(S(=O)(=O)c3ccc4ncsc4c3)nc2C1 ; O=C([C@@H](CO)c1ccccc1)N1Cc2cn(S(=O)(=O)c3ccc4ncsc4c3)nc2C1]

The title compound was made from 6-{2H,4H,5H,6H-pyrrolo[3,4-c]pyrazole-2-sulfonyl}-1,3-benzothiazole (Intermediate I-1) and Intermediate II-6 following a procedure similar as described for the synthesis of Compound 1. The crude product was purified by reverse phase preparative HPLC (Prep-C18, 20-45M, 120 g, Tianjin Bonna-Agela Technologies; gradient elution of 35% MeCN in water to 60% MeCN in water over a 10 min period, where both solvents contain 0.1% FA) to provide the title product as a white solid. ¹H NMR (300 MHz, DMSO-d₆) δ 9.71 (s, 1H), 9.02 (d, J=2.0 Hz, 1H), 8.34-8.26 (m, 2H), 8.03 (dd, J=8.7, 2.0 Hz, 1H), 7.37-7.17 (m, 5H), 4.90-4.75 (m, 2H), 4.50-4.25 (m, 3H), 4.03-3.92 (m, 2H), 3.45-3.52 (m, 1H). LCMS (ES) [M+1]⁺ m/z 455.0.

### Example 1.18

### Synthesis of (2R,3R)-1-[2-(1,3-benzothiazole-6-sulfonyl)-2H,4H,5H,6H-pyrrolo[3,4-c]pyrazol-5-yl]-3-hydroxy-2-phenylbutan-1-one or (2S,3S)-1-[2-(1,3-benzothiazole-6-sulfonyl)-2H,4H,5H,6H-pyrrolo[3,4-c]pyrazol-5-yl]-3-hydroxy-2-phenylbutan-1-one or (2R,3S)-1-[2-(1,3-benzothiazole-6-sulfonyl)-2H,4H,5H,6H-pyrrolo[3,4-c]pyrazol-5-yl]-3-hydroxy-2-phenylbutan-1-one or (2S,3R)-1-[2-(1,3-benzothiazole-6-sulfonyl)-2H,4H,5H,6H-pyrrolo[3,4-c]pyrazol-5-yl]-3-hydroxy-2-phenylbutan-1-one (Compound 3)

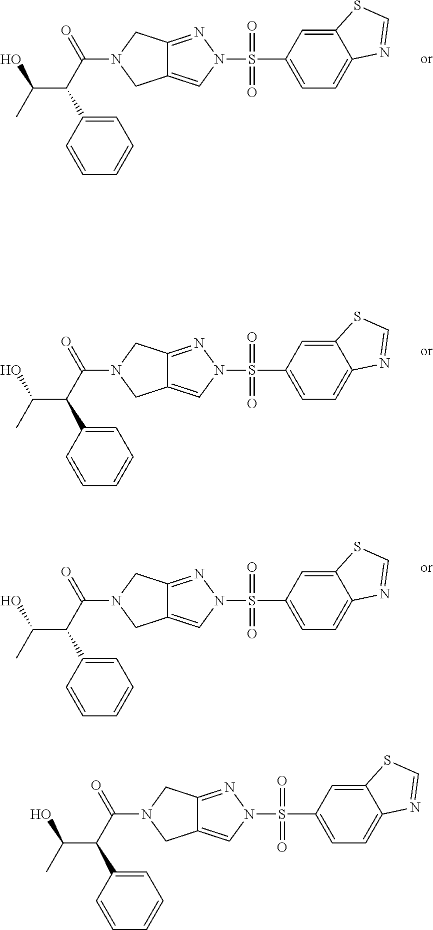 [SMILES: C[C@@H](O)[C@H](C(=O)N1Cc2cn(S(=O)(=O)c3ccc4ncsc4c3)nc2C1)c1ccccc1 ; C[C@H](O)[C@@H](C(=O)N1Cc2cn(S(=O)(=O)c3ccc4ncsc4c3)nc2C1)c1ccccc1 ; C[C@H](O)[C@H](C(=O)N1Cc2cn(S(=O)(=O)c3ccc4ncsc4c3)nc2C1)c1ccccc1 ; C[C@@H](O)[C@@H](C(=O)N1Cc2cn(S(=O)(=O)c3ccc4ncsc4c3)nc2C1)c1ccccc1]

The title compound was made from 6-{2H,4H,5H,6H-pyrrolo[3,4-c]pyrazole-2-sulfonyl}-1,3-benzothiazole (Intermediate I-1) and Intermediate II-1 following a procedure similar as described for the synthesis of Compound 1. The crude product was purified by reverse phase preparative HPLC (Prep-C18, 20-45M, 120 g, Tianjin Bonna-Agela Technologies; gradient elution of 35% MeCN in water to 60% MeCN in water over a 10 min period, where both solvents contained 0.1% FA) to provide the title product as a white solid. ¹H NMR (300 MHz, DMSO-d₆, ppm): δ 9.70 (s, 1H), 9.01 (s, 1H), 8.29 (d, J=8.7 Hz, 2H), 8.03 (d, J=8.8 Hz, 1H), 7.51-6.91 (m, 5H), 4.87 (t, J=15.2 Hz, 1H), 4.50-3.98 (m, 5H), 3.65-3.60 (m, 1H), 1.12 (d, J=6.4 Hz, 3H). LCMS (ES) [M+1]⁺ m/z 469.1.

### Example 1.19

### Synthesis of (2R,3R)-1-[2-(1,3-benzothiazole-6-sulfonyl)-2H,4H,5H,6H-pyrrolo[3,4-c]pyrazol-5-yl]-hydroxy-2-phenylbutan-1-one, or (2S,3S)-1-[2-(1,3-benzothiazole-6-sulfonyl)-2H,4H,5H,6H-pyrrolo[3,4-c]pyrazol-5-yl]-3-hydroxy-2-phenylbutan-1-one, or (2R,3S)-1-[2-(1,3-benzothiazole-6-sulfonyl)-2H,4H,5H,6H-pyrrolo[3,4-c]pyrazol-5yl]-3-hydroxy-2-phenylbutan-1-one, or (2S,3R)-1-[2-(1,3-benzothiazole-6-sulfonyl)-2H,4H,5H,6H-pyrrolo[3,4-c]pyrazol-5-yl]-3-hydroxy-2-phenylbutan-1-one (Compound 4)

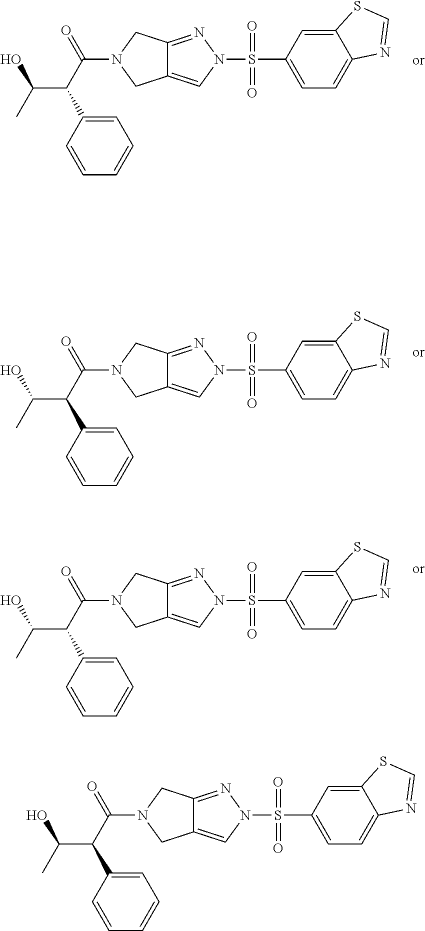 [SMILES: C[C@@H](O)[C@H](C(=O)N1Cc2cn(S(=O)(=O)c3ccc4ncsc4c3)nc2C1)c1ccccc1 ; C[C@H](O)[C@@H](C(=O)N1Cc2cn(S(=O)(=O)c3ccc4ncsc4c3)nc2C1)c1ccccc1 ; C[C@H](O)[C@H](C(=O)N1Cc2cn(S(=O)(=O)c3ccc4ncsc4c3)nc2C1)c1ccccc1 ; C[C@@H](O)[C@@H](C(=O)N1Cc2cn(S(=O)(=O)c3ccc4ncsc4c3)nc2C1)c1ccccc1]

The title compound was made from 6-{2H,4H,5H,6H-pyrrolo[3,4-c]pyrazole-2-sulfonyl}-1,3-benzothiazole (Intermediate I-1) and Intermediate II-2 following a procedure similar as described for the synthesis of Compound 1. The crude product was purified by reverse phase preparative HPLC (Prep-C18, 20-45M, 120 g, Tianjin Bonna-Agela Technologies; gradient elution of 35% MeCN in water to 60% MeCN in water over a 10 min period, where both solvents contained 0.1% FA) to provide the title product as a white solid. ¹H NMR (300 MHz, DMSO-d₆, ppm): δ 9.70 (s, 1H), 9.01 (s, 1H), 8.29 (d, J=8.7 Hz, 2H), 8.03 (d, J=8.8 Hz, 1H), 7.40-7.16 (m, 5H), 4.88 (t, J=15.1 Hz, 1H), 4.57-4.13 (m, 5H), 3.70-3.60 (m, 1H), 1.12 (d, J=6.0 Hz, 3H). LCMS (ES) [M+1]⁺ m/z 469.1.

### Example 1.20

### Synthesis of (2R,3R)-1-[2-(1,3-benzothiazole-6-sulfonyl)-2H,4H,5H,6H-pyrrolo[3,4-c]pyrazol-5-yl]-3-hydroxy-2-phenylbutan-1-one, or (2S,3S)-1-[2-(1,3benzothiazole-6-sulfonyl)-2H,4H,5H,6H-pyrrolo[3,4-c]pyrazol-5-yl]-3-hydroxy-2-phenylbutan-1-one, or (2R,3S)-1-[2-(1,3-benzothiazole-6-sulfonyl)-2H,4H,5H,6H-pyrrolo[3,4-c]pyrazol-5-3-hydroxy-2-phenylbutan-1-one, or (2S,3R)-1-[2-(1,3-benzothiazole-6-sulfonyl)-2H,4H,5H,6H-pyrrolo[3,4-c]pyrazol-5-yl]-3-hydroxy-2-phenylbutan-1-one (Compound 5)

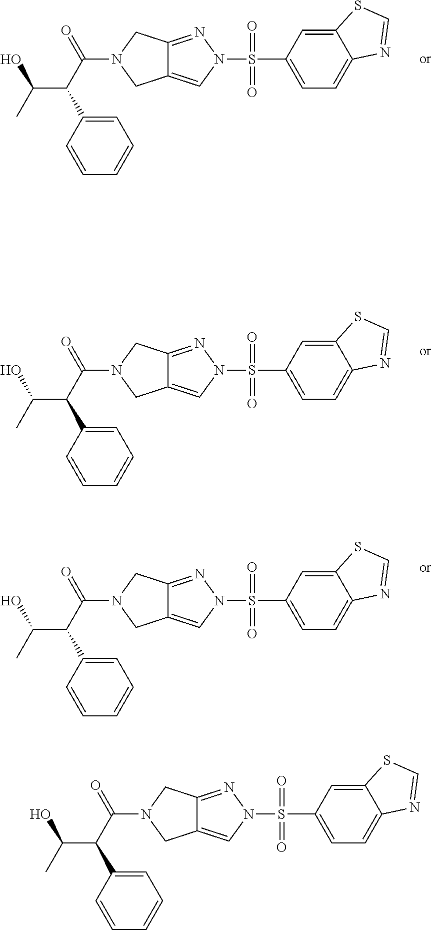 [SMILES: C[C@@H](O)[C@H](C(=O)N1Cc2cn(S(=O)(=O)c3ccc4ncsc4c3)nc2C1)c1ccccc1 ; C[C@H](O)[C@@H](C(=O)N1Cc2cn(S(=O)(=O)c3ccc4ncsc4c3)nc2C1)c1ccccc1 ; C[C@H](O)[C@H](C(=O)N1Cc2cn(S(=O)(=O)c3ccc4ncsc4c3)nc2C1)c1ccccc1 ; C[C@@H](O)[C@@H](C(=O)N1Cc2cn(S(=O)(=O)c3ccc4ncsc4c3)nc2C1)c1ccccc1]

The title compound was made from 6-{2H,4H,5H,6H-pyrrolo[3,4-c]pyrazole-2-sulfonyl}-1,3-benzothiazole (Intermediate I-1) and Intermediate II-3 following a procedure similar as described for the synthesis of Compound 1. The crude product was purified by reverse phase preparative HPLC (Prep-C18, 20-45M, 120 g, Tianjin Bonna-Agela Technologies; gradient elution of 35% MeCN in water to 60% MeCN in water over a 10 min period, where both solvents contain 0.1% FA) to provide the title product as a white solid. ¹H NMR (300 MHz, DMSO-d₆, ppm): 9.70 (s, 1H), 9.00 (s, 1H), 8.45-8.15 (m, 2H), 8.15-7.91 (m, 1H), 7.51-6.91 (m, 5H), 5.12-4.65 (m, 2H), 4.63-4.15 (m, 4H), 3.71-3.58 (m, 1H), 1.00-0.65 (m, 3H). LCMS (ES) [M+1]⁺ m/z 469.1.

### Example 1.21

### Synthesis of (2R,3R)-1-[2-(1,3-benzothiazole-6-sulfonyl)-2H,4H,5H,6H pyrrolo[3,4-c]pyrazol-5-yl]-3-hydroxy-2-phenylbutan-1-one, or (2S,3S)-1-[2-(1,3-benzothiazole-6-sulfonyl)-2H,4H,5H,6H-pyrrolo[3,4-c]pyrazol-5-yl]-3-hydroxy-2-phenylbutan-1-one, or (2R,3S)-1-[2-(1,3-benzothiazole-6-sulfonyl)-2H,4H,5H,6H-pyrrolo[3,4-c]pyrazol-5-yl]-3-hydroxy-2-phenylbutan-1-one, (2S,3R)-1-[2-(1,3-benzothiazole-6-sulfonyl)-2H,4H,5H,6H-pyrrolo[3,4-c]pyrazol-5-yl]-3-hydroxy-2-phenylbutan-1-one (Compound 6)

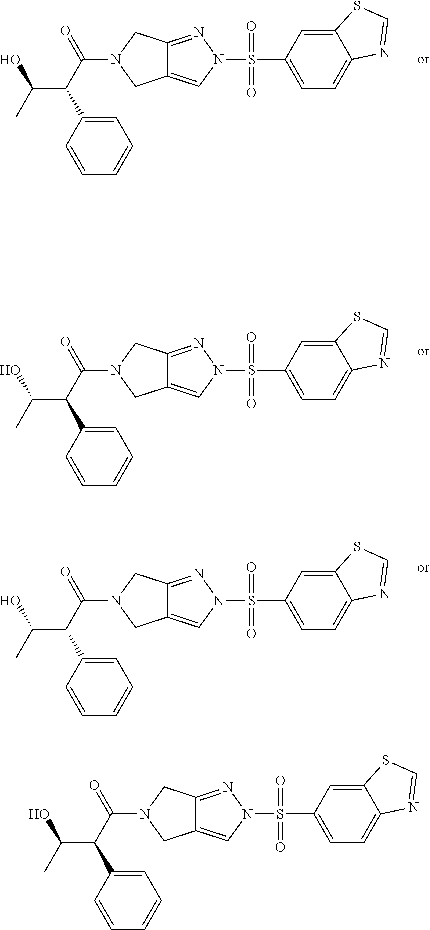 [SMILES: C[C@@H](O)[C@H](C(=O)N1Cc2cn(S(=O)(=O)c3ccc4ncsc4c3)nc2C1)c1ccccc1 ; C[C@H](O)[C@@H](C(=O)N1Cc2cn(S(=O)(=O)c3ccc4ncsc4c3)nc2C1)c1ccccc1 ; C[C@H](O)[C@H](C(=O)N1Cc2cn(S(=O)(=O)c3ccc4ncsc4c3)nc2C1)c1ccccc1 ; C[C@@H](O)[C@@H](C(=O)N1Cc2cn(S(=O)(=O)c3ccc4ncsc4c3)nc2C1)c1ccccc1]

The title compound was made from 6-{2H,4H,5H,6H-pyrrolo[3,4-c]pyrazole-2-sulfonyl}-1,3-benzothiazole (Intermediate I-1) and Intermediate II-4 following a procedure similar as described for the synthesis of Compound 1. The crude product was purified by reverse phase preparative HPLC (Prep-C18, 20-45M, 120 g, Tianjin Bonna-Agela Technologies; gradient elution of 35% MeCN in water to 60% MeCN in water over a 10 min period, where both solvents contained 0.1% FA) to provide the title product as a white solid. ¹H NMR (300 MHz, DMSO-d₆, ppm): δ 9.70 (s, 1H), 9.01 (d, J=2.0 Hz, 1H), 8.30 (d, J=9.0 Hz, 2H), 8.03 (dd, J=8.7, 2.0 Hz, 1H), 7.39-7.17 (m, 5H), 5.00-4.80 (m, 2H), 4.52-4.16 (m, 4H), 3.67 (t, J=8.4 Hz, 1H), 0.84 (d, J=6.2 Hz, 3H). LCMS (ES) [M+1]⁺ m/z 469.1.

### Example 1.22

### Synthesis of (2R)-1-{2-[4-(difluoromethoxy)benzenesulfonyl]-2H,4H,5H,6H-pyrrolo[3,4-c]pyrazol-5-yl}-3-hydroxy-2-phenylpropan-1-one, or (2S)-1-{2-[4-(difluoromethoxy)benzenesulfonyl]-2H,4H,5H,6H-pyrrolo[3,4-c]pyrazol-5-yl}-3-hydroxy-2-phenylpropan-1-one (Compound 7)

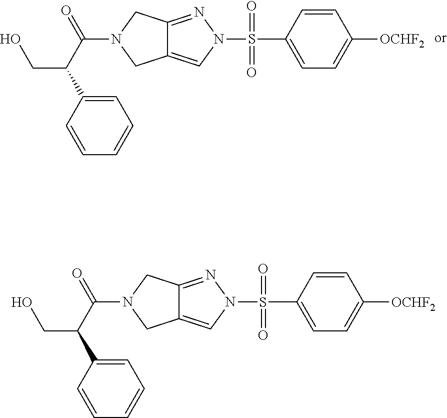 [SMILES: O=C([C@@H](CO)c1ccccc1)N1Cc2cn(S(=O)(=O)c3ccc(OC(F)F)cc3)nc2C1 ; O=C([C@H](CO)c1ccccc1)N1Cc2cn(S(=O)(=O)c3ccc(OC(F)F)cc3)nc2C1]

The title compound was made from 2-[4-(difluoromethoxy)benzenesulfonyl]-2H,4H,5H,6H-pyrrolo[3,4-c]pyrazole (Intermediate I-2) and Intermediate II-5 following a procedure similar as described for the synthesis of Compound 1. The crude product was purified by reverse phase preparative HPLC (Prep-C18, 20-45M, 120 g, Tianjin Bonna-Agela Technologies; gradient elution of 35% MeCN in water to 60% MeCN in water over a 10 min period, where both solvents contained 0.1% FA) to provide the title product as a white solid. ¹H NMR (300 MHz, DMSO-d₆) δ 8.26 (s, 1H), 8.06-8.00 (m, 2H), 7.68-7.15 (m, 8H), 4.91-4.85 (m, 1H), 4.50-4.26 (m, 3H), 4.01-3.96 (m, 2H), 3.58-3.48 (m, 1H). LCMS (ES) [M+1]⁺ m/z 464.0.

### Example 1.23

### Synthesis of (2R)-1-{2-[4-(difluoromethoxy)benzenesulfonyl]-2H,4H,5H,6H-pyrrolo[3,4-c]pyrazol-5-yl}-3-hydroxy-2-phenylpropan-1-one, or (2S)-1-{2-[4-(difluoromethoxy)benzenesulfonyl]-2H,4H,5H,6H-pyrrolo[3,4-c]pyrazol-5-yl}-3-hydroxy-2-phenylpropan-1-one (Compound 8)

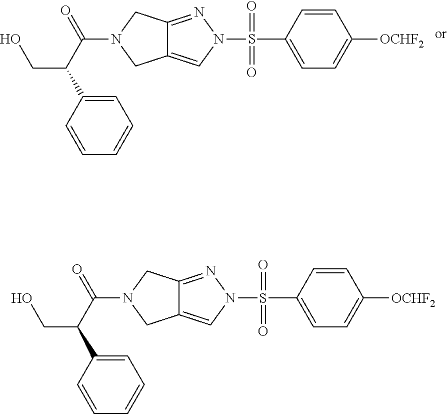 [SMILES: O=C([C@@H](CO)c1ccccc1)N1Cc2cn(S(=O)(=O)c3ccc(OC(F)F)cc3)nc2C1 ; O=C([C@H](CO)c1ccccc1)N1Cc2cn(S(=O)(=O)c3ccc(OC(F)F)cc3)nc2C1]

The title compound was made from 2-[4-(difluoromethoxy)benzenesulfonyl]-2H,4H,5H,6H-pyrrolo[3,4-c]pyrazole (Intermediate I-2) and Intermediate II-6 following a procedure similar as described for the synthesis of Compound 1. The crude product was purified by reverse phase preparative HPLC (Prep-C18, 20-45M, 120 g, Tianjin Bonna-Agela Technologies; gradient elution of 35% MeCN in water to 60% MeCN in water over a 10 min period, where both solvents contained 0.1% FA) to provide the title product as a white solid. ¹H NMR (300 MHz, DMSO-d₆) δ 8.26 (d, J=1.2 Hz, 1H), 8.03 (d, J=8.7 Hz, 2H), 7.68-7.15 (m, 8H), 4.90-4.84 (m, 1H), 4.50-4.26 (m, 3H), 4.01-3.97 (m, 2H), 3.58-3.48 (m, 1H). LCMS (ES) [M+1]⁺ m/z 464.0.

### Example 1.24

### Synthesis of (2R,3R)-1-{2-[4-(difluoromethoxy)benzenesulfonyl]-2H,4H,5H,6H-pyrrolo[3,4-c]pyrazol-5-yl}-3-hydroxy-2-phenylbutan-1-one, or (2R,3S)-1-{2-[4-(difluoromethoxy)benzenesulfonyl]-2H,4H,5H,6H-pyrrolo[3,4-c]pyrazol-5-yl}-3-hydroxy-2-phenylbutan-1-one; or (2S,3S)-1-{2-[4-(difluoromethoxy)benzenesulfonyl]-2H,4H,5H,6H-pyrrolo[3,4-c]pyrazol-5-yl}-3-hydroxy-2-phenylbutan-1-one, or (2S,3R)-1-{2-[4-(difluoromethoxy)benzenesulfonyl]-2H,4H,5H,6H-pyrrolo[3,4-c]pyrazol-5-yl}-3-hydroxy-2-phenylbutan-1-one (Compound 9)

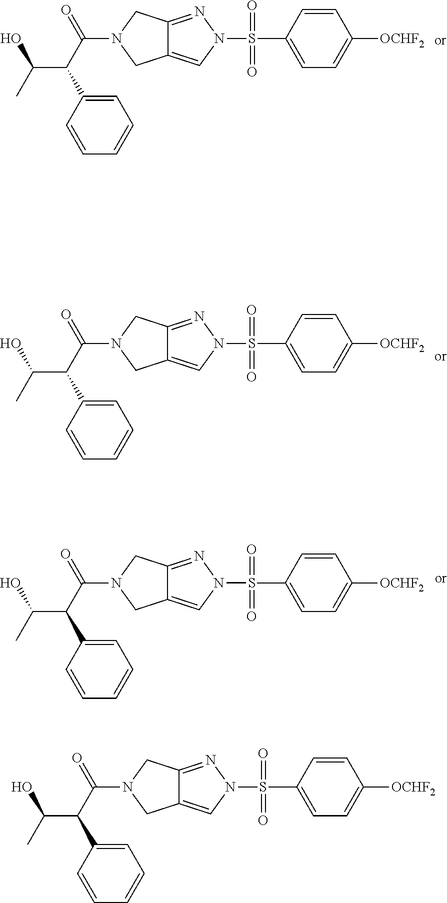 [SMILES: C[C@@H](O)[C@H](C(=O)N1Cc2cn(S(=O)(=O)c3ccc(OC(F)F)cc3)nc2C1)c1ccccc1 ; C[C@H](O)[C@H](C(=O)N1Cc2cn(S(=O)(=O)c3ccc(OC(F)F)cc3)nc2C1)c1ccccc1 ; C[C@H](O)[C@@H](C(=O)N1Cc2cn(S(=O)(=O)c3ccc(OC(F)F)cc3)nc2C1)c1ccccc1 ; C[C@@H](O)[C@@H](C(=O)N1Cc2cn(S(=O)(=O)c3ccc(OC(F)F)cc3)nc2C1)c1ccccc1]

The title compound was made from 2[4-(difluoromethoxy)benzenesulfonyl]-2H,4H,5H,6H-pyrrolo[3,4-c]pyrazole (Intermediate I-2) and Intermediate II-1 following a procedure similar as described for the synthesis of Compound 1. The crude product was purified by reverse phase preparative HPLC (Column, SunFire Prep C18 OBD Column, 19×150 mm, 5 um; mobile phase, phase A: H₂O (0.1% FA); phase B: CH₃CN (40% CH₃CN up to 60% CH₃CN in 10 min) to provide the title product as a white solid. 1H NMR (300 MHz, DMSO-d₆, ppm): δ 8.25 (d, J=1.2 Hz, 1H), 8.03 (d, J=9.0 Hz, 1H), 7.66-7.17 (m, 8H), 4.95-4.83 (m, 1H), 4.71-4.60 (m, 1H), 4.51-4.23 (m, 3H), 4.20-4.05 (m, 1H), 3.75-3.58 (m, 1H), 1.13 (d, J=6.1 Hz, 3H). LCMS (ES) [M+1]+ m/z 478.2.

### Example 1.25

### Synthesis of (2R,3R)-1-{2-[4-(difluoromethoxy)benzenesulfonyl]-2H,4H,5H,6H-pyrrolo[3,4-c]pyrazol-5-yl}-3-hydroxy-2-phenylbutan-1-one, or (2R,3S)-1-{2-[4-(difluoromethoxy)benzenesulfonyl]-2H,4H,5H,6H-pyrrolo[3,4-c]pyrazol-5-yl}-3-hydroxy-2-phenylbutan-1-one; or (2S,3S)-1-{2-[4-(difluoromethoxy)benzenesulfonyl]-2H,4H,5H,6H-pyrrolo[3,4-c]pyrazol-5-yl}-3-hydroxy-2-phenylbutan-1-one, or (2S,3R)-1-{2-[4-(difluoromethoxy)benzenesulfonyl]-2H,4H,5H,6H-pyrrolo[3,4-c]pyrazol-5-yl}-3-hydroxy-2-phenylbutan-1-one (Compound 10)

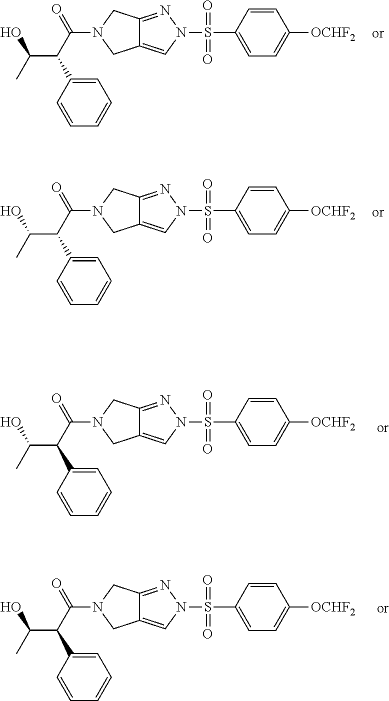 [SMILES: C[C@@H](O)[C@H](C(=O)N1Cc2cn(S(=O)(=O)c3ccc(OC(F)F)cc3)nc2C1)c1ccccc1 ; C[C@H](O)[C@H](C(=O)N1Cc2cn(S(=O)(=O)c3ccc(OC(F)F)cc3)nc2C1)c1ccccc1 ; C[C@H](O)[C@@H](C(=O)N1Cc2cn(S(=O)(=O)c3ccc(OC(F)F)cc3)nc2C1)c1ccccc1 ; C[C@@H](O)[C@@H](C(=O)N1Cc2cn(S(=O)(=O)c3ccc(OC(F)F)cc3)nc2C1)c1ccccc1]

The title compound was made from 2-[4-(difluoromethoxy)benzenesulfonyl]-2H,4H,5H,6H-pyrrolo[3,4-c]pyrazole (Intermediate I-2) and Intermediate II-3 following a procedure similar as described for the synthesis of Compound 1. The crude product was purified by reverse phase preparative HPLC (Column, SunFire Prep C18 OBD Column, 19×150 mm, 5 um; mobile phase, phase A: H₂O (0.1% FA); phase B: CH₃CN (40% CH₃CN up to 60% CH₃CN in 10 min) to provide the title product as a white solid. 1H NMR (300 MHz, D MSO-d₆, ppm): δ 8.27 (d, J=9.5 Hz, 1H), 8.03 (d, J=8.9 Hz, 2H), 7.66-7.17 (m, 8H), 4.95-4.23 (m, 6H), 3.71-3.68 (m, 1H), 0.85 (dd, J=6.2, 1.8 Hz, 3H). LCMS (ES) [M+1]+ m/z 478.2.

### Example 1.26

### Synthesis of (2R,3R)-1-{2-[4-(difluoromethoxy)benzenesulfonyl]-2H,4H,5H,6H-pyrrolo[3,4-c]pyrazol-5-yl}-3-hydroxy-2-phenylbutan-1-one, or (2R,3S)-1-{2-[4-(difluoromethoxy)benzenesulfonyl]-2H,4H,5H,6H-pyrrolo[3,4-c]pyrazol-5-yl}-3-hydroxy-2-phenylbutan-1-one; or (2S,3S)-1-{2-[4-(difluoromethoxy)benzenesulfonyl]-2H,4H,5H,6H-pyrrolo[3,4-c]pyrazol-5-yl}-3-hydroxy-2-phenylbutan-1-one, or (2S,3R)-1-{2-[4-(difluoromethoxy)benzenesulfonyl]-2H,4H,5H,6H-pyrrolo[3,4-c]pyrazol-5-yl}-3-hydroxy-2-phenylbutan-1-one (Compound 11)

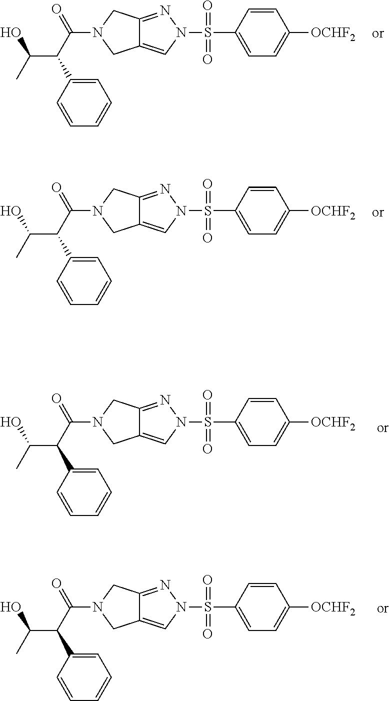 [SMILES: C[C@@H](O)[C@H](C(=O)N1Cc2cn(S(=O)(=O)c3ccc(OC(F)F)cc3)nc2C1)c1ccccc1 ; C[C@H](O)[C@H](C(=O)N1Cc2cn(S(=O)(=O)c3ccc(OC(F)F)cc3)nc2C1)c1ccccc1 ; C[C@H](O)[C@@H](C(=O)N1Cc2cn(S(=O)(=O)c3ccc(OC(F)F)cc3)nc2C1)c1ccccc1 ; C[C@@H](O)[C@@H](C(=O)N1Cc2cn(S(=O)(=O)c3ccc(OC(F)F)cc3)nc2C1)c1ccccc1]

The title compound was made from 2-[4-(difluoromethoxy)benzenesulfonyl]-2H,4H,5H,6H-pyrrolo[3,4-c]pyrazole (Intermediate I-2) and Intermediate II-2 following a procedure similar as described for the synthesis of Compound 1. The crude product was purified by reverse phase preparative HPLC (Column, SunFire Prep C18 OBD Column, 19×150 mm, 5 um; mobile phase, phase A: H₂O (0.1% FA); phase B: CH₃CN (40% CH₃CN up to 60% CH₃CN in 10 min) to provide the title product as a white solid. ¹H NMR (300 MHz, DMSO-d₆, ppm): δ8.27 (d, J=9.6 Hz, 1H), 8.03 (d, J=8.9 Hz, 2H), 7.66-7.17 (m, 8H), 4.95-4.82 (m, 2H), 4.58-4.13 (m, 4H), 3.75-3.62 (m, 1H), 0.85 (d, J=6.3 Hz, 3H). LCMS (ES) [M+1]⁺ m/z 478.2.

### Example 1.27

### Synthesis of 6-{[2-(1,3-benzothiazole-6-sulfonyl)-2H,4H,5H,6H-pyrrolo[3,4-c]pyrazol-5-yl]sulfonyl}-3,4-dihydro-2H-1,4-benzoxazine (Compound 12)

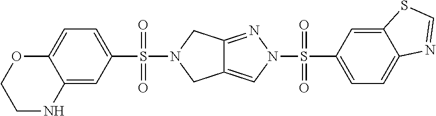 [SMILES: O=S(=O)(c1ccc2c(c1)NCCO2)N1Cc2cn(S(=O)(=O)c3ccc4ncsc4c3)nc2C1]

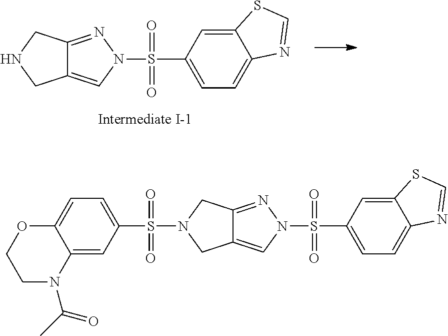 [SMILES: O=S(=O)(c1ccc2ncsc2c1)n1cc2c(n1)CNC2 ; CC(=O)N1CCOc2ccc(S(=O)(=O)N3Cc4cn(S(=O)(=O)c5ccc6ncsc6c5)nc4C3)cc21]

Step 1

A solution of 6-{2H,4H,5H,6H-pyrrolo[3,4-c]pyrazole-2-sulfonyl}-1,3-benzothiazole (Intermediate I-1) (280 mg, 0.75 mmol, 1.0 eq.) and 4-acetyl-3,4-dihydro-2H-benzo[b][1,4]oxazine-6-sulfonyl chloride (230 mg, 1.1 mmol, 0.83 eq) in a mixed solvent of DMF (4 mL) and pyridine (2.0 mL) was allowed to stir at rt overnight. The resulting mixture was treated with water (5 mL) and extracted with 10% MeOH/CH₂Cl₂ thrice (3 mL). The organic layers were combined. After removal of the volatiles under reduced pressure, the resulting crude product of 1-(6-{[2-(1,3-benzothiazole-6-sulfonyl)-2H,4H,5H,6H-pyrrolo[3,4-c]pyrazol-5-yl]sulfonyl}-3,4-dihydro-2H-1,4-benzoxazin-4-yl)ethan-1-one was left on high vacuum overnight before being used directly without purification.

Step 2

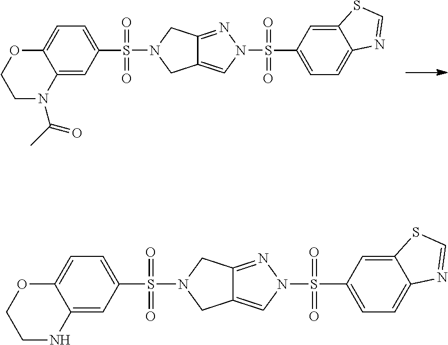 [SMILES: CC(=O)N1CCOc2ccc(S(=O)(=O)N3Cc4cn(S(=O)(=O)c5ccc6ncsc6c5)nc4C3)cc21 ; O=S(=O)(c1ccc2c(c1)NCCO2)N1Cc2cn(S(=O)(=O)c3ccc4ncsc4c3)nc2C1]

To a solution of crude 1-(6-{[2-(1,3-benzothiazole-6-sulfonyl)-2H,4H,5H,6H-pyrrolo[3,4-c]pyrazol-5-yl]sulfonyl}-3,4-dihydro-2H-1,4-benzoxazin-4-yl)ethan-1-one (390 mg, 0.71 mmol) in THF (4 mL) in an ice bath was added LiBH4 (55 mg, 2.5 mmol, 3.5 eq.). The resulting mixture was allowed to warm in the ice bath to rt and then stirred at rt for 5 hr. To the mixture was added water (8 mL) and aq. HCl (1.0 N) to pH=2.0, and extracted with 10% MeOH/CH₂Cl₂ thrice. The organic layers were combined. After removal of the volatiles under reduced pressure, the residue was purified by column chromatography on silica gel eluted with 0-100% solvent A in CH₂Cl₂ (solvent A: 0.1% NH₄OH/10% MeOH/89.9% CH₂Cl₂) to provide the title compound as a brown solid (155 mg, 44%). LCMS (ES) [M+1]⁺ m/z 503.8.

### Example 1.28

### Synthesis of 3-amino-1-[2-(1,3-benzothiazole-6-sulfonyl)-2H,4H,5H,6H-pyrrolo[3,4-c]pyrazol-5-yl]-2-phenylpropan-1-one (Compound 13)

Step 1

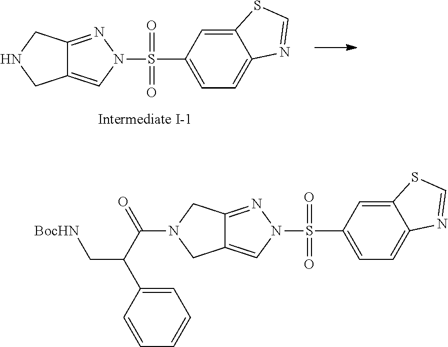 [SMILES: O=S(=O)(c1ccc2ncsc2c1)n1cc2c(n1)CNC2 ; CC(C)(C)OC(=O)NCC(C(=O)N1Cc2cn(S(=O)(=O)c3ccc4ncsc4c3)nc2C1)c1ccccc1]

Into a rt solution of 6-{2H,4H,5H,6H-pyrrolo[3,4-c]pyrazole-2-sulfonyl}-1,3-benzothiazole (Intermediate I-1) (280 mg, 0.75 mmol, 1.0 eq.) and 3-((tert-butoxycarbonyl)amino)-2-phenylpropanoic acid (262 mg, 0.99 mmol, 1.3 eq.) in DMF (3 mL) was added diisopropylethylamine (0.40 mL, 2.8 mmol, 3.00 eq.) followed by HATU (379 mg, 0.99 mmol, 1.3 eq.). The resulting mixture was allowed to stir at rt for 10 h, treated with water (8 mL) and extracted with 10% MeOH/CH₂Cl₂ thrice (3 mL). The organic layers were combined. After removal of the volatiles under reduced pressure, the remaining residue was purified by column chromatography on silica gel eluted with 0-100% solvent A in CH₂Cl₂ (solvent A: 0.1% NH₄OH/10% MeOH/89.9% CH₂Cl₂) provide tert-butyl N-{3-[2-(1,3-benzothiazole-6-sulfonyl)-2H,4H,5H,6H-pyrrolo[3,4-c]pyrazol-5-yl]-3-oxo-2-phenylpropyl}carbamate as a brown solid (232 mg, 56%). LCMS (ES) [M+1]⁺ m/z 554.5.

Step 2

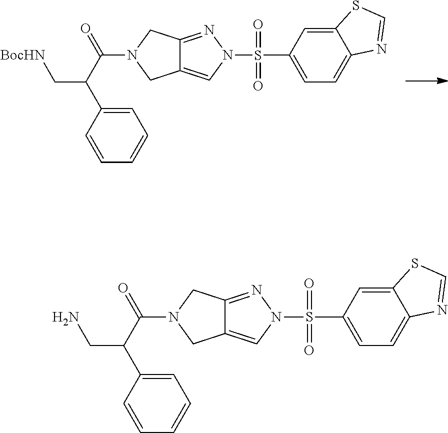 [SMILES: CC(C)(C)OC(=O)NCC(C(=O)N1Cc2cn(S(=O)(=O)c3ccc4ncsc4c3)nc2C1)c1ccccc1 ; NCC(C(=O)N1Cc2cn(S(=O)(=O)c3ccc4ncsc4c3)nc2C1)c1ccccc1]

Into a rt solution of tert-butyl N-{3-[2-(1,3-benzothiazole-6-sulfonyl)-2H,4H,5H,6H-pyrrolo[3,4-c]pyrazol-5-yl]-3-oxo-2-phenylpropyl}carbamate (232 mg, 0.42 mmol, 1.0 eq.) in ClCH₂CH₂Cl (5 mL) was added anhydrous ZnBr₂ (479 mg, 2.10 mmol, 5.00 eq.). The resulting mixture was allowed to stir at 60° C. for 5 h, cooled to rt, treated with 28% aqueous NH₄OH (5 mL) and water (5 mL), and extracted with 10% MeOH/CH₂Cl₂ thrice (8 mL). The organic layers were combined, and then washed with water and brine. After removal of the volatiles under reduced pressure, the remaining residue was purified by reverse phase preparative HPLC (Prep-C18, 5 μM XBridge column, 19×150 mm, Waters; gradient elution of 10-90% MeCN in water over a 20 min period, where both solvents contained 0.1% formic acid) to provide the title product as a white solid (88.3 mg, 46%). ¹H NMR (400 MHz, DMSO-d₆) δ 9.68 (s, 1H), 8.99 (s, 1H), 8.33 (s, 1H), 8.30-8.19 (m, 2H), 8.00 (dd, J=8.7, 2.0 Hz, 1H), 7.40-7.14 (m, 5H), 4.83 (dd, J=22.4, 14.3 Hz, 1H), 4.53-4.28 (m, 2H), 4.21-3.99 (m, 2H), 3.22 (ddd, J=12.2, 9.0, 2.8 Hz, 1H), 2.80 (dd, J=12.5, 4.9 Hz, 1H). LCMS (ES) [M+1]⁺ m/z 454.3.

### Example 1.29

### Synthesis of (2S)-2-amino-1[2-(1,3-benzothiazole-6-sulfonyl)-2H,4H,5H,6H-pyrrolo[3,4-c]pyrazol-5-yl]-2-phenylethan-1-one (Compound 14)

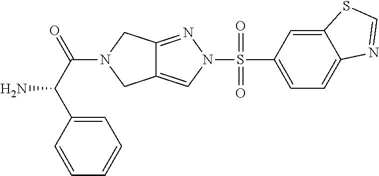 [SMILES: N[C@H](C(=O)N1Cc2cn(S(=O)(=O)c3ccc4ncsc4c3)nc2C1)c1ccccc1]

The title compound was made from 6-{2H,4H,5H,6H-pyrrolo[3,4-c]pyrazole-2-sulfonyl}-1,3-benzothiazole (Intermediate I-1) and (S)-2-((tert-butoxycarbonyl)amino)-2-phenylacetic acid following a 2-step procedure similar as described for the synthesis of (2R)-2-amino-1-[2-(1,3-benzothiazole-6-sulfonyl)-2H,4H,5H,6H-pyrrolo[3,4-c]pyrazol-5-yl]-2-phenylethan-1-one (Compound 15). The crude final product was purified by reverse phase preparative HPLC (Prep-C18, 5 μM XBridge column, 19×150 mm, Waters; gradient elution of 10-90% MeCN in water over a 20 min period, where both solvents contained 0.1% formic acid) to provide the title product as a pale yellow solid. 1H NMR (400 MHz, DMSO-d₆) δ 9.69 (s, 1H), 8.99 (s, 1H), 8.38-8.12 (m, 3H), 8.01 (dd, J=8.7, 2.0 Hz, 1H), 7.46-7.14 (m, 5H), 4.95-4.65 (m, 2H), 4.54-4.26 (m, 2H), 4.12 (t, J=14.4 Hz, 1H). LCMS (ES) [M+1]+ m/z 440.3.

### Example 1.30

### Synthesis of (2R)-2-amino-1-[2-(1,3-benzothiazole-6-sulfonyl)-2H,4H,5H,6H-pyrrolo[3,4-c]pyrazol-5-yl]-2-phenylethan-1-one (Compound 15)

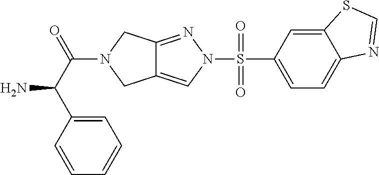 [SMILES: N[C@@H](C(=O)N1Cc2cn(S(=O)(=O)c3ccc4ncsc4c3)nc2C1)c1ccccc1]

Step 1

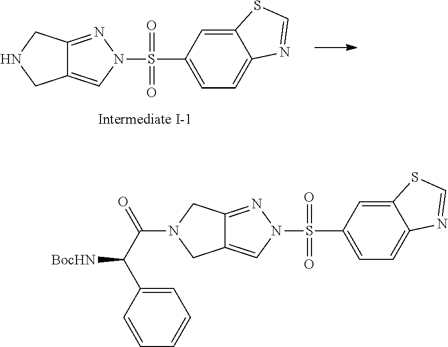 [SMILES: O=S(=O)(c1ccc2ncsc2c1)n1cc2c(n1)CNC2 ; CC(C)(C)OC(=O)N[C@@H](C(=O)N1Cc2cn(S(=O)(=O)c3ccc4ncsc4c3)nc2C1)c1ccccc1]

Into a rt solution of 6-{2H,4H,5H,6H-pyrrolo[3,4-c]pyrazole-2-sulfonyl}-1,3-benzothiazole (Intermediate I-1) (560 mg, 1.5 mmol, 1.0 eq.) and (R)-2-((tert-butoxycarbonyl)amino)-2-phenylacetic acid (512 mg, 2.0 mmol, 1.3 eq.) in DMF (6 mL) was added diisopropylethylamine (0.8 mL, 5.6 mmol, 3.00 eq.) followed by HATU (760 mg, 2.0 mmol, 1.3 eq.). The resulting mixture was allowed to stir at rt for 8 h, treated with water (12 mL) and extracted with 10% MeOH/CH₂Cl₂ thrice (3 mL). The organic layers were combined. After removal of the volatiles under reduced pressure, the remaining residue was purified by column chromatography on silica gel eluted with 0-100% solvent A in CH₂Cl₂ (solvent A: 0.1% NH₄OH/10% MeOH/89.9% CH₂Cl₂) provide tert-butyl N-[(1R)-2-[2-(1,3-benzothiazole-6-sulfonyl)-2H,4H,5H,6H-pyrrolo[3,4-c]pyrazol-5-yl]-2-oxo-1-phenylethyl]carbamate as a dark solid (552 mg, 67%). LCMS (ES) [M+1]⁺ m/z 540.2.

Step 2

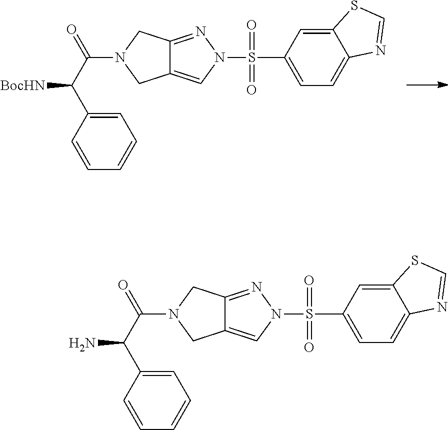 [SMILES: CC(C)(C)OC(=O)N[C@@H](C(=O)N1Cc2cn(S(=O)(=O)c3ccc4ncsc4c3)nc2C1)c1ccccc1 ; N[C@@H](C(=O)N1Cc2cn(S(=O)(=O)c3ccc4ncsc4c3)nc2C1)c1ccccc1]

Into a rt solution of tert-butyl N-[(1R)-2-[2-(1,3-benzothiazole-6-sulfonyl)-2H,4H,5H,6H-pyrrolo[3,4-c]pyrazol-5-yl]-2-oxo-1-phenylethyl]carbamate as a dark solid (552 mg, 1.0 mmoL, 1.0 eq.) in ClCH₂CH₂Cl (12 mL) was added anhydrous ZnBr₂ (960 mg, 4.2 mmol, 5.00 eq.). The resulting mixture was allowed to stir at 65° C. for 5 h, cooled to rt, treated with 28% aqueous NH₄OH (10 mL) and water (10 mL), and extracted with 10% MeOH/CH₂Cl₂ thrice (9 mL). The organic layers were combined, and then washed with water and brine. After removal of the volatiles under reduced pressure, the crude final product was purified by reverse phase preparative HPLC (Prep-C18, 5 μM XBridge column, 19×150 mm, Waters; gradient elution of 10-90% MeCN in water over a 20 min period, where both solvents contain 0.1% formic acid) to provide the title product as a yellow solid. ¹H NMR (400 MHz, DMSO-d₆) δ 9.68 (s, 1H), 8.99 (s, 1H), 8.27 (d, J=9.2 Hz, 3H), 8.01 (dd, J=8.7, 2.0 Hz, 1H), 7.47-7.16 (m, 5H), 4.94-4.76 (m, 2H), 4.53-4.28 (m, 2H), 4.09 (t, J=13.6 Hz, 1H). LCMS (ES) [M+1]⁺ m/z 440.3.

### Example 1.31

### Synthesis of (2R)-2-amino-1-[2-(3,4-dihydro-2H-1,4-benzoxazine-6-sulfonyl)-2H,4H,5H,6H-pyrrolo[3,4-c]pyrazol-5-yl]-2-phenylethan-1-one (Compound 16)

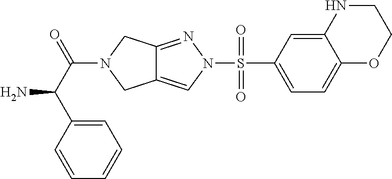 [SMILES: N[C@@H](C(=O)N1Cc2cn(S(=O)(=O)c3ccc4c(c3)NCCO4)nc2C1)c1ccccc1]

The title compound was made from 6-{2H,4H,5H,6H-pyrrolo[3,4-c]pyrazole-2-sulfonyl}-3,4-dihydro-2H-1,4-benzoxazine (Intermediate I-3) and (R)-2-((tert-butoxycarbonyl)amino)-2-phenylacetic acid following a 2-step procedure similar as described for the synthesis of (2R)-2-amino-1-[2-(1,3-benzothiazole-6-sulfonyl)-2H,4H,5H,6H-pyrrolo[3,4-c]pyrazol-5-yl]-2-phenylethan-1-one (Compound 15). The crude final product was purified by reverse phase preparative HPLC (Prep-C18, 5 μM XBridge column, 19×150 mm, Waters; gradient elution of 10-90% MeCN in water over a 20 min period, where both solvents contain 0.1% formic acid) to provide the title product as a yellow solid. LCMS (ES) [M+1]⁺ m/z 440.4.

### Example 1.32

### Synthesis of (2R)-2-amino-1-[2-(1,3-benzothiazole-6-sulfonyl)-2H,4H,5H,6H-pyrrolo[3,4-c]pyrazol-5-yl]-2-(2-fluorophenyl)ethan-1-one (Compound 17)

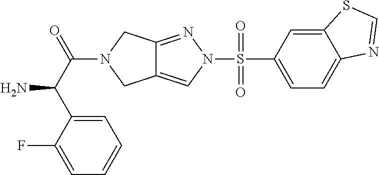 [SMILES: N[C@@H](C(=O)N1Cc2cn(S(=O)(=O)c3ccc4ncsc4c3)nc2C1)c1ccccc1F]

The title compound was made from 6-{2H,4H,5H,6H-pyrrolo[3,4-c]pyrazole-2-sulfonyl}-1,3-benzothiazole (Intermediate I-1) and (R)-2-((tert-butoxycarbonyl)amino)-2-(2-fluorophenyl)acetic acid following a 2-step procedure similar to that for the synthesis of (2R)-2-amino-1-[2-(1,3-benzothiazole-6-sulfonyl)-2H,4H,5H,6H-pyrrolo[3,4-c]pyrazol-5-yl]-2-phenylethan-1-one (Compound 15). The crude product was purified by reverse phase preparative HPLC (Prep-C18, 5 μM XBridge column, 19×150 mm, Waters; gradient elution of 10-90% MeCN in water over a 20 min period, where both solvents contain 0.1% formic acid) to provide the title product as a white solid. LCMS (ES) [M+1]⁺ m/z 440.4.

### Example 1.33

### Synthesis of 6-{[5-(3-phenyloxolane-3-carbonyl)-2H,4H,5H,6H-pyrrolo[3,4-c]pyrazol-2-yl]sulfonyl}-1,3-benzothiazole (Compound 18)

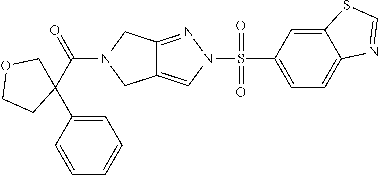 [SMILES: O=C(N1Cc2cn(S(=O)(=O)c3ccc4ncsc4c3)nc2C1)C1(c2ccccc2)CCOC1]

The title compound was made from 6-{2H,4H,5H,6H-pyrrolo[3,4-c]pyrazole-2-sulfonyl}-1,3-benzothiazole (Intermediate I-1) and 3-phenyltetrahydrofuran-3-carboxylic acid following a procedure similar as described for the synthesis of 6-{[5-(3-phenylmorpholine-3-carbonyl)-2H,4H,5H,6H-pyrrolo[3,4-c]pyrazol-2-yl]sulfonyl}-1,3-benzothiazole (Compound 21). The crude product was purified by reverse phase preparative HPLC (Prep-C18, 5 μM XBridge column, 19×150 mm, Waters; gradient elution of 10-90% MeCN in water over a 20 min period, where both solvents contain 0.1% formic acid) to provide the title product as a white solid. ¹H NMR (400 MHz, DMSO-d₆) δ 9.68 (d, J=2.4 Hz, 1H), 8.97 (dd, J=9.7, 1.9 Hz, 1H), 8.31-8.22 (m, 2H), 7.99 (ddd, J=8.6, 6.3, 2.0 Hz, 1H), 7.32 (q, J=7.4 Hz, 2H), 7.28-7.17 (m, 3H), 4.50-4.40 (m, 2H), 4.25 (t, J=8.4 Hz, 1H), 4.03 (dd, J=14.3, 7.7 Hz, 1H), 3.84 (ddd, J=16.9, 14.5, 8.3 Hz, 4H), 2.60 (dq, J=14.5, 7.5 Hz, 1H), 2.42-2.31 (m, 1H). LCMS (ES) [M+1]⁺ m/z 481.3.

### Example 1.34

### Synthesis of 6-({5-[(3S)-3-(pyridin-2-yl)oxolane-3-carbonyl]-2H,4H,5H,6H-pyrrolo[3,4-c]pyrazol-2-yl}sulfonyl)-1,3-benzothiazole or 6-({5-[(3R)-3-(pyridin-2-yl)oxolane-3-carbonyl]-2H,4H,5H,6H-pyrrolo[3,4-c]pyrazol-2-yl}sulfonyl)-1,3-benzothiazole (Compound 19 or Compound 20)

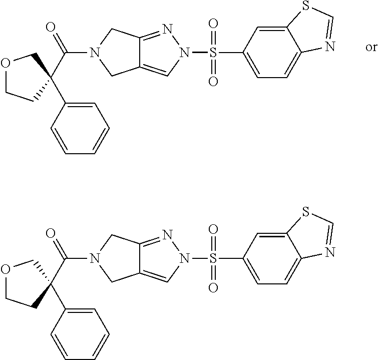 [SMILES: O=C(N1Cc2cn(S(=O)(=O)c3ccc4ncsc4c3)nc2C1)[C@]1(c2ccccc2)CCOC1 ; O=C(N1Cc2cn(S(=O)(=O)c3ccc4ncsc4c3)nc2C1)[C@@]1(c2ccccc2)CCOC1]

The title compounds were made from 6-{2H,4H,5H,6H-pyrrolo[3,4-c]pyrazole-2-sulfonyl}-1,3-benzothiazole (Intermediate I-1) and 3-(pyridin-2-yl)oxolane-3-carboxylic acid (Intermediate II-7) following a procedure similar as described for the synthesis of 6-{[5-(3-phenyloxolane-3-carbonyl)-2H,4H,5H,6H-pyrrolo[3,4-c]pyrazol-2-yl]sulfonyl}-1,3-benzothiazole (Compound 21). The crude product was purified by preparative HPLC (chiral PAK IC-3 column, 20*250 mm, 5 um, Phase A: dichloromethane, Phase B: methanol, Gradient: 0-50% methanol in dichloromethane within 5.5 min, Detector: UV 220 nm.) to provide the following two fractions:

The fraction collected at t=1.60 min was assigned as compound 19. ¹H NMR (300 MHz, DMSO-d₆, ppm): δ 9.71 (s, 1H), 9.01 (dd, J=4.5, 1.8 Hz, 1H), 8.55-8.49 (m, 1H), 8.30 (dd, J=2.4, 8.7 Hz, 1H), 8.27(s, 0.5H), 8.14 (s, 0.5H), 8.04-8.00 (m, 1H), 7.81-7.75 (m, 1H), 7.34-7.25 (m, 2H), 4.46 (d, J=3.6 Hz, 2H), 4.36 (t, J=3.6 Hz, 1H), 4.04-3.98 (m, 2H), 3.87-3.81(m, 2H), 3.73 (dd, J=15.0, 3.6 Hz, 1H), 2.70-2.57 (m, 2H); LCMS (ES) [M+1]⁺ m/z: [M+H]⁺: 482.1.

The fraction collected at t=1.35 min was assigned as compound 20: ^{H NMR (}300 MHz, DMSO-d₆, ppm): δ 9.71 (s, 1H), 9.01 (dd, J=4.5, 1.8 Hz, 1H), 8.55-8.49 (m, 1H), 8.30 (dd, J=2.4, 8.7 Hz, 1H), 8.27(s, 0.5H), 8.14 (s, 0.5H), 8.04-8.00 (m, 1H), 7.81-7.75 (m, 1H), 7.34-7.25 (m, 2H), 4.46 (d, J=3.6 Hz, 2H), 4.36 (t, J=3.6 Hz, 1H), 4.04-3.98 (m, 2H), 3.87-3.81(m, 2H), 3.73 (dd, J=15.0, 3.6 Hz, 1H), 2.70-2.57 (m, 2H); LCMS (ES) [M+1]⁺ m/z: 482.1.

### Example 1.35

### Synthesis of 6-{[5-(3-phenylmorpholine-3-carbonyl)-2H,4H,5H,6H-pyrrolo[3,4-c]pyrazol-2-yl]sulfonyl}-1,3-benzothiazole (Compound 21)

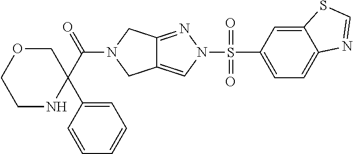 [SMILES: O=C(N1Cc2cn(S(=O)(=O)c3ccc4ncsc4c3)nc2C1)C1(c2ccccc2)COCCN1]

In a rt solution of 6-{2H,4H,5H,6H-pyrrolo[3,4-c]pyrazole-2-sulfonyl}-1,3-benzothiazole (Intermediate I-1) (1.48 g, 4.82 mmol, 1.00 eq.) in CH₂Cl₂ (20.0 mL) was added 3-phenylmorpholine-3-carboxylic acid (Intermediate II-8) (1.00 g, 4.82 mmol, 1.00 equiv), DIEA (1.87 g, 14.47 mmol, 3.00 equiv), HOBT (0.78 g, 5.79 mmol, 1.20 equiv) and EDCI (1.11 g, 5.79 mmol, 1.20 equiv). The resulting solution was stirred for 2 h at 45° C. The reaction mixture was cooled. The resulting solution was diluted with 50 mL of H₂O. The resulting solution was extracted with 3×50 mL of dichloromethane. The organic layers were combined and dried over anhydrous sodium sulfate and concentrated under reduced pressure. The crude product was purified by re-crystallization from CH₃CN to afford the titled compound as a white solid (160 mg, 6.7%). ¹H NMR (300 MHz, DMSO-d₆) δ 9.71 (s, 1H), 9.00 (s, 1H), 8.29 (d, J=8.8 Hz, 1H), 8.17 (s, 1H), 8.01 (d, J=8.7 Hz, 1H), 7.39-7.27 (m, 5H), 5.02-4.90 (m, 1H), 4.60-4.35 (m, 3H), 3.94-3.80 (m, 1H), 3.60 (d, J=10.7 Hz, 1H), 3.49-3.36 (m, 1H), 3.13-3.03 (m, 2H), 2.90-2.68 (m, 2H); LCMS (ES): [M+H]⁺: 496.1.

### Example 1.36

### Synthesis of (2R)-1-[2-(3,4-dihydro-2H-1,4-benzoxazine-6-sulfonyl)-2H,4H,5H,6H-pyrrolo[3,4-c]pyrazol-5-yl]-3-hydroxy-2-phenylpropan-1-one or (2S)-1-[2-(3,4-dihydro-2H-1,4-benzoxazine-6-sulfonyl)-2H,4H,5H,6H-pyrrolo[3,4-c]pyrazol-5-yl]-3-hydroxy-2-phenylpropan-1-one (Compound 22)

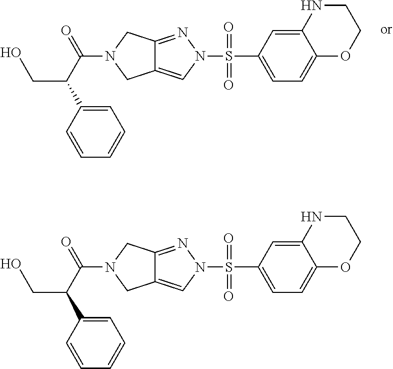 [SMILES: O=C([C@@H](CO)c1ccccc1)N1Cc2cn(S(=O)(=O)c3ccc4c(c3)NCCO4)nc2C1 ; O=C([C@H](CO)c1ccccc1)N1Cc2cn(S(=O)(=O)c3ccc4c(c3)NCCO4)nc2C1]

The title compound was made from 6-{2H,4H,5H,6H-pyrrolo[3,4-c]pyrazole-2-sulfonyl}-3,4-dihydro-2H-1,4-benzoxazine (Intermediate I-3) and Intermediate II-5 following a procedure similar to that described for the synthesis of Compound 1. The crude product was purified by reverse phase preparative HPLC (Prep-C18, 5 μM XBridge column, 19×150 mm, Waters; gradient elution of 10-90% MeCN in water over a 20 min period, where both solvents contain 0.1% formic acid) to provide the title product as a white solid. LCMS (ES) [M+1]⁺ m/z 455.4.

### Example 1.37

### Synthesis of (2R,3R)-1-[2-(3,4-dihydro-2H-1,4-benzoxazine-6-sulfonyl)-2H,4H,5H,6H-pyrrolo[3,4-c]pyrazol-5-yl]-3-hydroxy-2-phenylbutan-1-one, or (2R,3S)-1-[2-(3,4-dihydro-2H-1,4-benzoxazine-6-sulfonyl)-2H,4H,5H,6H-pyrrolo[3,4-c]pyrazol-5-yl]-3-hydroxy-2-phenylbutan-1-one, or (2S,3S)-1-[2-(3,4-dihydro-2H-1,4-benzoxazine-6-sulfonyl)-2H,4H,5H,6H-pyrrolo[3,4-c]pyrazol-5-yl]-3-hydroxy-2-phenylbutan-1-one, or (2S,3R)-1-[2-(3,4-dihydro-2H-1,4-benzoxazine-6-sulfonyl)-2H,4H,5H,6H-pyrrolo [3,4-c]pyrazol-5-yl]-3-hydroxy-2-phenylbutan-1-one (Compound 23)

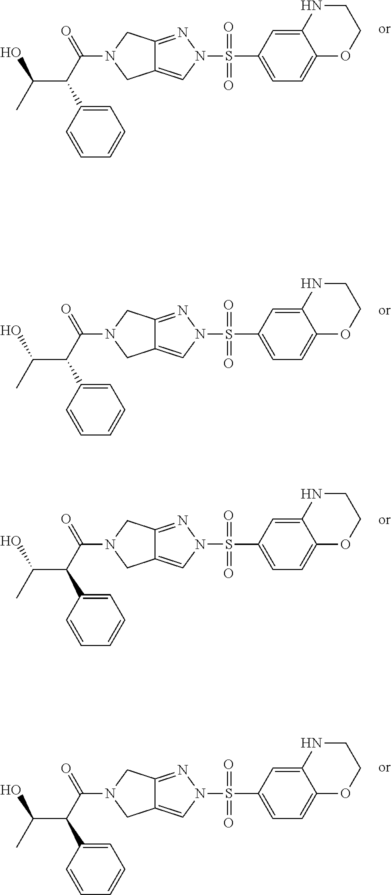 [SMILES: C[C@@H](O)[C@H](C(=O)N1Cc2cn(S(=O)(=O)c3ccc4c(c3)NCCO4)nc2C1)c1ccccc1 ; C[C@H](O)[C@H](C(=O)N1Cc2cn(S(=O)(=O)c3ccc4c(c3)NCCO4)nc2C1)c1ccccc1 ; C[C@H](O)[C@@H](C(=O)N1Cc2cn(S(=O)(=O)c3ccc4c(c3)NCCO4)nc2C1)c1ccccc1 ; C[C@@H](O)[C@@H](C(=O)N1Cc2cn(S(=O)(=O)c3ccc4c(c3)NCCO4)nc2C1)c1ccccc1]

The title compound was made from 6-{2H,4H,5H,6H-pyrrolo[3,4-c]pyrazole-2-sulfonyl}-3,4-dihydro-2H-1,4-benzoxazine (Intermediate I-3) and Intermediate II-1 following a procedure similar to that described for the synthesis of Compound 1. The crude product was purified by reverse phase preparative HPLC (Prep-C18, 5 μM XBridge column, 19×150 mm, Waters; gradient elution of 10-90% MeCN in water over a 20 min period, where both solvents contain 0.1% formic acid) to provide the title product as a white solid. LCMS (ES) [M+1]⁺ m/z 469.4.

### Example 1.38

### Synthesis of (2R,3R)-1-[2-(3,4-dihydro-2H-1,4-benzoxazine-6-sulfonyl)-2H,4H,5H,6H-pyrrolo[3,4-c]pyrazol-5-yl]-3-hydroxy-2-phenylbutan-1-one, or (2R,3S)-1-[2-(3,4-dihydro-2H-1,4-benzoxazine-6-sulfonyl)-2H,4H,5H,6H-pyrrolo[3,4-c]pyrazol-5-yl]-3-hydroxy-2-phenylbutan-1-one, or (2S,3S)-1-[2-(3,4-dihydro-2H-1,4-benzoxazine-6-sulfonyl)-2H,4H,5H,6H-pyrrolo[3,4-c]pyrazol-5-yl]-3-hydroxy-2-phenylbutan-1-one, or (2S,3R)-1-[2-(3,4-dihydro-2H-1,4-benzoxazine-6-sulfonyl)-2H,4H,5H,6H-pyrrolo [3,4-c]pyrazol-5-yl]-3-hydroxy-2-phenylbutan-1-one (Compound 24)

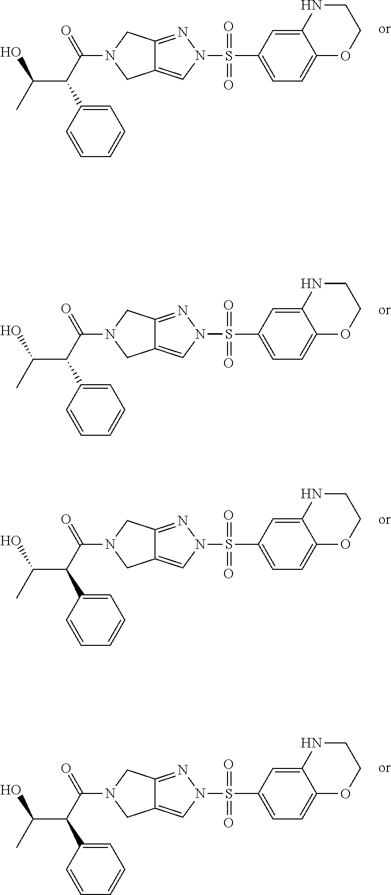 [SMILES: C[C@@H](O)[C@H](C(=O)N1Cc2cn(S(=O)(=O)c3ccc4c(c3)NCCO4)nc2C1)c1ccccc1 ; C[C@H](O)[C@H](C(=O)N1Cc2cn(S(=O)(=O)c3ccc4c(c3)NCCO4)nc2C1)c1ccccc1 ; C[C@H](O)[C@@H](C(=O)N1Cc2cn(S(=O)(=O)c3ccc4c(c3)NCCO4)nc2C1)c1ccccc1 ; C[C@@H](O)[C@@H](C(=O)N1Cc2cn(S(=O)(=O)c3ccc4c(c3)NCCO4)nc2C1)c1ccccc1]

The title compound was made from 6-{2H,4H,5H,6H-pyrrolo[3,4-c]pyrazole-2-sulfonyl}-3,4-dihydro-2H-1,4-benzoxazine (Intermediate I-3) and Intermediate II-3 following a procedure similar to that described for the synthesis of Compound 1. The crude product was purified by reverse phase preparative HPLC (Prep-C18, 5 μM XBridge column, 19×150 mm, Waters; gradient elution of 10-90% MeCN in water over a 20 min period, where both solvents contained 0.1% formic acid) to provide the title product as a white solid. 1H NMR (400 MHz, DMSO-d6) δ 8.13 (d, J=13.5 Hz, 1H), 7.44-7.24 (m, 5H), 7.24-7.16 (m, 1H), 7.10 (t, J=2.4 Hz, 1H), 7.01 (dd, J=8.4, 2.4 Hz, 1H), 6.80 (d, J=8.5 Hz, 1H), 4.95-4.80 (m, 2H), 4.76 (d, J=4.3 Hz, 1H), 4.49-4.08 (m, 4H), 3.66 (dd, J=9.2, 4.0 Hz, 2H), 3.26 (t, J=4.3 Hz, 2H), 0.83 (d, J=6.2 Hz, 3H). LCMS (ES) [M+1]+ m/z 469.2.

### Example 1.39

### Synthesis of (2R)-1-[2-(2,3-dihydro-1,4-benzodioxine-6-sulfonyl)-2H,4H,5H,6H-pyrrolo[3,4-c]pyrazol-5-yl]-3-hydroxy-2-phenylpropan-1-one or (2S)-1-[2-(2,3-dihydro-1,4-benzodioxine-6-sulfonyl)-2H,4H,5H,6H-pyrrolo[3,4-c]pyrazol-5-yl]-3-hydroxy-2-phenylpropan-1-one (Compound 25)

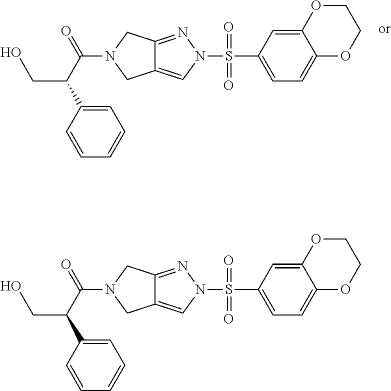 [SMILES: O=C([C@@H](CO)c1ccccc1)N1Cc2cn(S(=O)(=O)c3ccc4c(c3)OCCO4)nc2C1 ; O=C([C@H](CO)c1ccccc1)N1Cc2cn(S(=O)(=O)c3ccc4c(c3)OCCO4)nc2C1]

The title compound was made from 2-(2,3-dihydro-1,4-benzodioxine-6-sulfonyl)-2H,4H,5H,6H-pyrrolo[3,4-c]pyrazole (Intermediate I-4) and Intermediate II-5 following a procedure similar to that described for the synthesis of Compound 1. The crude product was purified by reverse phase preparative HPLC (Prep-C18, 5 μM XBridge column, 19×150 mm, Waters; gradient elution of 10-90% MeCN in water over a 20 min period, where both solvents contain 0.1% formic acid) to provide the title product as a racemic mixture as a white solid. ¹H NMR (400 MHz, DMSO-d₆) δ 8.19 (s, 1H), 7.47-7.16 (m, 7H), 7.06 (d, J=8.5 Hz, 1H), 4.84 (dd, J=22.3, 14.3 Hz, 1H), 4.55-4.38 (m, 2H), 4.38-4.20 (m, 5H), 4.12-3.86 (m, 2H), 3.51 (dd, J=7.7, 4.3 Hz, 1H). LCMS (ES) [M+1]⁺ m/z 456.1.

### Example 1.40

### Synthesis of (2R)-2-amino-1-[2-(1,3-benzothiazole-6-sulfonyl)-2H,4H,5H,6H-pyrrolo[3,4-c]pyrazol-5-yl]-2-(2-chlorophenyl)ethan-1-one (Compound 52)

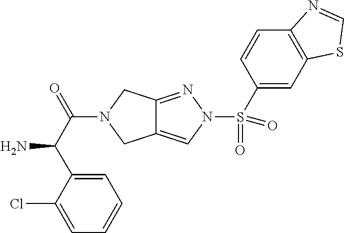 [SMILES: N[C@@H](C(=O)N1Cc2cn(S(=O)(=O)c3ccc4ncsc4c3)nc2C1)c1ccccc1Cl]

Step 1

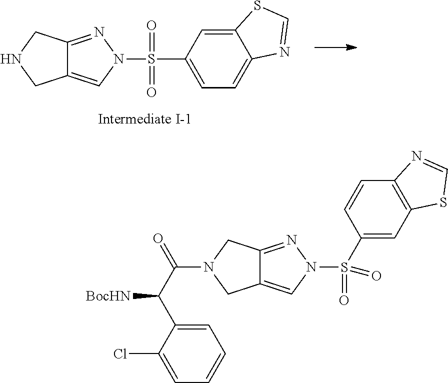 [SMILES: O=S(=O)(c1ccc2ncsc2c1)n1cc2c(n1)CNC2 ; CC(C)(C)OC(=O)N[C@@H](C(=O)N1Cc2cn(S(=O)(=O)c3ccc4ncsc4c3)nc2C1)c1ccccc1Cl]

Into a room temperature solution of 6-{2H,4H,5H,6H-pyrrolo[3,4-c]pyrazole-2-sulfonyl}-1,3-benzothiazole (Intermediate I-1) (1 000.00 mg; 3.26 mmol; 1.00 eq.) and (2R)-[(tert-butoxycarbonyl)amino](2-chlorophenyl)ethanoic acid (932.63 mg; 3.26 mmol; 1.00 eq.) in N,N-dimethylformamide (35.00 mL) was added diisopropylethylamine (1.71 mL; 9.79 mmol; 3.00 eq.) followed by HATU (1 303.16 mg; 3.43 mmol; 1.05 eq.). The resulting mixture was allowed to stir at room temperature overnight, then it was treated with water and extracted with EtOAc thrice. The organic layers were combined, washed with brine, and dried over MgSO₄. After removal of the volatiles under reduced pressure, the remaining residue was purified by column chromatography on silica gel eluted with 50% EtOAc/Heptanes to provide tert-butyl N-[(1R)-2-[2-(1,3-benzothiazole-6-sulfonyl)-2H,4H,5H,6H-pyrrolo[3,4-c]pyrazol-5-yl]-1-(2-chlorophenyl)-2-oxoethyl]carbamate as a white solid (1555 mg, 82.72%). ¹H NMR (400 MHz, DMSO-d6) δ 9.69 (s, 1H), 9.01 (d, J=2.0 Hz, 1H), 8.37-8.21 (m, 2H), 8.03 (dt, J=8.7, 2.3 Hz, 1H), 7.72 (t, J=8.1 Hz, 1H), 7.54-7.24 (m, 4H), 5.68 (dd, J=13.1, 8.3 Hz, 1H), 4.68 (dd, J=13.9, 6.7 Hz, 1H), 4.51-4.31 (m, 2H), 4.23 (d, J=14.4 Hz, 1H), 1.34 (d, J=4.7 Hz, 9H). LCMS (ES) [M+1]⁺ m/z 575.8.

Step 2

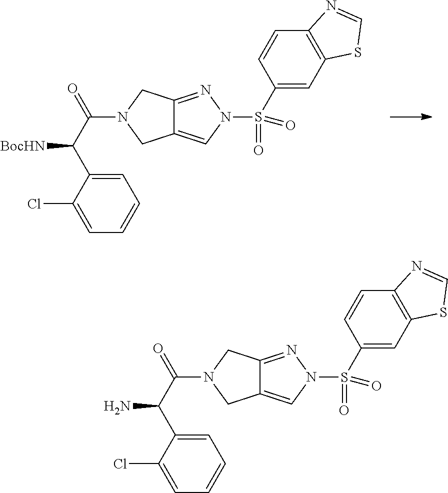 [SMILES: CC(C)(C)OC(=O)N[C@@H](C(=O)N1Cc2cn(S(=O)(=O)c3ccc4ncsc4c3)nc2C1)c1ccccc1Cl ; N[C@@H](C(=O)N1Cc2cn(S(=O)(=O)c3ccc4ncsc4c3)nc2C1)c1ccccc1Cl]

Into a room temperature solution of tert-butyl N-[(1R)-2-[2-(1,3-benzothiazole-6-sulfonyl)-2H,4H,5H,6H-pyrrolo[3,4-c]pyrazol-5-yl]-1-(2-chlorophenyl)-2-oxoethyl]carbamate as a dark solid (1 500.00 mg; 2.61 mmol; 1.00 eq.) in ClCH₂CH₂Cl (90.00 mL) was added anhydrous ZnBr₂ (1 765.19 mg; 7.84 mmol; 3.00 eq.). The resulting mixture was allowed to stir at 65° C. for 5 h, cooled to room temperature, treated with 28% aqueous NH₄OH (10 mL) and water (10 mL), and extracted with 25% IPA/CHCl₃ and dried over MgSO₄. After removal of the volatiles under reduced pressure, the crude final product was purified by reverse phase preparative HPLC (Prep-C18, 5 μM XBridge column, 19×150 mm, Waters; gradient elution of 10-90% MeCN in water over a 20 min period, where both solvents contain 0.1% formic acid) to provide the title product as a white solid (970 mg, 78.80%). ¹H NMR (400 MHz, DMSO-d6) δ 9.69 (s, 1H), 9.02 (t, J=2.0 Hz, 1H), 8.34-8.23 (m, 2H), 8.03 (ddd, J=8.7, 4.1, 2.0 Hz, 1H), 7.50-7.35 (m, 2H), 7.33-7.21 (m, 2H), 4.97-4.79 (m, 2H), 4.50-4.30 (m, 2H), 4.20 (dd, J=25.2, 14.0 Hz, 1H). LCMS (ES) [M+1]⁺ m/z 474.1.

### Example 1.41

### Synthesis of (2S)-3-hydroxy-2-phenyl-1-[2-(quinoline-6-sulfonyl)-2H,4H,5H,6H-pyrrolo[3,4-c]pyrazol-5-yl]propan-1-one (Compound 82)

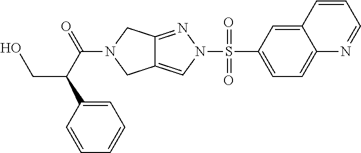 [SMILES: O=C([C@H](CO)c1ccccc1)N1Cc2cn(S(=O)(=O)c3ccc4ncccc4c3)nc2C1]

Step 1

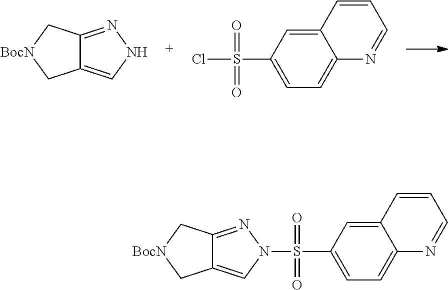 [SMILES: CC(C)(C)OC(=O)N1Cc2c[nH]nc2C1 ; O=S(=O)(Cl)c1ccc2ncccc2c1 ; CC(C)(C)OC(=O)N1Cc2cn(S(=O)(=O)c3ccc4ncccc4c3)nc2C1]

Sodium hydride (228 mg, 5.7 mmol, 1.3 eq.) was added to a pre-cooled solution of N-{2H,4H,5H,6H-pyrrolo[3,4-c]pyrazol-5-yl}(tert-butoxy)formamide (919 mg, 4.4 mmol, 1.0 eq.) in THF (22 mL) at 0° C. After 5 minutes, quinoline-6-sulfonyl chloride (1000 mg, 4.4 mmol, 1.0 eq.) was added to the reaction mixture. The reaction was warmed to room temperature and stirred for 30 minutes. The reaction was quenched with the addition of saturated NH₄Cl(aq) (50 mL). The aqueous layer was extracted with ethyl acetate (60 mL×2). The organic layers were combined, dried with MgSO₄, filtered, concentrated, and purified by Biotage silica gel column chromatography using 5% to 80% ethyl acetate in heptanes to afford tert-butyl 2-(quinolin-6-ylsulfonyl)-2,6-dihydropyrrolo[3,4-c]pyrazole-5(4H)-carboxylate (1700 mg, 97%). LCMS (ES) [M+1]⁺ m/z 401.2.

Step 2

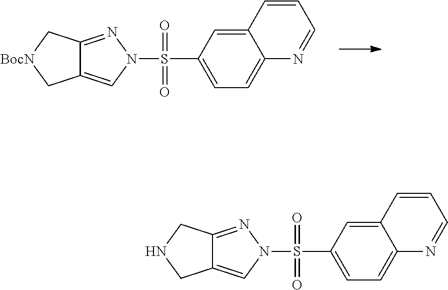 [SMILES: CC(C)(C)OC(=O)N1Cc2cn(S(=O)(=O)c3ccc4ncccc4c3)nc2C1 ; O=S(=O)(c1ccc2ncccc2c1)n1cc2c(n1)CNC2]

Tert-butyl 2-(quinolin-6-ylsulfonyl)-2,6-dihydropyrrolo[3,4-c]pyrazole-5(4H)-carboxylate (1700 mg, 4.2 mol, 1.0 eq.) was placed in acetonitrile (10 mL) and formic acid (10 mL). The mixture was heated to 50° C. for 2 h. The reaction was cooled to room temperature, concentrated under reduced pressure, and purified by prep HPLC C18 column using 0% to 10% MeCN in water to afford 6-((5,6-dihydropyrrolo[3,4-c]pyrazol-2(4H)-yl)sulfonyl)quinoline (320 mg, 25%). LCMS (ES) [M+1]⁺ m/z 301.0.

Step 3

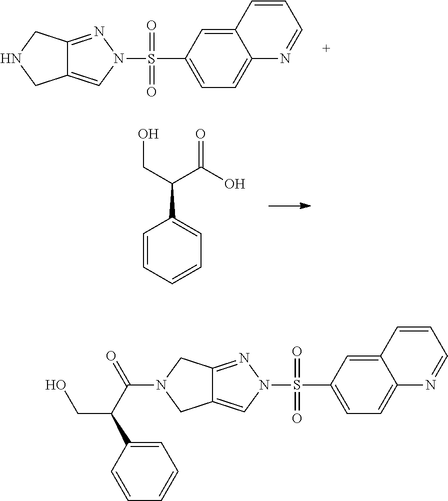

HATU (77 mg, 0.2 mmol, 1.0 eq.) was added to a solution containing 6-((5,6-dihydropyrrolo[3,4-c]pyrazol-2(4H)-yl)sulfonyl)quinoline (61 mg, 0.2 mmol, 1.0 eq.), (2S)-3-hydroxy-2-phenylpropanoic acid (34 mg, 0.2 mmol, 1.0 eq.), triethylamine (0.04 mL, 0.3 mmol, 1.5 eq.), and N,N-dimethylformamide (1 mL). The reaction stirred for 10 minutes, then it was directly purified by prep C18 HPLC using 5% to 70% MeCN in 0.1% formic acid in water to afford (2S)-1-[2-(2,3-dihydro-1,4-benzodioxine-6-sulfonyl)-2H,4H,5H,6H-pyrrolo[3,4-c]pyrazol-5-yl]-3-hydroxy-2-phenylpropan-1-one (70 mg, 77%). ¹H NMR (400 MHz, DMSO-d6) δ 9.08 (dd, J=4.2, 1.8 Hz, 1H), 8.83 (t, J=1.7 Hz, 1H), 8.67 (ddd, J=9.4, 2.2, 1.2 Hz, 1H), 8.29 (s, 1H), 8.18 (d, J=9.0 Hz, 1H), 8.08 (ddd, J=9.0, 2.3, 1.1 Hz, 1H), 7.70 (dd, J=8.4, 4.2 Hz, 1H), 7.32-7.14 (m, 5H), 4.87-4.69 (m, 2H), 4.45-4.21 (m, 3H), 3.99-3.85 (m, 2H), 3.52-3.39 (m, 1H). LCMS (ES) [M+1]⁺ m/z 449.1.

### Example 1.42

### Synthesis of (2R)-1-[2-(1,3-benzothiazole-6-sulfonyl)-2H,4H,5H,6H-pyrrolo [3,4-c]pyrazol-5-yl]-2-(2-chlorophenyl)-2-hydroxyethan-1-one (Compound 65)

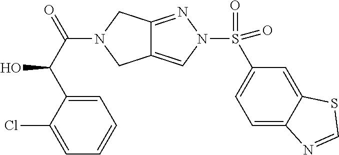 [SMILES: O=C([C@H](O)c1ccccc1Cl)N1Cc2cn(S(=O)(=O)c3ccc4ncsc4c3)nc2C1]

Into a room temperature solution of 6-{2H,4H,5H,6H-pyrrolo[3,4-c]pyrazole-2-sulfonyl}-1,3-benzothiazole (Intermediate I-I) (125.00 mg; 0.41 mmol; 1.00 eq.) and (2R)-(2-chlorophenyl)(hydroxy)ethanoic acid (76.13 mg; 0.41 mmol; 1.00 eq.) in DMF (4.38 mL) was added diisopropylethylamine (0.28 mL; 1.63 mmol; 4.00 eq.) followed by HATU (155.14 mg; 0.41 mmol; 1.00 eq.). The resulting mixture was allowed to stir at room temperature overnight, and treated with water. A precipitate formed that was removed by filtration. This crude compound was purified by reverse phase preparative HPLC (Prep-C18, 5 μM Xbridge column, 19×150 mm, Waters; gradient elution of 10-80% MeCN in water over a 20 min period, where both solvents contain 0.1% formic acid) to provide the title product as a white solid (40 mg, 20.64%). ¹H NMR (DMSO-d6) δ: 9.70 (s, 1H), 8.30 (d, J=8.2 Hz, 2H), 8.01-8.14 (m, 1H), 7.25-7.52 (m, 4H), 5.88-6.12 (m, 2H), 5.52 (dd, J=9.4, 6.7 Hz, 1H), 4.83 (br t, J=14.3 Hz, 1H), 4.27-4.49 (m, 3H). LCMS (ES) [M+1]⁺ m/z 475.1.

### Example 1.43

### Synthesis of (2S)-3-hydroxy-1-{2-[(6-methoxypyridin-3-yl)sulfonyl]-2H,4H,5H,6H-pyrrolo[3,4-c]pyrazol-5-yl}-2-phenylpropan-1-one (Compound 77)

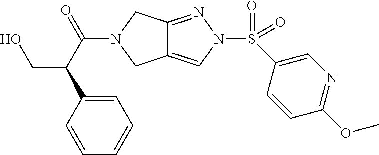 [SMILES: COc1ccc(S(=O)(=O)n2cc3c(n2)CN(C(=O)[C@H](CO)c2ccccc2)C3)cn1]

Step 1

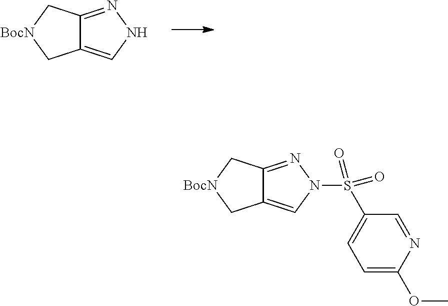 [SMILES: CC(C)(C)OC(=O)N1Cc2c[nH]nc2C1 ; COc1ccc(S(=O)(=O)n2cc3c(n2)CN(C(=O)OC(C)(C)C)C3)cn1]

To a suspension of sodium hydride (86.97 mg; 2.17 mmol; 1.30 eq.) in THF (14.00 mL) under N₂ in an ice bath was added a solution of tert-butyl 2H,4H,5H,6H-pyrrolo[3,4-c]pyrazole-5-carboxylate (350.00 mg; 1.67 mmol; 1.00 eq.) in THF (6 mL) over 5 min. The mixture was allowed to stir at 0° C. for 30 min. A solution of 6-methoxy-3-pyridinesulfonyl chloride (347.31 mg; 1.67 mmol; 1.00 eq.) in THF (4 mL) was then added slowly. The reaction flask was stirred in the ice bath for 45 min and then allowed to warm to room temperature and left stirring for 90 min. The reaction mixture was quenched with a saturated solution of NH₄Cl, to which 0.5 mL of AcOH was added. This solution was extracted twice with 25% IPA/CHCl₃. The combined organics were washed with water and brine and dried over Na₂SO₄. The crude was purified on a silica gel column using 0-20% solvent A in CH₂Cl₂ (solvent A: 0.1% NH₄OH/10% MeOH/CH₂Cl₂) to provide tert-butyl 2-[(6-methoxypyridin-3-yl)sulfonyl]-2H,4H,5H,6H-pyrrolo[3,4-c]pyrazole-5-carboxylate (350 mg, 55%). LCMS (ES) [M+1]⁺ m/z 381.7.

Step 2

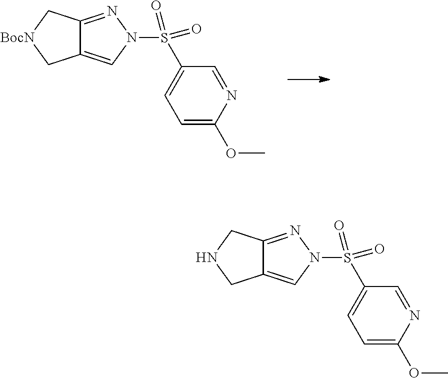 [SMILES: COc1ccc(S(=O)(=O)n2cc3c(n2)CN(C(=O)OC(C)(C)C)C3)cn1 ; COc1ccc(S(=O)(=O)n2cc3c(n2)CNC3)cn1]

Into a room temperature solution of t-butyl 2-[(6-methoxypyridin-3-yl)sulfonyl]-2H,4H,5H,6H-pyrrolo[3,4-c]pyrazole-5-carboxylate (350.00 mg; 0.92 mmol; 1.00 eq.) in ClCH₂CH₂Cl (21.00 mL) was added anhydrous ZnBr₂ (621.54 mg; 2.76 mmol; 3.00 eq.). The resulting mixture was allowed to stir at 65° C. overnight, cooled to room temperature, treated with 28% aqueous NH₄OH and water, and extracted with 25% IPA/CHCl₃ thrice (9 mL). The organic layers were combined and washed with water and brine. After removal of the volatiles under reduced pressure, the crude 2-methoxy-5-{2H,4H,5H,6H-pyrrolo[3,4-c]pyrazole-2-sulfonyl}pyridine (210 mg, 81%) was used as is in the next step. LCMS (ES) [M+1]⁺ m/z 281.5.

Step 3

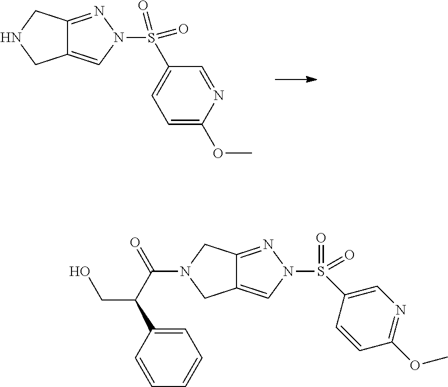 [SMILES: COc1ccc(S(=O)(=O)n2cc3c(n2)CNC3)cn1 ; COc1ccc(S(=O)(=O)n2cc3c(n2)CN(C(=O)[C@H](CO)c2ccccc2)C3)cn1]

Into a room temperature solution of 2-methoxy-5-{2H,4H,5H,6H-pyrrolo[3,4-c]pyrazole-2-sulfonyl}pyridine (80.00 mg; 0.29 mmol; 1.00 eq.) and (2S)-3-hydroxy-2-phenylpropanoic acid (47.43 mg; 0.29 mmol; 1.00 eq.) dissolved in DMF (5 ml), diisopropylethylamine (0.14 mL; 0.86 mmol; 3.00 eq.) was added followed by HATU (108.52 mg; 0.29 mmol; 1.00 eq.). The resulting mixture was allowed to stir at room temperature overnight, treated with water, and extracted with 25% IPA/CHCl₃ (trice). The organic layers were combined. After removal of the volatiles under reduced pressure, the remaining residue was purified by reverse phase preparative HPLC (Prep-C18, 5 μM XBridge column, 19×150 mm, Waters; gradient elution of 10-80% MeCN in water over a 20 min period, where both solvents contained 0.1% formic acid), to provide the title product as a white solid (70 mg, 57.24%). ¹H NMR (400 MHz, DMSO-d6) δ 8.74 (dt, J=2.7, 0.7 Hz, 1H), 8.21 (q, J=1.2 Hz, 1H), 8.12 (ddd, J=8.9, 2.7, 0.8 Hz, 1H), 7.34-7.23 (m, 4H), 7.23-7.16 (m, 1H), 7.00 (ddd, J=9.0, 1.5, 0.7 Hz, 1H), 4.90-4.71 (m, 2H), 4.49-4.22 (m, 3H), 3.99-3.92 (m, 2H), 3.90 (d, J=0.5 Hz, 3H), 3.53-3.44 (m, 1H). LCMS (ES) [M+1]⁺ m/z 429.8.

### Example 1.43

### Synthesis of (2S)-1-{2-[3-(difluoromethoxy)benzenesulfonyl]-2H,4H,5H,6H-pyrrolo[3,4-c]pyrazol-5-yl}-3-hydroxy-2-phenylpropan-1-one (Compound 83)

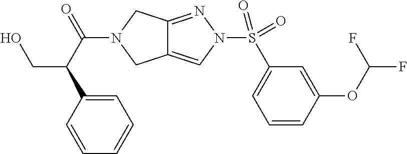 [SMILES: O=C([C@H](CO)c1ccccc1)N1Cc2cn(S(=O)(=O)c3cccc(OC(F)F)c3)nc2C1]

Step 1

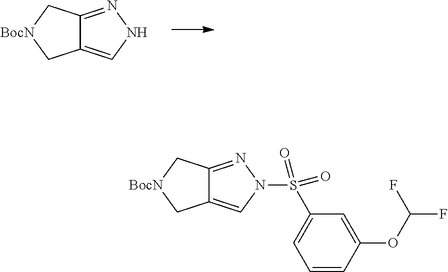 [SMILES: CC(C)(C)OC(=O)N1Cc2c[nH]nc2C1 ; CC(C)(C)OC(=O)N1Cc2cn(S(=O)(=O)c3cccc(OC(F)F)c3)nc2C1]

To a suspension of sodium hydride (152.92 mg; 3.82 mmol; 2.00 eq.) in THF (20.00 mL) under N₂ in an ice bath was added a solution of tert-butyl 2H,4H,5H,6H-pyrrolo[3,4-c]pyrazole-5-carboxylate (400.00 mg; 1.91 mmol; 1.00 eq.) in THF (2 mL) over 5 min. The mixture was allowed to stir at 0° C. for 30 min. A solution of 3-(difluoromethoxy)benzenesulfonyl chloride (695.72 mg; 2.87 mmol; 1.50 eq.) in THF (2 mL) was then added slowly. The mixture was allowed to stir in the ice bath for 45 min and then allowed to warm to room temperature. The reaction mixture was then quenched and diluted with NH4Cl (0.5 mL of AcOH was also added). The reaction mixture was quenched with a saturated solution of NH₄Cl, to which 0.5 mL of AcOH was added. This solution was extracted twice with 25% IPA/CHCl₃. The combined organics were washed with water and brine and dried over Na₂SO₄. The crude was purified on a silica gel column using 0-30% EtOAc/heptanes as eluent to give tert-butyl 2-[3-(difluoromethoxy)benzenesulfonyl]-2H,4H,5H,6H-pyrrolo[3,4-c]pyrazole-5-carboxylate (676 mg, 85.13%). LCMS (ES) [M+1]⁺ m/z 416.2.

Step 2

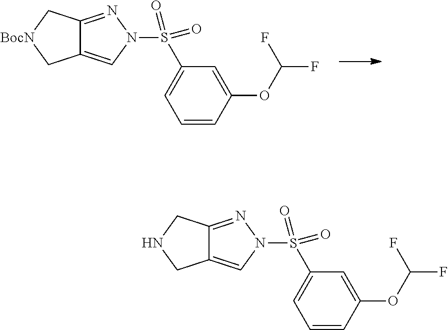 [SMILES: CC(C)(C)OC(=O)N1Cc2cn(S(=O)(=O)c3cccc(OC(F)F)c3)nc2C1 ; O=S(=O)(c1cccc(OC(F)F)c1)n1cc2c(n1)CNC2]

Into a room temperature solution of tert-butyl 2-[3-(difluoromethoxy)benzenesulfonyl]-2H,4H,5H,6H-pyrrolo[3,4-c]pyrazole-5-carboxylate (676.00 mg; 1.63 mmol; 1.00 eq.) in ClCH₂CH₂Cl (40.56 mL) was added anhydrous ZnBr₂ (1 099.35 mg; 4.88 mmol; 3.00 eq.). The resulting mixture was allowed to stir at 65° C. overnight. LC/MS indicated the reaction was not complete and an additional 3 eq of ZnBr₂ were added and the reaction was left for an additional 24 h at 65° C. The reaction mixture was cooled to room temperature, treated with 28% aqueous NH₄OH and water, and extracted with 25% IPA/CHCl₃ thrice. The organic layers were combined and washed with water and brine. After removal of the volatiles under reduced pressure, the crude 2-[3-(difluoromethoxy)benzenesulfonyl]-2H,4H,5H,6H-pyrrolo[3,4-c]pyrazole (440 mg, 85.76%) was used as is in the next step. LCMS (ES) [M+1]⁺ m/z 316.14.

Step 3

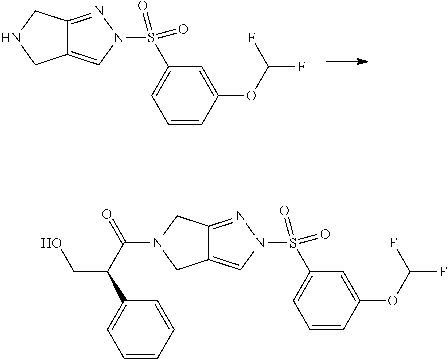 [SMILES: O=S(=O)(c1cccc(OC(F)F)c1)n1cc2c(n1)CNC2 ; O=C([C@H](CO)c1ccccc1)N1Cc2cn(S(=O)(=O)c3cccc(OC(F)F)c3)nc2C1]

Into a room temperature solution of 2-[3-(difluoromethoxy)benzenesulfonyl]-2H,4H,5H,6H-pyrrolo[3,4-c]pyrazole (80.00 mg; 0.25 mmol; 1.00 eq.) and (2S)-3-hydroxy-2-phenylpropanoic acid (42.16 mg; 0.25 mmol; 1.00 eq.) dissolved in DMF (2.8 ml) was added diisopropylethylamine (0.13 mL; 0.76 mmol; 3.00 eq.), followed by HATU (101.30 mg; 0.27 mmol; 1.05 eq.). The resulting mixture was allowed to stir at room temperature overnight, treated with water, and extracted with 25% IPA/CHCl₃ (trice). The organic layers were combined. After removal of the volatiles under reduced pressure, the remaining residue was purified by reverse phase silica gel using 0 to 80% MeCN/water as eluent. The fractions containing the product were combined and freeze dried to provide the title product as a white solid (72 mg, 61.23%). ¹H NMR (400 MHz, DMSO-d6) δ 8.26 (d, J=1.2 Hz, 1H), 7.82-7.76 (m, 1H), 7.76-7.63 (m, 2H), 7.59-7.52 (m, 1H), 7.39-7.13 (m, 6H), 4.90-4.72 (m, 2H), 4.49-4.18 (m, 3H), 4.02-3.88 (m, 2H), 3.52-3.45 (m, 1H). LCMS (ES) [M+1]⁺ m/z 464.3.

### Example 1.45

### Synthesis of (2R)-2-amino-1-[2-(1-benzofuran-5-sulfonyl)-2H,4H,5H,6H-pyrrolo[3,4-c]pyrazol-5-yl]-2-(2-fluorophenyl)ethan-1-one (Compound 64)

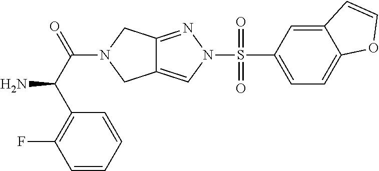 [SMILES: N[C@@H](C(=O)N1Cc2cn(S(=O)(=O)c3ccc4occc4c3)nc2C1)c1ccccc1F]

Step 1

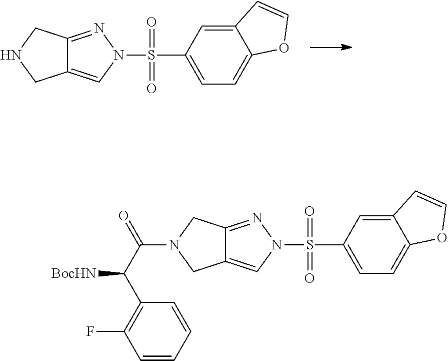 [SMILES: O=S(=O)(c1ccc2occc2c1)n1cc2c(n1)CNC2 ; CC(C)(C)OC(=O)N[C@@H](C(=O)N1Cc2cn(S(=O)(=O)c3ccc4occc4c3)nc2C1)c1ccccc1F]

To a solution of (R)-[(t-butoxycarbonyl)amino](2-fluorophenyl)acetic acid (240.00 mg, 0.891 mmol, 1.00 eq.) and 2-(1-benzofuran-5-sulfonyl)-2H,4H,5H,6H-pyrrolo[3,4-c]pyrazole (Intermediate I-5, 301.30 mg, 0.980 mmol, 1.1 eq.) in DMF (5 mL) was added DIEA (230.39 mg, 1.783 mmol, 2 eq.) at 0° C. After that, the reaction mixture was stirred at 0° C. for 5 min, and HATU (407 mg, 1.070 mmol, 1.2 eq.) was added in portions and the resulting solution was stirred for 2 h at 25° C. The reaction was then quenched by the addition of 10 mL of water, extracted with 3×30 mL of ethyl acetate, washed with 3×50 mL of brine, dried over anhydrous sodium sulfate, and concentrated under vacuum. The residue was applied onto a silica gel column and eluted with ethyl acetate/petroleum ether (1:100 to 1:1). This resulted in t-butyl N-[(1R)-2-[2-(1-benzofuran-5-sulfonyl)-4H,6H-pyrrolo[3,4-c]pyrazol-5-yl]-1-(2-fluorophenyl)-2-oxoethyl]carbamate (250 mg, 51.97%) as a white solid. LCMS (ES) [M+1]⁺ m/z 541.

Step 2

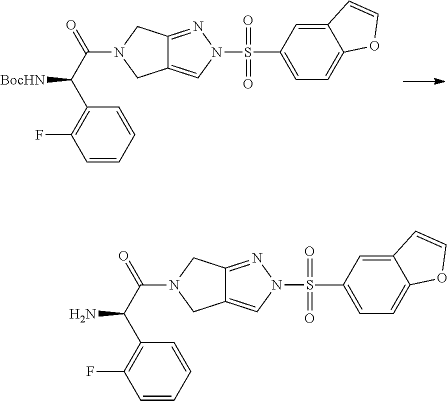 [SMILES: CC(C)(C)OC(=O)N[C@@H](C(=O)N1Cc2cn(S(=O)(=O)c3ccc4occc4c3)nc2C1)c1ccccc1F ; N[C@@H](C(=O)N1Cc2cn(S(=O)(=O)c3ccc4occc4c3)nc2C1)c1ccccc1F]

Into a 100-mL round-bottom flask, was placed t-butyl N-[(1R)-2-[2-(1-benzofuran-5-sulfonyl)-4H,6H-pyrrolo[3,4-c]pyrazol-5-yl]-1-(2-fluorophenyl)-2-oxoethyl]carbamate (250.00 mg, 0.462 mmol, 1.00 eq.), DCM (20.00 mL), ZnBr₂ (312.47 mg, 1.387 mmol, 3.00 eq.). The resulting solution was stirred overnight at 25° C. The resulting mixture was concentrated under vacuum. The residue was dissolved in 4 mL of MeOH. The crude product was further purified by Prep-HPLC with the following conditions (Waters I): Column, Xbridge Prep C18 OBD column, 5 um, 19*150 mm; mobile phase, Water (0.05% FA) and CH₃CN (20% CH₃CN up to 50% in 10 min); Detector, UV 254 nm. This resulted in 99.2 mg (48.70%) of (2R)-2-amino-1-[2-(1-benzofuran-5-sulfonyl)-2H,4H,5H,6H-pyrrolo [3,4-c]pyrazol-5-yl]-2-(2-fluorophenyl)ethan-1-one as a white solid. ¹H NMR (300 MHz, DMSO-d₆) δ 8.41-8.39 (m, 1H), 8.33-8.22 (m, 2H), 7.92-7.83 (m, 2H), 7.50-7.32 (m, 2H), 7.31-7.14 (m, 3H), 5.15 (d, J=18.3 Hz, 1H), 4.88 (dd, J=19.1, 13.9 Hz, 1H), 4.61-4.32 (m, 2H), 4.03 (dd, J=13.8, 10.7 Hz, 1H); LCMS (ES) [M+1]⁺ m/z 441.

### Example 1.46

### Synthesis of (2S,3R)-2-(2-chlorophenyl)-1-{2-[4-(difluoromethoxy)benzenesulfonyl]-2H,4H,5H,6H-pyrrolo[3,4-c]pyrazol-5-yl}-3-hydroxybutan-1-one or (2S,3S)-2-(2-chlorophenyl)-1-{2-[4-(difluoromethoxy)benzenesulfonyl]-2H,4H,5H,6H-pyrrolo[3,4-c]pyrazol-5-yl}-3-hydroxybutan-1-one or (2R,3S)-2-(2-chlorophenyl)-1-{2-[4-(difluoromethoxy)benzenesulfonyl]-2H,4H,5H,6H-pyrrolo[3,4-c]pyrazol-5-yl}-3-hydroxybutan-1-one or (2R,3R)-2-(2-chlorophenyl)-1-{2-[4-(difluoromethoxy)benzenesulfonyl]-2H,4H,5H,6H-pyrrolo[3,4-c]pyrazol-5-yl}-3-hydroxybutan-1-one (Compound 66)

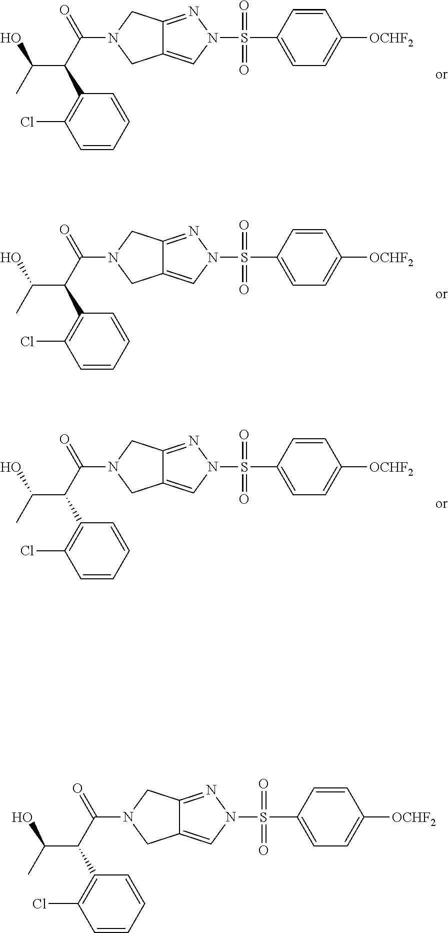 [SMILES: C[C@@H](O)[C@@H](C(=O)N1Cc2cn(S(=O)(=O)c3ccc(OC(F)F)cc3)nc2C1)c1ccccc1Cl ; C[C@H](O)[C@@H](C(=O)N1Cc2cn(S(=O)(=O)c3ccc(OC(F)F)cc3)nc2C1)c1ccccc1Cl ; C[C@H](O)[C@H](C(=O)N1Cc2cn(S(=O)(=O)c3ccc(OC(F)F)cc3)nc2C1)c1ccccc1Cl ; C[C@@H](O)[C@H](C(=O)N1Cc2cn(S(=O)(=O)c3ccc(OC(F)F)cc3)nc2C1)c1ccccc1Cl]

Into a 8-mL vial was added Intermediate II-9b (100.00 mg, 0.466 mmol, 1.00 eq.), 2-[4-(difluoromethoxy)benzenesulfonyl]-2H,4H,5H,6H-pyrrolo[3,4-c]pyrazole (Intermediate I-2, 161.57 mg, 0.512 mmol, 1.10 eq.), DMF (5.00 mL), DIEA (180.63 mg, 1.398 mmol, 3.00 eq.), and HATU (212.57 mg, 0.559 mmol, 1.20 eq.). The resulting solution was stirred for 2 h at 25° C. The crude product was purified by Prep-HPLC with the following conditions (2 #SHIMADZU (HPLC-01)): Column, Atlantis HILIC OBD Column, 19*150 mm*5 um; mobile phase, Water (0.1% FA) and ACN (30% Phase B up to 70% in 15 min); Detector, uv 254 nm. This resulted in 57.3 mg (24.03%) of (2R,3R)-2-(2-chlorophenyl)-1-[2-[4-(difluoromethoxy)benzenesulfonyl]-4H,6H-pyrrolo[3,4-c]pyrazol-5-yl]-3-hydroxybutan-1-one as a white solid. ¹H NMR (300 MHz, DMSO-d₆) δ 8.28 (d, J=8.7 Hz, 1H), 8.09-7.99 (m, 2H), 7.69-7.53 (m, 1H), 7.52-7.36 (m, 4H), 7.35-7.16 (m, 2H), 5.04-4.90 (m, 1H), 4.54-4.40 (m, 2H), 4.40-4.27 (m, 1H), 4.27-4.20 (m, 2H), 0.90 (q, J=2.2 Hz, 3H); LCMS (ES) [M+1]⁺ m/z 512.

### Example 1.47

### Synthesis of (2R)-2-(2-chlorophenyl)-2-hydroxy-1-[2-(1H-indazole-6-sulfonyl)-2H,4H,5H,6H-pyrrolo[3,4-c]pyrazol-5-yl]ethan-1-one (Compound 67)

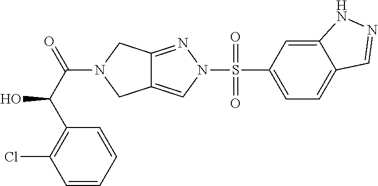 [SMILES: O=C([C@H](O)c1ccccc1Cl)N1Cc2cn(S(=O)(=O)c3ccc4cn[nH]c4c3)nc2C1]

To a stirred solution of (R)-(2-chlorophenyl)(hydroxy)acetic acid (60.00 mg, 0.322 mmol, 1.00 eq.), NMM (65.05 mg, 0.643 mmol, 2 eq.) and 6-{2H,4H,5H,6H-pyrrolo[3,4-c]pyrazole-2-sulfonyl}-1H-indazole (Intermediate I-13, 132.90 mg, 0.322 mmol, 1.00 eq., 70% purity) in DMF (1.00 mL, 12.922 mmol, 46.23 eq.) was added HATU (146.72 mg, 0.386 mmol, 1.2 eq.) in portions at room temperature under a nitrogen atmosphere. The resulting mixture was stirred for 1 h at room temperature under a nitrogen atmosphere. The crude product was purified by Prep-HPLC to provide (2R)-2-(2-chlorophenyl)-2-hydroxy-1-[2-(1H-indazole-6-sulfonyl)-4H,6H-pyrrolo[3,4-c]pyrazol-5-yl]ethanone (65 mg, 33.11%) as a white solid. ¹H NMR (300 MHz, DMSO-d₆) δ 13.73 (s, 1H), 8.35 (d, J=2.7 Hz, 1H), 8.31 (s, 1H), 8.25 (s, 1H), 8.05 (d, J=8.6 Hz, 1H), 7.60 (d, J=8.7 Hz, 1H), 7.55-7.46 (m, 1H), 7.46-7.39 (m, 1H), 7.39-7.30 (m, 2H), 6.04 (dd, J=15.6, 6.7 Hz, 1H), 5.54 (t, J=6.8 Hz, 1H), 4.85 (dd, J=14.3, 11.0 Hz, 1H), 4.52-4.40 (m, 2H), 4.40-4.24 (m, 1H); LCMS (ES [M+1]⁺ m/z: 458.

### Example 1.48

### Synthesis of (2S,3R)-2-(2-chlorophenyl)-1-{2-[4-(difluoromethoxy)benzenesulfonyl]-2H,4H,5H,6H-pyrrolo[3,4-c]pyrazol-5-yl}-3-hydroxybutan-1-one or (2S,3S)-2-(2-chlorophenyl)-1-{2-[4-(difluoromethoxy)benzenesulfonyl]-2H,4H,5H,6H-pyrrolo[3,4-c]pyrazol-5-yl}-3-hydroxybutan-1-one or (2R,3S)-2-(2-chlorophenyl)-1-{2-[4-(difluoromethoxy)benzenesulfonyl]-2H,4H,5H,6H-pyrrolo[3,4-c]pyrazol-5-yl}-3-hydroxybutan-1-one or (2R,3R)-2-(2-chlorophenyl)-1-{2-[4-(difluoromethoxy)benzenesulfonyl]-2H,4H,5H,6H-pyrrolo[3,4-c]pyrazol-5-yl}-3-hydroxybutan-1-one (Compound 68)

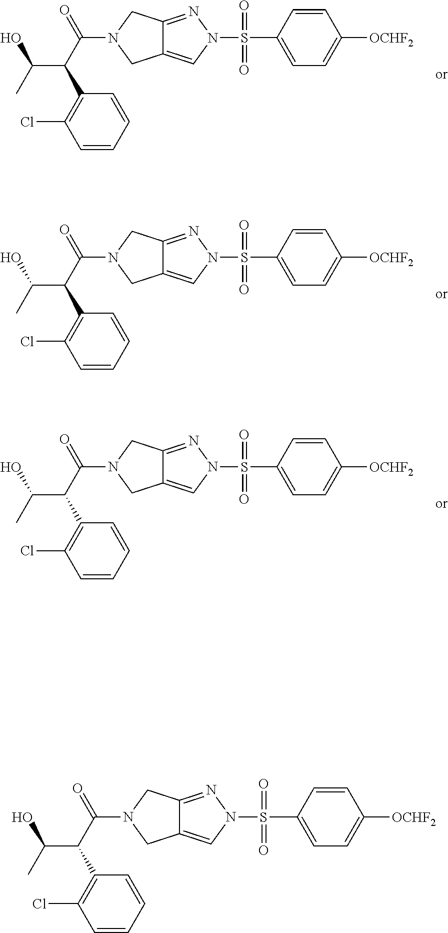 [SMILES: C[C@@H](O)[C@@H](C(=O)N1Cc2cn(S(=O)(=O)c3ccc(OC(F)F)cc3)nc2C1)c1ccccc1Cl ; C[C@H](O)[C@@H](C(=O)N1Cc2cn(S(=O)(=O)c3ccc(OC(F)F)cc3)nc2C1)c1ccccc1Cl ; C[C@H](O)[C@H](C(=O)N1Cc2cn(S(=O)(=O)c3ccc(OC(F)F)cc3)nc2C1)c1ccccc1Cl ; C[C@@H](O)[C@H](C(=O)N1Cc2cn(S(=O)(=O)c3ccc(OC(F)F)cc3)nc2C1)c1ccccc1Cl]

Into a 8-mL vial, was added Intermediate II-9a (100.00 mg, 0.466 mmol, 1.00 eq.), 2-[4-(difluoromethoxy)benzenesulfonyl]-2H,4H,5H,6H-pyrrolo[3,4-c]pyrazole (Intermediate I-2, 161.57 mg, 0.512 mmol, 1.10 eq.), DMF (5.00 mL), DIEA (180.63 mg, 1.398 mmol, 3.00 eq.), and HATU (212.57 mg, 0.559 mmol, 1.20 eq.). The resulting solution was stirred for 2 h at 25° C. The crude product was purified by Prep-HPLC with the following conditions (2 #SHIMADZU (HPLC-01)): Column, Atlantis HILIC OBD Column, 19*150 mm·5 um; mobile phase, Water (0.1% FA) and ACN (30% Phase B up to 70% in 15 min); Detector, uv 254 nm. This resulted in 83.3 mg (34.93%) of (2R,3S)-2-(2-chlorophenyl)-1-[2-[4-(difluoromethoxy)benzenesulfonyl]-4H,6H-pyrrolo[3,4-c]pyrazol-5-yl]-3-hydroxybutan-1-one as a white solid. ¹H NMR (300 MHz, DMSO-d₆) δ 8.27 (d, J=8.8 Hz, 1H), 8.09-7.99 (m, 2H), 7.69-7.54 (m, 1H), 7.48-7.37 (m, 4H), 7.36-7.15 (m, 2H), 4.94 (dd, J=23.4, 14.1 Hz, 1H), 4.66 (br, 1H), 4.53-4.40 (m, 1H), 4.38-4.25 (m, 2H), 4.25-4.14 (m, 2H), 1.17 (d, J=5.4 Hz, 3H); LCMS (ES) [M+1]⁺ m/z 512.

### Example 1.49

### Synthesis of (2S,3S)-2-(2-chlorophenyl)-1-{2-[4-(difluoromethoxy)benzenesulfonyl]-2H,4H,5H,6H-pyrrolo[3,4-c]pyrazol-5-yl}-3-hydroxybutan-1-one or (2S,3R)-2-(2-chlorophenyl)-1-{2-[4-(difluoromethoxy)benzenesulfonyl]-2H,4H,5H,6H-pyrrolo[3,4-c]pyrazol-5-yl}-3-hydroxybutan-1-one or (2R,3R)-2-(2-chlorophenyl)-1-{2-[4-(difluoromethoxy)benzenesulfonyl]-2H,4H,5H,6H-pyrrolo[3,4-c]pyrazol-5-yl}-3-hydroxybutan-1-one or (2R,3S)-2-(2-chlorophenyl)-1-{2-[4-(difluoromethoxy)benzenesulfonyl]-2H,4H,5H,6H-pyrrolo[3,4-c]pyrazol-5-yl}-3-hydroxybutan-1-one (Compound 69)

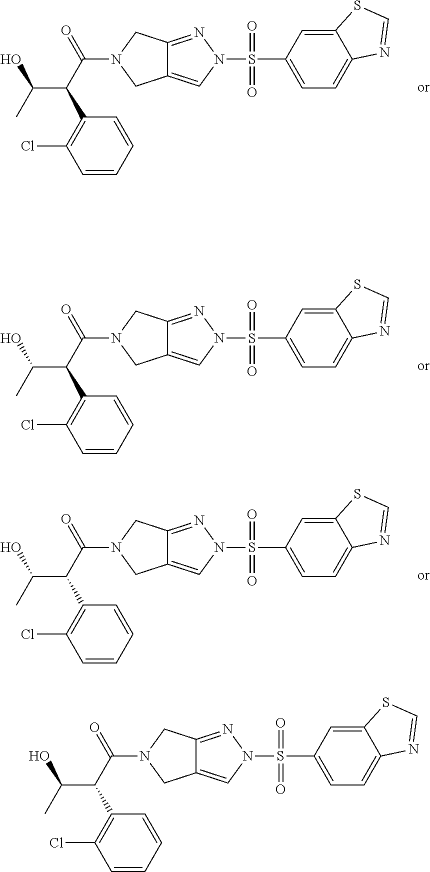 [SMILES: C[C@@H](O)[C@@H](C(=O)N1Cc2cn(S(=O)(=O)c3ccc4ncsc4c3)nc2C1)c1ccccc1Cl ; C[C@H](O)[C@@H](C(=O)N1Cc2cn(S(=O)(=O)c3ccc4ncsc4c3)nc2C1)c1ccccc1Cl ; C[C@H](O)[C@H](C(=O)N1Cc2cn(S(=O)(=O)c3ccc4ncsc4c3)nc2C1)c1ccccc1Cl ; C[C@@H](O)[C@H](C(=O)N1Cc2cn(S(=O)(=O)c3ccc4ncsc4c3)nc2C1)c1ccccc1Cl]

To a 0° C. solution of Intermediate II-9b (100 mg, 0.46 mmol, 1.0 eq.) and 6-{2H,4H,5H,6H-pyrrolo[3,4-c]pyrazole-2-sulfonyl}-1,3-benzothiazole (Intermediate I-1, 171 mg, 0.56 mmol, 1.2 eq.) in DMF (3 mL) was added NMM (117 mg, 1.16 mmol, 2.5 eq.) and HATU (221 mg, 0.58 mmol, 1.25 eq.) in one portion. The resulting mixture was stirred for 1 h at 0° C. then it was concentrated and purified by prep. HPLC (Column, C18; mobile phase, Mobile phase A: MeCN, B: Water=5/1 Flow rate: 20 mL/min Column: DAICEL CHIRALPAK IC, 250*20 mm, 220 nm Gradient: 50% B in 20 min; 220 nm) to give the title product as off-white solid (62 mg, 25.34%)]. ¹H NMR (300 MHz, DMSO-d₆) δ 9.71 (s, 1H), 9.02 (s, 1H), 8.31 (dd, J=9.2, 3.8 Hz, 2H), 8.04 (dt, J=8.8, 2.5 Hz, 1H), 7.57 (td, J=6.9, 6.1, 2.1 Hz, 1H), 7.45 (ddd, J=7.5, 5.5, 1.9 Hz, 1H), 7.37-7.18 (m, 2H), 4.96 (dd, J=14.0, 7.5 Hz, 1H), 4.57-4.39 (m, 2H), 4.36-4.20 (m, 3H), 0.9-0.83 (m, 3H); LCMS (ES) [M+1]⁺ m/z: 503.

### Example 1.50

### Synthesis of (2S,3R)-2-(2-chlorophenyl)-1-(2-[2H,3H-[1,4]dioxino[2,3-b]pyridine-7-sulfonyl]-4H,6H-pyrrolo[3,4-c]pyrazol-5-yl)-3-hydroxybutan-1-one or (2R,3R)-2-(2-chlorophenyl)-1-(2-[2H,3H-[1,4]dioxino[2,3-b]pyridine-7-sulfonyl]-4H,6H-pyrrolo[3,4-c]pyrazol-5-yl)-3-hydroxybutan-1-one or (2R,3S)-2-(2-chlorophenyl)-1-(2-[2H,3H-[1,4]dioxino[2,3-b]pyridine-7-sulfonyl]-4H,6H-pyrrolo[3,4-c]pyrazol-5-yl)-3-hydroxybutan-1-one or (2S,3S)-2-(2-chlorophenyl)-1-(2-[2H,3H-[1,4]dioxino[2,3-b]pyridine-7-sulfonyl]-4H,6H-pyrrolo[3,4-c]pyrazol-5-yl)-3-hydroxybutan-1-one (Compound 70)

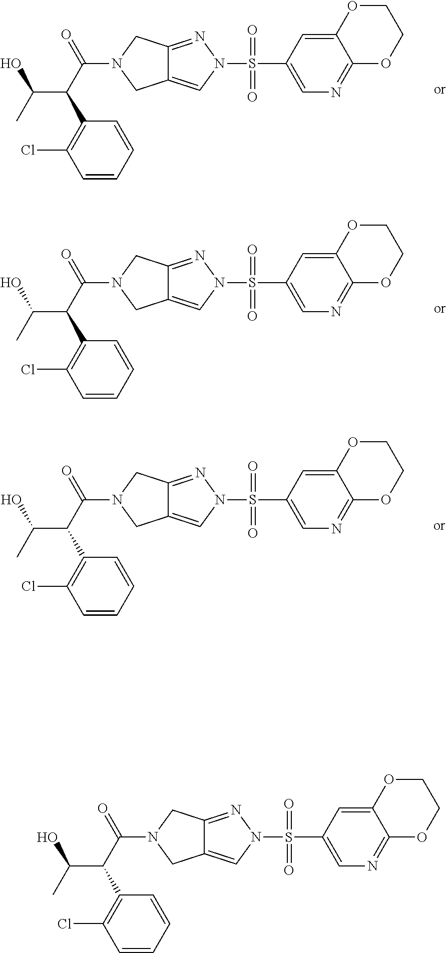 [SMILES: C[C@@H](O)[C@@H](C(=O)N1Cc2cn(S(=O)(=O)c3cnc4c(c3)OCCO4)nc2C1)c1ccccc1Cl ; C[C@H](O)[C@@H](C(=O)N1Cc2cn(S(=O)(=O)c3cnc4c(c3)OCCO4)nc2C1)c1ccccc1Cl ; C[C@H](O)[C@H](C(=O)N1Cc2cn(S(=O)(=O)c3cnc4c(c3)OCCO4)nc2C1)c1ccccc1Cl ; C[C@@H](O)[C@H](C(=O)N1Cc2cn(S(=O)(=O)c3cnc4c(c3)OCCO4)nc2C1)c1ccccc1Cl]

Into a 8-mL vial was added Intermediate II-9b (80 mg, 0.37 mmol, 1.00 eq.), 2-{2H,3H-[1,4]dioxino[2,3-b]pyridine-7-sulfonyl}-2H,4H,5H,6H-pyrrolo[3,4-c]pyrazole (Intermediate I-8, 115 mg, 0.37 mmol, 1.00 eq.), DMF (5 mL), DIEA (145 mg, 1.12 mmol, 3.00 eq.). This was followed by the addition of HATU (170 mg, 0.45 mmol, 1.20 eq.) at 0° C. The resulting solution was stirred for 2 h at 25° C. The crude product was purified by Prep-HPLC with the following conditions: (Prep-HPLC-001): Column, Sunfire Prep C18 OBD Column, 50*250 mm, 5 μm 10 nm; mobile phase, Water (0.1% FA) and ACN (20% Phase B up to 60% in 15 min); Detector, uv. 254 nm, to provide the titled compounds as a white solid. ¹H NMR (300 MHz, DMSO-d₆) δ 8.37-8.24 (m, 2H), 7.78-7.70 (m, 1H), 7.59 (ddd, J=7.7, 5.9, 2.0 Hz, 1H), 7.47 (dt, J=7.6, 1.7 Hz, 1H), 7.30 (dddd, J=14.9, 7.4, 5.7, 1.6 Hz, 2H), 4.98 (t, J=14.1 Hz, 1H), 4.60-4.43 (m, 4H), 4.43-4.15 (m, 6H), 0.90 (d, J=3.0 Hz, 3H); LCMS (ES) [M+1]⁺ m/z 505.

### Example 1.51

### Synthesis of (2S,3R)-2-(2-chlorophenyl)-1-(2-[2H,3H[1,4]dioxino[2,3-b]pyridine-7-sulfonyl]-4H,6H-pyrrolo[3,4-c]pyrazol-5-yl)-3-hydroxybutan-1-one or (2R,3R)-2-(2-chlorophenyl)-1-(2-[2H,3H-[1,4]dioxino[2,3-b]pyridine-7-sulfonyl]-4H,6H-pyrrolo[3,4-c]pyrazol-5-yl)-3-hydroxybutan-1-one (Compound 71)

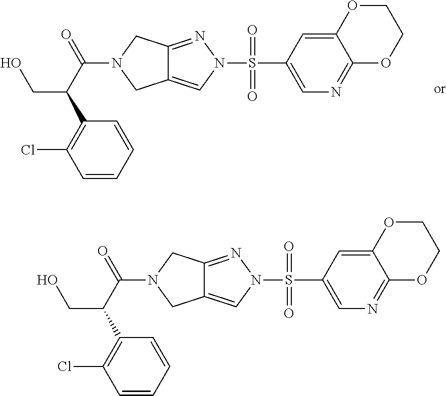 [SMILES: O=C([C@H](CO)c1ccccc1Cl)N1Cc2cn(S(=O)(=O)c3cnc4c(c3)OCCO4)nc2C1 ; O=C([C@@H](CO)c1ccccc1Cl)N1Cc2cn(S(=O)(=O)c3cnc4c(c3)OCCO4)nc2C1]

Step 1

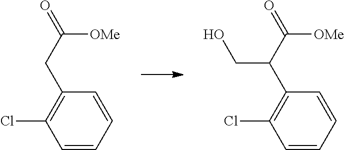 [SMILES: COC(=O)Cc1ccccc1Cl ; COC(=O)C(CO)c1ccccc1Cl]

A solution of methyl 2-(2-chlorophenyl)acetate (3.0 g, 16.25 mmol, 1.00 eq.) and paraformaldehyde (536 mg, 17.87 mmol, 1.1 eq.) was suspended in DMSO (40 mL) and treated with NaOMe (176 mg, 3.25 mmol, 0.2 eq.). The mixture was allowed to stir at room temperature for 2 h until completion by TLC analysis of the crude. The reaction was poured into ice-cold water (60 mL) and neutralized with the addition of 1M HCl solution. The aqueous layer was extracted with ethyl acetate (3×50 mL), and the organics were combined. The organic layer was washed with water (2×100 mL), brine (100 mL), separated, dried over MgSO₄, filtered, and concentrated in vacuum to afford the crude product as a yellow oil. The residue was loaded onto a large fritted filter loaded with silica gel and eluted with 9:1 PE: ethyl acetate, providing 1.5 g of methyl 2-(2-chlorophenyl)-3-hydroxypropanoate as a colorless oil. LCMS (ES) [M+1]⁺ m/z: 215.

Step 2

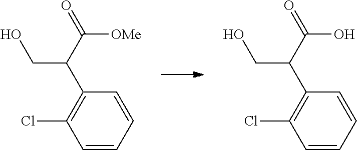 [SMILES: COC(=O)C(CO)c1ccccc1Cl ; O=C(O)C(CO)c1ccccc1Cl]

Into a 50-mL round-bottom flask, was placed 2-(2-chlorophenyl)-3-hydroxypropanoate (1.5 g, 7 mmol, 1.00 eq.) in THF (15 mL). This was followed by the addition of a solution of LiOH.H₂O (588 mg, 14 mmol, 2.00 eq.) in HO (5.0 mL) dropwise with stirring at 0° C. The mixture was stirred for 1 h at room temperature, then it was concentrated to remove THF. The residue was purified by HPLC with the following conditions: C18-120 g column, H₂O/CH₃CN from 0% to 60% within 10 min, Flow rate, 70 mL/min, Detector, 220 nm. The fraction was freeze dried, 1.2 g (86%) of lithium 2-(2-chlorophenyl)-3-hydroxypropanoic acid as a white solid. LCMS (ES) [M−1]⁻ m/z: 199.

Step 3

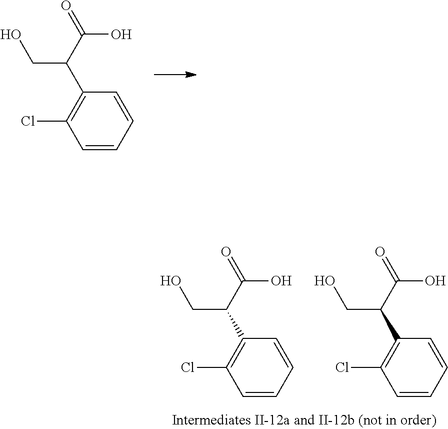 [SMILES: O=C(O)C(CO)c1ccccc1Cl ; O=C(O)[C@@H](CO)c1ccccc1Cl ; O=C(O)[C@H](CO)c1ccccc1Cl]

2-(2-chlorophenyl)-3-hydroxypropanoic acid (300 mg) was purified by Prep-CHIRAL-HPLC with the following conditions: Column: CHIRALPAK IG, 3*25 cm, 5 μm; Mobile Phase A: CO2, Mobile Phase B: MeOH (0.1% TFA)-HPLC; Flow rate: 70 mL/min; Gradient: isocratic 25% B; Wave Length: 220 nm; Sample Solvent: MeOH: DCM=1:1; Injection Volume: 2.5 mL;

The product with RT=4.02 minutes was collected and concentrated. This resulted in 140 mg (46.7%) of (R)-2-(2-chlorophenyl)-3-hydroxypropanoic acid or (S)-2-(2-chlorophenyl)-3-hydroxypropanoic acid as a white solid. LCMS (ES) [M−1]⁻ m/z: 199. Chiral analysis conditions (RT=2.174 min); this material was assigned as Intermediate II-12a

The product with RT=4.81 minutes was collected and concentrated; this resulted in 130 mg (43.3%) of (S)-2-(2-chlorophenyl)-3-hydroxypropanoic acid or (R)-2-(2-chlorophenyl)-3-hydroxypropanoic acid as a white solid. LCMS (ES) [M−1]⁻ m/z: 199. Chiral analysis conditions (RT=2.461 min); this material was assigned as Intermediate II-12b

Step 4

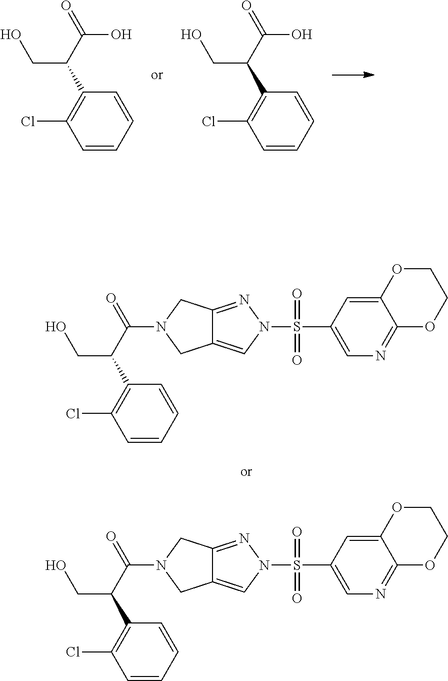 [SMILES: O=C(O)[C@@H](CO)c1ccccc1Cl ; O=C(O)[C@H](CO)c1ccccc1Cl ; O=C([C@@H](CO)c1ccccc1Cl)N1Cc2cn(S(=O)(=O)c3cnc4c(c3)OCCO4)nc2C1 ; O=C([C@H](CO)c1ccccc1Cl)N1Cc2cn(S(=O)(=O)c3cnc4c(c3)OCCO4)nc2C1]

A solution of Intermediate II-12a (70.00 mg, 0.349 mmol, 1.00 eq.), 2-{2H,3H-[1,4] dioxino[2,3-b]pyridine-7-sulfonyl}-2H,4H,5H,6H-pyrrolo [3,4-c]pyrazole (Intermediate I-8, 129.09 mg, 0.419 mmol, 1.2 eq.), NMM (70.58 mg, 0.698 mmol, 2 eq.) and HATU (159.20 mg, 0.419 mmol, 1.2 eq.) in DMF (2.00 mL, 25.844 mmol, 74.07 eq.) was stirred for 2 h at room temperature under nitrogen atmosphere. The crude product was subjected to purification by Prep-HPLC to afford the title compound as a white solid (92 mg, 53.71%). ¹H NMR (300 MHz, DMSO-d₆) δ 8.33 (dd, J=2.3, 1.5 Hz, 1H), 8.27 (d, J=2.2 Hz, 1H), 7.74 (dd, J=3.5, 2.3 Hz, 1H), 7.51-7.39 (m, 2H), 7.34-7.26 (m, 2H), 4.91 (dd, J=20.5, 14.0 Hz, 1H), 4.59-4.49 (m, 2H), 4.47 (d, J=8.3 Hz, 1H), 4.43-4.25 (m, 5H), 4.08-3.88 (m, 1H), 3.63-3.46 (m, 1H); LCMS (ES) [M+1]⁺ m/z: 491.

### Example 1.52

### Synthesis (2R)-1-[2-[4-(difluoromethoxy)benzenesulfonyl]-4H,6H-pyrrolo[3,4-c]pyrazol-5-yl]-2-(2-fluorophenyl)-2-hydroxyethanone (Compound 72)

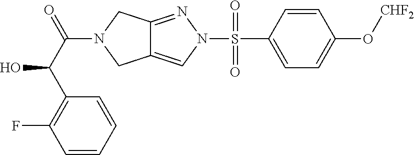 [SMILES: O=C([C@H](O)c1ccccc1F)N1Cc2cn(S(=O)(=O)c3ccc(OC(F)F)cc3)nc2C1]

Into a 50-mL round-bottom flask, was placed (R)-(2-fluorophenyl)(hydroxy)acetic acid (80.69 mg, 0.000 mmol, 1.00 eq.), 2-[4-(difluoromethoxy)benzenesulfonyl]-2H,4H,5H,6H-pyrrolo[3,4-c]pyrazole (Intermediate I-2, 150.00 mg, 0.474 mmol, 1.00 eq.), DMF (5.00 mL), DIEA (183.89 mg, 1.423 mmol, 3.00 eq.), and HATU (216.39 mg, 0.569 mmol, 1.20 eq.). The resulting solution was stirred for 2 h at 25° C. The crude product was purified by Prep-HPLC with the following conditions (Prep-HPLC-001): Column, Sunfire Prep C18 OBD Column, 50*250 mm, 5 μm 10 nm; mobile phase, Water (0.1% FA) and ACN (30% PhaseB up to 60% in 15 min); Detector, uv. 254 nm. This resulted in 117.6 mg (53.05%) of (2R)-1-[2-[4-(difluoromethoxy)benzenesulfonyl]-4H,6H-pyrrolo[3,4-c]pyrazol-5-yl]-2-(2-fluorophenyl)-2-hydroxyethanone as a white solid. ¹H NMR (300 MHz, DMSO-d₆) δ 8.27 (d, J=6.4 Hz, 1H), 8.04 (dd, J=8.9, 1.3 Hz, 2H), 7.49-7.32 (m, 5H), 7.25-7.15 (m, 2H), 5.88 (dd, J=13.7, 6.7 Hz, 1H), 5.53 (t, J=5.9 Hz, 1H), 4.83 (t, J=14.8 Hz, 1H), 4.56-4.31 (m, 2H), 4.31-4.18 (m, 1H), LCMS (ES) [M+1]⁺ m/z 468.

### Example 1.53

### Synthesis of (2R)-1-[2-(1,3-benzothiazole-6-sulfonyl)-2H,4H,5H,6H-pyrrolo[3,4-c]pyrazol-5-yl]-2-(3-fluoropyridin-2-yl)-2-hydroxyethan-1-one or (2S)-1-[2-(1,3-benzothiazole-6-sulfonyl)-2H,4H,5H,6H-pyrrolo[3,4-c]pyrazol-5-yl]-2-(3-fluoropyridin-2-yl)-2-hydroxyethan-1-one (Compound 74)

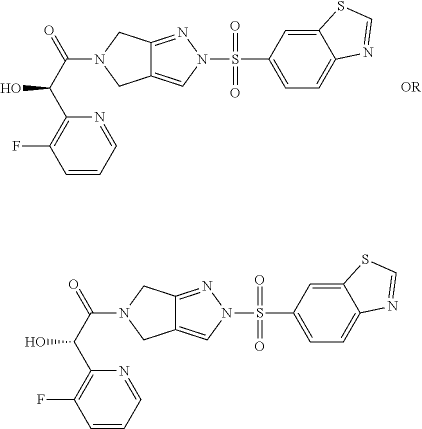 [SMILES: O=C([C@H](O)c1ncccc1F)N1Cc2cn(S(=O)(=O)c3ccc4ncsc4c3)nc2C1 ; O=C([C@@H](O)c1ncccc1F)N1Cc2cn(S(=O)(=O)c3ccc4ncsc4c3)nc2C1]

Into a 8-mL vial, was placed racemic (3-fluoropyridin-2-yl)(hydroxy)acetic acid hydrochloride (260 mg, 1.25 mmol, 1.00 eq.), DMF (3.00 mL) and 6-{2H,4H,5H,6H-pyrrolo[3,4-c]pyrazole-2-sulfonyl}-1,3-benzothiazole (Intermediate I-1, 576 mg, 1.87 mmol, 1.50 eq.), and DIEA (486 mg, 3.75 mmol, 3.00 eq.). This was followed by the addition of HATU (572 mg, 1.50 mmol, 1.20 eq.) at 0° C. The resulting solution was stirred for 1 h at room temperature. The reaction solution was pre-purified by Flash-Prep-HPLC, resulting in mixed product of (2R)-1-[2-(1,3-benzothiazole-6-sulfonyl)-2H,4H,5H,6H-pyrrolo[3,4-c]pyrazol-5-yl]-2-(3-fluoropyridin-2-yl)-2-hydroxyethan-1 -one and (2S)-1-[2-(1,3-benzothiazole-6-sulfonyl)-2H,4H,5H,6H-pyrrolo[3,4-c]pyrazol-5-yl]-2-(3-fluoropyridin-2-yl)-2-hydroxyethan-1-one (270 mg, 47%). LCMS (ES) [M+1]⁺ m/z: 460. This mixture was subjected to separation by Chiral-Prep-HPLC with the following conditions: Column, Lux 5 um Cellulose-4, 2.12×25 cm, 5 um, Mobile Phase: MeOH: CH₃CN=1:1, Flow rate: 35 mL/min, Detector, 220 nm.

The fraction collected at RT=8.2 min was concentrated to provide the title compound as a white solid (117 mg, 44%). ¹H NMR: (300 MHz, DMSO-d₆, ppm): δ 9.72 (s, 1H), 9.03 (d, J=2.1 Hz, 1H), 8.37-8.31 (m, 2H), 8.28 (d, J=10.8 Hz, 1H), 8.07-8.03 (m, 1H), 7.78-7.69 (m, 1H), 7.49-7.41 (m, 1H), 5.79-5.75 (m, 1H), 5.61-5.55 (m, 1H), 4.82 (dd, J=14.7, 10.8 Hz, 1H), 4.66-4.41 (m, 2H), 4.11 (dd, J=15.3, 16.8 Hz, 1H); LCMS (ES, m/z): [M+H]⁺: 460.

### Example 1.54

### Synthesis of 3-chloro-2-(3-{2-[4-(difluoromethoxy)benzenesulfonyl]-2H,4H,5H,6H-pyrrolo[3,4-c]pyrazole-5-carbonyl}oxetan-3-yl)pyridine (Compound 75)

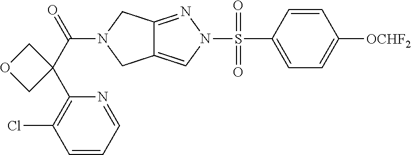 [SMILES: O=C(N1Cc2cn(S(=O)(=O)c3ccc(OC(F)F)cc3)nc2C1)C1(c2ncccc2Cl)COC1]

Step 1

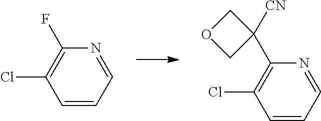 [SMILES: Fc1ncccc1Cl ; N#CC1(c2ncccc2Cl)COC1]

Into a 50-mL 3-necked round-bottom flask, was placed 3-chloro-2-fluoropyridine (1.20 g, 9.12 mmol, 1.00 eq.), oxetane-3-carbonitrile (839 mg, 10.10 mmol, 1.10 eq.) in toluene (20.00 mL), followed by the addition of KHMDS (1M in THF) (11.00 mL, 10.94 mmol, 1.2 eq.) at 0° C. After addition, the resulting solution was stirred for 30 min at room temperature. The reaction was then quenched by the addition of NH₄Cl (aq) (30.00 mL), and extracted with ethyl acetate (50.00 mL×2). The combined organic phase was dried over anhydrous sodium sulfate, filtered, and concentrated under reduced pressure. The residue was purified by silica gel column eluting with ethyl acetate/petroleum ether (1/3) to afford 3-(3-chloropyridin-2-yl)oxetane-3-carbonitrile as a yellow solid (1.38 g, 78%). LCMS (ES) [M+1]⁺ m/z: 195.

Step 2

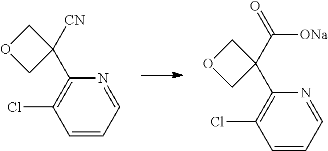

Into a 100-mL round-bottom flask, was placed 3-(3-chloropyridin-2-yl)oxetane-3-carbonitrile (1.38 g, 7.09 mmol, 1.00 eq.), H₂O (10.00 mL), EtOH (20.00 mL), and NaOH (1.14 g, 28.50 mmol, 4.00 eq.) and the mixture was stirred for 12 h at 80° C. The reaction was then cooled to room temperature and directly purified by Flash-Prep-HPLC with conditions: C18-120 g column, CH₃CN/H₂O, from 5% to 40% within 12 min, Flowrate, 70 mL/min, Detector, 254 nm. The fraction of target was freezing dried, this resulted in 1.0 g (60%) of sodium 3-(3-chloropyridin-2-yl)oxetane-3-carboxylate as a white solid. LCMS (ES) [M−Na+H+1]⁺ m/z: 214.

Step 3

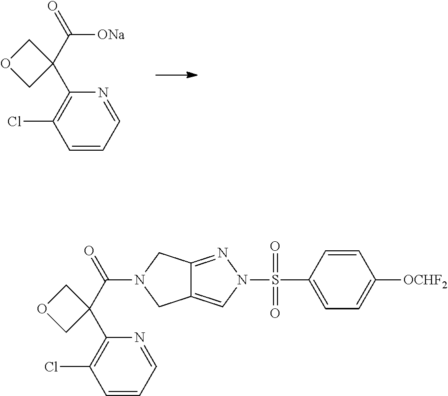

Into a 40-mL vial, was placed sodium 3-(3-chloropyridin-2-yl)oxetane-3-carboxylate (134 mg, 0.57 mmol, 1.00 eq.), 2-[4-(difluoromethoxy)benzenesulfonyl]-2H,4H,5H,6H-pyrrolo[3,4-c]pyrazole (Intermediate I-2, 265 mg, 0.75 mmol, 1.30 eq.), DMF (5.00 mL), and DIEA (243 mg, 1.88 mmol, 3.00 eq.). This was followed by the addition of HATU (287 mg, 0.76 mmol, 1.30 eq.) at 0° C. and the resulting solution was stirred for 2 h at room temperature. The reaction solution was then directly purified by Prep-HPLC with the following conditions: Sunfire Prep C18 OBD Column, 50*250 mm 5 um 10 nm, Mobile phase, Water (0.1% FA) and CH₃CN (30% Phase B up to 60% in 15 min), Detector, UV 254 nm. The fraction of target was freezing dried to provide the title compounds as a white solid (64.3 mg, 22%). ¹H-NMR: (300 MHz, DMSO-d₆, ppm): δ 8.70-8.67 (m, 1H), 8.19 (d, J=23.1 Hz, 1H), 8.03-7.94 (m, 3H), 7.66-7.18 (m, 4H), 5.21 (dd, J=6.3, 3.9 Hz, 2H), 5.11 (d, J=6.0 Hz, 2H), 4.49 (d, J=5.1 Hz, 2H), 3.68 (d, J=3.0 Hz, 2H); LCMS: (ES, m/z): [M+H]⁺: 511.

### Example 1.55

### Synthesis of (2S)-1-(2-{2H,3H-[1,4]dioxino[2,3-b]pyridine-7-sulfonyl}-2H,4H,5H,6H-pyrrolo[3,4-c]pyrazol-5-yl)-3-hydroxy-2-phenylpropan-1-one (Compound 76)

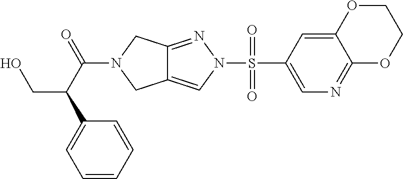 [SMILES: O=C([C@H](CO)c1ccccc1)N1Cc2cn(S(=O)(=O)c3cnc4c(c3)OCCO4)nc2C1]

Into a 8-mL sealed tube, was placed 2-{2H,3H-[1,4]dioxino[2,3-b]pyridine-7-sulfonyl}-2H,4H,5H,6H-pyrrolo[3,4-c]pyrazole (Intermediate I-8, 170.00 mg, 0.551 mmol, 1.00 eq.), (S)-tropic acid (91.63 mg, 0.551 mmol, 1.00 eq.), HATU (230.62 mg, 0.607 mmol, 1.1 eq.), DIEA (142.53 mg, 1.103 mmol, 2.0 eq.), and DMF (3 mL) and the resulting mixture was stirred for 2 h at room temperature. The crude product was directly purified by Prep-HPLC with the following conditions: SunFire Prep C18 OBD Column, 50*250 mm 5 um 10 nm; mobile phase, phase A: H₂O (0.1% FA); phase B: CH₃CN (20% CH₃CN up to 50% CH₃CN in 15 min). This resulted in the title compounds as a white solid (115 mg, 46%).

¹H NMR (300 MHz, DMSO-d₆, ppm): δ 8.32 (d, J=2.3 Hz, 1H), 8.25 (s, 1H), 7.73 (dd, J=2.3, 1.4 Hz, 1H), 7.41-7.26 (m, 5H), 4.96-4.75 (m, 2H), 4.57-4.48 (m, 2H), 4.46-4.38 (m, 2H), 4.35-4.29 (m, 3H), 4.01-3.97 (m, 2H), 3.52 (s, 1H); LCMS (ES) [M+1]⁺ m/z: 457.

### Example 1.56

### Synthesis of (2S)-1-[2-(1,3-benzothiazole-6-sulfonyl)-2H,4H,5H,6H-pyrrolo[3,4-c]pyrazol-5-yl]-2-(2-fluorophenyl)-3-hydroxypropan-1-one (Compound 79)

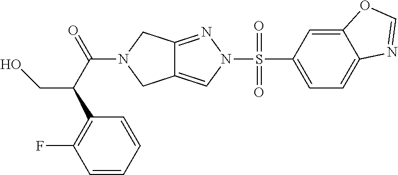 [SMILES: O=C([C@H](CO)c1ccccc1F)N1Cc2cn(S(=O)(=O)c3ccc4ncoc4c3)nc2C1]

Into a 50 mL 3-necked round-bottom flask, was placed (2S)-2-(2-fluorophenyl)-3-hydroxypropanoic acid (Intermediate-II-10; 60 mg, 0.33 mmol, 1.0 eq.), 6-{2H,4H,5H,6H-pyrrolo[3,4-c]pyrazole-2-sulfonyl}-1,3-benzothiazole (Intermediate I-1, 100 mg, 0.33 mmol, 1.0 eq.) and DMF (3 mL). The reaction was cooled to 0° C., NMM (82 mg, 0.81 mmol, 2.5 eq.) and HATU (149 mg, 0.39 mmol, 1.2 eq.) were added at 0° C. and the resulting solution was stirred for 1 h at 0° C. The reaction was then directly purified by Prep-HPLC with the following conditions (IntelFlash-1): Column, C18 silica gel; mobile phase, MeCN/H₂O=1:9 increasing to MeCN/H2O=1:1 within 15; Detector, 220. This resulted in (2S)-1-[2-(1,3-benzothiazole-6-sulfonyl)-4H,6H-pyrrolo[3,4-c]pyrazol-5-yl]-2-(2-fluorophenyl)-3-hydroxypropan-1-one (65 mg, 42.22%) as a white solid. ¹H NMR (300 MHz, DMSO-d6) δ 9.68 (s, 1H), 8.99 (d, J=2.0 Hz, 1H), 8.31-8.28 (m, 2H), 8.04 (dd, J=8.7, 2.0 Hz, 1H), 7.40-7.22 (m, 2H), 7.22-7.08 (m, 2H), 4.93-4.79 (m, 1H), 4.51-4.16 (m, 4H), 3.96 (dd, J=10.2, 8.2 Hz, 1H), 3.58-3.35 (m, 1H); LCMS (ES) [M+1]+m/z: 473.

### Example 1.57

### Synthesis of (2S)-1-(2-{2H,3H-[1,4]dioxino[2,3-b]pyridine-7-sulfonyl}-2H,4H,5H,6H pyrrolo[3,4-c]pyrazol-5-yl)-2-(2-fluorophenyl)-3-hydroxypropan-1-one (Compound 80)

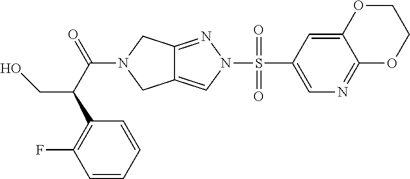 [SMILES: O=C([C@H](CO)c1ccccc1F)N1Cc2cn(S(=O)(=O)c3cnc4c(c3)OCCO4)nc2C1]

Into a 50 mL 3-necked round-bottom flask, was placed (2S)-2-(2-fluorophenyl)-3-hydroxypropanoic acid (Intermediate-II-10; 80 mg, 0.43 mmol, 1.0 eq.), 2-{2H,3H-[1,4]dioxino[2,3-b]pyridine-7-sulfonyl}-2H,4H,5H,6H-pyrrolo[3,4-c]pyrazole (Intermediate I-8, 133 mg, 0.43 mmol, 1.0 eq.) and DMF (4 mL). After the reaction was cooled to 0° C., NMM (109 mg, 1.08 mmol, 2.5 eq.) and HATU (198 mg, 0.52 mmol, 1.2 eq.) were added and the resulting solution was stirred for 1 h at 0° C. The reaction mixture was then directly purified by Prep-HPLC with the following conditions (IntelFlash-1): Column, C18 silica gel; mobile phase, MeCN/H20=1:9 increasing to MeCN/H2O=1:1 within 15; Detector, 220, to provide the title compounds as a white solid (90 mg, 43.67%). ¹H NMR (300 MHz, DMSO-d₆) δ 8.32 (dd, J=2.3, 0.8 Hz, 1H), 8.25 (d, J=3.4 Hz, 1H), 7.73 (dd, J=2.3, 1.4 Hz, 1H), 7.44-7.25 (m, 2H), 7.25-7.08 (m, 2H), 4.89 (dd, J=18.6, 14.1 Hz, 1H), 4.59-4.36 (m, 4H), 4.34-4.12 (m, 4H), 3.98 (dd, J=10.2, 8.2 Hz, 1H), 3.59 (dt, J=10.3, 5.3 Hz, 1H); LCMS (ES) [M+1]⁺ m/z: 475.

### Example 1.58

### Synthesis of (2S)-1-[2-(1-benzofuran-5-sulfonyl)-2H,4H,5H,6H-pyrrolo[3,4-c]pyrazol-5-yl]-2-(2-fluorophenyl)-3-hydroxypropan-1-one (Compound 84)

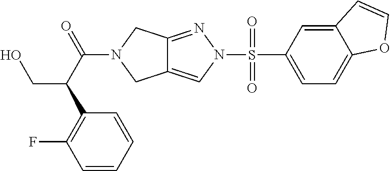 [SMILES: O=C([C@H](CO)c1ccccc1F)N1Cc2cn(S(=O)(=O)c3ccc4occc4c3)nc2C1]

Into a 8-mL vial, was placed a mixture of 2-(1-benzofuran-5-sulfonyl)-2H,4H,5H,6H-pyrrolo[3,4-c]pyrazole (Intermediate I-5, 120 mg, 0.415 mmol, 1.00 eq.), DMF (2.00 mL), (S)-2-(2-fluorophenyl)-3-hydroxypropanoic acid (Intermediate II-10; 76.3 mg, 0.415 mmol, 1.00 eq.), NMM (125 mg, 1.24 mmol, 3.00 eq.), and HATU (205 mg, 0.540 mmol, 1.30 eq.) and the resulting solution was stirred for 2 h at room temperature. The crude product was purified by Prep-HPLC with the following conditions: SunFire Prep C18 OBD Column, 19×150 mm, 5 um; mobile phase, phase A: H₂O (0.1% FA); phase B: CH₃CN/MeOH (1/1) (20% CH₃CN/MeOH up to 55% CH₃CN/Meoh in 15 min). This resulted in the title compound as a white solid (117.2 mg, 62.04%) ¹H NMR (300 MHz, DMSO-d₆, ppm): 8.40 (s, 1H), 8.28-8.21 (m, 2H), 7.94-7.85 (m, 2H), 7.45-7.07 (m, 5H), 4.87 (t, J=14.1 Hz, 1H), 4.56-4.19 (m, 4H), 3.97 (t, J=9.2 Hz, 1H), 3.67-3.63(m, 1H); LCMS (ES) [M+1]⁺ m/z 456.

### Example 1.59

### Synthesis of (2S)-1-(2-[6-(difluoromethoxy)pyridin-3-yl]sulfonyl-2H,4H,5H,6H-pyrrolo[3,4-c]pyrazol-5-yl)-2-(2-fluorophenyl)-3-hydroxypropan-1-one (Compound 85)

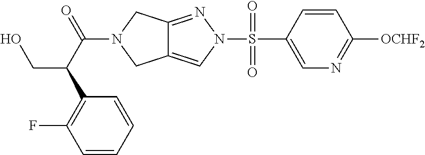 [SMILES: O=C([C@H](CO)c1ccccc1F)N1Cc2cn(S(=O)(=O)c3ccc(OC(F)F)nc3)nc2C1]

Step 1

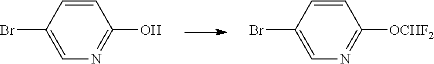 [SMILES: Oc1ccc(Br)cn1 ; FC(F)Oc1ccc(Br)cn1]

Into a 500-mL round-bottom flask purged and maintained with an inert atmosphere of nitrogen, was placed 5-bromopyridin-2-ol (20.00 g, 114.94 mmol, 1.00 eq.), CH₃CN (250.00 mL), difluoro(sulfo)acetic acid (20.47 g, 114.94 mmol, 1.00 eq.), and Na₂SO₄ (1.63 g, 11.49 mmol, 0.10 eq.) and the resulting mixture was stirred for 24 h at room temperature. After this, the mixture was filtered and the filtrate was concentrated under reduced pressure and purified by silica gel column eluting with ethyl acetate/petroleum ether (1/10). This resulted in 17 g (66%) of 5-bromo-2-(difluoromethoxy)pyridine as a light yellow liquid. LCMS (ES)[M+1]⁺ m/z: 224.

Step 2

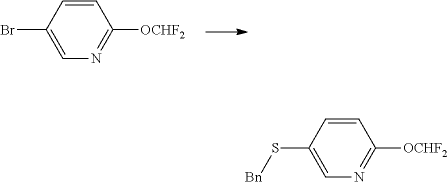 [SMILES: FC(F)Oc1ccc(Br)cn1 ; FC(F)Oc1ccc(SCc2ccccc2)cn1]

Into a 500-mL round-bottom flask purged and maintained with an inert atmosphere of nitrogen, was placed 5-bromo-2-(difluoromethoxy)pyridine (17.00 g, 75.89 mmol, 1.00 eq.), benzyl mercaptan (18.85 g, 151.78 mmol, 2.00 eq.), DIEA (29.43 g, 227.67 mmol, 3.00 eq.), dioxane (250.00 mL), XantPhos (8.78 g, 15.17 mmol, 0.20 eq.) and Pd₂(dba)₃ (6.95 g, 7.58 mmol, 0.10 eq.) and the mixture was stirred for 16 h at 100° C. The reaction mixture was then cooled to room temperature and concentrated under reduced pressure. The residue was purified by silica gel column eluting with ethyl acetate/petroleum ether (1/20). This resulted in 18.7 g (92%) of 5-(benzylsulfanyl)-2-(difluoromethoxy)pyridine as a light yellow oil. LCMS (ES)[M+1]⁺ m/z: 268.

Step 3

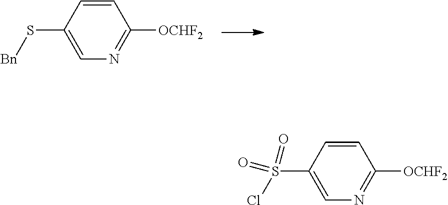 [SMILES: FC(F)Oc1ccc(SCc2ccccc2)cn1 ; O=S(=O)(Cl)c1ccc(OC(F)F)nc1]

Into a 500-mL round-bottom flask purged and maintained with an inert atmosphere of nitrogen, was placed 5-(benzylsulfanyl)-2-(difluoromethoxy)pyridine (18.70 g, 69.96 mmol, 1.00 eq.), AcOH (180.00 mL), H₂O (20 mL), and NCS (28.03 g, 209.88 mmol, 3.00 eq.) and the resulting solution was stirred for 16 h at room temperature. The reaction mixture was then concentrated and the residue was purified by silica gel column eluting with ethyl acetate/petroleum ether (1/10). This resulted in 15 g (88%) of 6-(difluoromethoxy)pyridine-3-sulfonyl chloride as a light yellow oil.

Step 4

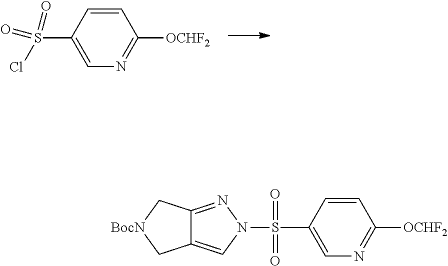 [SMILES: O=S(=O)(Cl)c1ccc(OC(F)F)nc1 ; CC(C)(C)OC(=O)N1Cc2cn(S(=O)(=O)c3ccc(OC(F)F)nc3)nc2C1]

Into a 500-mL round-bottom flask purged and maintained with an inert atmosphere of nitrogen, was placed a solution of t-butyl 2H,4H,6H-pyrrolo[3,4-c]pyrazole-5-carboxylate (12.88 g, 61.57 mmol, 1.00 eq.) in DMF (200.00 mL) followed by the addition of NaH (4.93 g, 123.14 mmol, 2.00 eq., 60% in mineral oil) at 0° C. The resulting solution was stirred for 0.5 h at 0° C., then a solution of 6-(difluoromethoxy)pyridine-3-sulfonyl chloride (15.00 g, 61.57 mmol, 1.00 eq.) in DMF (50.00 mL) was added dropwise with stirring at 0° C. The resulting solution was stirred for 0.5 h at 0° C. The reaction was then quenched by the addition of 200 mL of NH₄Cl (aq) and extracted with 3×150 mL of ethyl acetate. The combined organic layer was dried over anhydrous sodium sulfate, filtered, and the filtrate was concentrated. The residue was purified by silica gel column eluting with PE/EA=10/1. This resulted in 20 g (78%) of t-butyl 2-[6-(difluoromethoxy)139yridine-3-ylsulfonyl]-4H,6H-pyrrolo[3,4-c]pyrazole-5-carboxylate as a white solid. LCMS (ES) [M+1]⁺ m/z: 417.

Step 5

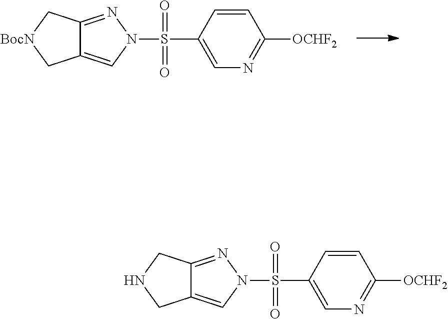 [SMILES: CC(C)(C)OC(=O)N1Cc2cn(S(=O)(=O)c3ccc(OC(F)F)nc3)nc2C1 ; O=S(=O)(c1ccc(OC(F)F)nc1)n1cc2c(n1)CNC2]

Into a 500-mL round-bottom flask purged and maintained with an inert atmosphere of nitrogen, was placed t-butyl 2-[6-(difluoromethoxy)139yridine-3-ylsulfonyl]-4H,6H-pyrrolo[3,4-c]pyrazole-5-carboxylate (20.00 g, 48.03 mmol, 1.00 eq.) in DCM (200.00 mL) followed by the addition of lutidine (10.29 g, 96.06 mmol, 2.00 eq.) at 0° C. and TMSOTf (32.03 g, 144.09 mmol, 3.00 eq.) at the same temperature. The resulting solution was stirred for 1 h at room temperature. The reaction was then quenched by the addition of 200 mL of water, the pH value of the solution was adjusted to 7-8 with NaHCO₃ (10% in H₂O) and the mixture was extracted with 3×150 mL of dichloromethane. The organic layers were combined, dried over anhydrous sodium sulfate, filtered, and concentrated under reduced pressure. The residue was triturated in PE/EA=5/1 (100.00 mL) resulting in 2-((6-(difluoromethoxy)140yridine-3-yl)sulfonyl)-2,4,5,6-tetrahydropyrrolo[3,4-c]pyrazole (14.0 g, 92%) as a white solid. LCMS (ES) [M+1]⁺ m/z: 317.

Step 6

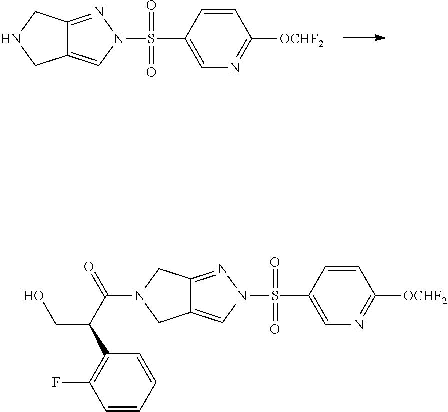 [SMILES: O=S(=O)(c1ccc(OC(F)F)nc1)n1cc2c(n1)CNC2 ; O=C([C@H](CO)c1ccccc1F)N1Cc2cn(S(=O)(=O)c3ccc(OC(F)F)nc3)nc2C1]

Into a 20-mL vail, was placed (S)-2-(2-fluorophenyl)-3-hydroxypropanoic acid (Intermediate II-10; 80 mg, 0.44 mmol, 1.00 eq.), DMF (2.00 mL), 2-(difluoromethoxy)-5-{2H,4H,5H,6H-pyrrolo[3,4-c]pyrazole-2-sulfonyl}pyridine (153 mg, 0.48 mmol, 1.10 eq.), and NMM (134 mg, 1.32 mmol, 3.00 eq.) followed by the addition of HATU (218 mg, 0.57 mmol, 1.30 eq.) at 0° C. The resulting solution was stirred for 1 h at room temperature. The reaction mixture was then directly purified by Prep-HPLC with conditions: Column, Sunfire Prep C18 OBD Column, 50*250 mm 5 um 10 nm, Mobile Phase A: Water (0.1% FA), Mobile Phase B: CH₃CN, Flow rate: 90 mL/min, Gradient: 20% B to 55% B in 15 min, Detector, 220 nm, to provide the title compound as white solid (96 mg, 45%). ¹HNMR (300 MHz, DMSO-d₆, ppm): δ 8.88 (d, J=2.4 Hz, 1H), 8.45-8.41 (m, 1H), 8.29 (d, J=4.2 Hz, 1H), 7.99 (td, J=71.4 Hz, 1H), 7.41-7.28 (m, 3H), 7.23-7.14 (m, 2H), 4.95 (dd, J=19.5, 14.5 Hz, 1H), 4.52-4.22 (m, 4H), 4.01-3.95 (m, 1H), 3.63-3.55 (m, 1H); LCMS-PH-GBT-MY-PK-409-0: (ES, m/z): [M+H]⁺: 483.

### Example 1.60

### Synthesis of (2S)-2-(2-chlorophenyl)-1-(2-{furo[2,3-b]pyridine-5-sulfonyl}-2H,4H,5H,6H-pyrrolo[3,4-c]pyrazol-5-yl)-3-hydroxypropan-1-one (Compound 86)

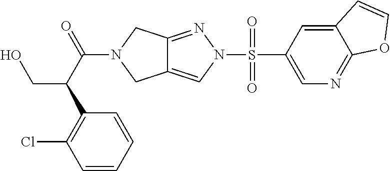 [SMILES: O=C([C@H](CO)c1ccccc1Cl)N1Cc2cn(S(=O)(=O)c3cnc4occc4c3)nc2C1]

Step 1

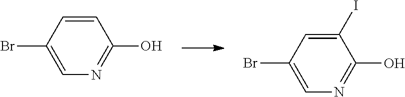 [SMILES: Oc1ccc(Br)cn1 ; Oc1ncc(Br)cc1I]

Into a 1000-mL 3-necked round-bottom flask, was placed 5-bromopyridin-2-ol (23 g, 132 mmol, 1.00 eq.), DMF (500.00 mL) followed by N-iodosuccinimide (32.7 g, 145 mmol, 1.10 eq.) under nitrogen, in portions at 0° C. The reaction mixture was stirred at 90° C. for 50 minutes then cooled to 25° C. The precipitate was filtered off and washed with methanol, dried under vacuum to give 37 g (93.4%) of 5-bromo-3-iodopyridin-2-ol as a pink-orange color solid. LCMS (ES) [M+1]⁺ m/z: 300.

Step 2

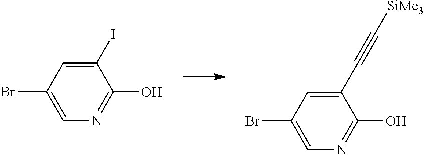 [SMILES: Oc1ncc(Br)cc1I ; C[Si](C)(C)C#Cc1cc(Br)cnc1O]

Into a 500-mL round-bottom flask purged and maintained with an inert atmosphere of nitrogen, was placed 5-bromo-3-iodopyridin-2-ol (37.0 g, 123 mmol, 1.00 eq.), Pd(Oac)₂ (1.38 g, 6 mmol, 0.05 eq.), PPh₃ (3.14 g, 12 mmol, 0.1 eq.), and CuI (2.28 g, 12 mmol, 0.1 eq). THF (350 mL) was then added and the solution was degassed 2 minutes with nitrogen. TMS-acetylene (18.12 g, 184.5 mmol, 1.50 eq.) was added followed with n-BuNH₂ (17.99 g, 246 mmol, 2.00 eq.). The homogeneous green solution was heated to 60° C. for 5 h. The reaction mixture was then cooled to 25° C., concentrated to dryness, and dissolved in EtOAc (400 mL). The solution was washed with saturated sodium potassium tartrate (200 mL), followed by 0.1 N HCl (100 mL), saturated NaHCO₃ (100 mL), and brine (100 mL), and dried over Na₂SO₄. Purification by column chromatography (SiO₂, 50% ethyl acetate/hexanes) afforded 14.4 g (69.57%) of 5-bromo-3-((trimethylsilyl)ethynyl)pyridin-2-ol as a green solid. LCMS (ES) [M+1]⁺ m/z: 270.

Step 3

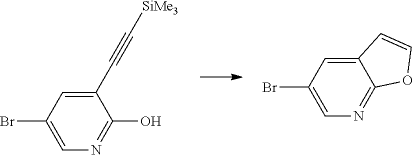 [SMILES: C[Si](C)(C)C#Cc1cc(Br)cnc1O ; Brc1cnc2occc2c1]

Into a 500-mL round-bottom flask, was placed 5-bromo-3-((trimethylsilyl)ethynyl)pyridin-2-ol (16 g, 59.25 mmol, 1.00 eq.). EtOH (200 mL) and CuI (563 mg, 2.96 mmol, 0.05 eq.) were added, followed by Et₃N (11.97 g, 118.5 mmol, 2.00 eq.), and the resulting mixture was stirred at 70° C. for 3 h. The reaction mixture was then allowed to cool to 25° C., concentrated to dryness, and dissolved in toluene (30 mL). The solution was washed with 0.1 N HCl (20 mL), saturated NaHCO₃ (20 mL), and brine (30 mL), and dried over Na₂SO₄. Purification by column chromatography (20% ethylacetate/hexanes) afforded 4.3 g (37%) of 5-bromofuro[2,3-b]pyridine as a white solid. LCMS (ES) [M+1]⁺ m/z: 198.

Step 4

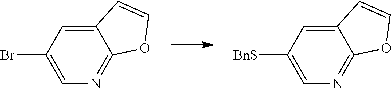

Into a 100-mL round-bottom flask purged and maintained with an inert atmosphere of nitrogen, was placed 5-bromofuro[2,3-b]pyridine (2.00 g, 10 mmol, 1.00 eq.), benzyl mercaptan (3.7 g, 30 mmol, 3.00 eq.), toluene (50.00 mL), DIEA (3.87 g, 30 mmol, 3.00 eq.), XantPhos (1.16 g, 2 mmol, 0.20 eq.), and Pd₂(dba)₃ (915 mg, 1 mmol, 0.10 eq.), and the resulting solution was stirred overnight at 100° C. The reaction mixture was then cooled to room temperature and concentrated. The residue was applied onto a silica gel column and eluted with THF/PE (5%). This resulted in 2.18 g (91%) of 5-(benzylthio)furo[2,3-b]pyridine as a yellow oil. LCMS (ES) [M+1]⁺ m/z: 242.

Step 5

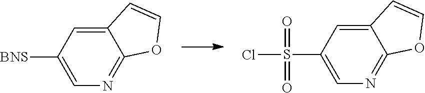

Into a 100-mL round-bottom flask, was placed 5-(benzylthio)furo[2,3-b]pyridine (3.00 g, 12.4 mmol, 1.00 eq.), and HOAc (36.00 mL), H₂O (4.00 mL). This was followed by the addition of NCS (4.95 g, 37.2 mmol, 3.00 eq.), in portions at 0° C. The resulting solution was stirred for 20 min at room temperature, then it was concentrated. The residue was applied onto a silica gel column with THF/PE (10%). This resulted in 1.8 g (67%) of furo[2,3-b]pyridine-5-sulfonyl chloride as a yellow solid. LCMS (ES) [M+1]⁺m/z: 218.

Step 6

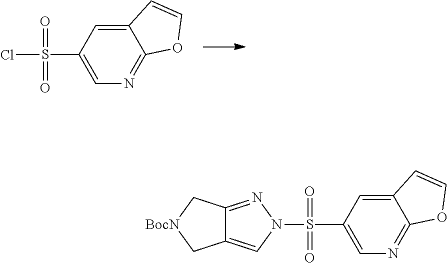 [SMILES: O=S(=O)(Cl)c1cnc2occc2c1 ; CC(C)(C)OC(=O)N1Cc2cn(S(=O)(=O)c3cnc4occc4c3)nc2C1]

Into a 100-mL 3-necked round-bottom flask, was placed t-butyl 2H,4H,6H-pyrrolo[3,4-c]pyrazole-5-carboxylate (2.5 g, 11.96 mmol, 1.00 eq.) in THF (30.00 mL), followed by the addition of NaH (60% in mineral oil) (0.62 g, 15.55 mmol, 1.30 eq.), in portions at 0° C. The mixture was stirred for 0.5 h, then furo[2,3-b]pyridine-5-sulfonyl chloride (2.86 g, 13.16 mmol, 1.10 eq.) was added dropwise with stirring at 0° C. The resulting solution was stirred for 1 h at 0° C., then it was quenched by the addition of 5 mL of HOAc. The resulting solution was extracted with 3×200 mL of dichloromethane and the organic layers were combined, dried over anhydrous sodium sulfate, and concentrated. The resulting solution was diluted with 200 mL of MTBE and the solids were collected by filtration. This resulted in 2.5 g (53.6%) of t-butyl 2-(furo[2,3-b]pyridin-5-ylsulfonyl)-2,6-dihydropyrrolo[3,4-c]pyrazole-5(4H)-carboxylate as a white solid. LCMS (ES) [M+1]⁺ m/z: 391.

Step 7

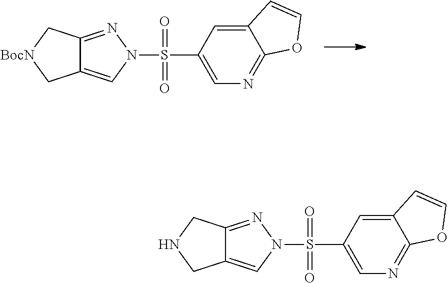 [SMILES: CC(C)(C)OC(=O)N1Cc2cn(S(=O)(=O)c3cnc4occc4c3)nc2C1 ; O=S(=O)(c1cnc2occc2c1)n1cc2c(n1)CNC2]

Into a 100-mL round-bottom flask, was placed t-butyl 2-(furo[2,3-b]pyridin-5-ylsulfonyl)-2,6-dihydropyrrolo[3,4-c]pyrazole-5(4H)-carboxylate (1.4 g, 3.6 mmol, 1.00 eq.), DCM (20.00 mL), and lutidine (1.54 g, 14.4 mmol, 4.00 eq.), followed by the addition of TMSOTf (2.4 g, 10.8 mmol, 3.00 eq.) dropwise with stirring at 0° C. The resulting solution was stirred for 1 h at 0° C., then it was quenched by the addition of water/ice. The resulting mixture was concentrated and the crude product (3 g) was purified by Prep-HPLC with the following conditions: Column, Xbridge Prep C18 OBD Column, 19 cm, 150 mm, 5 um; mobile phase, Water (0.1% FA) and can (5% Phase B up to 20% in 11 min); Detector, 254 nm. This resulted in 0.8 g (77%) of 5-((5,6-dihydropyrrolo[3,4-c]pyrazol-2(4H)-yl)sulfonyl)furo[2,3-b]pyridine. LCMS (ES) [M+1]⁺ m/z: 291.

Step 8

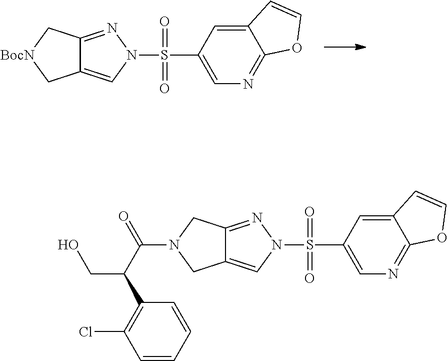 [SMILES: CC(C)(C)OC(=O)N1Cc2cn(S(=O)(=O)c3cnc4occc4c3)nc2C1 ; O=C([C@H](CO)c1ccccc1Cl)N1Cc2cn(S(=O)(=O)c3cnc4occc4c3)nc2C1]

Into a 20-mL vial, was placed (2S)-2-(2-chlorophenyl)-3-hydroxypropanoic acid (Intermediate II-11; 90.00 mg, 0.45 mmol, 1.00 eq.), 2-[furo[2,3-b]pyridine-5-sulfonyl]-4H,5H,6H-pyrrolo[3,4-c]pyrazole-trifluoromethanesulfonic acid (197.55 mg, 0.45 mmol, 1.00 eq.), DMF (5.00 mL), and NMM (136.13 mg, 1.35 mmol, 3.00 eq.), followed by the addition of HATU (204.69 mg, 0.54 mmol, 1.20 eq.), in portions at 0° C. The resulting solution was stirred for 2 h at room temperature. The crude product (0.3 g) was purified by Prep-HPLC with the following conditions: Column, XBridge Prep C18 OBD Column, 19 cm, 150 mm, 5 um; mobile phase, Water (0.1% FA) and CAN (5% Phase B up to 60% in 11 min); Detector, 254. This resulted in (2S)-2-(2-chlorophenyl)-1-(2-[furo[2,3-b]pyridine-5-sulfonyl]-4H,6H-pyrrolo[3,4-c]pyrazol-5-yl)-3-hydroxypropan-1-one as a white solid (157.8 mg, 74.4%). 1H-NMR (300 MHz, DMSO-d₆, ppm): δ 8.90 (d, J=2.0 Hz, 1H), 8.84-8.77 (m, 1H), 8.36 (d, J=2.5 Hz, 1H), 8.32 (s, 1H), 7.51-7.36 (m, 2H), 7.30-7.27 (m, 2H), 7.20 (d, J=1.9 Hz, 1H), 4.97-4.81 (m, 1H), 4.54-4.22 (m, 4H), 3.94 (t, J=9.3 Hz, 1H), 3.57-3.54 (m, 1H); LCMS (ES, m/z): [M+H]⁺: 473.

### Example 1.61

### Synthesis of (2R)-2-(3-fluoropyridin-2-yl)-2-hydroxy-1-[2-(4-methoxybenzenesulfonyl)-2H,4H,5H,6H-pyrrolo[3,4-c]pyrazol-5-yl]ethan-1-one or (2R)-2-(3-fluoropyridin-2-yl)-2-hydroxy-1-[2-(4-methoxybenzenesulfonyl)-2H,4H,5H,6H-pyrrolo[3,4-c]pyrazol-5-yl]ethan-1-one

(Compound 87)

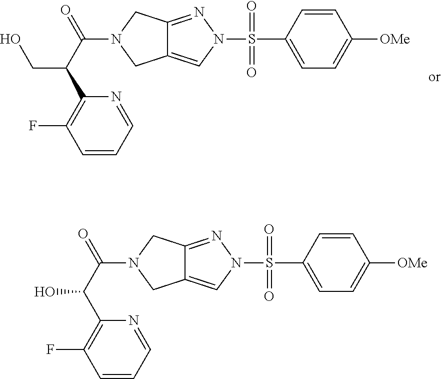 [SMILES: COc1ccc(S(=O)(=O)n2cc3c(n2)CN(C(=O)[C@H](CO)c2ncccc2F)C3)cc1 ; COc1ccc(S(=O)(=O)n2cc3c(n2)CN(C(=O)[C@@H](O)c2ncccc2F)C3)cc1]

Into a 40-mL vial, was placed racemic (3-fluoropyridin-2-yl)(hydroxy)acetic acid (130.00 mg, 0.76 mmol, 1.00 eq.), 2-(4-methoxybenzenesulfonyl)-4H,5H,6H-pyrrolo[3,4-c]pyrazole hydrochloride (239.88 mg, 0.76 mmol, 1.00 eq.), DMF (8.00 mL), and NMM (230.51 mg, 2.28 mmol, 3.00 eq.), followed by the addition of HATU (346.62 mg, 0.91 mmol, 1.20 eq.), in portions at 0° C. The resulting solution was stirred for 2 h at room temperature. The crude product (1 g) was purified by Prep-HPLC (Column, XBridge Prep C18 OBD Column, 19 cm, 150 mm, 5 um; mobile phase, Water (0.1% FA) and CAN (5% Phase B up to 60% in 11 min); Detector, 254) to provide a white product, which was purified by Prep-SFC (Column, CHIRAL RT Cellulose-SJ, 3*25 cm, 5 um; mobile phase, CO₂ (65%) and MeOH (0.1% 2M NH₃-MeOH) (35%); Detector, 254).

The fraction with RT=1.39 min was collected to provide the title compound as a white solid (69.8 mg, 38.78%). ¹H-NMR: (300 MHz, DMSO-d₆, ppm): δ 8.37 (ddt, J=4.7, 3.1, 1.5 Hz, 1H), 8.21 (d, J=16.2 Hz, 1H), 7.90 (d, J=8.7 Hz, 1H), 7.75 (dddd, J=10.0, 8.4, 4.9, 1.4 Hz, 1H), 7.47 (dq, J=8.5, 4.2 Hz, 1H), 7.18 (d, J=8.7 Hz, 2H), 5.78 (t, J=6.8 Hz, 1H), 5.59 (t, J=6.5 Hz, 1H), 4.82 (t, J=13.9 Hz, 1H), 4.48 (qd, J=15.9, 5.5 Hz, 2H), 4.10 (dd, J=19.6, 14.3 Hz, 1H), 3.85 (s, 3H); LCMS(ES, m/z): [M+H]⁺: 433.

### Example 1.62

### Synthesis of (2S)-2-(2-fluorophenyl)-3-hydroxy-1-[2-(4-methoxybenzenesulfonyl)-2H,4H,5H,6H-pyrrolo[3,4-c]pyrazol-5-yl]propan-1-one (Compound 88)

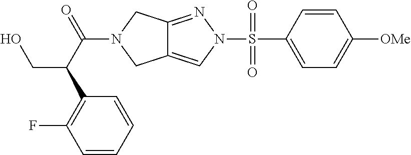 [SMILES: COc1ccc(S(=O)(=O)n2cc3c(n2)CN(C(=O)[C@H](CO)c2ccccc2F)C3)cc1]

Into a 20-mL vial, was placed (2S)-2-(2-fluorophenyl)-3-hydroxypropanoic acid (Intermediate II-10; 90.00 mg, 0.49 mmol, 1.00 eq.), 2-(4-methoxybenzenesulfonyl)-4H,5H,6H-pyrrolo[3,4-c]pyrazole; trifluoromethanesulfonic acid (Intermediate I-9; 209.84 mg, 0.49 mmol, 1.00 eq.), DMF (5.00 mL), and NMM (98.86 mg, 0.98 mmol, 2.00 eq.), followed by the addition of HATU (222.98 mg, 0.58 mmol, 1.20 eq.), in portions at 0° C. The resulting solution was stirred for 2 h at room temperature. The crude product (0.3 g) was purified by Prep-HPLC with the following conditions: Column, XBridge Prep C18 OBD Column, 19 cm, 150 mm, 5 um; mobile phase, Water (0.1% FA) and CAN (5% Phase B up to 60% in 11 min); Detector, 254. This resulted in the title compound as a white solid (168.7 mg, 77.49%). ¹H-NMR: (300 MHz, DMSO-d₆, ppm): δ 8.22 (d, J=3.0 Hz, 1H), 7.90 (d, J=8.7, 2H), 7.38 (ddd, J=9.5, 6.6, 2.0 Hz, 1H), 7.30 (dtt, J=7.5, 4.8, 2.5 Hz, 1H), 7.24-7.10 (m, 4H), 4.87 (dd, J=16.2, 14.0 Hz, 1H), 4.52-4.36 (m, 2H), 4.33-4.22 (m, 2H), 3.98 (dd, J=10.2, 8.2 Hz, 1H), 3.85 (s, 3H), 3.59 (dt, J=10.4, 5.4 Hz, 1H); LCMS (ES, m/z): [M+H]⁺: 446.

### Example 1.63

### Synthesis of (2S)-1-{2-[4-(difluoromethoxy)benzenesulfonyl]-2H,4H,5H,6H-pyrrolo [3,4-c]pyrazol-5-yl}-2-(3-fluoropyridin-2-yl)-3-hydroxypropan-1-one or (2R)-1-{2-[4-(difluoromethoxy)benzenesulfonyl]-2H,4H,5H,6H-pyrrolo[3,4-c]pyrazol-5-yl}-2-(3-fluoropyridin-2-yl)-3-hydroxypropan-1-one (Compound 89)

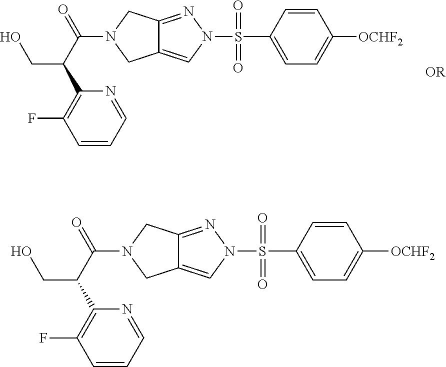 [SMILES: O=C([C@H](CO)c1ncccc1F)N1Cc2cn(S(=O)(=O)c3ccc(OC(F)F)cc3)nc2C1 ; O=C([C@@H](CO)c1ncccc1F)N1Cc2cn(S(=O)(=O)c3ccc(OC(F)F)cc3)nc2C1]

Step 1

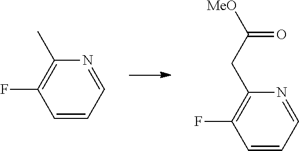

Into a 250-mL 3-necked round-bottom flask purged and maintained with an inert atmosphere of nitrogen, was placed 3-fluoro-2-methylpyridine (8.00 g, 71.99 mmol, 1.00 eq.) in THF (100.00 mL), and the mixture was cooled to −78° C. This was followed by the addition of TMEDA (12.55 g, 107.99 mmol, 1.50 eq.) and LDA (2M in THF) (54.00 mL, 108.00 mmol, 1.50 eq.) at −78° C., and the resulting solution was stirred for 1 h at the same temperature. To the mixture was then added dimethyl carbonate (25.94 g, 287.98 mmol, 4.00 eq.) at −78° C. and after this addition, the resulting solution was stirred for 3 h at room temperature. The reaction was then quenched by the addition of 200 mL of NH₄Cl (aq), and extracted with 3×100 mL of ethyl acetate. The organic layers were combined and dried over anhydrous sodium sulfate, filtered, and concentrated under reduced pressure. The residue was purified by silica gel column eluting with ethyl acetate/petroleum ether (1/2). This resulted in 4.0 g (33%) of methyl 2-(3-fluoropyridin-2-yl)acetate as a light yellow oil. LCMS (ES) [M+1]⁺ m/z: 170.

Step 2

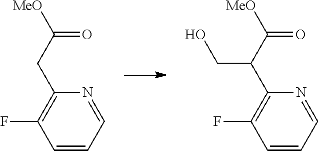 [SMILES: COC(=O)Cc1ncccc1F ; COC(=O)C(CO)c1ncccc1F]

Into a 100-mL 3-necked round-bottom flask purged and maintained with an inert atmosphere of nitrogen, was placed methyl 2-(3-fluoropyridin-2-yl)acetate (2.50 g, 14.77 mmol, 1.00 eq.), and DMF (30.00 mL) followed by the addition of NaOMe (79 mg, 1.47 mmol, 0.10 eq.) at 0° C. Polyoxymethylene (665 mg, 14.779 mmol, 1.00 eq.) was then added at the same temperature and the resulting solution was stirred for 2 h at 0° C. The reaction mixture was then quenched with 20 mL of H₂O, and directly purified by Flash-Prep-HPLC with the following conditions: C18-120 g column, Mobile phase, H₂O (0.1% HCOOH)/CH₃CN=10/1 increased to H₂O (0.1% HCOOH)/CH₃CN=3/1 within 15 min, Detector, UV 220 nm. This resulted in 1.6 g (54%) of methyl 2-(3-fluoropyridin-2-yl)-3-hydroxypropanoate as a colorless oil. LCMS (ES) [M+1]⁺ m/z: 200.

Step 3

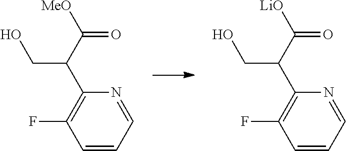

Into a 100-mL round-bottom flask, was placed methyl 2-(3-fluoropyridin-2-yl)-3-hydroxypropanoate (1.60 g, 8.03 mmol, 1.00 eq.), THF (20.00 mL), H₂O (5.00 mL), and LiOH (0.38 g, 16.06 mmol, 2.00 eq.) and the mixture was stirred for 2 h at room temperature. The reaction solution was purified by Flash-Prep-HPLC with the following conditions: Column, C18-120 g, Mobile phase, H₂O/CH₃CN=10/1 increased to H₂O/CH₃CN=4/1 within 15 min, Detector, UV 220 nm. This resulted in 1.2 g (78%) of lithio 2-(3-fluoropyridin-2-yl)-3-hydroxypropanoate as a white solid. LCMS (ES) [M−Li+H+1]⁺ m/z:186.

Step 4

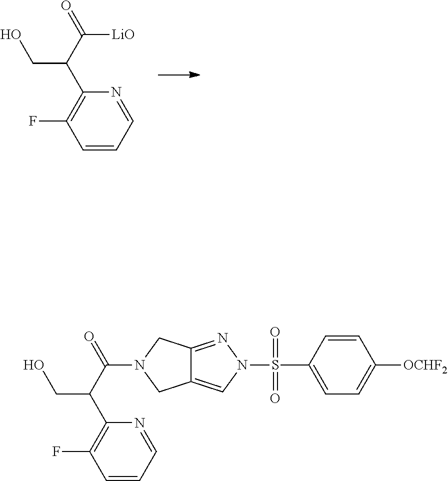

Into a 100-mL 3-necked round-bottom flask, was placed lithio 2-(3-fluoropyridin-2-yl)-3-hydroxypropanoate (600 mg, 3.14 mmol, 1.00 eq.), DMF (10.00 mL), Intermediate I-2 (989 mg, 3.14 mmol, 1.00 eq.), and DIEA (1.22 g, 9.42 mmol, 3.00 eq.) followed by the addition of T₃P (2.40 g, 3.77 mmol, 1.20 eq. 50% in ethyl acetate) dropwise with stirring at 0° C. The resulting solution was stirred for 2 h at room temperature. The reaction mixture was then diluted with 30 mL of H₂O and extracted with 3×30 mL of ethyl acetate. The organic layers were combined, dried over anhydrous sodium sulfate, filtered, and concentrated under reduced pressure. The crude product was purified by Prep-HPLC with the following conditions: Sunfire Prep C18 OBD Column, 50*250 mm 5 um 10 nm, Mobile phase, Water (0.1% FA) and CH₃CN (20% Phase B up to 55% in 15 min), Detector, UV 254 nm. This provided a white solid product (300 mg, 20%). This product was purified by Prep-SFC (Column, CHIRAL RT Cellulose-SJ, 3*25 cm, 5 um, Mobile phase, CO₂ (1) (75%) and MeOH (0.1% 2 M NH₃-MeOH) (25%), Flow rate, 85 mL/min, hold for 10 min, Detector, UV 254 nm).

The fraction at RT=7.07 min was concentrated in vacuum, to provide the title compound as a white solid (93.1 mg, 31.0%); ¹H NMR (300 MHz, DMSO-d₆) δ 8.38-8.34 (m, 1H), 8.27 (d, J=3.3 Hz, 1H), 8.09-8.02 (m, 2H), 7.74-7.17 (m, 5H), 4.95-4.77 (m, 2H), 4.54-4.30 (m, 3H), 4.23 (t, J=13.5 Hz, 1H), 4.16-4.02 (m, 1H), 3.97-3.87 (m, 1H). LCMS: (ES) [M+1]⁺ m/z: 483.

### Example 1.64

### Synthesis of N-[(1S)-2-{2-[4-(difluoromethoxy)benzenesulfonyl]-2H,4H,5H,6H-pyrrolo[3,4-c]pyrazol-5-yl}-2-oxo-1-phenylethyl]formamide (Compound 73)

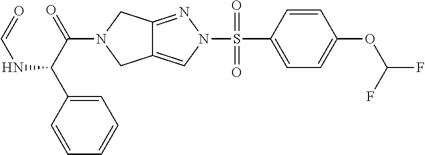 [SMILES: O=CN[C@H](C(=O)N1Cc2cn(S(=O)(=O)c3ccc(OC(F)F)cc3)nc2C1)c1ccccc1]

Step 1

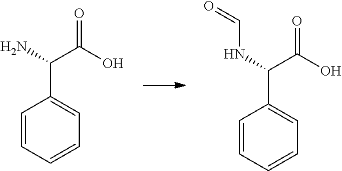 [SMILES: N[C@H](C(=O)O)c1ccccc1 ; O=CN[C@H](C(=O)O)c1ccccc1]

To a solution on (2S)-amino(phenyl)ethanoic acid (1 000.00 mg; 6.62 mmol; 1.00 eq.) in formic acid (50.00 mL; 50.00 V) at 0° C. was added acetic anhydride (18.76 mL; 198.46 mmol; 30.00 eq.) drop-wise. After complete addition, the solution was allowed to come to ambient temperature and stirred overnight. The mixture was diluted with water (36 mL) and the solvent was removed under reduced pressure.

The white residue was re-crystallized from boiling water, and the solid thus formed was collected by filtration and air dried to provide a white powder consistent with (2S)-2-formamido phenylacetic acid (570 mg). A second crop collected as described above provided an additional 324 mg of pure material for a total yield of 894 mg (75%). ¹H NMR (400 MHz, DMSO-d₆) δ 12.93 (s, 1H), 8.89 (d, J=7.6 Hz, 1H), 8.04 (dd, J=1.6, 0.9 Hz, 1H), 7.45-7.19 (m, 5H), 5.35 (dd, J=7.7, 0.9 Hz, 1H).

Step 2

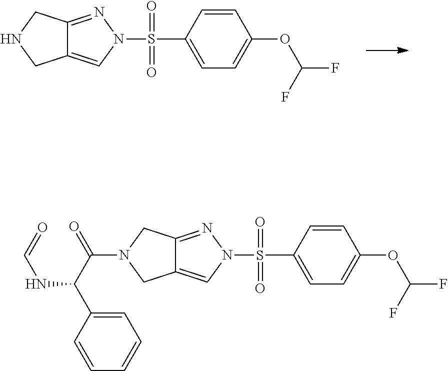 [SMILES: O=S(=O)(c1ccc(OC(F)F)cc1)n1cc2c(n1)CNC2 ; O=CN[C@H](C(=O)N1Cc2cn(S(=O)(=O)c3ccc(OC(F)F)cc3)nc2C1)c1ccccc1]

To a mixture of (2S)-2-formamido-2-phenylacetic acid (221.62 mg; 1.24 mmol; 1.30 eq.), 2-[4-(difluoromethoxy)benzenesulfonyl]-2H,4H,5H,6H-pyrrolo[3,4-c]pyrazole (Intermediate I-2, 300.00 mg; 0.95 mmol; 1.00 eq.) and Hunig's base (0.33 mL; 1.90 mmol; 2.00 eq.) in N,N-dimethylformamide (9.51 mL) was added HATU (470.32 mg; 1.24 mmol; 1.30 eq.) and the resulting mixture was stirred at ambient temperature. After 2 h, the mixture was diluted with 1:1 PhMe/EtOAc and washed with an equal amount of water. The organic phase was dried over MgSO₄, filtered, and concentrated. The residue was purified by column chromatography (12 G ISCO Gold) eluting with 0-80% EtOAc in heptane to provide N-[(1S)-2-{2-[4-(difluoromethoxy)benzenesulfonyl]-2H,4H,5H,6H-pyrrolo[3,4-c]pyrazol-5-yl}-2-oxo-1-phenylethyl]formamide as an off-white solid (261 mg, 57% yield). ¹H NMR (400 MHz, DMSO-d₆) δ 8.85 (ddd, J=7.9, 4.4, 1.6 Hz, 1H), 8.22 (dd, J=6.0, 1.2 Hz, 1H), 8.07-7.96 (m, 3H), 7.44-7.24 (m, 7H), 7.38 (dt, J=72.8, 3.4; OCHF₂), 5.70 (dd, J=16.0, 7.6 Hz, 1H), 4.86 (dd, J=23.7, 14.3 Hz, 1H), 4.51-4.30 (m, 2H), 4.23-4.15 (m, 1H). LCMS (ES) [M+1]⁺ m/z 476.99.

### Example 1.65

### Synthesis of N-[(1R)-2-{2-[4-(difluoromethoxy)benzenesulfonyl]-2H,4H,5H,6H-pyrrolo[3,4-c]pyrazol-5-yl}-1-(2-fluorophenyl)-2-oxoethyl]formamide (Compound 78)

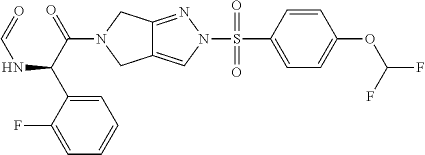 [SMILES: O=CN[C@@H](C(=O)N1Cc2cn(S(=O)(=O)c3ccc(OC(F)F)cc3)nc2C1)c1ccccc1F]

To a mixture of (2R)-2-(2-fluorophenyl)-2-formamidoacetic acid (synthesized from (R)-carboxy(2-fluorophenyl)methanaminium chloride as described for Compound 73, step 1; 101.62 mg; 0.52 mmol; 1.30 eq.), 2-[4-(difluoromethoxy)benzenesulfonyl]-2H,4H,5H,6H-pyrrolo[3,4-c]pyrazole (Intermediate I-2, 125.00 mg; 0.40 mmol; 1.00 eq.) and Hunig's base (0.14 mL; 0.79 mmol; 2.00 eq.) in N,N-dimethylformamide (3.96 mL) was added HATU (195.97 mg; 0.52 mmol; 1.30 eq.) and the resulting mixture was stirred at ambient temperature.

After 2 h, the mixture was diluted with 1:1 PhMe/EtOAc and washed with an equal amount of water. The organic phase was dried over MgSO₄, filtered, and concentrated. The residue was purified by column chromatography (12 G ISCO Gold) eluting with 0-80% EtOAc in heptane to provide N-[(1R)-2-{2-[4-(difluoromethoxy)benzenesulfonyl]-2H,4H,5H,6H-pyrrolo[3,4-c]pyrazol-5-yl}-1-(2-fluorophenyl)-2-oxoethyl]formamide as a white solid (77 mg, 39% yield). ¹H NMR (400 MHz, DMSO-d₆) δ 8.97 (ddd, J=13.5, 7.8, 1.5 Hz, 1H), 8.23 (dd, J=6.7, 1.2 Hz, 1H), 8.04-8.01 (m, 2H), 8.00 (p, J=1.8 Hz, 1H), 7.39 (tdd, J=72.8, 1.7, 0.8 Hz, 1H, OCHF₂), 7.43-7.31 (m, 4H), 7.23-7.15 (m, 2H), 5.94 (dd, J=18.6, 7.9 Hz, 1H), 4.83 (dd, J=20.2, 14.0 Hz, 1H), 4.51-4.33 (m, 2H), 4.19 (dd, J=13.8, 8.9 Hz, 1H). LCMS (ES) [M+1]⁺ m/z 494.98.

### Example 1.66

### Synthesis of N-[(1R)-2-[2-(1,3-benzothiazole-6-sulfonyl)-2H,4H,5H,6H-pyrrolo[3,4-c]pyrazol-5-yl]-1-(2-chlorophenyl)-2-oxoethyl]formamide (Compound 81)

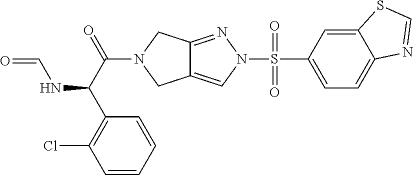 [SMILES: O=CN[C@@H](C(=O)N1Cc2cn(S(=O)(=O)c3ccc4ncsc4c3)nc2C1)c1ccccc1Cl]

To a solution of (2R)-2-amino-1-[2-(1,3-benzothiazole-6-sulfonyl)-2H,4H,5H,6H-pyrrolo[3,4-c]pyrazol-5-yl]-2-(2-chlorophenyl)ethan-1-one (Compound 52); 120.00 mg; 0.25 mmol; 1.00 eq.) in formic acid (6.00 mL; 50.00 V) at 0° C. was added acetic anhydride (0.72 mL; 7.60 mmol; 30.00 eq.) drop-wise. After complete addition, the solution was allowed to come to ambient temperature. After 2.5 h, the mixture was diluted with water (6 mL) and the solvent was removed under reduced pressure. The residue was purified by column chromatography (12 G ISCO Gold) eluting with 0-80% EtOAc in heptane to provide N-[(1R)-2-(1,3-benzothiazole-6-sulfonyl)-2H,4H,5H,6H-pyrrolo[3,4-c]pyrazol-5-yl]-1-(2-chlorophenyl)-2-oxoethyl]formamide as a white solid (74 mg, 58% yield). ¹H NMR (400 MHz, DMSO-d₆) δ 9.67 (q, J=0.8 Hz, 1H), 9.07-8.90 (m, 2H), 8.30-8.23 (m, 2H), 8.08-7.99 (m, 2H), 7.48-7.37 (m, 2H), 7.36-7.27 (m, 2H), 6.00 (dd, J=19.2, 8.2 Hz, 1H), 4.75 (t, J=14.3 Hz, 1H), 4.49-4.33 (m, 2H), 4.29-4.20 (m, 1H). LCMS (ES) [M+1]⁺ m/z 503.06.

### Example 1.67

### Synthesis of (2S)-2-(3-fluoropyridin-2-yl)-3-hydroxy-1-[2-(quinoline-6-sulfonyl)-2H,4H,5H,6H-pyrrolo[3,4-c]pyrazol-5-yl]propan-1-one or (2R)-2-(3-fluoropyridin-2-yl)-3-hydroxy-1-[2-(quinoline-6-sulfonyl)-2H,4H,5H,6H-pyrrolo[3,4-c]pyrazol-5-yl]propan-1-one (Compound 102 and Compound 103)

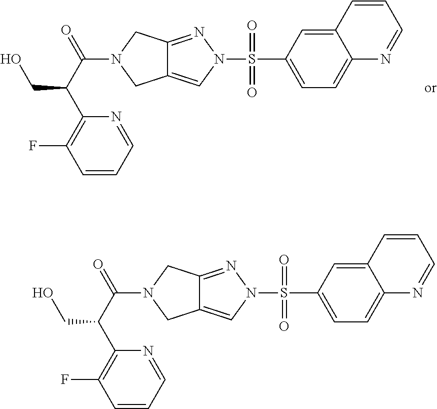 [SMILES: O=C([C@H](CO)c1ncccc1F)N1Cc2cn(S(=O)(=O)c3ccc4ncccc4c3)nc2C1 ; O=C([C@@H](CO)c1ncccc1F)N1Cc2cn(S(=O)(=O)c3ccc4ncccc4c3)nc2C1]

Step 1

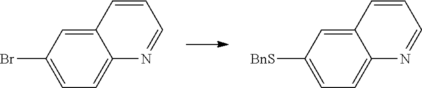

Into a 250 mL 3-necked round-bottom flask, a mixture of 6-bromoquinoline (10.00 g, 48.06 mmol, 1.00 eq.), XantPhos (5.56 g, 9.61 mmol, 0.2 eq.), DIEA (18.60 g, 144.18 mmol, 3 eq.), Pd₂(dba)₃CHCl₃ (4.98 g, 4.81 mmol, 0.1 eq.), and benzyl mercaptan (11.94 g, 96.13 mmol, 2 eq.) in Toluene (100 mL) was stirred overnight at 100° C. under a nitrogen atmosphere. The mixture was then cooled to ambient temperature and concentrated under vacuum. The residue was purified by silica gel column chromatography, eluting with PE/EA (10:1) to afford 6-(benzylsulfanyl)quinoline (11.6 g, 96.02%) as a yellow oil. LCMS (ES) [M+1]⁺ m/z 252.

Step 2

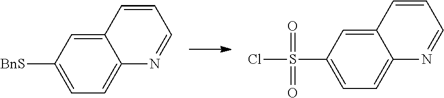

Into a 250 mL round-bottom flask were added 6-(benzylsulfanyl)quinoline (4 g, 15.91 mmol, 1.00 eq.), HOAc (36 mL), H₂O (4 mL), and NCS (6.38 g, 47.74 mmol, 3 eq.) at 0° C. The resulting mixture was stirred for 3 h at room temperature; then it was concentrated under vacuum. The residue was purified by silica gel column chromatography, eluting with PE/EA (8:1) to afford quinoline-6-sulfonyl chloride (3.3 g, 91.08%) as a yellow oil. LCMS (ES) [M+1]⁺ m/z 228.

Step 3

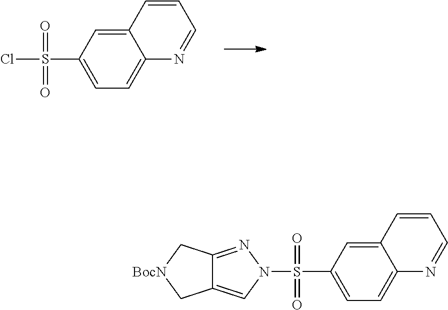 [SMILES: O=S(=O)(Cl)c1ccc2ncccc2c1 ; CC(C)(C)OC(=O)N1Cc2cn(S(=O)(=O)c3ccc4ncccc4c3)nc2C1]

Into a 100 mL 3-necked round-bottom flask were added tert-butyl 2H,4H,6H-pyrrolo[3,4-c]pyrazole-5-carboxylate (1.84 g, 8.78 mmol, 1 eq.) and THF (30 mL) at 0° C. To the above mixture was added NaH (0.42 g, 10.54 mmol, 1.2 eq.) in portions at 0° C. The resulting mixture was stirred for an additional 30 min at 0° C. Quinoline-6-sulfonyl chloride (2.00 g, 8.78 mmol, 1.00 eq.) was then added in portions at 0° C. and the resulting mixture was stirred for an additional 2 h at room temperature. The reaction was then quenched with water at room temperature and extracted with EtOAc (3×100 mL). The combined organic layers were washed with brine (3×100 mL), dried over anhydrous Na₂SO₄, filtered, and concentrated under reduced pressure. The crude product was re-crystallized from EtOAc/PE (1:3 100 mL) to afford tert-butyl 2-(quinoline-6-sulfonyl)-4H,6H-pyrrolo[3,4-c]pyrazole-5-carboxylate (2.5 g, 71.06%) as a white solid. LCMS (ES) [M+1]⁺ m/z 401.

Step 4

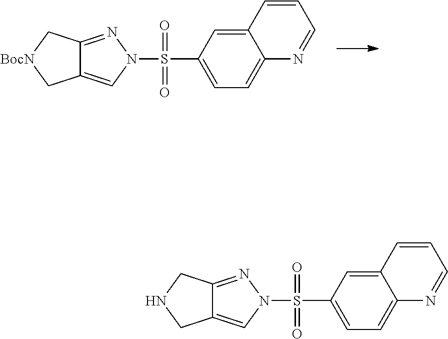 [SMILES: CC(C)(C)OC(=O)N1Cc2cn(S(=O)(=O)c3ccc4ncccc4c3)nc2C1 ; O=S(=O)(c1ccc2ncccc2c1)n1cc2c(n1)CNC2]

Into a 100 mL round-bottom flask were added tert-butyl 2-(quinoline-6-sulfonyl)-4H,6H-pyrrolo[3,4-c]pyrazole-5-carboxylate (2.1 g, 5.24 mmol, 1.00 eq.), DCM (20 mL), 2,6-dimethylpyridine (2.25 g, 20.98 mmol, 4 eq.), and trimethylsilyl triflate (3.50 g, 15.73 mmol, 3 eq.) at room temperature. The resulting mixture was stirred for 2 h at room temperature, then it was quenched with MeOH at room temperature. The crude product was re-crystallized from MeOH/DCM (1:1 30 mL) to afford 6-{4H,5H,6H-pyrrolo[3,4-c]pyrazole-2-sulfonyl}quinoline; trifluoromethanesulfonic acid (1.4 g, 59.27%) as a white solid. LCMS (ES) [M+1]⁺ m/z 301.

Step 5

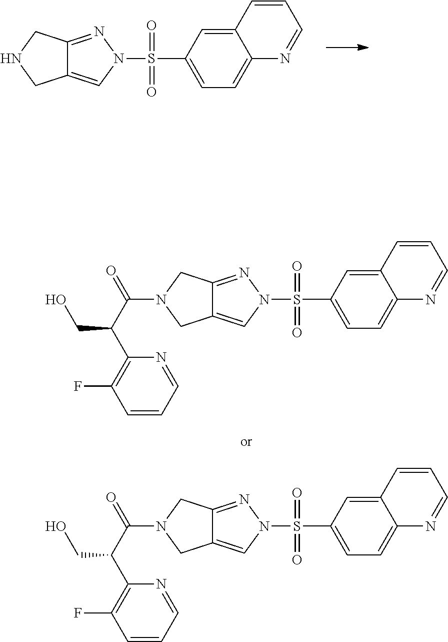 [SMILES: O=S(=O)(c1ccc2ncccc2c1)n1cc2c(n1)CNC2 ; O=C([C@H](CO)c1ncccc1F)N1Cc2cn(S(=O)(=O)c3ccc4ncccc4c3)nc2C1 ; O=C([C@@H](CO)c1ncccc1F)N1Cc2cn(S(=O)(=O)c3ccc4ncccc4c3)nc2C1]

Into a 40 mL vial were added 2-(3-fluoropyridin-2-yl)-3-hydroxypropanoic acid (385 mg, 2.08 mmol, 1.00 eq.), 6-{4H,5H,6H-pyrrolo[3,4-c]pyrazole-2-sulfonyl}quinoline; trifluoromethanesulfonic acid (702 mg, 1.56 mmol, 0.750 eq.), DMF (10 mL), and DIEA (806 mg, 6.24 mmol, 3 eq.) at room temperature. To the above mixture was added T₃P (661 mg, 2.08 mmol, 1.0 eq.) drop-wise at 0° C. The resulting mixture was stirred for an additional 2 h at 0° C. The crude product was purified by Prep-HPLC with the following conditions (Prep-HPLC-003): Column, SunFire Prep C18 OBD Column, 19*150 mm, 5 μm 10 nm; mobile phase, Water (0.05% FA) and ACN (15% ACN up to 45% in 10 min). The resulting mixture was concentrated under vacuum. The product was purified by Chiral-Prep-HPLC with the following conditions (XA-Prep SFC150-1): Column: CHIRAL ART Cellulose-SJ, 3*25 cm, 5 μm; Mobile Phase A: CO2, Mobile Phase B: MeOH (0.1% 2M NH₃-MeOH); Flow rate: 80 mL/min; Gradient: isocratic 30% B; Column Temperature (° C.): 35; Back Pressure(bar): 100; Wave Length: 220 nm; RT1 (8.63 min); RT2 (10.10 min): Sample Solvent: MeOH-Preparative; Injection Volume: 8 mL; This resulted in (2S)-2-(3-fluoropyridin-2-yl)-3-hydroxy-1-[2-(quinoline-6-sulfonyl)-4H,6H-pyrrolo[3,4-c]pyrazol-5-yl]propan-1-one or (2R)-2-(3-fluoropyridin-2-yl)-3-hydroxy-1-[2-(quinoline-6-sulfonyl)-4H,6H-pyrrolo[3,4-c]pyrazol-5-yl]propan-1-one (51.5 mg, 10.60%, RT₁=8.63 min) and the corresponding enantiomer (52.1 mg, 10.72%, RT₂=10.10 min) as a white solid. LCMS (ES) [M+1]⁺ m/z 468. ¹H NMR (300 MHz, DMSO-d6) δ9.12 (dd, J=4.4, 2.0 Hz, 1H), 8.88 (d, J=2.6 Hz, 1H), 8.71 (d, J=8.7 Hz, 1H), 8.34 (s, 2H), 8.24 (d, J=8.8 Hz, 1H), 8.18-8.09 (m, 1H), 7.74 (dd, J=8.2, 4.1 Hz, 1H), 7.66 (d, J=9.2 Hz, 1H), 7.38-7.34 (m, 1H), 4.88-4.75 (m, 2H), 4.49-4.31 (m, 3H), 4.29-4.15 (m, 1H), 4.13-4.00 (m, 1H), 3.98-3.82 (m, 1H); CHIRAL_HPLC Analysis conditions: Retention time 1.387 min.

Enantiomer: CHIRAL_HPLC Analysis conditions: Retention time 1.235 min.

### Example 1.68

### Synthesis of (2R)-2-(2-fluorophenyl)-2-[(2-hydroxyethyl)amino]-1-[2-(quinoline-6-sulfonyl)-2H,4H,5H,6H-pyrrolo[3,4-c]pyrazol-5-yl]ethan-1-one (Compound 106)

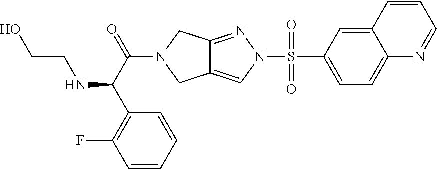 [SMILES: O=C([C@H](NCCO)c1ccccc1F)N1Cc2cn(S(=O)(=O)c3ccc4ncccc4c3)nc2C1]

Step 1

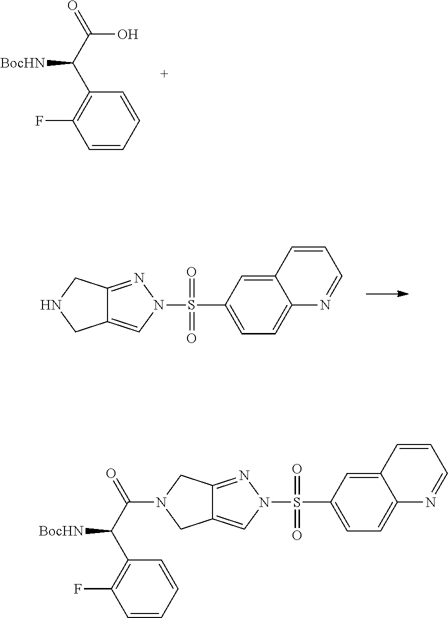 [SMILES: CC(C)(C)OC(=O)N[C@@H](C(=O)O)c1ccccc1F ; O=S(=O)(c1ccc2ncccc2c1)n1cc2c(n1)CNC2 ; CC(C)(C)OC(=O)N[C@@H](C(=O)N1Cc2cn(S(=O)(=O)c3ccc4ncccc4c3)nc2C1)c1ccccc1F]

Into a 50 mL 3-necked round-bottom flask were added (R)-[(tert-butoxycarbonyl)amino](2-fluorophenyl)acetic acid (655 mg, 2.43 mmol, 1.00 eq.), 6-{4H,5H,6H-pyrrolo[3,4-c]pyrazole-2-sulfonyl}quinoline (synthesized as described in the preparation of compounds 102 and 103; steps 1-4; 730 mg, 2.43 mmol, 1.00 eq.), DMF (10 mL), DIEA (629 mg, 4.87 mmol, 2.00 eq.) at room temperature, followed by the addition HATU (1.11 g, 2.92 mmol, 1.20 eq.) at 0° C. The resulting mixture was stirred for additional 1 h at room temperature, then it was quenched by the addition of water (15 mL) and extracted with ethyl acetate (30 mL×2). The combined organic phase was washed with brine (20 mL×2), dried over anhydrous sodium sulfate, filtered, and concentrated under reduced pressure. The residue was purified by silica gel column chromatography, eluting with PE/EA (from 20% to 100%) to afford tert-butyl (R)-(1-(2-fluorophenyl)-2-oxo-2-(2-(quinolin-6-ylsulfonyl)-2,6-dihydropyrrolo[3,4-c]pyrazol-5(4H)-yl)ethyl)carbamate (1.26 g, 94%) as a yellow oil. LCMS (ES) [M+1]⁺ m/z: 552.

Step 2

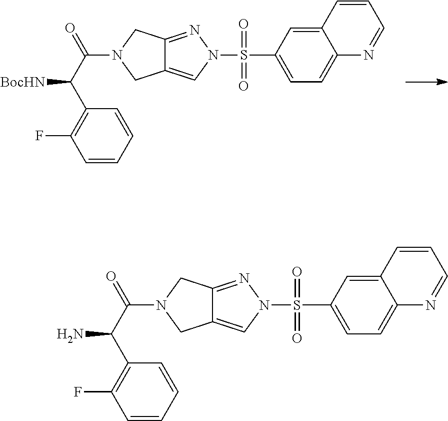 [SMILES: CC(C)(C)OC(=O)N[C@@H](C(=O)N1Cc2cn(S(=O)(=O)c3ccc4ncccc4c3)nc2C1)c1ccccc1F ; N[C@@H](C(=O)N1Cc2cn(S(=O)(=O)c3ccc4ncccc4c3)nc2C1)c1ccccc1F]

Into a 40 mL vial were added tert-butyl (R)-(1-(2-fluorophenyl)-2-oxo-2-(2-(quinolin-6-ylsulfonyl)-2,6-dihydropyrrolo[3,4-c]pyrazol-5(4H)-yl)ethyl)carbamate (1.26 g, 2.28 mmol, 1.00 eq.), DCM (20 mL), and ZnBr₂ (1.54 g, 6.85 mmol, 3.00 eq.) at room temperature. The resulting mixture was stirred for 15 h at room temperature. The mixture was then basified to pH 7 with sat.NaHCO₃ and the precipitated solid was collected by filtration and washed with CH₂Cl₂. This resulted in \hat{R}-2-amino-2-(2-fluorophenyl)-1-(153thenone153lin-6-ylsulfonyl)-2,6-dihydropyrrolo[3,4-c]pyrazol-5(4H)-yl)ethan-1-one (652 mg, 63.22%) as a yellow solid. LCMS (ES) [M+1]⁺ m/z: 452.

Step 3

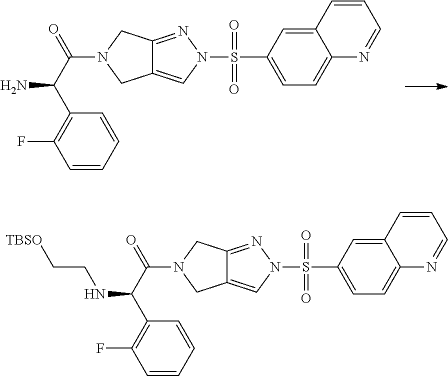 [SMILES: N[C@@H](C(=O)N1Cc2cn(S(=O)(=O)c3ccc4ncccc4c3)nc2C1)c1ccccc1F ; CC(C)(C)[Si](C)(C)OCCN[C@@H](C(=O)N1Cc2cn(S(=O)(=O)c3ccc4ncccc4c3)nc2C1)c1ccccc1F]

Into a 40 mL vial were added (2R)-2-amino-2-(2-fluorophenyl)-1-[2-(quinoline-6-sulfonyl)-4H,6H-pyrrolo[3,4-c]pyrazol-5-yl]ethanone (652 mg, 1.44 mmol, 1.00 eq.), 2-[(tert-butyldimethylsilyl)oxy]acetaldehyde (151 mg, 0.87 mmol, 0.60 eq.), MeOH (10 mL), and a drop of HOAc at room temperature. The resulting mixture was stirred for 20 min at room temperature then NaBH₃CN (272 mg, 4.33 mmol, 3.00 eq.) was added at room temperature. The resulting mixture was stirred for additional 3 h at room temperature, then it was concentrated under reduced pressure. The residue was diluted by the addition of water (10 mL), neutralized to pH 7 with NaHCO₃ (aq.), and extracted with CH₂Cl₂ (30 mL×2). The combined organic layer was dried over anhydrous sodium sulfate, filtered, and concentrated under reduced pressure. The residue was purified by silica gel column chromatography, eluting with PE/EA (2:1) to afford (R)-2-((2-((tert-butyldimethylsilyl)oxy)ethyl)amino)-2-(2-fluorophenyl)-1-(2-(quinolin-6-ylsulfonyl)-2,6-dihydropyrrolo[3,4-c]pyrazol-5(4H)-yl)ethan-1-one (200 mg, 23%) as a yellow oil. LCMS (ES) [M+1]⁺ m/z: 610.

Step 4

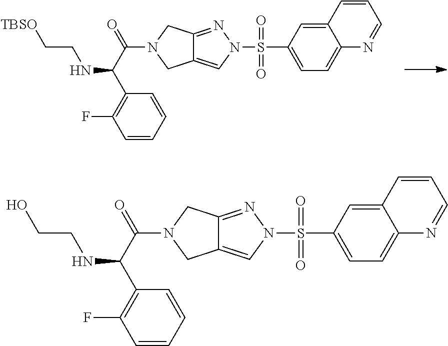 [SMILES: CC(C)(C)[Si](C)(C)OCCN[C@@H](C(=O)N1Cc2cn(S(=O)(=O)c3ccc4ncccc4c3)nc2C1)c1ccccc1F ; O=C([C@H](NCCO)c1ccccc1F)N1Cc2cn(S(=O)(=O)c3ccc4ncccc4c3)nc2C1]

Into a 20 mL vial were added (R)-2-((2-((tert-butyldimethylsilyl)oxy)ethyl)amino)-2-(2-fluorophenyl)-1-(2-(quinolin-6-ylsulfonyl)-2,6-dihydropyrrolo[3,4-c]pyrazol-5(4H)-yl)ethan-1-one (200 mg, 0.33 mmol, 1.00 eq.), THF (5 mL), H₂O (0.5 mL) and TFA (0.5 mL) at room temperature. The resulting mixture was stirred for 1 h at room temperature. The reaction solution was directly purified by Prep-HPLC with the following conditions: Sunfire Prep C18 OBD Column, 50*250 mm, 5 μm, 10 nm, mobile phase, water (0.1% FA) and CH₃CN (5% up to 25% in 15 min), Detector, UV 254 nm. This resulted in (R)-2-(2-fluorophenyl)-2-((2-hydroxyethyl)amino)-1-(2-(quinolin-6-ylsulfonyl)-2,6-dihydropyrrolo[3,4-c]pyrazol-5(4H)-yl)ethan-1-one formate (73.6 mg, 42%) as a white solid. ¹H-NMR: (300 MHz, DMSO-d₆, ppm): δ 9.12 (dd, J=4.3, 1.7 Hz, 1H), 8.88 (d, J=2.2 Hz, 1H), 8.71 (dd, J=8.5, 1.8 Hz, 1H), 8.34 (s, 1H), 8.30-8.19 (m, 2H), 8.13 (ddd, J=9.0, 3.5, 2.2 Hz, 1H), 7.74 (dd, J=8.4, 4.2 Hz, 1H), 7.45-7.24 (m, 2H), 7.24-7.09 (m, 2H), 4.94 (dd, J=18.7, 14.2 Hz, 1H), 4.83 (d, J=11.7 Hz, 1H), 4.49 (d, J=16.0 Hz, 1H), 4.36 (dd, J=16.0, 6.2 Hz, 1H), 4.20 (dd, J=14.1, 6.3 Hz, 1H), 3.42 (h, J=3.0 Hz, 2H), 2.49-2.38 (m, 2H). LCMS: (ES, m/z): [M−HCOOH+H]⁺: 496.

### Example 1.69

### Synthesis of 1-[(1R)-2-{2[4-(difluoromethoxy)benzenesulfonyl]-2H,4H,5H,6H-pyrrolo[3,4-c]pyrazol-5-yl}-2-oxo-1-phenylethyl]azetidin-2-one or 1-[(1S)-2-{2-[4-(difluoromethoxy)benzenesulfonyl]-2H,4H,5H,6H-pyrrolo[3,4-c]pyrazol-5-yl}-2-oxo-1-phenylethyl]azetidin-2-one (Compound 99)

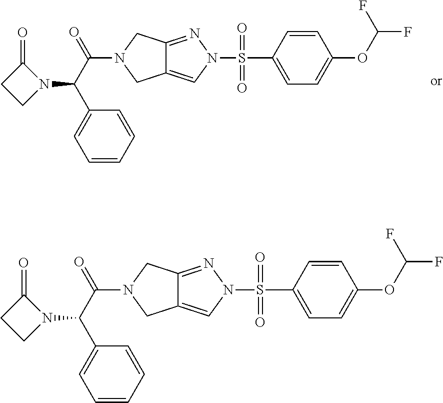 [SMILES: O=C([C@@H](c1ccccc1)N1CCC1=O)N1Cc2cn(S(=O)(=O)c3ccc(OC(F)F)cc3)nc2C1 ; O=C([C@H](c1ccccc1)N1CCC1=O)N1Cc2cn(S(=O)(=O)c3ccc(OC(F)F)cc3)nc2C1]

Step 1

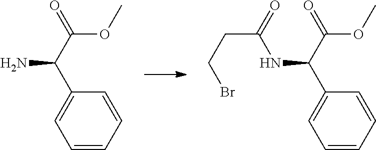 [SMILES: COC(=O)[C@H](N)c1ccccc1 ; COC(=O)[C@H](NC(=O)CCBr)c1ccccc1]

Into a 250 mL 3-necked round-bottom flask were added methyl(2R)-2-amino-2-phenylacetate (3.00 g, 18.16 mmol, 1.00 eq.), DCM (70 mL) and N,N-dimethylaniline (7.92 g, 65.36 mmol, 3.60 eq.) at room temperature, followed by the drop-wise addition of 3-bromopropanoyl chloride (3.58 g, 20.89 mmol, 1.15 eq.) at −20° C. The resulting mixture was stirred for additional 2 h at room temperature, then it was concentrated under vacuum. The residue was purified by silica gel column chromatography, eluting with PE/EA (3:1) to afford methyl (2R)-2-(3-bromopropanamido)-2-phenylacetate (4 g, 73.38%) as a light yellow oil. LCMS (ES) [M+1]⁺ m/z 300, 302.

Step 2

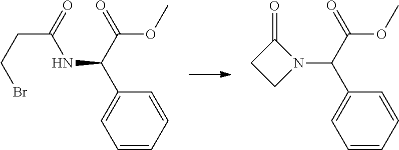 [SMILES: COC(=O)[C@H](NC(=O)CCBr)c1ccccc1 ; COC(=O)C(c1ccccc1)N1CCC1=O]

Into a 250 mL 3-necked round-bottom flask were added methyl (2R)-2-(3-bromopropanamido)-2-phenylacetate (3.60 g, 11.99 mmol, 1.00 eq.) and DMF (15 mL), DCM (60 mL) at room temperature. To the above mixture was added NaH (0.53 g, 13.19 mmol, 1.1 eq.) at 0° C. and the resulting mixture was stirred for additional 3 h at room temperature. The reaction was then quenched with water at room temperature and extracted with CH₂Cl₂ (3×100 mL). The combined organic layers were washed with brine (3×100 mL), dried over anhydrous Na₂SO₄, filtered, and concentrated under reduced pressure. The residue was purified by reverse flash chromatography with the following conditions: column, C18 silica gel; mobile phase, FA (0.1%) in water and ACN, 10% to 50% gradient in 15 min; detector, UV 220 nm. This resulted in completely racemized methyl-2-(2-oxoazetidin-1-yl)-2-phenylacetate (2 g, 76.06%) as a light yellow oil. LCMS (ES) [M+1]⁺ m/z 220.

Step 3

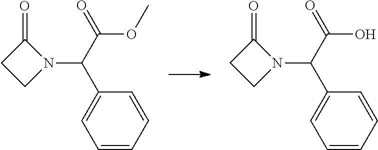 [SMILES: COC(=O)C(c1ccccc1)N1CCC1=O ; O=C(O)C(c1ccccc1)N1CCC1=O]

Into a 100 mL round-bottom flask were added methyl-2-(2-oxoazetidin-1-yl)-2-phenylacetate (2 g, 9.12 mmol, 1.00 eq.) and MeOH (15 mL) at room temperature, followed by the drop-wise addition of LiOH.H₂O (0.44 g, 18.24 mmol, 2 eq.) in H₂O (5 mL) at room temperature. The resulting mixture was stirred for additional 3 h at room temperature, then it was concentrated. The residue was purified by reverse flash chromatography with the following conditions: column, C18 silica gel; mobile phase, FA (0.1%) in water and ACN, 10% to 20% gradient in 10 min; detector, UV 220 nm. This resulted in (2-oxoazetidin-1-yl)(phenyl)acetic acid (800 mg, 42.73%) as an off-white semi-solid. LCMS (ES) [M−1]⁺ m/z 204.

Step 4

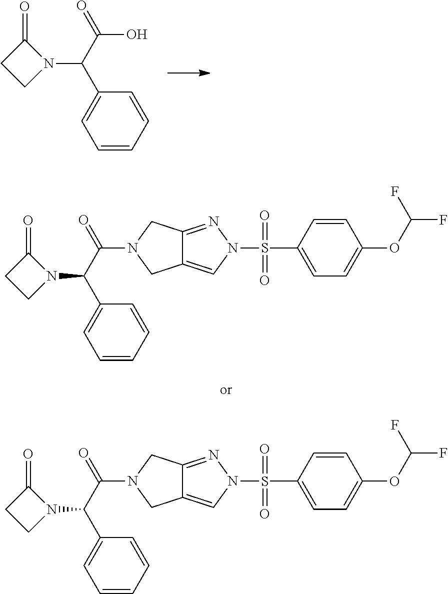 [SMILES: O=C(O)C(c1ccccc1)N1CCC1=O ; O=C([C@@H](c1ccccc1)N1CCC1=O)N1Cc2cn(S(=O)(=O)c3ccc(OC(F)F)cc3)nc2C1 ; O=C([C@H](c1ccccc1)N1CCC1=O)N1Cc2cn(S(=O)(=O)c3ccc(OC(F)F)cc3)nc2C1]

Into a 40 mL vial were added (2-oxoazetidin-1-yl)(phenyl)acetic acid (500 mg, 2.44 mmol, 1.00 eq.), 2-[4-(difluoromethoxy)benzenesulfonyl]-4H,5H,6H-pyrrolo[3,4-c]pyrazole hydrochloride (514 mg, 1.46 mmol, 0.6 eq.), DMF (5 mL), and DIEA (630 mg, 4.87 mmol, 2 eq.) at room temperature. To the above mixture was added T₃P (930 mg, 2.92 mmol, 1.2 eq.) dropwise at 0° C. and the resulting mixture was stirred for an additional 3 h at 0° C. The crude product was purified by Prep-HPLC with the following conditions (Prep-HPLC-003): Column, SunFire Prep C18 OBD Column, 19*150 mm, 5 μm 10 nm; mobile phase, Water (0.05% FA) and ACN (15% ACN up to 45% in 7 min). The resulting mixture was concentrated under vacuum. The product was purified by Chiral-Prep-HPLC with the following conditions Column: (R, R)-WHELK-O1-Kromasil, 2.12*25 cm, 5 μm; Mobile Phase A: HEX: DCM=3: 1-HPLC, Mobile Phase B: EtOH-HPLC; Flow rate: 35 mL/min; Gradient: 40% B to 40% B in 22 min; Wave Length: 220/254 nm; RT1 (10 min); RT2 (16 min); Sample Solvent: EtOH: DCM=3: 1-HPLC; Injection Volume: 5 mL; This resulted in 1-[(1R)-2-{2-[4-(difluoromethoxy)benzenesulfonyl]-4H,6H-pyrrolo [3,4-c]pyrazol-5-yl}-2-oxo-1-phenylethyl]azetidin-2-one or 1-[(1S)-2-{2-[4-(difluoromethoxy)benzenesulfonyl]-4H,6H-pyrrolo[3,4-c]pyrazol-5-yl}-2-oxo-1-phenylethyl]azetidin-2-one (58.8 mg, 9.61%, RT₁=10 min/22 min) and its related enantiomer (60.9 mg, 9.95%, RT₂=16 min/22 min) as a white solid. LCMS (ES) [M+1]⁺ m/z 503. ¹H NMR (300 MHz, DMSO-d₆): δ 8.26 (d, J=8.9 Hz, 1H), 8.08-7.98 (m, 2H), 7.72-7.12 (m, 8H), 5.79 (d, J=8.8 Hz, 1H), 4.82 (dd, J=19.1, 14.3 Hz, 1H), 4.59-4.36 (m, 2H), 4.01 (dd, J=14.5, 3.5 Hz, 1H), 3.58-3.46 (m, 1H), 2.99-2.89 (m, 1H), 2.89-2.71 (m, 2H). CHIRAL_HPLC Analysis: Retention time 2.666 min.

Enantiomer: CHIRAL_HPLC Analysis: Retention time 1.970 min.

### Example 1.70

### Synthesis of (2S)-3-hydroxy-2-phenyl-1-[2-(quinoxaline-6-sulfonyl)-2H,4H,5H,6H-pyrrolo[3,4-c]pyrazol-5-yl]propan-1-one (Compound 94)

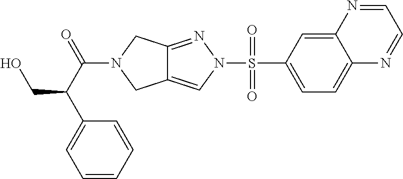 [SMILES: O=C([C@H](CO)c1ccccc1)N1Cc2cn(S(=O)(=O)c3ccc4nccnc4c3)nc2C1]

The title compound was synthesized from commercial quinoxaline-6-sulfonyl chloride and (2S)-3-hydroxy-2-phenylpropanoic acid. The crude final product was purified by reverse phase preparative HPLC (Prep-C18, 5 μM Luna C18 column, 22×250 mm, Phenomenex; gradient elution of 10-70% MeCN in water over a 20 min period, where both solvents contained 0.1% formic acid) to provide the title product as a white solid. ¹H NMR (400 MHz, dmso) δ 9.13-9.08 (m, 2H), 8.66 (d, J=2.2 Hz, 1H), 8.47-8.28 (m, 3H), 8.21 (dd, J=8.9, 2.2 Hz, 1H), 7.30-7.25 (m, 5H), 4.82 (dd, J=17.1, 14.4 Hz, 2H), 4.46-4.37 (m, 2H), 4.34 (d, J=14.1 Hz, 2H), 3.93-3.89 (m, 2H).LCMS (ES) [M+1]⁺ m/z 450.2.

### Example 1.71

### Synthesis of (2S)-1-(2-{[1-(difluoromethyl)-1H-indazol-5-yl]sulfonyl}-2H,4H,5H,6H-pyrrolo[3,4-c]pyrazol-5-yl)-3-hydroxy-2-phenylpropan-1-one (Compound 91)

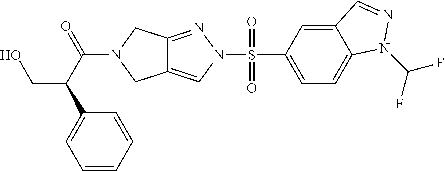 [SMILES: O=C([C@H](CO)c1ccccc1)N1Cc2cn(S(=O)(=O)c3ccc4c(cnn4C(F)F)c3)nc2C1]

The title compound was synthesized from 1-(difluoromethyl)-1H-indazole-5-sulfonyl chloride (synthesized from commercial 5-bromo-1H-indazole as described in steps 1-3 below) and commercial (2S)-3-hydroxy-2-phenylpropanoic acid. The crude final product was purified by reverse phase preparative HPLC (Prep-C18, 5 μM Luna C18 column, 22×250 mm, Phenomenex; gradient elution of 10-70% MeCN in water over a 20 min period, where both solvents contain 0.1% formic acid) to provide the title product as a white solid. ¹H NMR (400 MHz, dmso) δ 9.20 (d, J=1.0 Hz, 1H), 8.72 (td, J=2.1, 0.9 Hz, 1H), 8.41-8.16 (m, 2H), 7.90 (dt, J=9.3, 1.0 Hz, 1H), 7.66 (dt, J=9.4, 1.9 Hz, 1H), 7.30-7.17 (m, 5H), 4.87-4.68 (m, 2H), 4.46-4.21 (m, 3H), 3.99-3.86 (m, 2H), 3.52-3.41 (m, 1H). LCMS (ES) [M+1]⁺ m/z 488.1.

Step 1

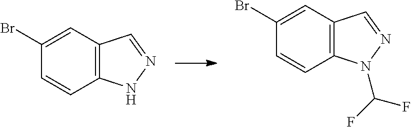 [SMILES: Brc1ccc2[nH]ncc2c1 ; FC(F)n1ncc2cc(Br)ccc21]

Diethyl bromo(difluoro)methylphosphonate (5.80 g; 21.9 mmol; 2.0 eq.) was added to a solution containing 5-bromo-1H-indazole (2.20 g; 11.0 mmol; 1.0 eq.) and potassium fluoride (1.27 g; 21.9 mmol; 2.0 eq.) in acetonitrile (37 mL). The reaction was heated to 35° C. for 18 hours. The reaction was quenched with water. The aqueous layer was extracted with ethyl acetate. The combined organics were dried with MgSO₄, filtered, and concentrated under reduced pressure. The resulting crude product was purified by silica gel column using 25% ethyl acetate in heptanes as the eluting solvents to afford 5-bromo-1-(difluoromethyl)-1H-indazole (2.6 g; 96%). LCMS (ES) [M+1]⁺ m/z 248.8.

Step 2

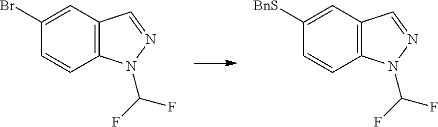

In a capped 40 mL vial, combined benzyl hydrosulfide (686 mg; 5.5 mmol; 1.1 eq.), 5-bromo-1-(difluoromethyl)-1H-indazole (1.24 g; 5.0 mmol; 1.0 eq.), Pd₂(dba)₃ (230 mg; 0.3 mmol; 0.05 eq.), Xantphos (290 mg; 0.50 mmol; 0.1 eq.) and Hunig's base (2.6 mL; 15.0 mmol; 3.0 eq.) in toluene (13 mL). Heated the reaction to 100° C. for 18 hours. Let the reaction cool to room temperature. Filtered the reaction mixture thru a plug of Celite. Concentrated the filtrate and purified the resulting crude by silica gel chromatography using 30% ethyl acetate in heptanes as the eluting solvents to afford 5-(158enzylsulfinyl)-1-(difluoromethyl)-1H-indazole (1.5 g; 99%). LCMS (ES) [M+1]⁺ m/z 290.9.

Step 3

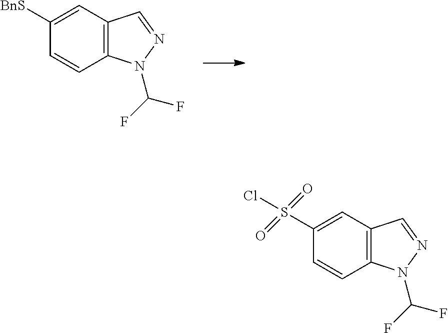

1,3-Dichloro-5,5-dimethyl-2,4-imidazolidinedione (2.96 g; 15.0 mmol; 3.0 eq.) was added to a pre-cooled solution of 5-(158enzylsulfinyl)-1-(difluoromethyl)-1H-indazole (1.46 g; 5.02 mmol; 1.0 eq.), acetic acid (9 mL), water (17 mL), and tetrahydrofuran (50 mL) at 0° C. The reaction was stirred for 2 hours. The reaction was quenched with saturated sodium bicarbonate solution. The aqueous layer was extracted with ethyl acetate. The organic layers were combined, dried with MgSO₄, filtered, and concentrated under reduced pressure to afford the crude. The resulting crude was purified by silica gel column using 50% ethyl acetate in heptanes to afford 1-(difluoromethyl)-1H-indazole-5-sulfonyl chloride (1.34 g; 99%). LCMS (ES) [M+1]⁺ m/z 266.8.

### Example 1.72

### Synthesis of 2-amino-1-[2-(1,3-benzothiazole-6-sulfonyl)-2H,4H,5H,6H-pyrrolo[3,4-c]pyrazol-5-yl]-2-[3-(trifluoromethyl)phenyl]ethan-1-one (Compound 26; Racemic)

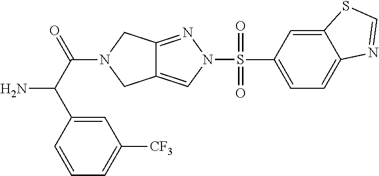 [SMILES: NC(C(=O)N1Cc2cn(S(=O)(=O)c3ccc4ncsc4c3)nc2C1)c1cccc(C(F)(F)F)c1]

The title compound was made from 6-{2H,4H,5H,6H-pyrrolo[3,4-c]pyrazole-2-sulfonyl}-1,3-benzothiazole (Intermediate I-1) and 2-{[(tert-butoxy)carbonyl]amino}-2-[3-(trifluoromethyl)phenyl]acetic acid following a 2-step procedure similar to that described for the synthesis of (2R)-2-amino-1-[2-(1,3-benzothiazole-6-sulfonyl)-2H,4H,5H,6H-pyrrolo[3,4-c]pyrazol-5-yl]-2 phenylethan-1-one (Compound 15). The crude product was purified by reverse phase preparative HPLC (Prep-C18, 5 μM XBridge column, 19×150 mm, Waters; gradient elution of 0-80% MeCN in water over a 20 min period, where both solvents contained 0.1% formic acid) to provide the title product as a racemic mixture as a white solid. ¹H NMR (400 MHz, DMSO-d6) δ 9.69 (s, 1H), 9.02 (d, J=2.0 Hz, 1H), 8.30 (dd, J=9.5, 6.5 Hz, 2H), 8.03 (dt, J=8.8, 1.9 Hz, 1H), 7.74 (s, 1H), 7.67-7.45 (m, 3H), 4.90 (dd, J=17.8, 14.3 Hz, 1H), 4.79 (d, J=16.5 Hz, 1H), 4.49-4.27 (m, 3H). LCMS (ES) [M+1]+ m/z 508.0.

### Example 1.73

### Synthesis of 1-[2-(1,3-benzothiazole-6-sulfonyl)-2H,4H,5H,6H-pyrrolo[3,4-c]pyrazol-5-yl]-3-hydroxy-2-(pyridin-4-yl)propan-1-one (Compound 27; Racemic)

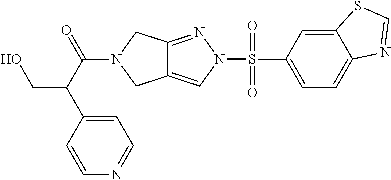 [SMILES: O=C(C(CO)c1ccncc1)N1Cc2cn(S(=O)(=O)c3ccc4ncsc4c3)nc2C1]

Step 1

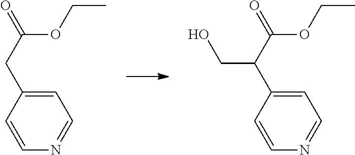

Into a 40-mL vial, was placed ethyl 2-(pyridin-4-yl)acetate (2.0 g, 12.11 mmol, 1.0 eq.), DMSO (20.0 mL), CH₃ONa (65 mg, 1.20 mmol, 0.1 eq.), and (CH₂O)ₙ (364 mg, 12.12 mmol, 1.0 eq.). The reaction mixture was stirred for 12 h at room temperature; then, it was quenched by the addition of water (30 mL) and extracted with 2×30 mL of ethyl acetate. The combined organic phase was dried over anhydrous sodium sulfate, filtered, and concentrated under reduced pressure. The residue was purified by silica gel column with ethyl acetate/petroleum ether (70%). 412 mg (17%) of ethyl 3-hydroxy-2-(pyridin-4-yl)propanoate was obtained as a yellow oil. LCMS (ES) [M+1]⁺m/z: 196.

Step 2

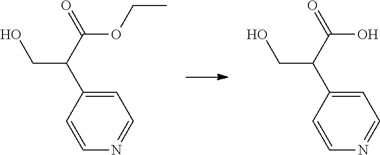

Into a 40-mL vial, was placed ethyl 3-hydroxy-2-(pyridin-4-yl)propanoate (412 mg, 2.11 mmol, 1.00 eq.), EtOH (3.0 mL), LiOH.H₂O (177 mg, 4.22 mmol, 2.0 eq.), and H₂O (6.0 mL) and the reaction solution was stirred for 1 h at room temperature. The pH value of the solution was then adjusted to 7 with 4 N HCl and this mixture was directly purified by Prep-HPLC with the following conditions: Column, C18-120 g, Mobile phase, CH₃CN/H₂O from 0% to 30% within 8 min, Flow rate: 70 mL/min, Detector, 254 nm. 194 mg (55%) of 3-hydroxy-2-(pyridin-4-yl)propanoic acid was obtained as a white solid. LCMS (ES) [M+1]⁺ m/z: 168.

Step 3

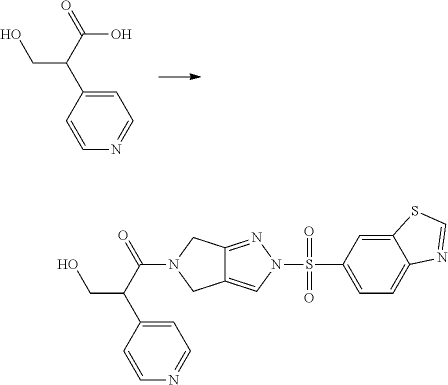 [SMILES: O=C(O)C(CO)c1ccncc1 ; O=C(C(CO)c1ccncc1)N1Cc2cn(S(=O)(=O)c3ccc4ncsc4c3)nc2C1]

Into a 40-mL vial, was placed 3-hydroxy-2-(pyridin-4-yl)propanoic acid (194 mg, 1.16 mmol, 1.0 eq.), DMF (10.0 mL), 6-[4H,5H,6H-pyrrolo[3,4-c]pyrazole-2-sulfonyl]-1,3-benzothiazole hydrochloride (Intermediate I-1; 397 mg, 1.16 mmol, 1.0 eq.), and DIEA (450 mg, 3.48 mmol, 3.0 eq.), followed by the addition of HATU (530 mg, 1.39 mmol, 1.2 eq.) at 0° C. The resulting mixture was stirred for 2 h at room temperature. Then, it was quenched by the addition of water (20 mL) and extracted with 3×20 mL of ethyl acetate. The combined organic phase was dried over anhydrous sodium sulfate, filtered, and concentrated under reduced pressure. The residue was purified by silica gel column with THF/PE (100%). The crude product was further purified by Prep-HPLC with the following conditions: Column, Atlantis HILIC OBD Column, 19*150 mm*5 um, Mobile phase, Water (0.1% FA) and CH₃CN (40% Phase B up to 60% in 8 min), Detector, UV 254 nm. 11.2 mg (2%) of 1-(2-(benzo[d]thiazol-6-ylsulfonyl)-2,6-dihydropyrrolo[3,4-c]pyrazol-5(4H)-yl)-3-hydroxy-2-(pyridin-4-yl)propan-1-one was obtained as white solid. ¹H-NMR: (300 MHz, DMSO-d₆, ppm): δ 9.71 (s, 1H), 9.02 (d, J=1.8 Hz, 1H), 8.49-8.46 (m, 2H), 8.31 (d, J=7.5 Hz, 2H), 8.04 (dd, J=8.7, 2.1 Hz, 1H), 7.35 (d, J=6.0 Hz, 2H), 4.92-4.81 (m, 2H), 4.49-4.37 (m, 3H), 4.04-3.91(m, 2H), 3.62-3.56 (m, 1H). LCMS: (ES, m/z): [M+H]⁺: 456.

### Example 1.74

### Synthesis of (2R)-2-amino-1-[2-(1,3-benzothiazole-6-sulfonyl)-2H,4H,5H,6H-pyrrolo[3,4-c]pyrazol-5-yl]-2-(4-fluorophenyl)ethan-1-one (Compound 28)

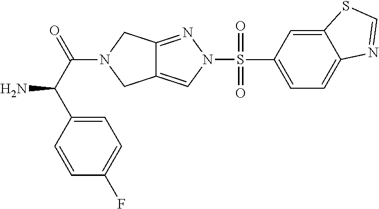 [SMILES: N[C@@H](C(=O)N1Cc2cn(S(=O)(=O)c3ccc4ncsc4c3)nc2C1)c1ccc(F)cc1]

The title compound was made from 6-{2H,4H,5H,6H-pyrrolo[3,4-c]pyrazole-2-sulfonyl}-1,3-benzothiazole (Intermediate I-1) and (2R)-[(tert-butoxycarbonyl)amino](4-fluorophenyl)ethanoic acid following a 2-step procedure similar to that for the synthesis of (2R)-2 amino-1-[2-(1,3-benzothiazole-6-sulfonyl)-2H,4H,5H,6H-pyrrolo[3,4-c]pyrazol-5-yl]-2 phenylethan-1-one (Compound 15). The crude product was purified by reverse phase preparative HPLC (Prep-C18, 5 μM XBridge column, 19×150 mm, Waters; gradient elution of 0-60% MeCN in water over a 20 min period, where both solvents contain 0.1% formic acid) to provide the title product as a white solid. ¹H NMR (400 MHz, DMSO-d6) δ 9.69 (s, 1H), 9.00 (d, J=2.0 Hz, 1H), 8.34-8.21 (m, 2H), 8.02 (ddd, J=8.7, 2.1, 0.7 Hz, 1H), 7.37 (ddt, J=7.1, 5.5, 1.7 Hz, 2H), 7.21-7.00 (m, 2H), 4.95-4.62 (m, 2H), 4.52-4.10 (m, 3H). LCMS (ES) [M+1]+ m/z 458.1.

### Example 1.75

### Synthesis of (2R)-2-amino-1-{2-[4-(difluoromethoxy)benzenesulfonyl]-2H,4H,5H,6H-pyrrolo[3,4-c]pyrazol-5-yl}-2-(3-fluorophenyl)ethan-1-one (Compound 29)

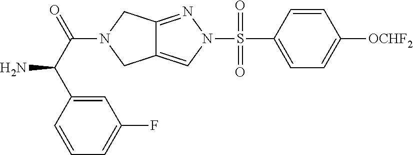 [SMILES: N[C@@H](C(=O)N1Cc2cn(S(=O)(=O)c3ccc(OC(F)F)cc3)nc2C1)c1cccc(F)c1]

Step 1

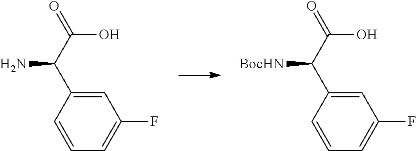 [SMILES: N[C@@H](C(=O)O)c1cccc(F)c1 ; CC(C)(C)OC(=O)N[C@@H](C(=O)O)c1cccc(F)c1]

Into a 100-mL round-bottom flask, was placed (S)-2-amino-2-(3-fluorophenyl)acetic acid (1.00 g, 5.912 mmol, 1.00 eq.), dioxane (30.00 mL), H₂O (10.00 mL), NaOH (473.00 mg, 11.824 mmol, 2.00 eq.), and Boc₂O (1.55 g, 7.102 mmol, 1.20 eq.), and the resulting solution was stirred for 2 h at room temperature. The pH value of the solution was adjusted to 4 with 4N HCl and the resulting solution was extracted with 3×50 mL of ethyl acetate. The residue was applied onto a silica gel column with THF/PE (50%) to provide 1.4 g (87.95%) of (S)-2-((tert-butoxycarbonyl)amino)-2-(3-fluorophenyl)acetic acid as a yellow oil. LCMS (ES) [M−1]-m/z: 268.

Step 2

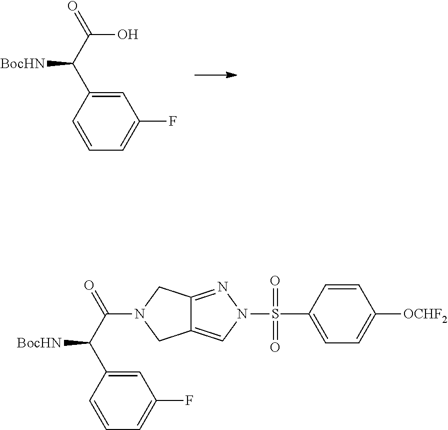 [SMILES: CC(C)(C)OC(=O)N[C@@H](C(=O)O)c1cccc(F)c1 ; CC(C)(C)OC(=O)N[C@@H](C(=O)N1Cc2cn(S(=O)(=O)c3ccc(OC(F)F)cc3)nc2C1)c1cccc(F)c1]

Into a 40-mL vial, was placed (S)-2-((tert-butoxycarbonyl)amino)-2-(3-fluorophenyl)acetic acid (278.00 mg, 1.032 mmol, 1.00 eq.), 2-[4-(difluoromethoxy)benzenesulfonyl]-4H,5H,6H-pyrrolo[3,4-c]pyrazole hydrochloride (Intermediate I-2; 362.00 mg, 1.029 mmol, 1.00 eq.), DMF (8.00 mL), DIEA (400.00 mg, 3.095 mmol, 3.00 eq.), and HATU (471.00 mg, 1.239 mmol, 1.20 eq.), and the resulting solution was stirred for 2 h at room temperature. The reaction was then quenched by the addition of water and the mixture was extracted with 3×20 mL of ethyl acetate. The residue was applied onto a silica gel column with THF/PE (70%) to provide 380 mg (65.08%) of tert-butyl (S)-(2-(2-((4-(difluoromethoxy)phenyl)sulfonyl)-2,6-dihydropyrrolo[3,4-c]pyrazol-5(4H)-yl)-1-(3-fluorophenyl)-2-oxoethyl)carbamate as a yellow oil. LCMS (ES) [M+1]⁺ m/z: 567.

Step 3

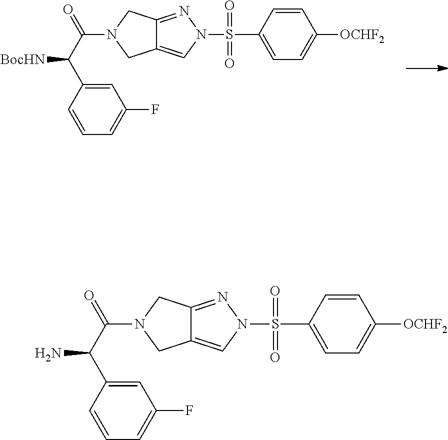 [SMILES: CC(C)(C)OC(=O)N[C@@H](C(=O)N1Cc2cn(S(=O)(=O)c3ccc(OC(F)F)cc3)nc2C1)c1cccc(F)c1 ; N[C@@H](C(=O)N1Cc2cn(S(=O)(=O)c3ccc(OC(F)F)cc3)nc2C1)c1cccc(F)c1]

Into a 50-mL round-bottom flask, was placed tert-butyl N-(2-[2-[4-(difluoromethoxy)benzenesulfonyl]-4H,6H-pyrrolo[3,4-c]pyrazol-5-yl]-1-(3-fluorophenyl)-2-oxoethyl)carbamate (380.00 mg, 0.671 mmol, 1.00 eq.), DCM (10.00 mL), and ZnBr₂ (453.00 mg, 2.011 mmol, 3.00 eq.) and the resulting solution was stirred at room temperature overnight. The mixture was then concentrated and the crude product was purified by Prep-HPLC with the following conditions (2 #SHIMADZU (HPLC-01)): Column, Atlantis HILIC OBD Column, 19*150 mm*5 um; mobile phase, Water (0.1% FA) and ACN (40% PhaseB up to 60% in 8 min); Detector, uv 254 nm. The crude product was purified by Chiral-Prep-HPLC with the following conditions: Column,Cellulose-4, 21.2*150 mm*5 um ; mobile phase, ACN (0.1% DEA) and EtOH (30% in 5.5 min) ; Detector, uv 254 nm to provide 9.5 mg (2.76%) of (R)-2-amino-1-(2-((4-(difluoromethoxy)phenyl)sulfonyl)-2,6-dihydropyrrolo[3,4-c]pyrazol-5(4H)-yl)-2-(3-fluorophenyl)ethan-1-one and 61.4 mg (17.86%) of the enantiomer. ¹H-NMR-(R-enantiomer): (300 MHz, DMSO-d6, ppm): δ 8.28 (s, 1H), 8.05 (d, J=9.0 Hz, 2H), 7.67-7.17 (m, 6H), 7.13-7.04 (m, 1H), 4.89 (dd, J=19.7, 14.3 Hz, 1H), 4.75 (d, J=10.1 Hz, 1H), 4.56-4.22 (m, 3H). LCMS: (ES, m/z): [M+H]⁺: 467.

### Example 1.76

### Synthesis of (2R)-2-amino-1-{2-[4-(difluoromethoxy)benzenesulfonyl]-2H,4H,5H,6H-pyrrolo[3,4-c]pyrazol-5-yl}-2-[3-(trifluoromethoxy)phenyl]ethan-1-one (Compound 30)

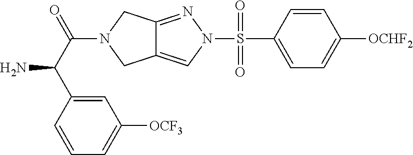 [SMILES: N[C@@H](C(=O)N1Cc2cn(S(=O)(=O)c3ccc(OC(F)F)cc3)nc2C1)c1cccc(OC(F)(F)F)c1]

The title compound was made from 2-[4-(difluoromethoxy)benzenesulfonyl]-4H,5H,6H-pyrrolo[3,4-c]pyrazole (Intermediate I-2) and 2(S)-amino[3-(trifluoromethoxy)phenyl]acetic acid following a 3-step procedure similar to that described for the synthesis of (2R)-2-amino-1-{2-[4-(difluoromethoxy)benzenesulfonyl]-2H,4H,5H,6H-pyrrolo[3,4-c]pyrazol-5-yl}-2-(3-fluorophenyl)ethan-1-one (Compound 29). The crude product was purified by Prep-HPLC with the following conditions: SunFire Prep C18 OBD Column, 19×150 mm, 5 um; mobile phase, phase A: H₂O (0.1% FA); phase B: CH₃CN (20% CH₃CN up to 50% CH₃CN in 15 min). This resulted in 115 mg of the desired product albeit with 70% ee. This material was further purified by chiral HPLC with the following conditions. Column: chiralPAK-AD-3; mobile phase: A: n-Hexane (0.1% DEA), B: Ethanol; gradient elution of 30% B to 30% B in 24 min to provide (R)-2-amino-1-(2-((4-(difluoromethoxy)phenyl)sulfonyl)-2,6-dihydropyrrolo[3,4-c]pyrazol-5(4H)-yl)-2-(3-(trifluoromethoxy)phenyl)ethan-1-one as an off-white solid. ¹H NMR (300 MHz, DMSO-d₆, ppm): δ 8.29-8.27 (m, 1H), 8.06-8.03 (m, 2H), 7.67-7.18 (m, 7H), 4.96-4.85 (m, 1H), 4.81-4.75 (m, 1H), 4.55-4.27 (m, 3H). LCMS (ES) [M+1]⁺ m/z 533.

### Example 1.77

### Synthesis of 6-({5-[(3S)-3-(6-methylpyridin-3-yl)oxolane-3-carbonyl]-2H,4H,5H,6H-pyrrolo[3,4-c]pyrazol-2-yl}sulfonyl)-1,3-benzothiazole or 6-({5-[(3R)-3-(6-methylpyridin-3-yl)oxolane-3-carbonyl]-2H,4H,5H,6H-pyrrolo[3,4-c]pyrazol-2-yl}sulfonyl)-1,3-benzothiazole (Compound 31)

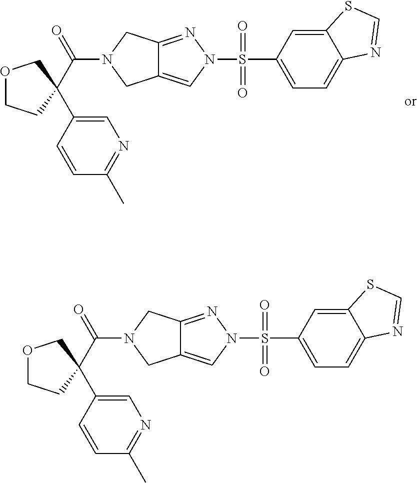 [SMILES: Cc1ccc([C@@]2(C(=O)N3Cc4cn(S(=O)(=O)c5ccc6ncsc6c5)nc4C3)CCOC2)cn1 ; Cc1ccc([C@@]2(C(=O)N3Cc4cn(S(=O)(=O)c5ccc6ncsc6c5)nc4C3)CCOC2)cn1]

Step 1

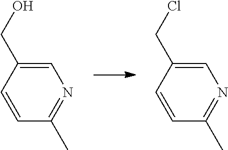

A solution of (6-methylpyridin-3-yl)methanol (11.0 g, 89.43 mmol, 1.0 eq.) in THF (100 mL) was cooled to 0° C., and thionyl chloride (7.1 mL, 98.37 mmol, 1.1 eq.) was added drop-wise. After the addition, the mixture was refluxed for 2 h. The solid was collected, washed with ether (30 mL×2), and suspended in ether (50 mL). Aqueous sodium hydroxide (10%) was added with stirring to pH 8. The ether solution was dried over anhydrous Na₂SO₄, filtered, and evaporated. 11.0 g (87%) 5-(chloromethyl)-2-methylpyridine was obtained as a yellow oil. LCMS (ESI): [M]⁺:142

Step 2

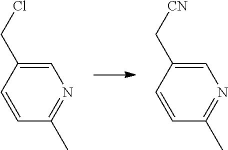

Into a 250-mL round bottom flask, was placed 5-(chloromethyl)-2-methylpyridine (11.0 g, 78.01 mmol, 1.0 eq.), and DMSO (100.0 mL), followed by the addition of NaCN (4.59 g, 93.62 mmol, 1.2 eq.) at room temperature. After the addition, the reaction was heated to 80° C. and stirred for 2 h. The reaction was then cooled to room temperature, quenched with H₂O (100.0 mL), and extracted with ethyl acetate (100 mL×2). The combined organic phase was washed with brine (50 mL×3), dried over anhydrous sodium sulfate filtered, and concentrated under reduced pressure. The residue was purified by silica gel column with ethyl acetate/petroleum ether (60%), to provide 8.6 g (83%) of 2-(6-methylpyridin-3-yl)acetonitrile. LCMS (ES): [M+H]⁺: 133.

Step 3

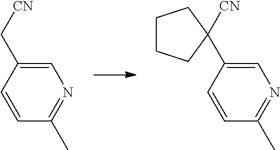

Into a 250-mL three-necked round bottom flask, was placed 2-(6-methylpyridin-3-yl)acetonitrile (8.2 g, 62.12 mmol, 1.0 eq.) and DMF (120.0 mL), followed by the addition of NaH (60% in mineral oil) (5.5 g, 136.67 mmol, 2.2 eq.) at 0° C. carefully, and stirred for 0.5 h. This was followed by the drop-wise addition of 1-chloro-2-(chloromethoxy)ethane (8.75 g, 68.33 mmol, 1.1 eq.), and the resulting mixture was stirred for 2 h at 0° C. The reaction solution was quenched with H₂O (100 mL), and extracted with ethyl acetate (80 mL×2). The combined organic phase was washed with brine (50 mL×3), dried over anhydrous sodium sulfate, filtered, and concentrated under reduced pressure. The residue was purified by silica gel column with ethyl acetate/petroleum ether (1/3) to provide 1.5 g (13%) of 3-(6-methylpyridin-3-yl)tetrahydrofuran-3-carbonitrile as a brown oil. LCMS (ES):[M+H]⁺: 189.

Step 4

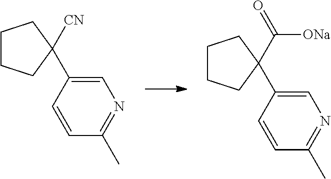

Into a 40-mL vial, was placed 3-(6-methylpyridin-3-yl)tetrahydrofuran-3-carbonitrile (500 mg, 2.66 mmol, 1.0 eq.) and THF/H₂O (1:1) (10.0 mL). NaOH (320 mg, 7.98 mmol, 3.0 eq.) was added at room temperature and the resulting mixture was stirred for 24 h at 60° C. After concentration, the residue was purified by Prep-HPLC with the following conditions: Column, C18-120 g, Phase A, H₂O, Phase B CH₃CN, from 0% to 60% within 10 min, Flow rate, 70 mL/min, Detector, 254 nm to provide 450 mg (74%) of sodium 3-(6-methylpyridin-3-yl)tetrahydrofuran-3-carboxylate was obtained as a white solid. LCMS (ES): [M−Na+H+H]⁺:208.

Step 5

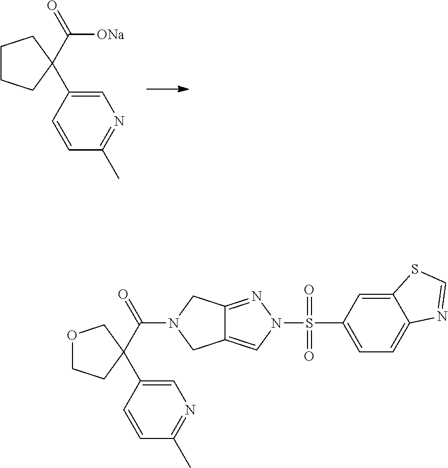

Into a 40-mL vial, was placed sodium 3-(6-methylpyridin-3-yl)tetrahydrofuran-3-carboxylate (450 mg, 1.96 mmol, 1.0 eq.), 6-((5,6-dihydropyrrolo[3,4-c]pyrazol-2(4H)-yl)sulfonyl)benzo[d]thiazole hydrochloride (Intermediate I-1; 672 mg, 1.96 mmol, 1.0 eq.), DIEA (759 mg, 5.88 mmol, 3.0 eq.), and DMF (10.0 mL), followed by the addition of HATU (894 mg, 2.35 mmol, 1.2 eq.) at 0° C. After the addition, the reaction was allowed to stir at room temperature for 2 h. The reaction solution was quenched with H₂O (20 mL), and extracted with ethyl acetate (30 mL×2). The combined organic phase was dried over anhydrous sodium sulfate, filtered, and concentrated under reduced pressure. The residue was purified by silica gel column with ethyl acetate/petroleum ether (70%), to provide 360 mg (37%) of (2-(benzo[d]thiazol-6-ylsulfonyl)-2,6-dihydropyrrolo[3,4-c]pyrazol-5(4H)-yl)(3-(6-methylpyridin-3-yl)tetrahydrofuran-3-yl)methanone as a white solid. LCMS (ESI):[M+H]⁺: 496.

Step 6

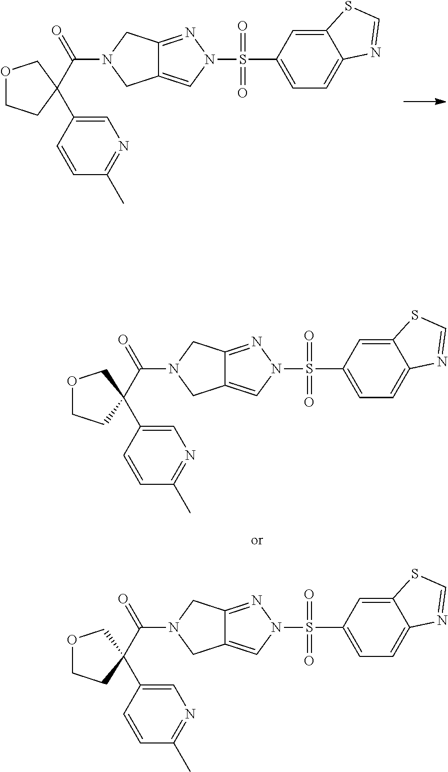 [SMILES: Cc1ccc(C2(C(=O)N3Cc4cn(S(=O)(=O)c5ccc6ncsc6c5)nc4C3)CCOC2)cn1 ; Cc1ccc([C@@]2(C(=O)N3Cc4cn(S(=O)(=O)c5ccc6ncsc6c5)nc4C3)CCOC2)cn1 ; Cc1ccc([C@]2(C(=O)N3Cc4cn(S(=O)(=O)c5ccc6ncsc6c5)nc4C3)CCOC2)cn1]

(2-(benzo[d]thiazol-6-ylsulfonyl)-2,6-dihydropyrrolo[3,4-c]pyrazol-5(4H)-yl)(3-(6-methylpyridin-3-yl)tetrahydrofuran-3-yl)methanone was separated with the following conditions: chiral PAK IC column, 20*250 mm, 5 um, Phase A: dichloromethane, Phase B: ethanol Gradient: 70% phase B within 20 min, Detector: 254 nm to provide 69.6 mg of (R)-(2-(benzo[d]thiazol-6-ylsulfonyl)-2,6-dihydropyrrolo[3,4-c]pyrazol-5(4H)-yl)(3-(6-methylpyridin-3-yl)tetrahydrofuran-3-ylmethanone or (S)-(2-(benzo[d]thiazol-6-ylsulfonyl)-2,6-dihydropyrrolo[3,4-c]pyrazol-5(4H)-yl)(3-(6-methylpyridin-3-yl)tetrahydrofuran-3-yl)methanone (RT=1.93 min). ¹H-NMR: (300 MHz, DMSO-d₆, ppm): δ 9.06(d, J=1.2 Hz, 1H), 9.01(dd, J=7.2, 2.1 Hz, 1H), 8.35-8.15(m, 3H), 8.05-7.99(m, 1H), 7.55(dd, J=8.1, 2.4 Hz, 1H), 7.21 (dd, J=8.1, 5.7 Hz, 1H), 4.56-4.48(m, 2H), 4.31-4.18(m, 1H), 4.10(d, J=14.4 Hz, 1H), 3.94-3.82(m, 4H), 2.68-2.58(m, 1H), 2.45-2.36(m, 4H). LC-MS (ES, m/z): [M+H]⁺: 496.

### Example 1.78

### Synthesis of (2R)-2-amino-1-[2-(1,3-benzothiazole-6-sulfonyl)-2H,4H,5H,6H-pyrrolo[3,4-c]pyrazol-5-yl]-2-(3-fluorophenyl)ethan-1-one (Compound 32)

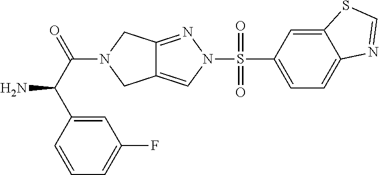 [SMILES: N[C@@H](C(=O)N1Cc2cn(S(=O)(=O)c3ccc4ncsc4c3)nc2C1)c1cccc(F)c1]

The title compound was made from 6-{2H,4H,5H,6H-pyrrolo[3,4-c]pyrazole-2-sulfonyl}-1,3-benzothiazole (Intermediate I-1) and (2R)-2-{[(tert-butoxy)carbonyl]amino}-2-(3-fluorophenyl)acetic acid following a 2-step procedure similar to that described for the synthesis of (2R)-2 amino-1-[2-(1,3-benzothiazole-6-sulfonyl)-2H,4H,5H,6H-pyrrolo[3,4-c]pyrazol-5-yl]-2 phenylethan-1-one (Compound 15). The crude product was purified by reverse phase preparative HPLC (Prep-C18, 5 μM XBridge column, 19×150 mm, Waters; gradient elution of 0-80% MeCN in water over a 20 min period, where both solvents contain 0.1% formic acid) to provide the title product as a white solid. ¹H NMR (400 MHz, DMSO-d6) δ 12.70 (s, 1H), 9.53 (s, 1H), 8.79 (dd, J=11.4, 9.5 Hz, 1H), 8.51 (dd, J=6.1, 1.8 Hz, 1H), 8.04 (d, J=8.7 Hz, 1H), 7.80 (ddd, J=8.6, 3.3, 1.9 Hz, 1H), 7.49 (d, J=18.7 Hz, 1H), 7.23-7.03 (m, 3H), 6.95-6.77 (m, 1H), 5.32 (d, J=9.4 Hz, 1H), 4.78 (dd, J=12.8, 5.3 Hz, 1H), 4.37 (dd, J=12.6, 5.6 Hz, 1H), 4.20 (dd, J=14.7, 5.5 Hz, 1H), 4.04 (d, J=14.6 Hz, 1H). LCMS (ES) [M+1]+ m/z 458.6.

### Example 1.79

### Synthesis of (2R)-1-[2-(1-benzofuran-5-sulfonyl)-2H,4H,5H,6H-pyrrolo[3,4-c]pyrazol-5-yl]-3-hydroxy-3-methyl-2-phenylbutan-1-one (Compound 33)

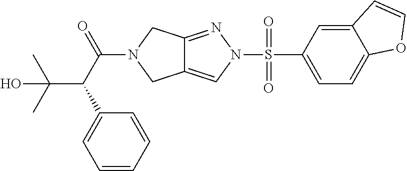 [SMILES: CC(C)(O)[C@H](C(=O)N1Cc2cn(S(=O)(=O)c3ccc4occc4c3)nc2C1)c1ccccc1]

The title compound was made from 2-(1-benzofuran-5-sulfonyl)-4H,5H,6H-pyrrolo[3,4-c]pyrazole (Intermediate I-5) and (2R)-3-hydroxy-3-methyl-2-phenylbutanoic acid following a procedure similar to that described for the synthesis of (2R)-1-[2-(1,3-benzothiazole-6-sulfonyl)-2H,4H,5H,6Hpyrrolo[3,4-c]pyrazol-5-yl]-3-hydroxy-2-phenylpropan-1-one or (2S)-1-[2-(1,3-benzothiazole-6-sulfonyl)-2H,4H,5H,6H-pyrrolo[3,4-c]pyrazol-5-yl]-3-hydroxy-2-phenylpropan-1-one (Compound 1). The crude product was purified by Prep-HPLC with the following conditions (2 #SHIMADZU (HPLC-01)): Column, XBridge Prep C18 OBD Column, 19*100 mm 5 um 13 nm; mobile phase, Water (20 mmol/L NH₄HCO₃) and ACN (35% ACN up to 60% in 10 min). This resulted in 58.4 mg (34.81%) of (2R)-1-[2-(1-benzofuran-5-sulfonyl)-4H,6H-pyrrolo[3,4-c]pyrazol-5-yl]-3-hydroxy-3-methyl-2-phenylbutan-1-one as a white solid. ¹H NMR (300 MHz, DMSO-d₆) δ¹H NMR (300 MHz, DMSO-d₆) δ 8.38 (d, J=1.4 Hz, 1H), 8.27 (d, J=2.2 Hz, 1H), 8.24 (d, J=2.2 Hz, 1H), 7.89-7.85 (m, 2H), 7.41 (d, J=7.4 Hz, 2H), 7.35-7.19 (m, 3H), 7.16 (d, J=2.2 Hz, 1H), 5.03 (s, 1H), 4.90 (t, J=14.3 Hz, 1H), 4.54-4.42 (m, 1H), 4.41-4.27 (m, 1H), 4.17 (d, J=14.3 Hz, 1H), 3.85 (d, J=10.1 Hz, 1H), 1.21 (s, 3H), 0.97 (d, J=3.7 Hz, 3H). LCMS (ES, m/z): [M+H]⁺: 466.

### Example 1.80

### Synthesis of (2S)-1-[2-(1-benzofuran-5-sulfonyl)-2H,4H,5H,6H-pyrrolo[3,4-c]pyrazol-5-yl]-3-hydroxy-3-methyl-2-phenylbutan-1-one (Compound 34)

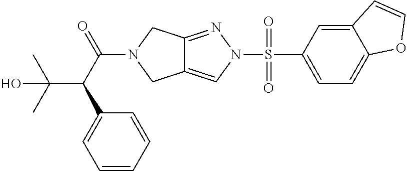 [SMILES: CC(C)(O)[C@@H](C(=O)N1Cc2cn(S(=O)(=O)c3ccc4occc4c3)nc2C1)c1ccccc1]

The title compound was made from 2-(1-benzofuran-5-sulfonyl)-4H,5H,6H-pyrrolo[3,4-c]pyrazole (Intermediate I-5) and (2S)-3-hydroxy-3-methyl-2-phenylbutanoic acid following a procedure similar to that described for the synthesis of (2R)-1-[2-(1,3-benzothiazole-6-sulfonyl)-2H,4H,5H,6Hpyrrolo[3,4-c]pyrazol-5-yl]-3-hydroxy-2-phenylpropan-1-one or (2S)-1-[2-(1,3-benzothiazole-6-sulfonyl)-2H,4H,5H,6H-pyrrolo[3,4-c]pyrazol-5-yl]-3-hydroxy-2-phenylpropan-1-one (Compound 1). The crude product was purified by Prep-HPLC with the following conditions (2 #SHIMADZU (HPLC-01)): Column, XBridge Prep C18 OBD Column, 19*100 mm 5 um 13 nm; mobile phase, Water (20 mmol/L NH₄HCO₃) and ACN (35% ACN up to 60% in 10 min). This resulted in 56.1 mg (33.44%) of (2S)-1-[2-(1-benzofuran-5-sulfonyl)-4H,6H-pyrrolo[3,4-c]pyrazol-5-yl]-3-hydroxy-3-methyl-2-phenylbutan-1-one as a white solid. ¹H NMR (300 MHz, DMSO-d₆) δ 8.38 (d, J=1.4 Hz, 1H), 8.27 (d, J=2.2 Hz, 1H), 8.24 (d, J=2.2 Hz, 1H), 7.89-7.85 (m, 2H), 7.41 (d, J=7.4 Hz, 2H), 7.35-7.19 (m, 3H), 7.16 (d, J=2.2 Hz, 1H), 5.03 (s, 1H), 4.90 (t, J=14.3 Hz, 1H), 4.54-4.42 (m, 1H), 4.41-4.27 (m, 1H), 4.17 (d, J=14.3 Hz, 1H), 3.85 (d, J=10.1 Hz, 1H), 1.21 (s, 3H), 0.97 (d, J=3.7 Hz, 3H). LCMS (ES, m/z): [M+H]⁺: 466.

### Example 1.81

### Synthesis of 6-({5-[(3S)-3-(6-methylpyridin-2-yl)oxolane-3-carbonyl]-2H,4H,5H,6H-pyrrolo[3,4-c]pyrazol-2-yl}sulfonyl)-1,3-benzothiazole or 6-({5-[(3R)-3-(6-methylpyridin-2-yl)oxolane-3-carbonyl]-2H,4H,5H,6H-pyrrolo[3,4-c]pyrazol-2-yl}sulfonyl)-1,3-benzothiazole (Compound 35)

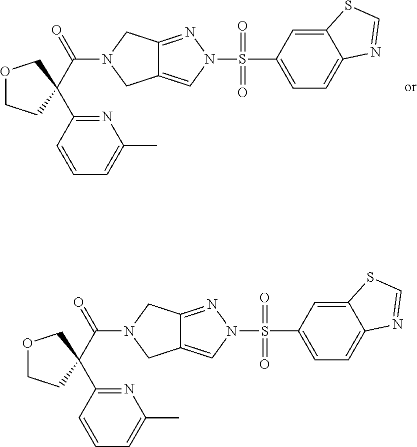 [SMILES: Cc1cccc([C@@]2(C(=O)N3Cc4cn(S(=O)(=O)c5ccc6ncsc6c5)nc4C3)CCOC2)n1 ; Cc1cccc([C@@]2(C(=O)N3Cc4cn(S(=O)(=O)c5ccc6ncsc6c5)nc4C3)CCOC2)n1]

The title compound was made from 6-{2H,4H,5H,6H-pyrrolo[3,4-c]pyrazole-2-sulfonyl}-1,3-benzothiazole (Intermediate I-1) and 2-(bromomethyl)-6-methylpyridine following a 5-step procedure similar to that described for the synthesis of (6-({5-[(3S)-3-(6-methylpyridin-3-yl)oxolane-3-carbonyl]-2H,4H,5H,6H-pyrrolo[3,4-c]pyrazol-2-yl}sulfonyl)-1,3-benzothiazole or 6-({5-[(3R)-3-(6-methylpyridin-3-yl)oxolane-3-carbonyl]-2H,4H,5H,6H-pyrrolo[3,4-c]pyrazol-2-yl}sulfonyl)-1,3-benzothiazole (Compound 31; steps 2-6). The racemic crude product was purified by Prep-CHIRAL-HPLC with the following conditions: Column, Lux Cellulose-4, 100*4.6 mm, 3 um H19-059418; mobile phase, Ethanol and ACN (30% in 11 min); Detector, 254. This resulted in 92.7 mg (30.90%) of 6-({5-[(3S)-3-(6-methylpyridin-2-yl)oxolane-3-carbonyl]-2H,4H,5H,6H-pyrrolo[3,4-c]pyrazol-2-yl}sulfonyl)-1,3-benzothiazole or 6-({5-[(3R)-3-(6-methylpyridin-2-yl)oxolane-3-carbonyl]-2H,4H,5H,6H-pyrrolo[3,4-c]pyrazol-2-yl}sulfonyl)-1,3-benzothiazole (RT=2.76 min) as a white solid. ¹H NMR (300 MHz, DMSO-d₆, ppm): δ 9.71 (s, 1H), 9.00 (dd, J=5.2, 2.0 Hz, 1H), 8.29 (dd, J=8.8, 2.4 Hz, 1H), 8.27 (s, 0.5H), 8.15 (s, 0.5H), 8.02 (dt, J=8.7, 2.2 Hz, 1H), 7.64 (td, J=7.8, 2.7 Hz, 1H), 7.16-7.03 (m, 2H), 4.46 (d, J=5.0 Hz, 2H), 4.35 (t, J=8.7 Hz, 1H), 4.08-3.93 (m, 2H), 3.86-3.70 (m, 3H), 2.70-2.62 (m, 1H), 2.59-2.50 (m, 1H), 2.40 (d, J=12.7 Hz, 3H). LCMS (ES, m/z): [M+H]⁺: 496.

### Example 1.82

### Synthesis of 6-({5-[(3S)-3-(6-methylpyridin-2-yl)oxolane-3-carbonyl]-2H,4H,5H,6H-pyrrolo[3,4-c]pyrazol-2-yl}sulfonyl)-1,3-benzothiazole or 6-({5-[(3R)-3-(6-methylpyridin-2-yl)oxolane-3-carbonyl]-2H,4H,5H,6H-pyrrolo[3,4-c]pyrazol-2-yl}sulfonyl)-1,3-benzothiazole (Compound 36)

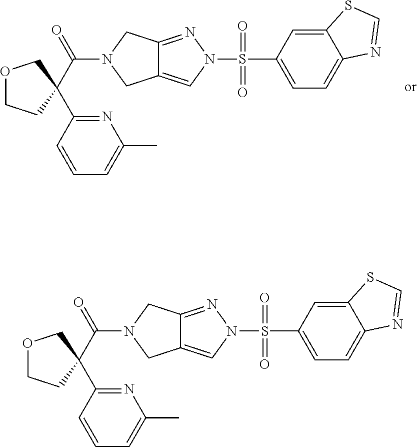 [SMILES: Cc1cccc([C@@]2(C(=O)N3Cc4cn(S(=O)(=O)c5ccc6ncsc6c5)nc4C3)CCOC2)n1 ; Cc1cccc([C@@]2(C(=O)N3Cc4cn(S(=O)(=O)c5ccc6ncsc6c5)nc4C3)CCOC2)n1]

The title compound was prepared by the procedure described for 6-({5-[(3S)-3-(6-methylpyridin-2-yl)oxolane-3-carbonyl]-2H,4H,5H,6H-pyrrolo[3,4-c]pyrazol-2-yl}sulfonyl)-1,3-benzothiazole or 6-({5-[(3R)-3-(6-methylpyridin-2-yl)oxolane-3-carbonyl]-2H,4H,5H,6H-pyrrolo[3,4-c]pyrazol-2-yl}sulfonyl)-1,3-benzothiazole (Compound 35). Purification of the racemic product by the condition described therein provided 81.7 mg (27.23%) of 6-({5-[(3S)-3-(6-methylpyridin-2-yl)oxolane-3-carbonyl]-2H,4H,5H,6H-pyrrolo[3,4-c]pyrazol-2-yl}sulfonyl)-1,3-benzothiazole or 6-({5-[(3R)-3-(6-methylpyridin-2-yl)oxolane-3-carbonyl]-2H,4H,5H,6H-pyrrolo[3,4-c]pyrazol-2-yl}sulfonyl)-1,3-benzothiazole (RT=2.26 min) as a white solid. ¹H-NMR (300 MHz, DMSO-d₆, ppm): δ 9.71 (s, 1H), 9.00 (dd, J=5.2, 2.0 Hz, 1H), 8.29 (dd, J=8.7, 2.4 Hz, 1H), 8.27 (s, 0.5H), 8.15 (s, 0.5H), 8.02 (dt, J=8.7, 2.2 Hz, 1H), 7.64 (td, J=7.7, 2.7 Hz, 1H),7.18-7.02 (m, 2H), 4.46 (d, J=5.1 Hz, 2H), 4.35 (t, J=8.7 Hz, 1H), 4.08-3.94 (m, 2H), 3.86-3.69 (m, 3H), 2.70-2.62 (m, 1H), 2.55-5.52 (m, 1H), 2.40 (d, J=12.7 Hz, 3H). LCMS (ES, m/z): [M+H]⁺: 496.

### Example 1.83

### Synthesis of (2R)-2-amino-1-[2-(1,3-benzothiazole-6-sulfonyl)-2H,4H,5H,6H-pyrrolo[3,4-c]pyrazol-5-yl]-2-(3-methylphenyl)ethan-1-one (Compound 37)

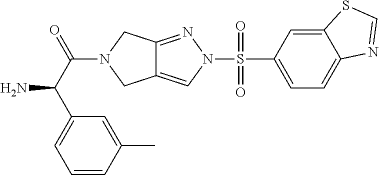 [SMILES: Cc1cccc([C@@H](N)C(=O)N2Cc3cn(S(=O)(=O)c4ccc5ncsc5c4)nc3C2)c1]

The title compound was made from 6-{2H,4H,5H,6H-pyrrolo[3,4-c]pyrazole-2-sulfonyl}-1,3-benzothiazole (Intermediate I-1) and (R)-amino(3-methylphenyl)acetic acid following a 3-step procedure similar to that described for the synthesis of (2R)-2-amino-1-{2-[4-(difluoromethoxy)benzenesulfonyl]-2H,4H,5H,6H-pyrrolo[3,4-c]pyrazol-5-yl}-2-(3-fluorophenyl)ethan-1-one (Compound 29). The crude product was purified by Prep-HPLC with the following conditions: Column, Atlantis HILIC OBD Column, 19*150 mm*5 um, Mobile phase, Water (0.1% FA) and CH₃CN (40% Phase B up to 60% in 8 min); Detector, UV 254 nm to provide 144.7 mg (38%) of (R)-2-amino-1-(2-(benzo[d]thiazol-6-ylsulfonyl)-2,6-dihydropyrrolo[3,4-c]pyrazol-5(4H)-yl)-2-(m-tolyl)ethan-1-one (formate salt) as a white solid. ¹H-NMR (300 MHz, DMSO-d₆, ppm): δ 9.71(s, 1H), 9.02(t, J=1.8 Hz, 1H), 8.31-8.25(m, 3H), 8.04 (dd, J=8.7, 1.8 Hz, 1H), 7.24-7.19(m, 3H), 7.12-7.07(m, 1H), 4.95-4.80(m, 2H), 4.53-4.33(m, 2H), 4.15-4.07(m, 1H), 2.27(d, J=9.0 Hz, 3H). LCMS (ES, m/z): [M−HCOOH+H]⁺: 454.

### Example 1.84

### Synthesis of (2S)-2-amino-1-[2-(1,3-benzothiazole-6-sulfonyl)-2H,4H,5H,6H-pyrrolo[3,4-c]pyrazol-5-yl]-2-(2-fluorophenyl)ethan-1-one (Compound 38)

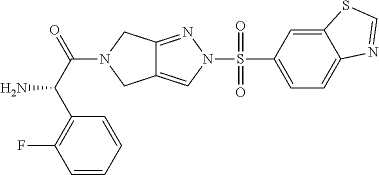 [SMILES: N[C@H](C(=O)N1Cc2cn(S(=O)(=O)c3ccc4ncsc4c3)nc2C1)c1ccccc1F]

The title compound was made from 6-{2H,4H,5H,6H-pyrrolo[3,4-c]pyrazole-2-sulfonyl}-1,3-benzothiazole (Intermediate I-1) and (S)-[(tert-butoxycarbonyl)amino](2-fluorophenyl)acetic acid following a 2-step procedure similar to that described for the synthesis of (2R)-2-amino-1-{2-[4-(difluoromethoxy)benzenesulfonyl]-2H,4H,5H,6H-pyrrolo[3,4-c]pyrazol-5-yl}-2-(3-fluorophenyl)ethan-1-one (Compound 29; steps 2-3). The crude product was purified by Prep-HPLC with the following conditions: Column, Atlantis HILIC OBD Column, 19*150 mm*5 um, Mobile phase, Water (0.1% FA) and CH₃CN (40% Phase B up to 60% in 8 min), Detector, UV 254 nm to provide 165.4 mg (46%) of (S)-2-amino-1-(2-(benzo[d]thiazol-6-ylsulfonyl)-2,6-dihydropyrrolo[3,4-c]pyrazol-5(4H)-yl)-2-(2-fluorophenyl)ethan-1-one (formate salt) as a white solid. ¹H-NMR (300 MHz, DMSO-d₆, ppm): 9.71(s, 1H), 9.03(d, J=2.1 Hz, 1H), 8.32-8.29(m, 2H), 8.15(s, 1H), 8.07-8.02(m, 1H), 7.45-7.29(m, 2H), 7.20-7.15(m, 2H), 4.99-4.84(m, 2H), 4.52-4.34(m, 2H), 4.23-4.09(m, 1H). LCMS (ES, m/z): [M+H]⁺: 458.1.

### Example 1.85

### Synthesis of 2-amino-1-[2-(1,3-benzothiazole-6-sulfonyl)-2H,4H,5H,6H-pyrrolo[3,4-c]pyrazol-5-yl]-2-(4-fluoro-3-methylphenyl)ethan-1-one (Compound 39; Racemic)

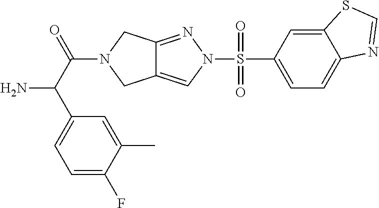 [SMILES: Cc1cc(C(N)C(=O)N2Cc3cn(S(=O)(=O)c4ccc5ncsc5c4)nc3C2)ccc1F]

The title compound was made from 6-{2H,4H,5H,6H-pyrrolo[3,4-c]pyrazole-2-sulfonyl}-1,3-benzothiazole (Intermediate I-1) and 2-{[(tert-butoxy)carbonyl]amino}-2-(4-fluoro-3-methylphenyl)acetic acid following a 2-step procedure similar to that for the synthesis of (2R)-2 amino-1-[2-(1,3-benzothiazole-6-sulfonyl)-2H,4H,5H,6H-pyrrolo[3,4-c]pyrazol-5-yl]-2 phenylethan-1-one (Compound 15). The crude product was purified by reverse phase preparative HPLC (Prep-C18, 5 μM XBridge column, 19×150 mm, Waters; gradient elution of 0-80% MeCN in water over a 20 min period, where both solvents contain 0.1% formic acid) to provide the title product as a white solid. ¹H NMR (400 MHz, DMSO-d6) δ 9.69 (s, 1H), 9.00 (t, J=1.5 Hz, 1H), 8.34-8.21 (m, 2H), 8.02 (ddd, J=8.7, 2.1, 0.8 Hz, 1H), 7.31-7.12 (m, 2H), 7.03 (dt, J=9.8, 8.1 Hz, 1H), 4.82 (dd, J=22.7, 14.3 Hz, 1H), 4.63 (d, J=17.2 Hz, 1H), 4.50-4.28 (m, 2H), 4.20 (dd, J=20.8, 14.3 Hz, 1H), 2.16 (dd, J=3.8, 1.9 Hz, 3H). LCMS (ES) [M+1]+ m/z 472.2.

### Example 1.86

### Synthesis of 6-{[5-(3-phenyloxetane-3-carbonyl)-2H,4H,5H,6H-pyrrolo[3,4-c]pyrazol-2-yl]sulfonyl}-1,3-benzothiazole (Compound 40)

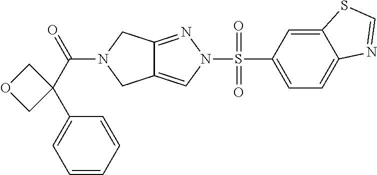 [SMILES: O=C(N1Cc2cn(S(=O)(=O)c3ccc4ncsc4c3)nc2C1)C1(c2ccccc2)COC1]

The title compound was made from 6-{2H,4H,5H,6H-pyrrolo[3,4-c]pyrazole-2-sulfonyl}-1,3-benzothiazole (Intermediate I-1) and 3-phenyloxetane-3-carboxylic acid following a procedure similar as described for the synthesis of Compound 1. The crude product was purified by reverse phase preparative HPLC (Prep-C18, 5 μM XBridge column, 19×150 mm, Waters; gradient elution of 0-80% MeCN in water over a 20 min period, where both solvents contain 0.1% formic acid) to provide the title product as a white solid. ¹H NMR (400 MHz, DMSO-d6) δ 9.69 (d, J=3.3 Hz, 1H), 8.99 (dd, J=9.6, 2.0 Hz, 1H), 8.32-8.23 (m, 1H), 8.01 (ddd, J=8.7, 6.0, 2.0 Hz, 1H), 7.50-7.35 (m, 4H), 7.34-7.23 (m, 1H), 5.17 (dd, J=9.7, 6.3 Hz, 2H), 4.68 (dd, J=6.4, 2.5 Hz, 2H), 4.50 (d, J=11.7 Hz, 2H), 3.95-3.80 (m, 2H). LCMS (ES) [M+1]+ m/z 467.2.

### Example 1.87

### Synthesis of (2R)-1-[2-(1,3-benzothiazole-6-sulfonyl)-2H,4H,5H,6H-pyrrolo[3,4-c]pyrazol-5-yl]-2-hydroxy-2-phenylethan-1-one (Compound 42)

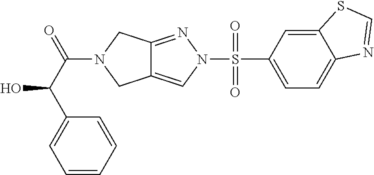 [SMILES: O=C([C@H](O)c1ccccc1)N1Cc2cn(S(=O)(=O)c3ccc4ncsc4c3)nc2C1]

The title compound was made from 6-{2H,4H,5H,6H-pyrrolo[3,4-c]pyrazole-2-sulfonyl}-1,3-benzothiazole (Intermediate I-1) and (2R)-hydroxy(phenyl)ethanoic acid following a procedure similar as described for the synthesis of Compound 1. The crude product was purified by reverse phase preparative HPLC (Prep-C18, 5 μM XBridge column, 19×150 mm, Waters; gradient elution of 0-65% MeCN in water over a 20 min period, where both solvents contain 0.1% formic acid) to provide the title product as a white solid. ¹H NMR (400 MHz, DMSO-d6) δ 9.69 (s, 1H), 9.00 (t, J=1.6 Hz, 1H), 8.27 (dd, J=9.3, 6.6 Hz, 2H), 8.01 (ddd, J=8.7, 2.1, 1.1 Hz, 1H), 7.46-7.12 (m, 5H), 5.72 (dd, J=6.4, 3.3 Hz, 1H), 5.26 (dd, J=13.1, 6.2 Hz, 1H), 4.68 (dd, J=26.8, 14.5 Hz, 1H), 4.52-4.16 (m, 3H). LCMS (ES) [M+1]+ m/z 441.2.

### Example 1.88

### Synthesis (2R)-1-[2-(1,3-benzothiazole-6-sulfonyl)-2H,4H,5H,6H-pyrrolo[3,4-c]pyrazol-5-yl]-3-hydroxy-2-methyl-2-(pyridin-2-yl)propan-1-one or (2S)-1-[2-(1,3-benzothiazole-6-sulfonyl)-2H,4H,5H,6H-pyrrolo[3,4-c]pyrazol-5-yl]-3-hydroxy-2-methyl-2-(pyridin-2-yl)propan-1-one (Compound 43)

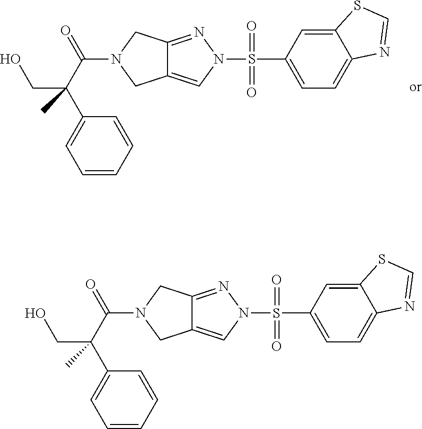 [SMILES: C[C@@](CO)(C(=O)N1Cc2cn(S(=O)(=O)c3ccc4ncsc4c3)nc2C1)c1ccccc1 ; C[C@](CO)(C(=O)N1Cc2cn(S(=O)(=O)c3ccc4ncsc4c3)nc2C1)c1ccccc1]

Step 1

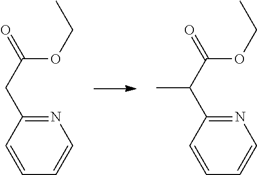

Into a 250-mL 3 neck round-bottom flask, was placed ethyl 2-(pyridin-2-yl)acetate (10.00 g, 60.536 mmol, 1.00 eq.), tetrahydrofuran (30 mL), and HMPA (10.85 g, 60.536 mmol, 1.00 eq.) followed by the drop-wise addition of LDA (30.00 mL, 60.500 mmol, 1.00 eq., 2M) at −78° C. The resulting solution was stirred for 0.5 h at −78° C., then dimethyl sulfate (7.64 g, 60.536 mmol, 1.00 eq.) was added drop-wise at −78° C. After 1 h, the reaction was quenched by the addition of 100 mL of water and extracted with 2×200 mL of ethyl acetate. The organic layer was dried over anhydrous sodium sulfate and concentrated under vacuum. The residue was applied onto a silica gel column with ethyl acetate/petroleum ether (1:10) to provide 9.5 g (87.56%) of ethyl 2-(pyridin-2-yl)propanoate as a light yellow oil. LCMS (ES) [M+1]⁺ m/z:180.

Step 2

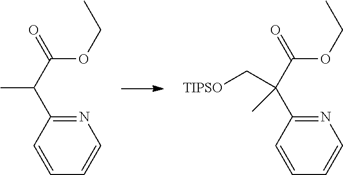

Into a 250-mL round-bottom flask, was placed ethyl 2-(pyridin-2-yl)propanoate (6.00 g, 33.479 mmol, 1.00 eq.), and tetrahydrofuran (60 mL), followed by the drop-wise addition of LDA (33.50 mL, 66.9 mmol, 1.00 eq., 2M) at −78° C. The resulting solution was stirred for 1 h at −78° C., then (chloromethoxy)triisopropylsilane (14.92 g, 66.957 mmol, 2.00 eq.) was added. The resulting solution was stirred for 16 hr at 25° C. The reaction was then quenched by the addition of 100 mL of water and extracted with 2×2 00 mL of ethyl acetate. The organic layer was dried over anhydrous sodium sulfate and concentrated under vacuum. The residue was applied onto a silica gel column with ethyl acetate/petroleum ether (1:10) to provide 9.8 g (80.07%) of ethyl 2-methyl-2-(pyridin-2-yl)-3-[(triisopropylsilyl)oxy]propanoate as a light yellow solid. LCMS (ES) [M+1]⁺ m/z: 366.

Step 3

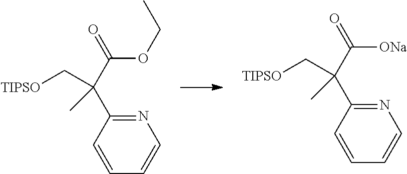

Into a 250-mL round-bottom flask, was placed ethyl 2-methyl-2-(pyridin-2-yl)-3-[(triisopropylsilyl)oxy]propanoate (8.90 g, 24.344 mmol, 1.00 eq.), tetrahydrofuran (20 mL), methanol (20 mL), water (20 mL), and NaOH (3.89 g, 97.377 mmol, 4.00 eq.), and the resulting solution was stirred for 16 h at 80° C. The crude reaction mixture was filtered and subjected to reverse phase preparative HPLC (Prep-C18, 20-45M, 120 g, Tianjin Bonna-Agela Technologies; gradient elution of 50% MeCN in water to 60% MeCN in water over a 10 min period to provide the title compound as a yellow solid (5.4 g, 61.70%). LCMS (ES) [M−Na+H+1]⁺ m/z:338.

Step 4

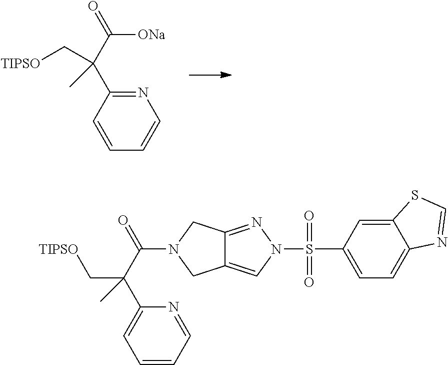

Into a 50-mL round-bottom flask, was placed sodium 2-methyl-2-(pyridin-2-yl)-3-[(triisopropylsilyl)oxy]propanoate (500.00 mg, 1.391 mmol, 1.00 eq.), dimethylformamide (10 mL), 6-[4H,5H,6H-pyrrolo[3,4-c]pyrazole-2-sulfonyl]-1,3-benzothiazole hydrochloride (Intermediate I-1; 476.78 mg, 1.391 mmol, 1.00 eq.), HATU (793.21 mg, 2.086 mmol, 1.50 eq.), and DIEA (539.24 mg, 4.172 mmol, 3.00 eq.). The resulting solution was stirred for 6 h at 25° C. The crude reaction mixture was filtered and subjected to reverse phase preparative HPLC (Prep-C18, 20-45M, 120 g, Tianjin Bonna-Agela Technologies; gradient elution of 60% MeCN in water to 70% MeCN in water over a 10 min period, water contains 0.1% NH₃H₂O) to provide the title compound as a white solid (450 mg, 51.70%). LCMS (ES) [M+1]⁺ m/z: 626.

Step 5

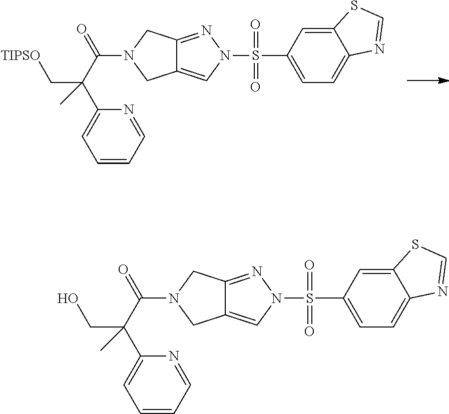

Into a 50-mL round-bottom flask, was placed 1-[2-(1,3-benzothiazole-6-sulfonyl)-4H,6H-pyrrolo[3,4-c]pyrazol-5-yl]-2-methyl-2-(pyridin-2-yl)-3-[(triisopropylsilyl)oxy]propan-1-one (450.00 mg, 0.719 mmol, 1.00 eq.), acetonitrile (9.00 mL), and formic acid (3.00 mL). The resulting solution was stirred for 16 h at 40° C. The crude reaction mixture was filtered and subjected to reverse phase preparative HPLC (Prep-C18, 20-45M, 120 g, Tianjin Bonna-Agela Technologies; gradient elution of 30% MeCN in water to 40% MeCN in water over a 10 min period, water contains 0.1% NH₃H₂O) to provide the title compound as a yellow solid (240 mg, 71.09%). LCMS (ES) [M+1]⁺ m/z: 470.

Step 6

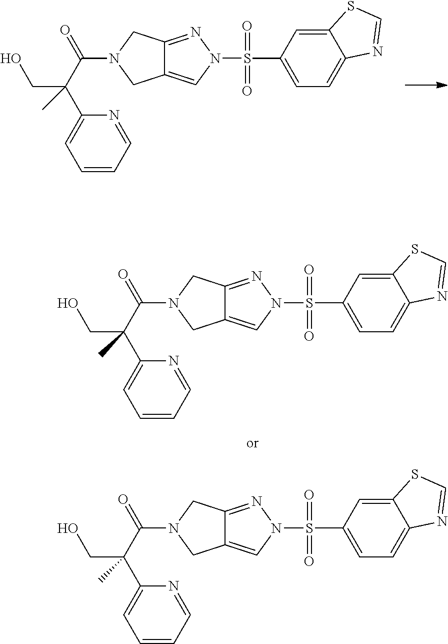 [SMILES: CC(CO)(C(=O)N1Cc2cn(S(=O)(=O)c3ccc4ncsc4c3)nc2C1)c1ccccn1 ; C[C@@](CO)(C(=O)N1Cc2cn(S(=O)(=O)c3ccc4ncsc4c3)nc2C1)c1ccccn1 ; C[C@](CO)(C(=O)N1Cc2cn(S(=O)(=O)c3ccc4ncsc4c3)nc2C1)c1ccccn1]

The racemic 1-[2-(1,3-benzothiazole-6-sulfonyl)-4H,6H-pyrrolo[3,4-c]pyrazol-5-yl]-3-hydroxy-2-methyl-2-(pyridin-2-yl)propan-1-one (240.00 mg) was separated by Chiral Prep-HPLC with the following conditions. Column: CHIRALPAK IA-3, 50*4.6 mm, 3 um IA30CC-UL005; mobile phase: A: n-Hexane, B: Ethanol, gradient elution 50% B in 7 min. This resulted in 97.7 mg (40.71%) of (2R)-1-[2-(1,3-benzothiazole-6-sulfonyl)-2H,4H,5H,6H-pyrrolo[3,4-c]pyrazol-5-yl]-3-hydroxy-2-methyl-2-(pyridin-2-yl)propan-1-one or (2S)-1-[2-(1,3-benzothiazole-6-sulfonyl)-2H,4H,5H,6H-pyrrolo[3,4-c]pyrazol-5-yl]-3-hydroxy-2-methyl-2-(pyridin-2-yl)propan-1-one (RT=3.887 min) as a white solid. ¹H NMR (300 MHz, DMSO-d₆) δ 9.71 (s, 1H), 8.99 (s, 1H), 8.51-8.44 (m,1H), 8.29 (d, J=8.7 Hz, 1H), 8.25 (s, 0.5H), 8.11 (s, 0.5H), 8.01 (dd, J=8.7, 2.0 Hz, 1H), 7.77 (t, J=7.7 Hz, 1H), 7.34 (d, J=8.0 Hz, 1H), 7.29-7.18(m, 1H), 4.69-4.60 (m, 1H), 4.49-4.39 (m, 2H), 3.95-3.72 (m, 3H), 3.50-3.46 (m, 1H), 1.57 (s, 3H). LCMS (ES, m/z): [M+H]⁺: 470.1.

### Example 1.89

### Synthesis (2R)-1-[2-(1,3-benzothiazole-6-sulfonyl)-2H,4H,5H,6H-pyrrolo[3,4-c]pyrazol-5-yl]-3-hydroxy-2-methyl-2-(pyridin-2-yl)propan-1-one or (2S)-1-[2-(1,3-benzothiazole-6-sulfonyl)-2H,4H,5H,6H-pyrrolo[3,4-c]pyrazol-5-yl]-3-hydroxy-2-methyl-2-(pyridin-2-yl)propan-1-one (Compound 44)

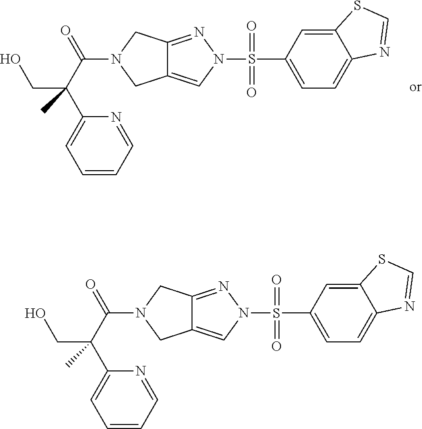 [SMILES: C[C@@](CO)(C(=O)N1Cc2cn(S(=O)(=O)c3ccc4ncsc4c3)nc2C1)c1ccccn1 ; C[C@](CO)(C(=O)N1Cc2cn(S(=O)(=O)c3ccc4ncsc4c3)nc2C1)c1ccccn1]

The title compound was prepared by the procedure described for the synthesis of (2R)-1-[2-(1,3-benzothiazole-6-sulfonyl)-2H,4H,5H,6H-pyrrolo[3,4-c]pyrazol-5-yl]-3-hydroxy-2-methyl-2-(pyridin-2-yl)propan-1-one or (2S)-1-[2-(1,3-benzothiazole-6-sulfonyl)-2H,4H,5H,6H-pyrrolo[3,4-c]pyrazol-5-yl]-3-hydroxy-2-methyl-2-(pyridin-2-yl)propan-1-one (Compound 43). The racemic product was purified by the conditions described therein to provide (2R)-1-[2-(1,3-benzothiazole-6-sulfonyl)-2H,4H,5H,6H-pyrrolo[3,4-c]pyrazol-5-yl]-3-hydroxy-2-methyl-2-(pyridin-2-yl)propan-1-one or (2S)-1-[2-(1,3-benzothiazole-6-sulfonyl)-2H,4H,5H,6H-pyrrolo[3,4-c]pyrazol-5-yl]-3-hydroxy-2-methyl-2-(pyridin-2-yl)propan-1-one (RT=2.488 min) as a white solid. ¹H NMR (300 MHz, DMSO-d₆) δ 9.71 (s, 1H), 8.99 (s, 1H), 8.51-8.44 (m,1H), 8.29 (d, J=8.7 Hz, 1H), 8.25 (s, 0.5H), 8.11 (s, 0.5H), 8.01 (dd, J=8.6, 1.7 Hz, 1H), 7.77 (t, J=7.5 Hz, 1H), 7.34 (d, J=8.0 Hz, 1H), 7.29-7.18(m, 1H), 4.69-4.60 (m, 1H), 4.49-4.39 (m, 2H), 3.95-3.72 (m, 3H), 3.50-3.46 (m, 1H), 1.57 (s, 3H). LCMS (ES, m/z): [M+H]⁺: 470.1.

### Example 1.90

### Synthesis 6-({5-[(3S)-3-(pyridin-2-yl)oxolane-3-carbonyl]-2H,4H,5H,6H-pyrrolo[3,4-c]pyrazol-2-yl}sulfonyl)-3,4-dihydro-2H-1,4-benzoxazine or 6-({5-[(3R)-3-(pyridin-2-yl)oxolane-3-carbonyl]-2H,4H,5H,6H-pyrrolo[3,4-c]pyrazol-2-yl}sulfonyl)-3,4-dihydro-2H-1,4-benzoxazine (Compound 45)

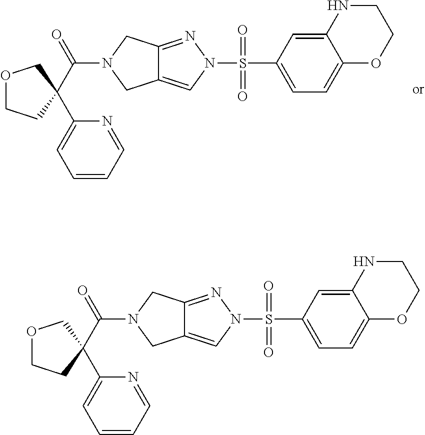 [SMILES: O=C(N1Cc2cn(S(=O)(=O)c3ccc4c(c3)NCCO4)nc2C1)[C@]1(c2ccccn2)CCOC1 ; O=C(N1Cc2cn(S(=O)(=O)c3ccc4c(c3)NCCO4)nc2C1)[C@@]1(c2ccccn2)CCOC1]

The title compound was made from 6-((5,6-dihydropyrrolo[3,4-c]pyrazol-2(4H)-yl)sulfonyl)-3,4-dihydro-2H-benzo[b][1,4]oxazine (Intermediate I-3) and sodium 3-(pyridin-2-yl)tetrahydrofuran-3-carboxylate (Intermediate II-7 following a procedure similar to the one described for the synthesis of Compound 1. The racemic material was chiral separated with conditions: chiral PAK IC column, 20*250 mm, 5 um, Phase A: hexane/dichloromethane (1:1), Phase B: ethanol (0.1% DEA), Gradient: 40% phase B within 20 min, Detector: 220 nm to provide 80.9 mg of 6-({5-[(3S)-3-(pyridin-2-yl)oxolane-3-carbonyl]-2H,4H,5H,6H-pyrrolo[3,4-c]pyrazol-2-yl}sulfonyl)-3,4-dihydro-2H-1,4-benzoxazine or 6-({5-[(3R)-3-(pyridin-2-yl)oxolane-3-carbonyl]-2H,4H,5H,6H-pyrrolo[3,4-c]pyrazol-2-yl}sulfonyl)-3,4-dihydro-2H-1,4-benzoxazine (RT=2.15 min). ¹H-NMR (300 MHz, DMSO-d₆, ppm): δ 8.56-8.52 (m, 1H), 8.12 (s, 0.5H), 8.00 (s, 0.5 H), 7.83-7.77 (m, 1H),7.36-7.30 (m, 2H), 7.10 (dd, J=6.3, 2.4 Hz, 1H), 7.01 (dd, J=8.1, 2.1 Hz, 1H), 6.82 (dd, J=8.4, 2.1 Hz, 1H), 6.45 (br, 1H), 4.48 (d, J=9.6 Hz, 2H), 4.36 (dd, J=8.7, 6.6 Hz, 1H), 4.17 (t, J=3.6 Hz, 2H), 4.05-3.99 (m, 2H), 3.86(t, J=6.6 Hz, 2H), 3.70 (d, J=13.8 Hz, 1H), 3.40-3.32 (m, 2H), 2.71-2.55 (m, 2H). LC-MS (ES, m/z): [M+H]⁺: 482.

### Example 1.91

### Synthesis 6-({5-[(3S)-3-(pyridin-2-yl)oxolane-3-carbonyl]-2H,4H,5H,6H-pyrrolo[3,4-c]pyrazol-2-yl}sulfonyl)-3,4-dihydro-2H-1,4-benzoxazine or 6-({5-[(3R)-3-(pyridin-2-yl)oxolane-3-carbonyl]-2H,4H,5H,6H-pyrrolo[3,4-c]pyrazol-2-yl}sulfonyl)-3,4-dihydro-2H-1,4-benzoxazine (Compound 46)

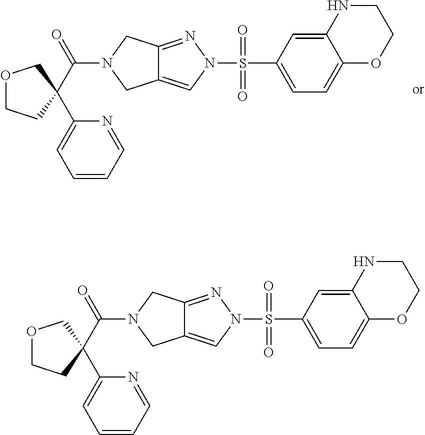 [SMILES: O=C(N1Cc2cn(S(=O)(=O)c3ccc4c(c3)NCCO4)nc2C1)[C@]1(c2ccccn2)CCOC1 ; O=C(N1Cc2cn(S(=O)(=O)c3ccc4c(c3)NCCO4)nc2C1)[C@@]1(c2ccccn2)CCOC1]

The title compound was made by the procedure described for 6-({5-[(3S)-3-(pyridin-2-yl)oxolane-3-carbonyl]-2H,4H,5H,6H-pyrrolo[3,4-c]pyrazol-2-yl}sulfonyl)-3,4-dihydro-2H-1,4-benzoxazine or 6-({5-[(3R)-3-(pyridin-2-yl)oxolane-3-carbonyl]-2H,4H,5H,6H-pyrrolo[3,4-c]pyrazol-2-yl}sulfonyl)-3,4-dihydro-2H-1,4-benzoxazine (Compound 45). The racemic material was chiral separated with conditions: chiral PAK IC column, 20*250 mm, 5 um, Phase A: hexane/dichloromethane (1:1), Phase B: ethanol (0.1% DEA), Gradient: 40% phase B within 20 min, Detector: 220 nm to provide 75.8 mg of 6-({5-[(3S)-3-(pyridin-2-yl)oxolane-3-carbonyl]-2H,4H,5H,6H-pyrrolo[3,4-c]pyrazol-2-yl}sulfonyl)-3,4-dihydro-2H-1,4-benzoxazine or 6-({5-[(3R)-3-(pyridin-2-yl)oxolane-3-carbonyl]-2H,4H,5H,6H-pyrrolo[3,4-c]pyrazol-2-yl}sulfonyl)-3,4-dihydro-2H-1,4-benzoxazine (RT=2.96 min). ¹H-NMR (300 MHz, DMSO-d₆, ppm): δ 8.56-8.52 (m, 1H), 8.12 (s, 0.5H), 8.00 (s, 0.5H),7.83-7.77(m, 1H),7.36-7.30 (m, 2H), 7.10 (dd, J=6.3, 2.4 Hz, 1H), 7.01 (dd, J=8.1, 2.1 Hz, 1H), 6.82 (dd, J=8.4, 2.1 Hz, 1H), 6.45 (br, 1H), 4.48 (d, J=9.6 Hz, 2H), 4.36 (dd, J=8.7, 6.6 Hz, 1H), 4.17 (t, J=3.6 Hz, 2H), 4.05-3.99 (m, 2H), 3.86(t, J=6.6 Hz, 2H), 3.70 (d, J=13.8 Hz, 1H), 3.40-3.32 (m, 2H), 2.71-2.55 (m, 2H). LC-MS (ES, m/z): [M+H]⁺: 482.

### Example 1.92

### Synthesis of (2R)-2-amino-1-[2-(1,3-benzothiazole-6-sulfonyl)-2H,4H,5H,6H-pyrrolo[3,4-c]pyrazol-5-yl]-2-(2-methylpyridin-3-yl)ethan-1-one or (2S)-2-amino-1-[2-(1,3-benzothiazole-6-sulfonyl)-2H,4H,5H,6H-pyrrolo[3,4-c]pyrazol-5-yl]-2-(2-methylpyridin-3-yl)ethan-1-one

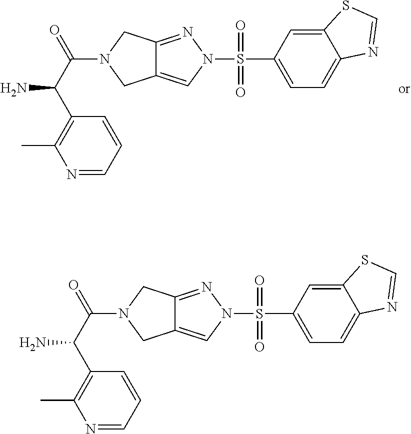 [SMILES: Cc1ncccc1[C@@H](N)C(=O)N1Cc2cn(S(=O)(=O)c3ccc4ncsc4c3)nc2C1 ; Cc1ncccc1[C@H](N)C(=O)N1Cc2cn(S(=O)(=O)c3ccc4ncsc4c3)nc2C1]

### (Compound 47)

Step 1

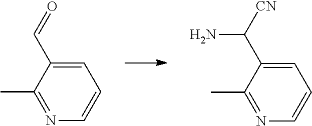

Into a 250-mL round-bottom flask, was placed NaCN (4.05 g, 82.64 mmol, 2.00 eq.), NH₄Cl (4.42 g, 82.55 mmol, 2.00 eq.), and NH₃.H₂O (50.00 mL) followed by the drop-wise addition of a solution of 2-methylpyridine-3-carbaldehyde (5.00 g, 41.27 mmol, 1.00 eq.) in MeOH (5 mL) with stirring at room temperature. After 4 h the solution was extracted with 3×50 mL of ethyl acetate and the organic layers were combined and dried over anhydrous sodium sulfate and concentrated. This resulted in 3.5 g (57.61%) of 2-amino-2-(2-methylpyridin-3-yl)acetonitrile as a yellow solid. LCMS (ES) [M+1]⁺ m/z: 148.

Step 2

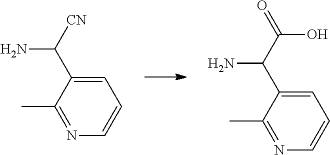

Into a 100-mL round-bottom flask, was placed 2-amino-2-(2-methylpyridin-3-yl)acetonitrile (3.50 g, 23.78 mmol, 1.00 eq.), and 6M HCl (50.00 mL) and the resulting solution was stirred at 100° C. overnight. The reaction mixture was then cooled and concentrated. This resulted in 3 g (75.91%) of amino(2-methylpyridin-3-yl)acetic acid as a yellow solid. LCMS (ES) [M+1]⁺ m/z: 167.

Step 3

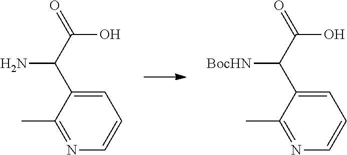 [SMILES: Cc1ncccc1C(N)C(=O)O ; Cc1ncccc1C(NC(=O)OC(C)(C)C)C(=O)O]

Into a 100-mL round-bottom flask, was placed amino(2-methylpyridin-3-yl)acetic acid (3.00 g, 18.05 mmol, 1.00 eq.), MeOH (40.00 mL), and Et₃N (3.65 g, 36.10 mmol, 2.00 eq.), followed by the addition of di-tert-butyl dicarbonate (4.73 g, 21.67 mmol, 1.20 eq.), in portions at 0° C. The resulting solution was stirred overnight at room temperature. The crude product (6 g) was purified by Prep-HPLC with the following conditions: Column, XBridge Prep C18 OBD Column, 19 cm, 150 mm, 5 um; mobile phase, Water (0.1% NH₄HCO₃) and CAN (5% Phase B up to 20% in 11 min); Detector, 254 to provide 3 g (62.40%) of [(tert-butoxycarbonyl)amino](2-methylpyridin-3-yl)acetic acid as a yellow oil. LCMS (ES) [M+1]+ m/z: 267.

Step 4

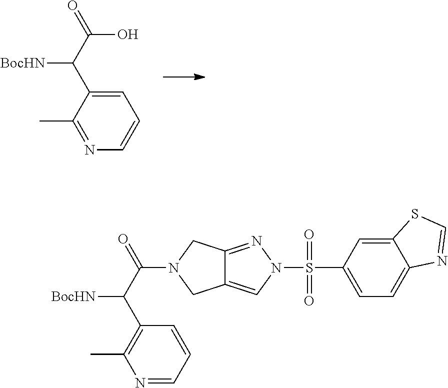 [SMILES: Cc1ncccc1C(NC(=O)OC(C)(C)C)C(=O)O ; Cc1ncccc1C(NC(=O)OC(C)(C)C)C(=O)N1Cc2cn(S(=O)(=O)c3ccc4ncsc4c3)nc2C1]

Into a 40-mL vial, was placed [(tert-butoxycarbonyl)amino](2-methylpyridin-3-yl)acetic acid (600.00 mg, 2.25 mmol, 1.00 eq.), DMF (10.00 mL), DIEA (582.40 mg, 4.51 mmol, 2.00 eq.), and 6-[4H,5H,6H-pyrrolo[3,4-c]pyrazole-2-sulfonyl]-1,3-benzothiazole (828.32 mg, 2.70 mmol, 1.20 eq.), followed by the addition of HATU (1028.05 mg, 2.70 mmol, 1.20 eq.), in portions at 0° C. The resulting solution was stirred for 1.5 h at room temperature. The crude product (1 g) was purified by Prep-HPLC with the following conditions: Column, XBridge Prep C18 OBD Column, 19 cm, 150 mm, 5 um; mobile phase, Water (0.1% NH₄HCO₃) and CAN (40% Phase B up to 60% in 8 min); Detector, 254. This resulted in 500 mg (40.01%) of tert-butyl N-[2-[2-(1,3-benzothiazole-6-sulfonyl)-4H,6H-pyrrolo[3,4-c]pyrazol-5-yl]-1-(2-methylpyridin-3-yl)-2-oxoethyl]carbamate as a yellow solid. LCMS (ES) [M+1]⁺ m/z: 555.

Step 5

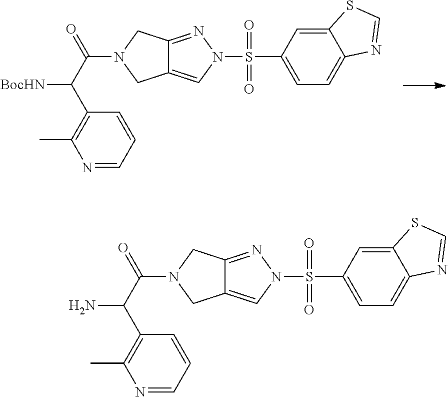 [SMILES: Cc1ncccc1C(NC(=O)OC(C)(C)C)C(=O)N1Cc2cn(S(=O)(=O)c3ccc4ncsc4c3)nc2C1 ; Cc1ncccc1C(N)C(=O)N1Cc2cn(S(=O)(=O)c3ccc4ncsc4c3)nc2C1]

Into a 40-mL vial, was placed tert-butyl N-[2-[2-(1,3-benzothiazole-6-sulfonyl)-4H,6H-pyrrolo[3,4-c]pyrazol-5-yl]-1-(2-methylpyridin-3-yl)-2-oxoethyl]carbamate (500.00 mg, 0.90 mmol, 1.00 eq.), DCM (10.00 mL), and ZnBr₂ (609.09 mg, 2.70 mmol, 3.00 eq.) and the resulting solution was stirred overnight at room temperature. The mixture was then concentrated providing 250 mg (61.01%) of 2-amino-1-[2-(1,3-benzothiazole-6-sulfonyl)-4H,6H-pyrrolo[3,4-c]pyrazol-5-yl]-2-(2-methylpyridin-3-yl)ethanone as a yellow solid. LCMS (ES) [M+1]⁺ m/z: 455.

Step 5

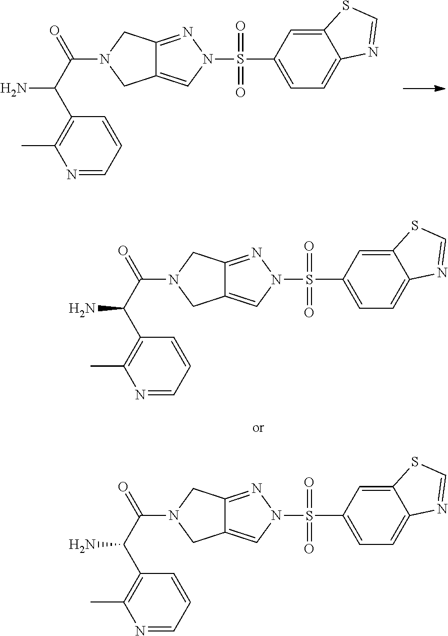 [SMILES: Cc1ncccc1C(N)C(=O)N1Cc2cn(S(=O)(=O)c3ccc4ncsc4c3)nc2C1 ; Cc1ncccc1[C@@H](N)C(=O)N1Cc2cn(S(=O)(=O)c3ccc4ncsc4c3)nc2C1 ; Cc1ncccc1[C@H](N)C(=O)N1Cc2cn(S(=O)(=O)c3ccc4ncsc4c3)nc2C1]

The racemic product (250 mg) was purified by Prep-CHIRAL-HPLC with the following conditions: Column, CHIRALPAK IA-3, 4.6*50 mm, 3 μm; mobile phase, n-Hexane/DCM=3/1 and Ethanol (0.1% DEA) (30% in 15 min); Detector, 254. This resulted in 107.5 mg (43.00%) of (2R)-2-amino-1-[2-(1,3-benzothiazole-6-sulfonyl)-2H,4H,5H,6H-pyrrolo[3,4-c]pyrazol-5-yl]-2-(2-methylpyridin-3-yl)ethan-1-one or (2S)-2-amino-1-[2-(1,3-benzothiazole-6-sulfonyl)-2H,4H,5H,6H-pyrrolo[3,4-c]pyrazol-5-yl]-2-(2-methylpyridin-3-yl)ethan-1-one (RT=1.96 min) as a white solid. ¹H-NMR (300 MHz, DMSO-d₆, ppm): δ 9.71 (d, J=0.9 Hz, 1H), 9.03 (d, J=2.0 Hz, 1H), 8.36-8.22 (m, 3H), 8.04 (dd, J=8.7, 2.0 Hz, 1H), 7.56-7.49 (m, 1H), 7.15 (dd, J=7.8, 4.8 Hz, 1H), 4.95-4.73 (m, 2H), 4.56-4.36 (m, 2H), 4.08-3.96 (m, 1H), 2.52 (s, 3H), 2.30 (s, 2H). LCMS (ES, m/z): [M+H]⁺: 455.

### Example 1.93

### Synthesis of (2R)-2-amino-1-[2-(1,3-benzothiazole-6-sulfonyl)-2H,4H,5H,6H-pyrrolo[3,4-c]pyrazol-5-yl]-2-(2-methylpyridin-3-yl)ethan-1-one or (2S)-2-amino-1-[2-(1,3-benzothiazole-6-sulfonyl)-2H,4H,5H,6H-pyrrolo[3,4-c]pyrazol-5-yl]-2-(2-methylpyridin-3-yl)ethan-1-one (Compound 55)

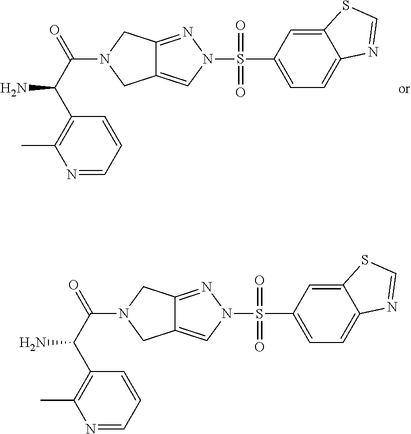 [SMILES: Cc1ncccc1[C@@H](N)C(=O)N1Cc2cn(S(=O)(=O)c3ccc4ncsc4c3)nc2C1 ; Cc1ncccc1[C@H](N)C(=O)N1Cc2cn(S(=O)(=O)c3ccc4ncsc4c3)nc2C1]

The title compound was made by the procedure described for (2R)-2-amino-1-[2-(1,3-benzothiazole-6-sulfonyl)-2H,4H,5H,6H-pyrrolo[3,4-c]pyrazol-5-yl]-2-(2-methylpyridin-3-yl)ethan-1-one or (2S)-2-amino-1-[2-(1,3-benzothiazole-6-sulfonyl)-2H,4H,5H,6H-pyrrolo[3,4-c]pyrazol-5-yl]-2-(2-methylpyridin-3-yl)ethan-1-one Chiral purification by the conditions described therein provided 110 mg (44.00%) of (2R)-2-amino-1-[2-(1,3-benzothiazole-6-sulfonyl)-2H,4H,5H,6H-pyrrolo[3,4-c]pyrazol-5-yl]-2-(2-methylpyridin-3-yl)ethan-1-one or (2S)-2-amino-1-[2-(1,3-benzothiazole-6-sulfonyl)-2H,4H,5H,6H-pyrrolo[3,4-c]pyrazol-5-yl]-2-(2-methylpyridin-3-yl)ethan-1-one (RT=2.75 min) as a white solid. ¹H-NMR (300 MHz, DMSO-d₆, ppm): δ 9.71 (d, J=0.9 Hz, 1H), 9.03 (d, J=1.9 Hz, 1H), 8.36-8.22 (m, 3H), 8.04 (dd, J=8.7, 2.0 Hz, 1H), 7.56-7.49 (m, 1H), 7.14 (dd, J=7.8, 4.8 Hz, 1H), 4.92-4.75 (m, 2H), 4.56-4.36 (m, 2H), 4.08-3.96 (m, 1H), 2.52 (s, 3H), 2.21 (s, 2H). LCMS (ES, m/z): [M+H]⁺: 455.

### Example 1.94

### Synthesis of (2R)-1-[2-(2,3-dihydro-1,4-benzodioxine-6-sulfonyl)-2H,4H,5H,6H-pyrrolo[3,4-c]pyrazol-5-yl]-3-hydroxy-2-methyl-2-(pyridin-2-yl)propan-1-one or (2S)-1-[2-(2,3-dihydro-1,4-benzodioxine-6-sulfonyl)-2H,4H,5H,6H-pyrrolo[3,4-c]pyrazol-5-yl]-3-hydroxy-2-methyl-2-(pyridin-2-yl)propan-1-one (Compound 48)

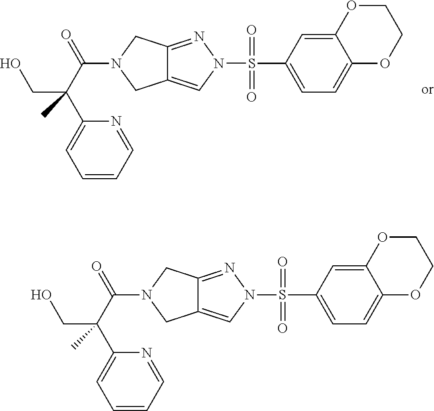 [SMILES: C[C@@](CO)(C(=O)N1Cc2cn(S(=O)(=O)c3ccc4c(c3)OCCO4)nc2C1)c1ccccn1 ; C[C@](CO)(C(=O)N1Cc2cn(S(=O)(=O)c3ccc4c(c3)OCCO4)nc2C1)c1ccccn1]

The title compound was made from 2-((2,3-dihydrobenzo[b][1,4]dioxin-6-yl)sulfonyl)-2,4,5,6-tetrahydropyrrolo[3,4-c]pyrazole (Intermediate I-4) and sodium 2-methyl-2-(pyridin-2-yl)-3-[(triisopropylsilyl)oxy]propanoate (synthesized as described for (2R)-1-[2-(1,3-benzothiazole-6-sulfonyl)-2H,4H,5H,6H-pyrrolo[3,4-c]pyrazol-5-yl]-3-hydroxy-2-methyl-2-(pyridin-2-yl)propan-1-one or (2S)-1-[2-(1,3-benzothiazole-6-sulfonyl)-2H,4H,5H,6H-pyrrolo[3,4-c]pyrazol-5-yl]-3-hydroxy-2-methyl-2-(pyridin-2-yl)propan-1-one (Compound 43) following a procedure similar to the one described for the same compound. The racemic material was separated by Chiral Prep-HPLC with the following conditions: Column: CHIRALPAK IA, 20*250 mm, 5 um; Mobile phase: A: n-Hexane, B: Ethanol; Flow rate: 90 mL/min; Gradient: 50% B in 10 min, 220 nm. This resulted in 92.4 mg of (2R)-1-[2-(2,3-dihydro-1,4-benzodioxine-6-sulfonyl)-2H,4H,5H,6H-pyrrolo[3,4-c]pyrazol-5-yl]-3-hydroxy-2-methyl-2-(pyridin-2-yl)propan-1-one or (2S)-1-[2-(2,3-dihydro-1,4-benzodioxine-6-sulfonyl)-2H,4H,5H,6H-pyrrolo[3,4-c]pyrazol-5-yl]-3-hydroxy-2-methyl-2-(pyridin-2-yl)propan-1-one (RT=1.26 min) as a white solid. ¹H NMR (300 MHz, DMSO-d₆) δ 8.52 (d, J=4.7 Hz, 1H), 8.18 (s, 1H), 8.04 (s, 0H), 7.79 (td, J=7.7, 1.9 Hz, 1H), 7.45-7.32 (m, 3H), 7.27 (ddd, J=7.4, 4.8, 1.1 Hz, 1H), 7.08 (d, J=8.6 Hz, 1H), 4.67 (t, J=5.8 Hz, 1H), 4.44 (d, J=7.9 Hz, 2H), 4.34 (d, J=5.4 Hz, 2H), 4.30 (d, J=5.5 Hz, 2H), 4.01-3.73 (m, 3H), 3.53-3.35 (m, 1H), 1.55 (s, 3H). LCMS (ES, m/z): [M+H]⁺: 471.

### Example 1.95

### Synthesis of (2R)-2-amino-1-{2-[4-(difluoromethoxy)benzenesulfonyl]-2H,4H,5H,6H-pyrrolo[3,4-c]pyrazol-5-yl}-2-(4-fluorophenyl)ethan-1-one (Compound 49)

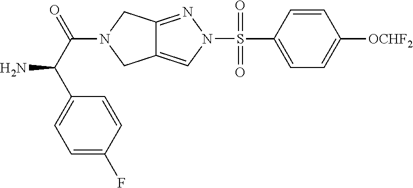 [SMILES: N[C@@H](C(=O)N1Cc2cn(S(=O)(=O)c3ccc(OC(F)F)cc3)nc2C1)c1ccc(F)cc1]

The title compound was made from 2-[4-(difluoromethoxy)benzenesulfonyl]-4H,5H,6H-pyrrolo[3,4c]pyrazole (Intermediate I-2) and (R)-[(tert-butoxycarbonyl)amino](4-fluorophenyl)acetic acid following a procedure similar to the one described for (2R)-2-amino-1-{2-[4-(difluoromethoxy)benzenesulfonyl]-2H,4H,5H,6H-pyrrolo[3,4-c]pyrazol-5-yl}-2-(3-fluorophenyl)ethan-1-one (Compound 29; steps 2-3). The crude material was purified by Flash-Prep-HPLC with the following conditions (IntelFlash-1): Column, C18; mobile phase, Mobile phase: MeCN=5/1B: Water Flow rate: 20 mL/min Column: DAICEL CHIRALPAK IC, 250*20 mm, 220 nm Gradient: 50% B in 20 min; 220 nm. This resulted in (2R)-2-amino-1-{2-[4-(difluoromethoxy)benzenesulfonyl]-2H,4H,5H,6H-pyrrolo[3,4-c]pyrazol-5-yl}-2-(4-fluorophenyl)ethan-1-one (formate salt) (150 mg, 52.06%) as a white solid. ¹H-NMR: (300 MHz, DMSO-d6, ppm): δ 8.27 (s, 1H), 8.20 (s, 1H), 8.09-8.01 (m, 2H), 7.68-7.13 (m, 7H), 4.93-4.76 (m, 2H), 4.62-4.38 (m, 2H), 4.26-4.16 (m, 1H). LCMS (ES) [M+1]⁺ m/z: 467.1.

### Example 1.96

### Synthesis of (2R)-2-amino-2-(3-chlorophenyl)-1-{2-[4-(difluoromethoxy)benzenesulfonyl]-2H,4H,5H,6H-pyrrolo[3,4-c]pyrazol-5-yl}ethan-1-one (Compound 50)

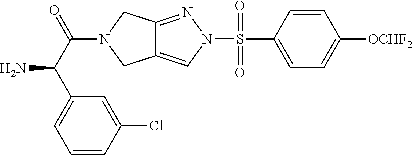 [SMILES: N[C@@H](C(=O)N1Cc2cn(S(=O)(=O)c3ccc(OC(F)F)cc3)nc2C1)c1cccc(Cl)c1]

The title compound was made from 2-[4-(difluoromethoxy)benzenesulfonyl]-4H,5H,6H-pyrrolo[3,4c]pyrazole (Intermediate I-2) and (R)-[(tert-butoxycarbonyl)amino](3-chlorophenyl)acetic acid following a procedure similar to the one described for (2R)-2-amino-1-{2-[4-(difluoromethoxy)benzenesulfonyl]-2H,4H,5H,6H-pyrrolo[3,4-c]pyrazol-5-yl}-2-(3-fluorophenyl)ethan-1-one (Compound 29; steps 2-3). The crude product was purified by Prep-HPLC with the following conditions: Column, Kinetex EVO C18 Column, 21.2*150, 5 um, Mobile phase, Water (0.1% FA) and CH₃CN (10% Phase B up to 50% in 15 min), Detector, UV 254 nm to provide 136.5 mg (55%) of (2R)-2-amino-2-(3-chlorophenyl)-1-{2-[4-(difluoromethoxy)benzenesulfonyl]-2H,4H,5H,6H-pyrrolo[3,4-c]pyrazol-5-yl}ethan-1-one (formate salt) as a white solid. ¹H NMR (300 MHz, DMSO-d₆) δ 8.28 (d, J=0.9 Hz, 1H), 8.17 (s, 1H), 8.13-7.99 (m, 2H), 7.72-7.17 (m, 7H), 5.03-4.81 (m, 2H), 4.59-4.39 (m, 2H), 4.38-4.12 (m, 1H). LCMS(ES)[M−HCOOH+1]⁺ m/z: 483.

### Example 1.97

### Synthesis of (2R)-2-amino-2-(4-chlorophenyl)-1-{2-[4-(difluoromethoxy)benzenesulfonyl]-2H,4H,5H,6H-pyrrolo[3,4-c]pyrazol-5-yl}ethan-1-one (Compound 51)

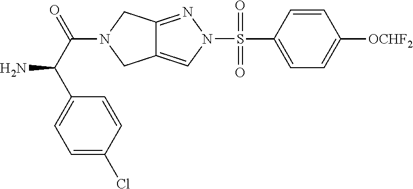 [SMILES: N[C@@H](C(=O)N1Cc2cn(S(=O)(=O)c3ccc(OC(F)F)cc3)nc2C1)c1ccc(Cl)cc1]

The title compound was made from 2-[4-(difluoromethoxy)benzenesulfonyl]-4H,5H,6H-pyrrolo[3,4c] pyrazole (Intermediate I-2) and (R)-[(tert-butoxycarbonyl)amino](4-chlorophenyl)acetic acid following a procedure similar to the one described for (2R)-2-amino-1-{2-[4-(difluoromethoxy)benzenesulfonyl]-2H,4H,5H,6H-pyrrolo[3,4-c]pyrazol-5-yl}-2-(3-fluorophenyl)ethan-1-one (Compound 29; steps 2-3). The crude material was purified by Flash-Prep-HPLC with the following conditions (IntelFlash-1): Column, C18; mobile phase, Mobile phase: MeCN=5/1B: Water Flow rate: 20 mL/min Column: DAICEL CHIRALPAK IC, 250*20 mm, 220 nm Gradient: 50% B in 20 min; 220 nm; This resulted in (2R)-2-amino-2-(4-chlorophenyl)-1-{2-[4-(difluoromethoxy)benzenesulfonyl]-2H,4H,5H,6H-pyrrolo[3,4-c]pyrazol-5-yl}ethan-1-one (formate salt) (160 mg, 55.2%) as a white solid. ¹H-NMR: (300 MHz, DMSO-d6, ppm): δ 8.27 (s, 1H), 8.20 (s, 1H), 8.10-7.99 (m, 2H), 7.67-7.20 (m, 7H), 6.48 (br, 2H), 5.00-4.88 (m, 2H), 4.56-4.35 (m, 2H), 4.21-4.10 (m, 1H). LCMS (ES) [M−56+1]⁺ m/z: 483.0.

### Example 1.98

### Synthesis of (2R)-2-amino-1-[2-(1,3-benzothiazole-6-sulfonyl)-2H,4H,5H,6H-pyrrolo[3,4-c]pyrazol-5-yl]-2-(3-chlorophenyl)ethan-1-one (Compound 53)

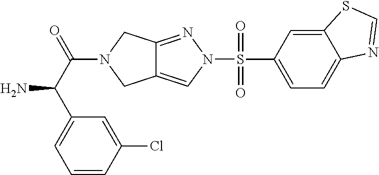 [SMILES: N[C@@H](C(=O)N1Cc2cn(S(=O)(=O)c3ccc4ncsc4c3)nc2C1)c1cccc(Cl)c1]

The title compound was made from 6-{2H,4H,5H,6H-pyrrolo[3,4-c]pyrazole-2-sulfonyl}-1,3-benzothiazole (Intermediate I-1) and (2R)-2-{[(tert-butoxy)carbonyl]amino}-2-(3-chlorophenyl)acetic acid following a 2-step procedure similar to that for the synthesis of (2R)-2-amino-1-[2-(1,3-benzothiazole-6-sulfonyl)-2H,4H,5H,6H-pyrrolo[3,4-c]pyrazol-5-yl]-2-phenylethan-1-one (Compound 15). The crude product was purified by reverse phase preparative HPLC (Prep-C18, 5 μM XBridge column, 19×150 mm, Waters; gradient elution of 0-80% MeCN in water over a 20 min period, where both solvents contained 0.1% formic acid) to provide the title product as a white solid. ¹H NMR (400 MHz, DMSO-d6) δ 9.69 (s, 1H), 9.01 (d, J=2.0 Hz, 1H), 8.36-8.24 (m, 2H), 8.03 (dt, J=8.7, 1.7 Hz, 1H), 7.42 (d, J=1.9 Hz, 1H), 7.29 (qq, J=4.7, 2.6, 2.1 Hz, 3H), 4.86 (dd, J=20.7, 14.3 Hz, 1H), 4.68 (d, J=15.7 Hz, 1H), 4.49-4.18 (m, 3H). LCMS (ES) [M+1]+ m/z 474.6.

### Example 1.99

### Synthesis of 6-({5-[(2S)-2-phenylpyrrolidine-2-carbonyl]-2H,4H,5H,6H-pyrrolo[3,4-c]pyrazol-2-yl}sulfonyl)-1,3-benzothiazole or 6-({2-[(2R)-2-phenylpyrrolidine-2-carbonyl]-2H,4H,5H,6H-pyrrolo[3,4-c]pyrazol-2-yl}sulfonyl)-1,3-benzothiazole (Compound 54)

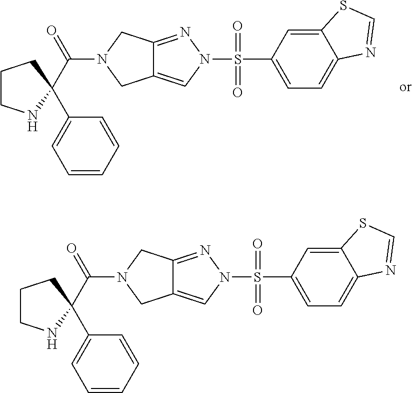 [SMILES: O=C(N1Cc2cn(S(=O)(=O)c3ccc4ncsc4c3)nc2C1)[C@@]1(c2ccccc2)CCCN1 ; O=C(N1Cc2cn(S(=O)(=O)c3ccc4ncsc4c3)nc2C1)[C@@]1(c2ccccc2)CCCN1]

The title compound was made from 6-{2H,4H,5H,6H-pyrrolo[3,4-c]pyrazole-2-sulfonyl}-1,3-benzothiazole (Intermediate I-1) and 2-phenylpyrrolidine-2-carboxylic acid following a procedure similar to the one described for the synthesis of Compound 1. The crude material was purified by Prep-HPLC with the following conditions: Column, C18-120 g, Mobile phase, Phase A: H₂O (0.05% FA), Phase B: CH₃CN, from 5% to 70% within 12 min, Flow rate, 70 mL/min, Detector, UV 254 nm. The racemic material was separated by Chiral-Prep-HPLC with the following conditions: Column, CHIRALPAK-IA, 20*250 mm*5 um, Mobile phase, Hexane/DCM=3/1 (0.1% DEA) and EtOH (50% in 7 min), Detector, UV 254 nm to provide 77.7 mg (7.8%) of 6-({5[(2S)-2-phenylpyrrolidine-2-carbonyl]-2H,4H,5H,6H-pyrrolo[3,4-c]pyrazol-2-yl}sulfonyl)-1,3-benzothiazole or 6-({5-[(2R)-2-phenylpyrrolidine-2-carbonyl]-2H,4H,5H,6H-pyrrolo[3,4-c]pyrazol-2-yl}sulfonyl)-1,3-benzothiazole (RT=4.29 min) as a white solid. ¹H-NMR (300 MHz, DMSO-d₆, ppm): 9.71(s, 1H), 9.00-8.98(m, 1H), 8.29 (dd, J=8.7, 2.5 Hz, 1H), 8.25(s, 0.5H), 8.17(s, 0.5H), 8.01 (dt, J=8.7, 1.7 Hz, 2H), 7.35-7.22(m, 5H), 4.70-4.65(m, 1H), 4.43(d, J=3.6 Hz, 2H), 3.78-3.63(m, 1H), 3.03-2.95(m, 1H), 2.93-2.86(m, 1H), 2.77-2.73(m, 1H), 1.72-1.65(m, 3H). LCMS (ES, m/z): [M+H]⁺: 480.

### Example 1.100

### Synthesis of 6-({5-[(2S)-2-phenylpyrrolidine-2-carbonyl]-2H,4H,5H,6H-pyrrolo[3,4-c]pyrazol-2-yl}sulfonyl)-1,3-benzothiazole or 6-({5-[(2R)-2-phenylpyrrolidine-2-carbonyl]-2H,4H,5H,6H-pyrrolo[3,4-c]pyrazol-2-yl}sulfonyl)-1,3-benzothiazole (Compound 61)

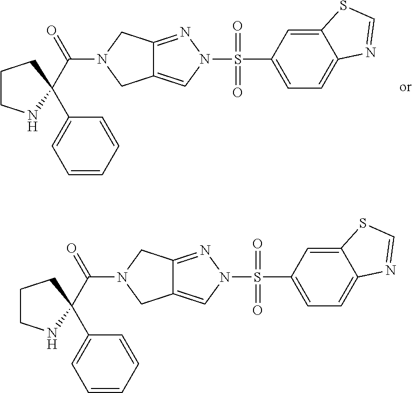 [SMILES: O=C(N1Cc2cn(S(=O)(=O)c3ccc4ncsc4c3)nc2C1)[C@@]1(c2ccccc2)CCCN1 ; O=C(N1Cc2cn(S(=O)(=O)c3ccc4ncsc4c3)nc2C1)[C@@]1(c2ccccc2)CCCN1]

The title compound was synthesized by the procedure described for 6-({5-[(2S)-2-phenylpyrrolidine-2-carbonyl]-2H,4H,5H,6H-pyrrolo[3,4-c]pyrazol-2-yl}sulfonyl)-1,3-benzothiazole or 6-({5-[(2R)-2-phenylpyrrolidine-2-carbonyl]-2H,4H,5H,6H-pyrrolo[3,4-c]pyrazol-2-yl}sulfonyl)-1,3-benzothiazole (Compound 54) 95 mg of material were obtained by the conditions described therein (RT=5.79 min). This material was further purified by Prep-HPLC with the following conditions: Column, Atlantis HILIC OBD Column, 19*150 mm*5 um, Mobile phase, Water (0.1% FA) and CH₃CN (40% Phase B up to 60% in 8 min), Detector, UV 254 nm to provide 74.3 mg (7.7%) of 6-({5-[(2S)-2-phenylpyrrolidine-2-carbonyl]-2H,4H,5H,6H-pyrrolo[3,4-c]pyrazol-2-yl}sulfonyl)-1,3-benzothiazole or 6-({5-[(2R)-2-phenylpyrrolidine-2-carbonyl]-2H,4H,5H,6H-pyrrolo[3,4-c]pyrazol-2-yl}sulfonyl)-1,3-benzothiazole (formate salt) as a white solid. ¹H-NMR (300 MHz, DMSO-d₆, ppm): 9.71(s, 1H), 9.00-8.98(m, 1H), 8.29 (dd, J=8.7, 2.7 Hz, 1H), 8.25 (s, 0.5H), 8.17 (s, 1.5H), 8.01 (dt, J=8.7, 1.9 Hz, 1H), 7.35-7.22(m, 5H), 4.70-4.65(m, 1H), 4.43(d, J=3.6 Hz, 2H), 3.80-3.72 (m, 1H), 3.03-2.95(m, 1H), 2.93-2.86(m, 1H), 2.77-2.73(m, 1H), 1.72-1.65(m, 3H). LCMS (ES, m/z): [M+H]⁺: 480.

### Example 1.101

### Synthesis of (2R)-2-amino-1-{2-[4-(difluoromethoxy)benzenesulfonyl]-2H,4H,5H,6H-pyrrolo[3,4-c]pyrazol-5-yl}-2-(2-fluorophenyl)ethan-1-one (Compound 56)

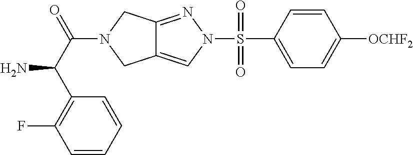 [SMILES: N[C@@H](C(=O)N1Cc2cn(S(=O)(=O)c3ccc(OC(F)F)cc3)nc2C1)c1ccccc1F]

The title compound was made from 2-[4-(difluoromethoxy)benzenesulfonyl]-2H,4H,5H,6H-pyrrolo[3,4c] pyrazole (Intermediate I-2) and ((R)-amino(2-fluorophenyl)acetic acid following a 3-step procedure similar to the one described for (2R)-2-amino-1-{2-[4-(difluoromethoxy)benzenesulfonyl]-2H,4H,5H,6H-pyrrolo[3,4-c]pyrazol-5-yl}-2-(3-fluorophenyl)ethan-1-one (Compound 29). The crude material was purified by Flash-Prep-HPLC with the following conditions (IntelFlash-1): Column, C18 silica gel; mobile phase, ACN:H₂O(FA 0.05%)=15% increasing to ACN:H₂O(FA 0.05%)=22%. This resulted in 113 mg (49.02%) of (2R)-2-amino-1-{2-[4-(difluoromethoxy)benzenesulfonyl]-2H,4H,5H,6H-pyrrolo[3,4-c]pyrazol-5-yl}-2-(2-fluorophenyl)ethan-1-one as an off-white solid. ¹H NMR (300 MHz, DMSO-d₆) δ 8.27 (d, J=4.8 Hz, 1H), 8.04 (dd, J=8.9, 1.7 Hz, 2H), 7.52-7.10 (m, 7H), 5.00-4.83 (m, 2H), 4.51-4.32 (m, 2H), 4.21-4.09 (m, 1H). LCMS (ES) [M+1]⁺ m/z: 467.1.

### Example 1.102

### Synthesis of (2R)-2-amino-1-[2-(3,4-dihydro-2H-1,4-benzoxazine-6-sulfonyl)-2H,4H,5H,6H-pyrrolo[3,4-c]pyrazol-5-yl]-2-(2-fluorophenyl)ethan-1-one (Compound 57)

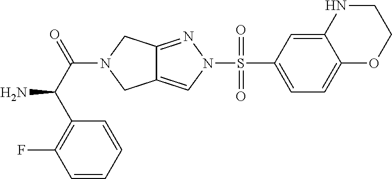 [SMILES: N[C@@H](C(=O)N1Cc2cn(S(=O)(=O)c3ccc4c(c3)NCCO4)nc2C1)c1ccccc1F]

The title compound was made from 6-[4H,5H,6H-pyrrolo[3,4-c]pyrazole-2-sulfonyl]-3,4-dihydro-2H-1,4-benzoxazine (Intermediate I-3) and (R)-[(tert-butoxycarbonyl)amino](2-fluorophenyl)acetic acid following a 2-step procedure similar to the one described for (2R)-2-amino-1-{2-[4-(difluoromethoxy)benzenesulfonyl]-2H,4H,5H,6H-pyrrolo[3,4-c]pyrazol-5-yl}-2-(3-fluorophenyl)ethan-1-one (Compound 29; steps 2-3). The crude material was purified by Flash-Prep-HPLC with the following conditions (IntelFlash-1): Column, C18 silica gel; mobile phase, ACN:H₂O(FA 0.05%)=15% increasing to ACN:H₂O(FA 0.05%)=25%. This resulted in 62.9 mg (30.67%) of (2R)-2-amino-1-[2-(3,4-dihydro-2H-1,4-benzoxazine-6-sulfonyl)-2H,4H,5H,6H-pyrrolo[3,4-c]pyrazol-5-yl]-2-(2-fluorophenyl)ethan-1-one as a white solid. ¹H NMR (300 MHz, DMSO-d₆) δ 8.15 (d, J=2.4 Hz, 1H), 7.42 (dtd, J=9.7, 7.8, 2.0 Hz, 1H), 7.39-7.25 (m, 1H), 7.24-7.10 (m, 3H), 7.04 (dt, J=8.5, 2.3 Hz, 1H), 6.83 (d, J=8.5 Hz, 1H), 6.46 (s, 1H), 5.02-4.80 (m, 2H), 4.56-4.30 (m, 2H), 4.28-4.10 (m, 3H), 3.40-3.31 (m, 2H). LCMS (ES) [M+1]⁺ m/z: 458.2.

### Example 1.103

### Synthesis of (2R)-2-amino-1-[2-(2,3-dihydro-1,4-benzodioxine-6-sulfonyl)-2H,4H,5H,6H-pyrrolo[3,4-c]pyrazol-5-yl]-2-(2-fluorophenyl)ethan-1-one (Compound 58)

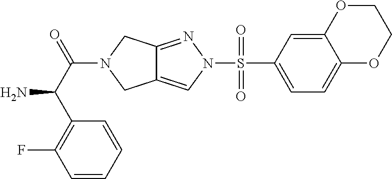 [SMILES: N[C@@H](C(=O)N1Cc2cn(S(=O)(=O)c3ccc4c(c3)OCCO4)nc2C1)c1ccccc1F]

The title compound was made from 2-((2,3-dihydrobenzo[b][1,4]dioxin-6-yl)sulfonyl)-2,4,5,6-tetrahydropyrrolo[3,4-c]pyrazole (Intermediate I-4) and (R)-[(tert-butoxycarbonyl)amino](2-fluorophenyl)acetic acid following a 2-step procedure similar to the one described for (2R)-2-amino-1-{2-[4-(difluoromethoxy)benzenesulfonyl]-2H,4H,5H,6H-pyrrolo[3,4-c]pyrazol-5-yl}-2-(3-fluorophenyl)ethan-1-one (Compound 29; steps 2-3). The crude material was purified by Flash-Prep-HPLC with the following conditions (IntelFlash-1): Column, C18; mobile phase, Mobile phase: MeCN=5/1B: Water Flow rate: 20 mL/min Column: DAICEL CHIRALPAK IC, 250*20 mm, 220 nm Gradient: 50% B in 20 min; 220 nm. This resulted in (2R)-2-amino-1-[2-(2,3-dihydro-1,4-benzodioxine-6-sulfonyl)-2H,4H,5H,6H-pyrrolo[3,4-c]pyrazol-5-yl]-2-(2-fluorophenyl)ethan-1-one (130.1 mg, 52.8%) as a white solid. ¹H NMR (300 MHz, DMSO-d₆) δ 8.23 (dd, J=7.3, 2.8 Hz, 1H), 7.51-7.33 (m, 4H), 7.24 (tt, J=9.8, 4.6 Hz, 2H), 7.10 (dd, J=8.7, 2.9 Hz, 1H), 5.13 (dd, J=13.7, 3.0 Hz, 1H), 4.90 (dd, J=23.3, 14.8 Hz, 1H), 4.49 (ddd, J=22.0, 15.9, 11.9 Hz, 2H), 4.34 (d, J=5.2 Hz, 2H), 4.30 (d, J=5.6 Hz, 2H), 4.07 (td, J=14.4, 13.9, 3.0 Hz, 1H). LCMS (ES) [M+1]⁺ m/z: 459.1.

### Example 1.104

### Synthesis of (2R)-2-amino-1-[2-(1,3-benzothiazole-6-sulfonyl)-2H,4H,5H,6H-pyrrolo[3,4-c]pyrazol-5-yl]-2-(2-methylphenyl)ethan-1-one (Compound 59)

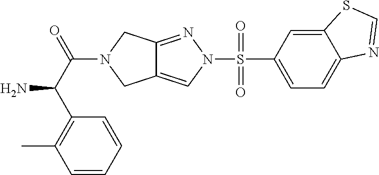 [SMILES: Cc1ccccc1[C@@H](N)C(=O)N1Cc2cn(S(=O)(=O)c3ccc4ncsc4c3)nc2C1]

The title compound was made from 6-[4H,5H,6H-pyrrolo[3,4-c]pyrazole-2-sulfonyl]-1,3-benzothiazole hydrochloride (Intermediate I-1) and (R)-amino (2-methylphenyl)acetic acid following a 3-step procedure similar to the one described for (2R)-2-amino-1-{2-[4-(difluoromethoxy)benzenesulfonyl]-2H,4H,5H,6H-pyrrolo[3,4-c]pyrazol-5-yl}-2-(3-fluorophenyl)ethan-1-one (Compound 29). The crude material was purified by Prep-HPLC with the following conditions: Column, Kinetex EVO C18 Column, 21.2*150, 5 um, Mobile phase, Water (0.1% FA) and CH₃CN (10% Phase B up to 50% in 15 min); Detector, UV 254 nm to provide 152.7 mg (53%) of (2R)-2-amino-1-[2-(1,3-benzothiazole-6-sulfonyl)-2H,4H,5H,6H-pyrrolo[3,4-c]pyrazol-5-yl]-2-(2-methylphenyl)ethan-1-one (formate salt) as a white solid. ¹H NMR (300 MHz, DMSO-d₆) δ 9.71 (d, J=1.5 Hz, 1H), 9.02 (dd, J=3.6, 2.1 Hz, 1H), 8.32-8.21 (m, 3H), 8.09-7.98 (m, 1H), 7.29-7.09 (m, 4H), 5.50 (br, 2H), 5.02-4.83 (m, 1H), 4.82-4.72 (m, 1H), 4.58-4.36 (m, 2H), 3.88-3.53 (m, 1H), 2.42 (d, J=9.6 Hz, 3H). LCMS (ES)[M−HCOOH+1]⁺ m/z: 454.

### Example 1.105

### Synthesis of (2R)-2-amino-1-[2-(1,3-benzothiazole-6-sulfonyl)-2H,4H,5H,6H-pyrrolo[3,4-c]pyrazol-5-yl]-2-phenylpropan-1-one (Compound 60)

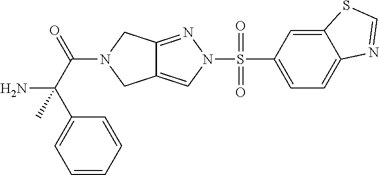 [SMILES: C[C@](N)(C(=O)N1Cc2cn(S(=O)(=O)c3ccc4ncsc4c3)nc2C1)c1ccccc1]

The title compound was made from 6-[4H,5H,6H-pyrrolo[3,4-c]pyrazole-2-sulfonyl]-1,3-benzothiazole hydrochloride (Intermediate I-1) and (2R)-2-amino-2-phenylpropanoic acid following a 3-step procedure similar to the one described for (2R)-2-amino-1-{2-[4-(difluoromethoxy)benzenesulfonyl]-2H,4H,5H,6H-pyrrolo[3,4-c]pyrazol-5-yl}-2-(3-fluorophenyl)ethan-1-one (Compound 29). The crude product was purified by Flash-Prep-HPLC with the following conditions (IntelFlash-1): Column, C18 silica gel; mobile phase, ACN:H₂O(NH₃ 0.05%)=20% increasing to ACN:H₂O(NH₃ 0.05%)=35%. This resulted in 120 mg (48.83%) of (2R)-2-amino-1-[2-(1,3-benzothiazole-6-sulfonyl)-2H,4H,5H,6H-pyrrolo[3,4-c]pyrazol-5yl]-2-phenylpropan-1-one (formate salt) as an off-white solid. ¹H NMR (300 MHz, DMSO-d₆) δ 9.66 (s, 1H), 8.99-8.88 (m, 1H), 8.33-8.07 (m, 3H), 8.05-7.96 (m, 1H), 7.39 (s, 5H), 4.54-4.40 (m, 2H), 3.92-3.84 (m, 2H), 1.67 (s, 3H). LCMS (ES) [M+1]⁺ m/z: 454.

### Example 1.106

### Synthesis of (2R)-1-{2-[4-(difluoromethoxy)benzenesulfonyl]-2H,4H,5H,6H-pyrrolo[3,4-c]pyrazol-5-yl}-2-(morpholin-4-yl)-2-phenylethan-1-one or (2S)-1-{2-[4-(difluoromethoxy)benzenesulfonyl]-2H,4H,5H,6H-pyrrolo[3,4-c]pyrazol-5-yl}-2-(morpholin-4-yl)-2-phenylethan-1-one (Compound 107)

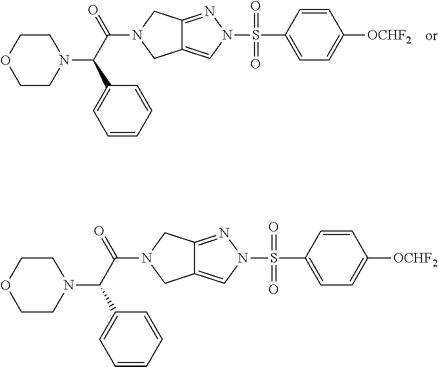 [SMILES: O=C([C@@H](c1ccccc1)N1CCOCC1)N1Cc2cn(S(=O)(=O)c3ccc(OC(F)F)cc3)nc2C1 ; O=C([C@H](c1ccccc1)N1CCOCC1)N1Cc2cn(S(=O)(=O)c3ccc(OC(F)F)cc3)nc2C1]

The title compound was made from 2-[4-(difluoromethoxy)benzenesulfonyl]-2H,4H,5H,6H-pyrrolo[3,4-c]pyrazole (Intermediate I-2) and 4-morpholinyl(phenyl)acetic acid following a procedure similar as described for the synthesis of Compound 1. The crude material was purified using reverse phase preparative HPLC (Prep-C18, 5 uM Luna C18 column, 22×250 mm, Phenomenex; gradient elution of 10-70% MeCN in water over a 20 min period, where both solvents contain 0.1% formic acid) to afford a racemic product.

Chiral separation was performed using Phenomenex column (Amylose 2, 21.2×250 mm using isocratic 20% EtOH (0.1% diethylamine) in hexanes (0.1% diethylamine) with flow rate=25 mL/min to afford (2R)-1-{2-[4-(difluoromethoxy)benzenesulfonyl]-2H,4H,5H,6H-pyrrolo[3,4-c]pyrazol-5-yl}-2-(morpholin-4-yl)-2-phenylethan-1-one or (2S)-1-{2-[4-(difluoromethoxy)benzenesulfonyl]-2H,4H,5H,6H-pyrrolo[3,4-c]pyrazol-5-yl}-2-(morpholin-4-yl)-2-phenylethan-1-one (RT=27 min) (30 mg; 11%) ¹H NMR (400 MHz, dmso) δ 8.28-8.18 (m, 1H), 8.03-7.96 (m, 2H), 7.42 (q, J=3.7 Hz, 2H), 7.39-7.36 (m, 2H), 7.34-7.23 (m, 3H), 4.90 (dd, J=25.1, 14.4 Hz, 1H), 4.47-4.26 (m, 5H), 3.49 (t, J=4.6 Hz, 4H), 2.38 (s, 4H). LCMS (ES) [M+1]⁺ m/z 519.1.

### Example 1.107

### Synthesis of (2R)-1-{2-[4-(difluoromethoxy)benzenesulfonyl]-2H,4H,5H,6H-pyrrolo[3,4-c]pyrazol-5-yl}-2-(morpholin-4-yl)-2-phenylethan-1-one or (2S)-1-{2-[4-(difluoromethoxy)benzenesulfonyl]-2H,4H,5H,6H-pyrrolo[3,4-c]pyrazol-5-yl}-2-(morpholin-4-yl)-2-phenylethan-1-one (Compound 108)

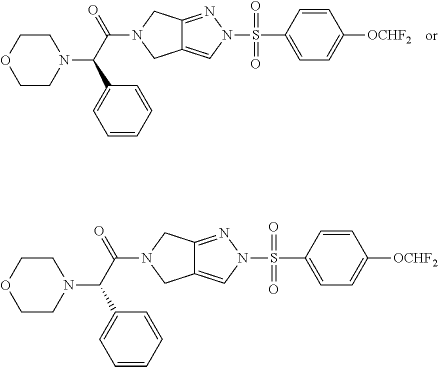 [SMILES: O=C([C@@H](c1ccccc1)N1CCOCC1)N1Cc2cn(S(=O)(=O)c3ccc(OC(F)F)cc3)nc2C1 ; O=C([C@H](c1ccccc1)N1CCOCC1)N1Cc2cn(S(=O)(=O)c3ccc(OC(F)F)cc3)nc2C1]

The title compound was synthesized by the procedure described for (2R)-1-{2-[4-(difluoromethoxy)benzenesulfonyl]-2H,4H,5H,6H-pyrrolo[3,4-c]pyrazol-5-yl}-2-(morpholin-4-yl)-2-phenylethan-1-one or (2S)-1-{2-[4-(difluoromethoxy)benzenesulfonyl]-2H,4H,5H,6H-pyrrolo[3,4-c]pyrazol-5-yl}-2-(morpholin-4-yl)-2-phenylethan-1-one. Chiral purification by the conditions described therein provided (2R)-1-{2-[4-(difluoromethoxy)benzenesulfonyl]-2H,4H,5H,6H-pyrrolo[3,4-c]pyrazol-5-yl}-2-(morpholin-4-yl)-2-phenylethan-1-one or (2S)-1-{2-[4-(difluoromethoxy)benzenesulfonyl]-2H,4H,5H,6H-pyrrolo[3,4-c]pyrazol-5-yl}-2-(morpholin-4-yl)-2-phenylethan-1-one (RT=33 min) (28 mg; 10%). ¹H NMR (400 MHz, dmso) δ 8.28-8.18 (m, 1H), 8.03-7.96 (m, 2H), 7.42 (q, J=3.7 Hz, 2H), 7.39-7.36 (m, 2H), 7.34-7.23 (m, 3H), 4.90 (dd, J=25.1, 14.4 Hz, 1H), 4.47-4.26 (m, 5H), 3.49 (t, J=4.6 Hz, 4H), 2.38 (s, 4H). LCMS (ES) [M+1]⁺ m/z 519.2.

### Example 1.108

### Synthesis of (2S)-3-hydroxy-1-{2-[(1-methyl-1H-indazol-6-yl)sulfonyl]-2H,4H,5H,6H-pyrrolo[3,4-c]pyrazol-5-yl}-2-phenylpropan-1-one (Compound 90)

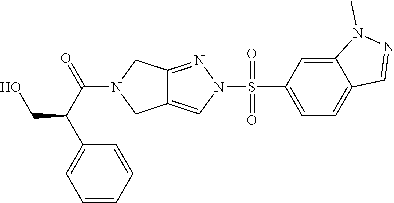 [SMILES: Cn1ncc2ccc(S(=O)(=O)n3cc4c(n3)CN(C(=O)[C@H](CO)c3ccccc3)C4)cc21]

The title compound was made from crude 1-methyl-6-{2H,4H,5H,6H-pyrrolo[3,4-c]pyrazole-2-sulfonyl}-1H-indazole (synthesized following a procedure similar as described for the synthesis of Intermediate I-1, except that 1-methyl-1H-indazole-6-sulfonyl fluoride was used in the place of 1,3 benzothiazole-6-sulfonyl chloride) and (2S)-3-hydroxy-2-phenylpropanoic acid (Intermediate II-6) following a procedure similar to that described for the synthesis of Compound 1. The crude product was purified by reverse phase preparative HPLC (Prep-C18, 5 μM XBridge column, 19×150 mm, Waters; gradient elution of 0-80% MeCN in water over a 20 min period, where both solvents contain 0.1% formic acid) to provide the title product as a white solid. ¹H NMR (400 MHz, DMSO-d6) δ 8.42 (dt, J=1.7, 0.9 Hz, 1H), 8.26 (d, J=1.1 Hz, 1H), 8.23-8.21 (m, 1H), 7.97 (dt, J=8.6, 0.7 Hz, 1H), 7.54 (dd, J=8.6, 1.6 Hz, 1H), 7.31-7.14 (m, 5H), 4.87-4.70 (m, 2H), 4.45-4.22 (m, 3H), 4.13 (s, 3H), 3.96-3.88 (m, 2H), 3.52-3.44 (m, 1H). LCMS (ES) [M+1]+ m/z 452.2.

### Example 1.109

### Synthesis of 1-[(1R)-2-{2-[4-(difluoromethoxy)benzenesulfonyl]-2H,4H,5H,6H-pyrrolo[3,4-c]pyrazol-5-yl}-2-oxo-1-phenylethyl]azetidin-3-one or 1-[(1S)-2-{2-[4-(difluoromethoxy)benzenesulfonyl]-2H,4H,5H,6H-pyrrolo[3,4-c]pyrazol-5-yl}-2-oxo-1-phenylethyl]azetidin-3-one (Compound 92)

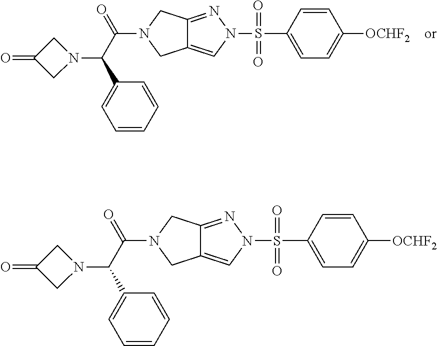 [SMILES: O=C1CN([C@@H](C(=O)N2Cc3cn(S(=O)(=O)c4ccc(OC(F)F)cc4)nc3C2)c2ccccc2)C1 ; O=C1CN([C@H](C(=O)N2Cc3cn(S(=O)(=O)c4ccc(OC(F)F)cc4)nc3C2)c2ccccc2)C1]

Step 1

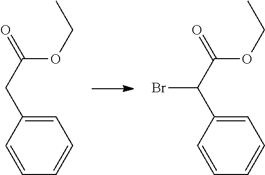

Into a 100-mL round-bottom flask, was placed ethyl 2-phenylacetate (6.00 g, 36.6 mmol, 1.00 eq.), CCl₄ (40.0 mL), NBS (7.80 g, 43.8 mmol, 1.20 eq.), and BPO (0.89 g, 3.66 mmol, 0.10 eq.) and the resulting solution was stirred for 16 hours at 80° C. The mixture was then cooled to RT, filtered and the filter cake was washed with EA (50 mL×3). The organic phase was concentrated under reduced pressure and the residue was applied onto a silica gel column with PE/EA ether (5/1). This resulted in 8.0 g (90.40%) of ethyl 2-bromo-2-phenylacetate as a yellow oil. No Mass signal could be detected.

Step 2

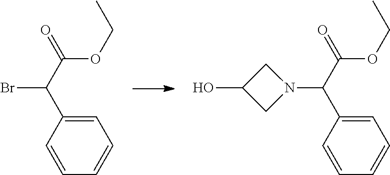 [SMILES: CCOC(=O)C(Br)c1ccccc1 ; CCOC(=O)C(c1ccccc1)N1CC(O)C1]

Into a 250-mL round-bottom flask, was placed ethyl 2-bromo-2-phenylacetate (8.00 g, 33.1 mmol, 1.20 eq.), DMF (100 mL), azetidin-3-ol (2.26 g, 31.0 mmol, 1.0 eq.), and Cs₂CO₃ (20.2 g, 62.0 mmol, 2.0 eq.) and the resulting solution was stirred for 2 hours at room temperature. The reaction was then quenched by the addition of 500 mL of water and the resulting solution was extracted with 3×500 mL of EA and washed with 1×500 mL of brine. The organic phase was dried over anhydrous sodium sulfate and concentrated and the residue was applied onto a silica gel column with PE/EA ether (3/1). This resulted in 3.7 g (50.89%) of ethyl 2-(3-hydroxyazetidin-1-yl)-2-phenylacetate1-oxide as a yellow solid. LCMS (ES) [M+1]⁺ m/z: 236.

Step 3

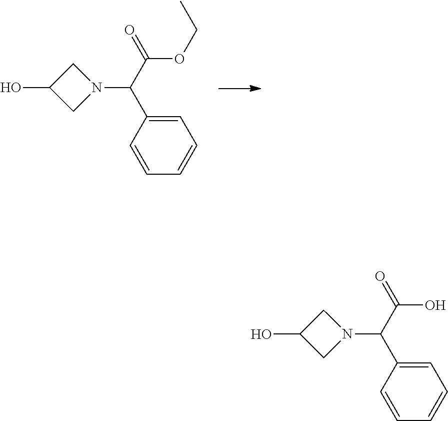 [SMILES: CCOC(=O)C(c1ccccc1)N1CC(O)C1 ; O=C(O)C(c1ccccc1)N1CC(O)C1]

Into a 100-mL round-bottom flask, was placed a mixture of ethyl 2-(3-hydroxyazetidin-1-yl)-2-phenylacetate1-oxide (2.0 g, 8.51 mmol, 1.00 eq.), THF (32.0 mL), H₂O (16.0 ml), and LiOH-H₂O (464.7 mg, 11.06 mmol, 1.3 eq.), and the resulting solution was stirred for 2 hours at room temperature. The pH value of the solution was then adjusted to 2˜3 with HCl(2N) and the resulting solution was extracted with 3×50 mL of DCM/MeOH (10/1). The organic phase was concentrated under reduced pressure providing 1.08 g (60.34%) of 2-(3-hydroxyazetidin-1-yl)-2-phenylacetic acid as a yellow solid. LCMS (ES) [M+1]⁺ m/z: 208.

Step 4

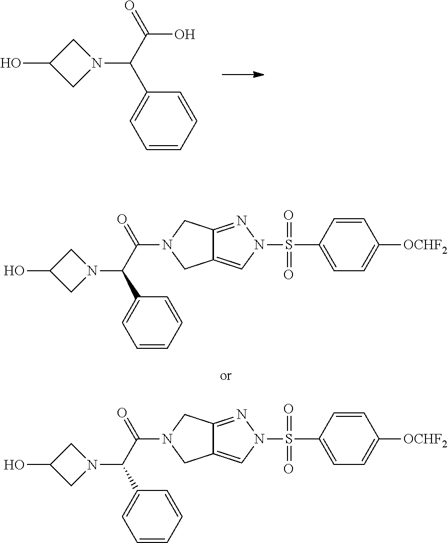 [SMILES: O=C(O)C(c1ccccc1)N1CC(O)C1 ; O=C([C@@H](c1ccccc1)N1CC(O)C1)N1Cc2cn(S(=O)(=O)c3ccc(OC(F)F)cc3)nc2C1 ; O=C([C@H](c1ccccc1)N1CC(O)C1)N1Cc2cn(S(=O)(=O)c3ccc(OC(F)F)cc3)nc2C1]

Into a 40-mL vial purged, was placed a mixture of 2-(3-hydroxyazetidin-1-yl)-2-phenylacetic acid (1.00 g, 4.76 mmol, 1.00 eq.), DMF (10 mL), 2-((4-(difluoromethoxy)phenyl)sulfonyl)-2,4,5,6-tetrahydropyrrolo[3,4-c]pyrazole (Intermediate I-2; 1.80 g, 5.71 mmol, 1.20 eq.), DIEA (1.23 g, 9.53 mmol, 2.0 eq.), and T₃P (50%) (1.82 g, 5.72 mmol, 1.2 eq.), and the resulting solution was stirred for 2 hours at room temperature. The crude product was purified by Prep-HPLC with the following conditions: SunFire Prep C18 OBD Column, 50*250 mm, 10 μm 10 nm; Mobile Phase A: Water (0.1% FA), Mobile Phase B: ACN; Flow rate: 90 mL/min; Gradient: 10% B to 45% B in 12 min, 45% B; Wave Length: 220 nm nm; RT1 (min): 12; Number Of Runs: 0. This resulted in 1.2 g (50.21%) of 1-(2-((4-(difluoromethoxy)phenyl)sulfonyl)-2,6-dihydropyrrolo[3,4-c]pyrazol-5(4H)-yl)-2-(3-hydroxyazetidin-1-yl)-2-phenylethan-1-one as a white solid.

The racemic material was purified by Prep-SFC with the following Column: CHIRAL ART Amylose-C NEO, 3*25 cm, 5 μm; Mobile Phase A: CO₂, Mobile Phase B: MeOH (0.1% 2M NH₃-MeOH); Flow rate: 85 mL/min; Gradient: isocratic 50% B; Column Temperature(° C.): 35; Back Pressure(bar): 100; Wave Length: 220 nm; RT₁ (2.45 min); RT₂ (4.39 min); Sample Solvent: MeOH-Preparative; Injection Volume: 5 mL; Number Of Runs: 7. This resulted in 600 mg (25.00%) of (R)-1-(2-((4-(difluoromethoxy)phenyl)sulfonyl)-2,6-dihydropyrrolo[3,4-c]pyrazol-5(4H)-yl)-2-(3-hydroxyazetidin-1-yl)-2-phenylethan-1-one or (S)-1-(2-((4-(difluoromethoxy)phenyl)sulfonyl)-2,6-dihydropyrrolo[3,4-c]pyrazol-5(4H)-yl)-2-(3-hydroxyazetidin-1-yl)-2-phenylethan-1-one as a white solid (ANAL-SFC) RT: 2.746 min and also 600 mg (25.00%) of (S)-1-(2-((4-(difluoromethoxy)phenyl)sulfonyl)-2,6-dihydropyrrolo[3,4-c]pyrazol-5(4H)-yl)-2-(3-hydroxyazetidin-1-yl)-2-phenylethan-1-one or (R)-1-(2-((4-(difluoromethoxy)phenyl)sulfonyl)-2,6-dihydropyrrolo[3,4-c]pyrazol-5(4H)-yl)-2-(3-hydroxyazetidin-1-yl)-2-phenylethan-1-one as a white solid (ANAL-SFC) RT: 3.943 min. LCMS (ES) [M+1]⁺ m/z 505.

Step 5

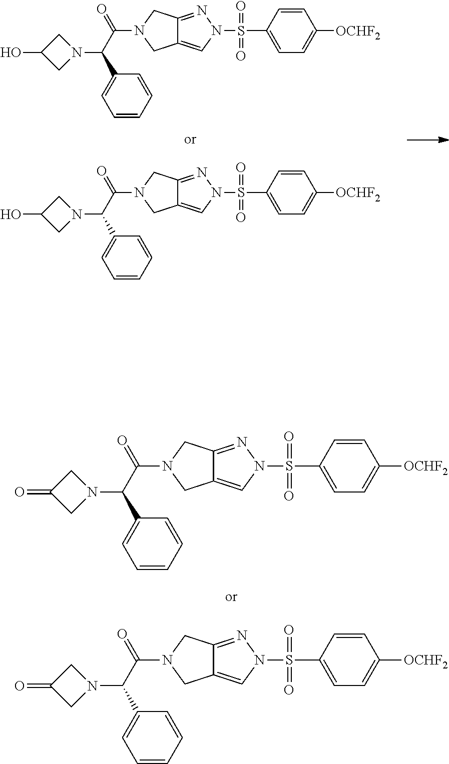 [SMILES: O=C([C@@H](c1ccccc1)N1CC(O)C1)N1Cc2cn(S(=O)(=O)c3ccc(OC(F)F)cc3)nc2C1 ; O=C([C@H](c1ccccc1)N1CC(O)C1)N1Cc2cn(S(=O)(=O)c3ccc(OC(F)F)cc3)nc2C1 ; O=C1CN([C@@H](C(=O)N2Cc3cn(S(=O)(=O)c4ccc(OC(F)F)cc4)nc3C2)c2ccccc2)C1 ; O=C1CN([C@H](C(=O)N2Cc3cn(S(=O)(=O)c4ccc(OC(F)F)cc4)nc3C2)c2ccccc2)C1]

Into a 40-mL vial purged and maintained with an inert atmosphere of nitrogen, was placed a mixture of (S)-1-(2-((4-(difluoromethoxy)phenyl)sulfonyl)-2,6-dihydropyrrolo[3,4-c]pyrazol-5(4H)-yl)-2-(3-hydroxyazetidin-1-yl)-2-phenylethan-1-one or (R)-1-(2-((4-(difluoromethoxy)phenyl)sulfonyl)-2,6-dihydropyrrolo[3,4-c]pyrazol-5(4H)-yl)-2-(3-hydroxyazetidin-1-yl)-2-phenylethan-1-one (RT=3.943 min; 200 mg, 0.40 mmol, 1.00 eq.), DCM (15.0 mL), and Dess-Martin reagent (505.8 mg, 1.19 mmol, 3.0 eq.) and the resulting solution was stirred for 2 hours at 0° C. The crude product was purified by Prep-HPLC with the following Column: Sunfire Prep C18 OBD Column, 50*250 mm, 5 μm 10 nm; Mobile Phase A: Water (0.05% NH3H2O), Mobile Phase B: ACN; Flow rate: 90 mL/min; Gradient: 25% B to 60% B in 12 min, 60% B; Wave Length: 220 nm nm; RT1 (min): 15; Number Of Runs: 0. This resulted in 50 mg (25.13%) of 1-[(1R)-2-{2-[4-(difluoromethoxy)benzenesulfonyl]-2H,4H,5H,6H-pyrrolo[3,4-c]pyrazol-5-yl}-2-oxo-1-phenylethyl]azetidin-3-one or 1-[(1S)-2-{2-[4-(difluoromethoxy)benzenesulfonyl]-2H,4H,5H,6H-pyrrolo[3,4-c]pyrazol-5-yl}-2-oxo-1-phenylethyl]azetidin-3-one as a white solid. ¹H NMR (300 MHz, DMSO-d6, ppm): 8.25 (d, J=4.8 Hz, 1H), 8.03-8.00 (m, 2H), 7.59-7.22 (m, 8H), 4.92-4.74 (m, 2H), 4.51-4.46 (m, 1H), 4.37-4.29 (m, 1H), 4.14-4.02 (m, 5H). (ANAL-SFC) RT: 2.347 min. LCMS (ES) [M+1]⁺ m/z 503.

### Example 1.110

### Synthesis of 1-[(1R)-2-{2-[4-(difluoromethoxy)benzenesulfonyl]-2H,4H,5H,6H-pyrrolo[3,4-c]pyrazol-5-yl}-2-oxo-1-phenylethyl]azetidin-3-one or 1-[(1S)-2-{2-[4-(difluoromethoxy)benzenesulfonyl]-2H,4H,5H,6H-pyrrolo[3,4-c]pyrazol-5-yl}-2-oxo-1-phenylethyl]azetidin-3-one (Compound 93)

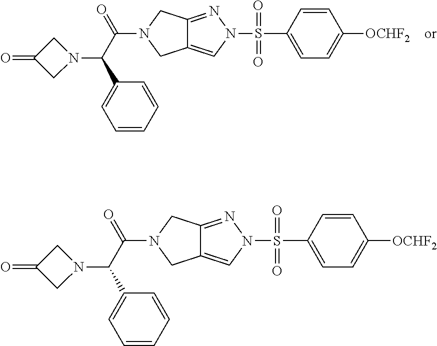 [SMILES: O=C1CN([C@@H](C(=O)N2Cc3cn(S(=O)(=O)c4ccc(OC(F)F)cc4)nc3C2)c2ccccc2)C1 ; O=C1CN([C@H](C(=O)N2Cc3cn(S(=O)(=O)c4ccc(OC(F)F)cc4)nc3C2)c2ccccc2)C1]

The tile compound was synthesized by the procedure described for Compound 92 using (R)-1-(2-((4-(difluoromethoxy)phenyl)sulfonyl)-2,6-dihydropyrrolo[3,4-c]pyrazol-5(4H)-yl)-2-(3-hydroxyazetidin-1-yl)-2-phenylethan-1-one or (S)-1-(2-((4-(difluoromethoxy)phenyl)sulfonyl)-2,6-dihydropyrrolo[3,4-c]pyrazol-5(4H)-yl)-2-(3-hydroxyazetidin-1-yl)-2-phenylethan-1-one [(ANAL-SFC) RT: 2.746 min] obtained in step 4 as the starting material for step 5. The crude product was purified by Prep-HPLC with the following Column: Sunfire Prep C18 OBD Column, 50*250 mm, 5 μm 10 nm; Mobile Phase A: Water (0.05% NH₃H₂O), Mobile Phase B: ACN; Flow rate: 90 mL/min; Gradient: 25% B to 60% B in 12 min, 60% B; Wave Length: 220 nm nm; RT₁ (min): 15; Number Of Runs: 0. This resulted in 80 mg (40.20%) of (R)-1-(2-(2-((4-(difluoromethoxy)phenyl)sulfonyl)-2,6-dihydropyrrolo[3,4-c]pyrazol-5(4H)-yl)-2-oxo-1-phenylethyl)azetidin-3-one as a white solid. ¹H NMR (300 MHz, DMSO-d6, ppm): 8.24-8.23 (m, 1H), 8.03-8.01 (m, 2H), 7.59-7.22 (m, 8H), 4.92-4.74 (m, 2H), 4.52-4.46 (m, 1H), 4.42-4.25 (m, 1H), 4.21-4.02 (m, 5H). (ANAL-SFC) RT: 2.127 min. LCMS (ES) [M+1]⁺ m/z 503.

### Example 1.111

### Synthesis of (2S)-3-hydroxy-1-(2-{[6-(oxetan-3-yloxy)pyridin-3-yl]sulfonyl}-2H,4H,5H,6H-pyrrolo[3,4-c]pyrazol-5-yl)-2-phenylpropan-1-one (Compound 95)

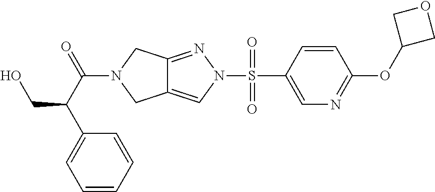 [SMILES: O=C([C@H](CO)c1ccccc1)N1Cc2cn(S(=O)(=O)c3ccc(OC4COC4)nc3)nc2C1]

The title compound was synthesized following a similar procedure as the one described for (2S)-1-{2-[(6-cyclopropoxypyridin-3-yl)sulfonyl]-2H,4H,5H,6H-pyrrolo[3,4-c]pyrazol-5-yl}-3-hydroxy-2-phenylpropan-1-one (Compound 97) but using oxetan-3-ol instead of cyclopropanol in step 2. The crude material was purified by column chromatography (4 G ISCO Gold) eluting with 0-100% EtOAc in heptane to provide a colorless solid consistent with the desired product. ¹H NMR (400 MHz, dmso) δ 8.90 (ddt, J=2.6, 1.8, 0.8 Hz, 1H), 8.48 (d, J=1.2 Hz, 1H), 8.46-8.39 (m, 1H), 8.07-7.97 (m, 1H), 7.40-7.19 (m, 5H), 5.46 (dtd, J=6.2, 5.0, 3.8 Hz, 1H), 4.94 (dd, J=32.1, 14.1 Hz, 1H), 4.82 (q, J=5.6 Hz, 1H; OH), 4.73-4.64 (m, 2H), 4.56-4.49 (m, 3H), 4.48-4.32 (m, 2H), 4.00 (d, J=9.0 Hz, 2H), 3.52 (dq, J=7.6, 4.4, 2.7 Hz, 1H). LCMS (ES) [M+1]+ m/z 470.99.

### Example 1.112

### Synthesis of 6-({5-[(2S)-3-hydroxy-2-phenylpropanoyl]-2H,4H,5H,6H-pyrrolo[3,4-c]pyrazol-2-yl}sulfonyl)-3-methyl-3,4-dihydro-2H-1,3-benzoxazin-2-one (Compound 96)

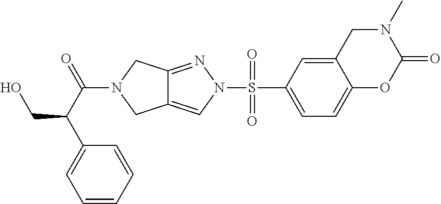 [SMILES: CN1Cc2cc(S(=O)(=O)n3cc4c(n3)CN(C(=O)[C@H](CO)c3ccccc3)C4)ccc2OC1=O]

The title compound was made from 6-((5,6-dihydropyrrolo[3,4-c]pyrazol-2(4H)-yl)sulfonyl)-3-methyl-3,4-dihydro-2H-benzo[e][1,3]oxazin-2-one (synthesized following a procedure similar as described for the synthesis of Intermediate I-1, except that 3-methyl-2-oxo-3,4-dihydro-2H-1,3-benzoxazine-6-sulfonyl chloride was used in the place of 1,3 benzothiazole-6-sulfonyl chloride) and (2S)-3-hydroxy-2-phenylpropanoic acid (Intermediate II-6) following a procedure similar to that described for the synthesis of Compound 1. The crude final product was purified by reverse phase preparative HPLC (Prep-C18, 5 uM Luna C18 column, 22×250 mm, Phenomenex; gradient elution of 10-70% MeCN in water over a 20 min period, where both solvents contain 0.1% formic acid) to provide the title product as a white solid. ¹H NMR (400 MHz, dmso) δ 8.20 (d, J=1.1 Hz, 1H), 7.90 (dt, J=2.4, 1.1 Hz, 1H), 7.83 (ddd, J=8.7, 2.4, 0.8 Hz, 1H), 7.35-7.24 (m, 4H), 7.24-7.16 (m, 2H), 4.82 (dd, J=21.1, 14.3 Hz, 1H), 4.51 (s, 2H), 4.47-4.20 (m, 3H), 4.01-3.88 (m, 2H), 3.56-3.42 (m, 2H), 2.91 (s, 3H). LCMS (ES) [M+1]⁺ m/z 483.1.

### Example 1.113

### Synthesis of (2S)-1-{2-[(6-cyclopropoxypyridin-3-yl)sulfonyl]-2H,4H,5H,6H-pyrrolo[3,4-c]pyrazol-5-yl}-3-hydroxy-2-phenylpropan-1-one (Compound 97)

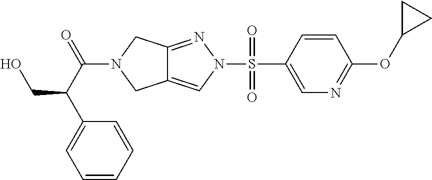 [SMILES: O=C([C@H](CO)c1ccccc1)N1Cc2cn(S(=O)(=O)c3ccc(OC4CC4)nc3)nc2C1]

Step 1

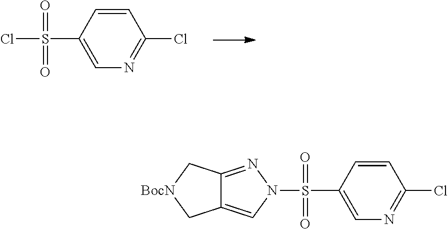 [SMILES: O=S(=O)(Cl)c1ccc(Cl)nc1 ; CC(C)(C)OC(=O)N1Cc2cn(S(=O)(=O)c3ccc(Cl)nc3)nc2C1]

To a suspension of sodium hydride (248.49 mg; 6.21 mmol; 1.30 eq.) in THF (30 mL) under N₂ in an ice bath was added a solution of tert-butyl 2H,4H,5H,6H-pyrrolo[3,4-c]pyrazole-5-carboxylate (1 000.00 mg; 4.78 mmol; 1.00 eq.) in THF (6 mL) over 5 min. The mixture was allowed to stir in an ice bath for 30 min. A solution of 6-chloro-3-pyridinesulfonyl chloride (1 013.42 mg; 4.78 mmol; 1.00 eq.) in THF (4 mL) was then added slowly. The mixture was allowed to stir in the ice bath for 45 min while warming up to RT. The reaction mixture was then quenched and diluted with NH₄C₁ (2 mL of AcOH was also added) and the organic solvent was removed under vacuum. The aqueous residue was extracted twice with a 1:3 mixture of IPA:CHCl3. The combined organics were washed with brine, dried over Na₂SO₄, filtered, and concentrated to provide a light tan solid consistent with the desired product. A portion was collected as is (1450 mg) and the rest of the crude material was absorbed onto a plug of silica gel and purified by column chromatography (12 G ISCO Gold) eluting with 0-50% EtOAc in heptane to provide the desired pure material as a colorless solid (135 mg). LCMS (ES) [M+1]⁺ m/z 385.

Step 2

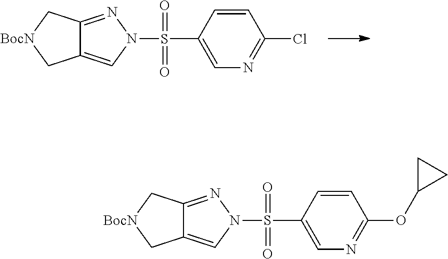 [SMILES: CC(C)(C)OC(=O)N1Cc2cn(S(=O)(=O)c3ccc(Cl)nc3)nc2C1 ; CC(C)(C)OC(=O)N1Cc2cn(S(=O)(=O)c3ccc(OC4CC4)nc3)nc2C1]

To a suspension of sodium hydride (9.86 mg; 0.25 mmol; 1.30 eq.) in 0.5 mL DMF at 0° C. was added cyclopropanol (14.32 mg; 0.25 mmol; 1.30 eq.) in 0.5 mL DMF and the mixture was stirred at 0° C. for 30 minutes. Tert-butyl 2-[(6-chloropyridin-3-yl)sulfonyl]-2H,4H,5H,6H-pyrrolo[3,4-c]pyrazole-5-carboxylate (73.00 mg; 0.19 mmol; 1.00 eq.) as a suspension in 1 mL DMF was then added drop-wise and after complete addition the mixture was stirred at RT. After 1.5 h the mixture was diluted with 1:1 PhMe/EtOAc, washed with an equal amount of water, dried over MgSO₄, filtered, and concentrated. The white solid residue was purified by column chromatography (4G ISCO Gold) eluting with 0-30% EtOAc in heptane to provide a white solid consistent with the desired product (23.5 mg; 30% yield); LCMS (ES) [M+1]⁺ m/z 407.

Step 3

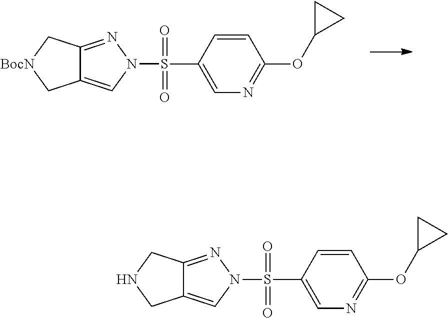 [SMILES: CC(C)(C)OC(=O)N1Cc2cn(S(=O)(=O)c3ccc(OC4CC4)nc3)nc2C1 ; O=S(=O)(c1ccc(OC2CC2)nc1)n1cc2c(n1)CNC2]

To a mixture of tert-butyl 2-[(6-cyclopropoxypyridin-3-yl)sulfonyl]-2H,4H,5H,6H-pyrrolo[3,4-c]pyrazole-5-carboxylate (23.00 mg; 0.06 mmol; 1.00 eq.) in 1,2-dichloroethane (0.46 mL) was added dibromozinc (38.23 mg; 0.17 mmol; 3.00 eq.). This mixture was allowed to stir at 55° C. After 48 h, the mixture was cooled to RT, diluted with water, quenched with 1 mL of aqueous ammonium hydroxide, and extracted with a 3:1 mix of CHCl3:IPA. The combined organics were washed with brine, dried over MgSO₄, filtered, and concentrated to provide a colorless solid. This material was taken on to the next step without further purification assuming 100% yield (17 mg).

Step 4

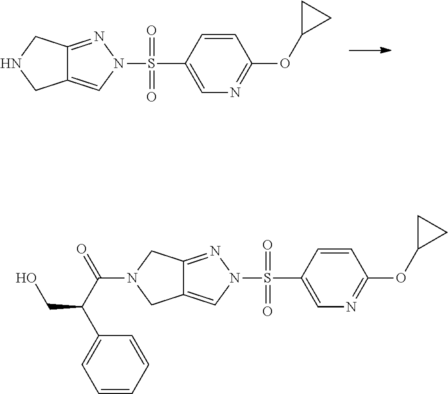 [SMILES: O=S(=O)(c1ccc(OC2CC2)nc1)n1cc2c(n1)CNC2 ; O=C([C@H](CO)c1ccccc1)N1Cc2cn(S(=O)(=O)c3ccc(OC4CC4)nc3)nc2C1]

To a mixture of (2S)-3-hydroxy-2-phenylpropanoic acid (Intermediate II-6; 11.99 mg; 0.07 mmol; 1.30 eq.), 2-cyclopropoxy-5-{2H,4H,5H,6H-pyrrolo[3,4-c]pyrazole-2-sulfonyl}pyridine (17.00 mg; 0.06 mmol; 1.00 eq.) and Hunig's base (0.02 mL; 0.11 mmol; 2.00 eq.) in N,N-dimethylformamide (0.55 mL) was added HATU (27.43 mg; 0.07 mmol; 1.30 eq.) and the resulting mixture was stirred at RT. After 2.5 h the mixture was diluted with 1:1 PhMe/EtOAc and washed with an equal amount of water. The organic phase was dried over MgSO₄, filtered and concentrated. The crude material was purified by column chromatography (4 G ISCO Gold) eluting with 0-80% EtOAc in heptane to provide a colorless solid consistent with the desired product. ¹H NMR (400 MHz, dmso) δ 8.96-8.88 (m, 1H), 8.51-8.45 (m, 1H), 8.44-8.38 (m, 1H), 8.08-8.00 (m, 1H), 7.39-7.27 (m, 4H), 7.26-7.19 (m, 1H), 4.94 (dd, J=31.8, 14.1 Hz, 1H), 4.85-4.79 (m, 1H), 4.64-4.29 (m, 3H), 4.20-4.11 (m, 1H), 4.05-3.94 (m, 2H), 3.52 (p, J=5.4, 4.9 Hz, 1H), 0.68-0.58 (m, 4H). LCMS (ES) [M+1]⁺ m/z 455.06.

### Example 1.114

### Synthesis of 1-{2-[4-(difluoromethoxy)benzenesulfonyl]-2H,4H,5H,6H-pyrrolo[3,4-c]pyrazol-5-yl}-2-(2-fluorophenyl)-2-methoxyethan-1-one (Compound 98; Racemic)

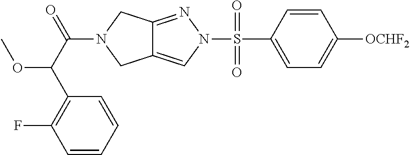 [SMILES: COC(C(=O)N1Cc2cn(S(=O)(=O)c3ccc(OC(F)F)cc3)nc2C1)c1ccccc1F]

The title compound was made from 6-{2H,4H,5H,6H-pyrrolo[3,4-c]pyrazole-2-sulfonyl}-1,3-benzothiazole (Intermediate I-2) and 2-(2-fluorophenyl)-2-methoxyacetic acid following a procedure similar as described for the synthesis of compound 1. The crude product was purified by reverse phase preparative HPLC (Prep-C18, 5 μM XBridge column, 19×150 mm, Waters; gradient elution of 0-65% MeCN in water over a 20 min period, where both solvents contain 0.1% formic acid) to provide the title product as a racemic mixture as a white solid. ¹H NMR (400 MHz, DMSO-d6) δ 8.25 (dd, J=2.9, 1.5 Hz, 1H), 8.04-7.97 (m, 2H), 7.60-7.07 (m, 7H), 5.29 (d, J=9.7 Hz, 1H), 4.83 (dd, J=19.1, 14.2 Hz, 1H), 4.51-4.22 (m, 3H), 3.28 (d, J=2.5 Hz, 3H).LCMS (ES) [M+1]+ m/z 482.2.

### Example 1.115

### Synthesis of 1-[(1R)-2-{2-[4-(difluoromethoxy)benzenesulfonyl]-2H,4H,5H,6H-pyrrolo[3,4-c]pyrazol-5-yl}-2-oxo-1-phenylethyl]azetidin-2-one or 1-[(1S)-2-{2-[4-(difluoromethoxy)benzenesulfonyl]-2H,4H,5H,6H-pyrrolo[3,4-c]pyrazol-5-yl}-2-oxo-1-phenylethyl]azetidin-2-one (Compound 100)

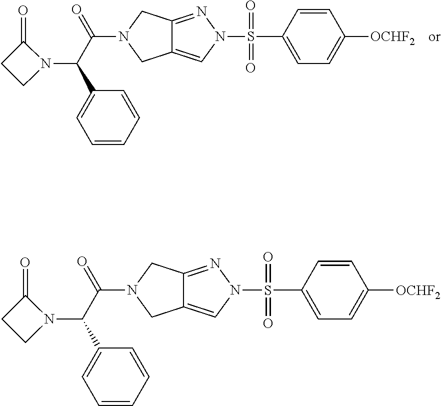 [SMILES: O=C([C@@H](c1ccccc1)N1CCC1=O)N1Cc2cn(S(=O)(=O)c3ccc(OC(F)F)cc3)nc2C1 ; O=C([C@H](c1ccccc1)N1CCC1=O)N1Cc2cn(S(=O)(=O)c3ccc(OC(F)F)cc3)nc2C1]

Step 1

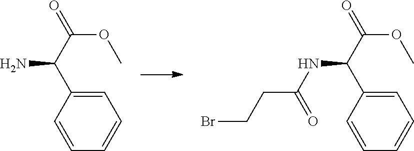 [SMILES: COC(=O)[C@H](N)c1ccccc1 ; COC(=O)[C@H](NC(=O)CCBr)c1ccccc1]

Into a 250 mL 3-necked round-bottom flask were added methyl (2R)-2-amino-2-phenylacetate (3.00 g, 18.16 mmol, 1.00 eq.), DCM (70 mL) and N,N-dimethylaniline (7.92 g, 65.36 mmol, 3.60 eq.) at room temperature. To the above mixture was added 3-bromopropanoyl chloride (3.58 g, 20.89 mmol, 1.15 eq.) dropwise at −20° C. The resulting mixture was stirred for additional 2 h at room temperature then it was concentrated under vacuum. The residue was purified by silica gel column chromatography, eluting with PE/EA (3:1) to afford methyl (2R)-2-(3-bromopropanamido)-2-phenylacetate (4 g, 73.38%) as a light yellow oil. LCMS (ES) [M+1]⁺ m/z 300, 302.

Step 2

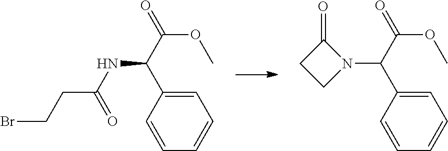 [SMILES: COC(=O)[C@H](NC(=O)CCBr)c1ccccc1 ; COC(=O)C(c1ccccc1)N1CCC1=O]

Into a 250 mL 3-necked round-bottom flask were added methyl (2R)-2-(3-bromopropanamido)-2-phenylacetate (3.60 g, 11.99 mmol, 1.00 eq.) and DMF (15 mL), DCM (60 mL) at room temperature. To the above mixture was added NaH (0.53 g, 13.19 mmol, 1.1 eq.) at 0° C. and the resulting mixture was stirred for additional 3 h at room temperature. The reaction was then quenched with water at room temperature and extracted with CH₂Cl₂ (3×100 mL). The combined organic layers were washed with brine (3×100 mL), dried over anhydrous Na₂SO₄, filtered, and concentrated under reduced pressure. The residue was purified by reverse flash chromatography with the following conditions: column, C18 silica gel; mobile phase, FA (0.1%) in water and ACN, 10% to 50% gradient in 15 min; detector, UV 220 nm. This resulted in methyl-2-(2-oxoazetidin-1-yl)-2-phenylacetate (2 g, 76.06%) as a light yellow oil. LCMS (ES) [M+1]⁺ m/z 220.

Step 3

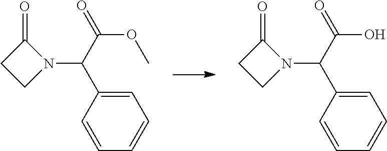 [SMILES: COC(=O)C(c1ccccc1)N1CCC1=O ; O=C(O)C(c1ccccc1)N1CCC1=O]

Into a 100 mL round-bottom flask were added methyl-2-(2-oxoazetidin-1-yl)-2-phenylacetate (2 g, 9.12 mmol, 1.00 eq.) and MeOH (15 mL) at room temperature. To the above mixture was added LiOH.H₂O (0.44 g, 18.24 mmol, 2 eq.) in H₂O (5 mL) drop-wise at room temperature, and the resulting mixture was stirred for additional 3 h at room temperature. The residue was purified by reverse flash chromatography with the following conditions: column, C18 silica gel; mobile phase, FA (0.1%) in water and ACN, 10% to 20% gradient in 10 min; detector, UV 220 nm. This resulted in (2-oxoazetidin-1-yl)(phenyl)acetic acid (800 mg, 42.73%) as an off-white semi-solid. LCMS (ES) [M−1]⁺ m/z 204.

Step 4

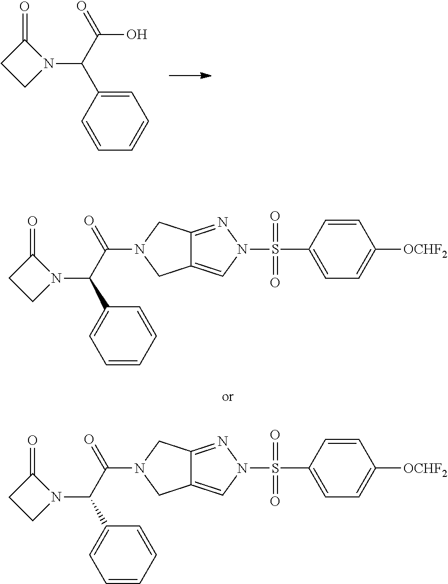 [SMILES: O=C(O)C(c1ccccc1)N1CCC1=O ; O=C([C@@H](c1ccccc1)N1CCC1=O)N1Cc2cn(S(=O)(=O)c3ccc(OC(F)F)cc3)nc2C1 ; O=C([C@H](c1ccccc1)N1CCC1=O)N1Cc2cn(S(=O)(=O)c3ccc(OC(F)F)cc3)nc2C1]

Into a 40 mL vial were added (2-oxoazetidin-1-yl)(phenyl)acetic acid (500 mg, 2.44 mmol, 1.00 equiv), 2-[4-(difluoromethoxy)benzenesulfonyl]-4H,5H,6H-pyrrolo[3,4-c]pyrazole hydrochloride (514 mg, 1.46 mmol, 0.6 eq.), DMF (5 mL), and DIEA (630 mg, 4.87 mmol, 2 eq.) at room temperature. To the above mixture was added T₃P (930 mg, 2.92 mmol, 1.2 eq.) dropwise at 0° C., and the resulting mixture was stirred for additional 3 h at 0° C. The crude product was purified by Prep-HPLC with the following conditions (Prep-HPLC-003): Column, SunFire Prep C18 OBD Column, 19*150 mm, 5 μm 10 nm; mobile phase, Water (0.05% FA) and ACN (15% ACN up to 45% in 7 min); The resulting mixture was concentrated under vacuum. The product was purified by Chiral-Prep-HPLC with the following conditions Column: (R, R)-WHELK-O1-Kromasil, 2.12*25 cm, 5 μm; Mobile Phase A: HEX: DCM=3: 1-HPLC, Mobile Phase B: EtOH-HPLC; Flow rate: 35 mL/min; Gradient: 40% B to 40% B in 22 min; Wave Length: 220/254 nm; RT1 (10 min); RT2 (16 min); Sample Solvent: EtOH: DCM=3: 1-HPLC; Injection Volume: 5 mL; This resulted in 58.8 mg, 9.61%, of 1-[(1R)-2-{2-[4-(difluoromethoxy)benzenesulfonyl]-2H,4H,5H,6H-pyrrolo[3,4-c]pyrazol-5-yl}-2-oxo-1-phenylethyl]azetidin-2-one or 1-[(1S)-2-{2-[4-(difluoromethoxy)benzenesulfonyl]-2H,4H,5H,6H-pyrrolo[3,4-c]pyrazol-5-yl}-2-oxo-1-phenylethyl]azetidin-2-one (RT=10 min/22 min) and 1-[(1S)-2-{2-[4-(difluoromethoxy)benzenesulfonyl]-4H,6H-pyrrolo[3,4-c]pyrazol-5-yl}-2-oxo-1-phenylethyl]azetidin-2-one (60.9 mg, 9.95%, RT₂=16 min/22 min) as a white solid. ¹H NMR (300 MHz, DMSO-d₆): δ 8.26 (d, J=8.9 Hz, 1H), 8.08-7.98 (m, 2H), 7.72-7.12 (m, 8H), 5.79 (d, J=8.8 Hz, 1H), 4.82 (dd, J=19.1, 14.3 Hz, 1H), 4.59-4.36 (m, 2H), 4.01 (dd, J=14.5, 3.5 Hz, 1H), 3.58-3.46 (m, 1H), 2.99-2.89 (m, 1H), 2.89-2.71 (m, 2H). LCMS (ES) [M+1]⁺ m/z 503. CHIRAL_HPLC Analysis: Retention time 1.970 min.

### Example 1.116

### Synthesis of (2S)-1-{2-[(6-cyclopropylpyridin-3-yl)sulfonyl]-2H,4H,5H,6H-pyrrolo[3,4-c]pyrazol-5-yl}-3-hydroxy-2-phenylpropan-1-one (Compound 101)

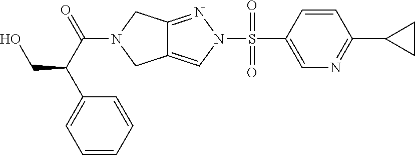 [SMILES: O=C([C@H](CO)c1ccccc1)N1Cc2cn(S(=O)(=O)c3ccc(C4CC4)nc3)nc2C1]

Step 1

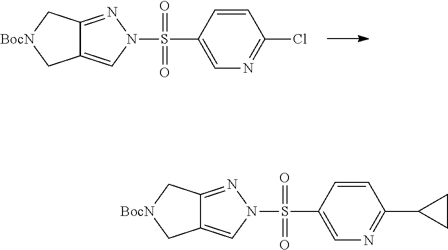 [SMILES: CC(C)(C)OC(=O)N1Cc2cn(S(=O)(=O)c3ccc(Cl)nc3)nc2C1 ; CC(C)(C)OC(=O)N1Cc2cn(S(=O)(=O)c3ccc(C4CC4)nc3)nc2C1]

A mixture of tert-butyl 2-[(6-chloropyridin-3-yl)sulfonyl]-2H,4H,5H,6H-pyrrolo[3,4-c]pyrazole-5-carboxylate (Synthesized as described for compound 97; 62.00 mg; 0.16 mmol; 1.00 eq.), cyclopropylboronic acid (48.44 mg; 0.56 mmol; 3.50 eq.), tricyclohexylphosphane (4.52 mg; 0.02 mmol; 0.10 eq.) and potassium phosphate, tribasic (68.40 mg; 0.32 mmol; 2.00 eq.) in toluene (0.81 mL) and water (0.04 mL) was sparged with nitrogen for 10 minutes. Palladium acetate (3.62 mg; 0.02 mmol; 0.10 eq.) was then added and the mixture was heated to 100° C. After 3 h, the mixture was cooled to RT, diluted with EtOAc, and washed with water and brine. The organic layer was dried over MgSO₄, filtered, and concentrated. The residue was purified by column chromatography (12 G ISCO Gold) eluting with 0-40% in heptane to obtain a colorless solid consistent with the desired product. (56 mg; 89%); LCMS (ES) [M+1]⁺ m/z 391.

Step 2

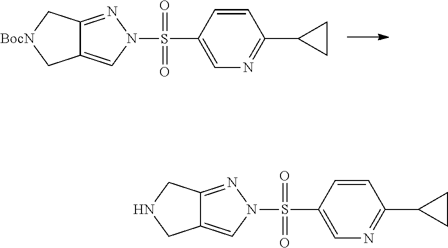 [SMILES: CC(C)(C)OC(=O)N1Cc2cn(S(=O)(=O)c3ccc(C4CC4)nc3)nc2C1 ; O=S(=O)(c1ccc(C2CC2)nc1)n1cc2c(n1)CNC2]

To a mixture of tert-butyl 2-[(6-cyclopropylpyridin-3-yl)sulfonyl]-2H,4H,5H,6H-pyrrolo[3,4-c]pyrazole-5-carboxylate (56.00 mg; 0.14 mmol; 1.00 eq.) in 1,2-dichloroethane (1.12 mL) was added dibromozinc (96.89 mg; 0.43 mmol; 3.00 eq.). This mixture was allowed to stir at 55° C. After 48 h, the mixture was cooled to RT, diluted with water, quenched with 1.5 mL of aqueous ammonium hydroxide, and extracted with a 3:1 mix of CHCl₃:IPA. The combined organics were washed with brine, dried over MgSO₄, filtered, and concentrated to provide an off-white solid. This material was taken on to the next step without further purification assuming 100% yield (41 mg).

Step 3

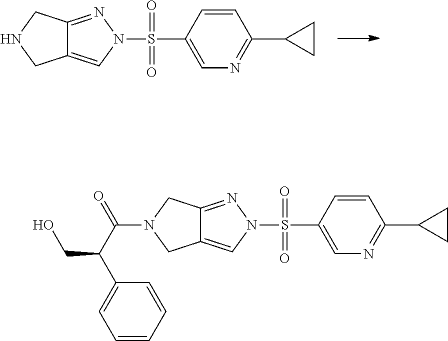 [SMILES: O=S(=O)(c1ccc(C2CC2)nc1)n1cc2c(n1)CNC2 ; O=C([C@H](CO)c1ccccc1)N1Cc2cn(S(=O)(=O)c3ccc(C4CC4)nc3)nc2C1]

To a mixture of (2S)-3-hydroxy-2-phenylpropanoic acid (Intermediate II-6; 30.51 mg; 0.18 mmol; 1.30 eq.), 2-cyclopropyl-5-{2H,4H,5H,6H-pyrrolo[3,4-c]pyrazole-2-sulfonyl}pyridine (41.00 mg; 0.14 mmol; 1.00 eq.) and Hunig's base (0.05 mL; 0.28 mmol; 2.00 eq.) in N,N-dimethylformamide (1.41 mL) was added HATU (69.80 mg; 0.18 mmol; 1.30 eq.) and the resulting mixture was stirred at RT. After 2.5 h, the mixture was diluted with 1:1 PhMe/EtOAc and washed with an equal amount of water. The organic phase was dried over MgSO₄, filtered, and concentrated. The crude material was purified by column chromatography (12 G ISCO Gold) eluting with 0-80% EtOAc in heptane to isolate consistent with the desired product. ¹H NMR (400 MHz, dmso) δ 8.86 (dd, J=2.5, 0.8 Hz, 1H), 8.22 (q, J=1.2 Hz, 1H), 8.11 (ddd, J=8.4, 2.5, 1.6 Hz, 1H), 7.54 (ddd, J=8.4, 1.8, 0.8 Hz, 1H), 7.32-7.18 (m, 5H), 4.88-4.78 (m, 1H), 4.78-4.75 (m, 1H), 4.46-4.23 (m, 3H), 3.98-3.89 (m, 2H), 3.52-3.44 (m, 1H), 2.25-2.17 (m, 1H), 1.05 (dt, J=8.0, 3.0 Hz, 2H), 0.98 (dt, J=4.6, 2.9 Hz, 2H). LCMS (ES) [M+1]⁺ m/z 438.98.

### Example 1.117

### Synthesis of (2R)-2-(3-fluoropyridin-2-yl)-2-hydroxy-1-{2-[(5-methoxypyridin-2-yl)sulfonyl]-2H,4H,5H,6H-pyrrolo[3,4-c]pyrazol-5-yl}ethan-1-one or (2S)-2-(3-fluoropyridin-2-yl)-2-hydroxy-1-{2-[(5-methoxypyridin-2-yl)sulfonyl]-2H,4H,5H,6H-pyrrolo [3,4-c]pyrazol-5-yl}ethan-1-one (Compound 104)

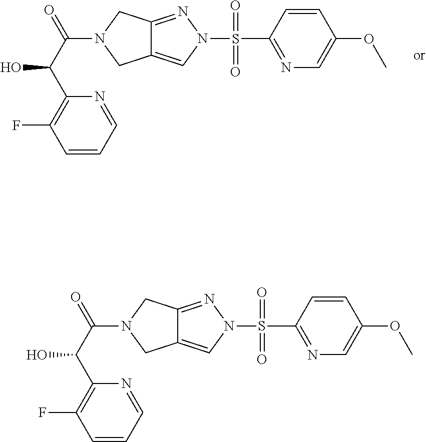 [SMILES: COc1ccc(S(=O)(=O)n2cc3c(n2)CN(C(=O)[C@H](O)c2ncccc2F)C3)nc1 ; COc1ccc(S(=O)(=O)n2cc3c(n2)CN(C(=O)[C@@H](O)c2ncccc2F)C3)nc1]

Step 1

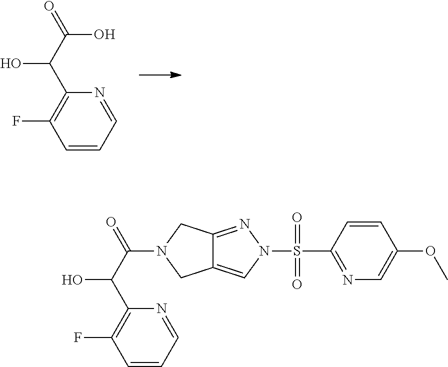 [SMILES: O=C(O)C(O)c1ncccc1F ; COc1ccc(S(=O)(=O)n2cc3c(n2)CN(C(=O)C(O)c2ncccc2F)C3)nc1]

Into a 40-mL vial, was placed (3-fluoropyridin-2-yl)(hydroxy)acetic acid (260.00 mg, 1.52 mmol, 1.00 eq.), 2-((5-methoxypyridin-2-yl)sulfonyl)-2,4,5,6-tetrahydropyrrolo[3,4-c]pyrazole hydrochloride (Synthesized as described for Intermediate I-2 but using 5-methoxypyridine-2-sulfonyl chloride instead of 4-(difluoromethoxy)benzenesulfonyl chloride; 480.32 mg, 1.52 mmol, 1.00 eq.), DMF (15.00 mL), and NMM (461.02 mg, 4.56 mmol, 3.00 eq.) followed by the addition of HATU (693.24 mg, 1.82 mmol, 1.20 eq.), in portions at 0° C. The resulting solution was stirred for 2 h at room temperature. The crude product (1.2 g) was purified by Prep-HPLC with the following conditions: Column, XBridge Prep C18 OBD Column, 19 cm, 150 mm, 5 um; mobile phase, Water (0.1% FA) and CAN (5% Phase B up to 60% in 11 min); Detector, 254. This resulted in 330 mg (50.18%) of 2-(3-fluoropyridin-2-yl)-2-hydroxy-1-(2-((5-methoxypyridin-2-yl)sulfonyl)-2,6-dihydropyrrolo[3,4-c]pyrazol-5(4H)-yl)ethan-1-one as a yellow oil. LCMS (ES) [M+1]⁺ m/z: 433.

Step 2

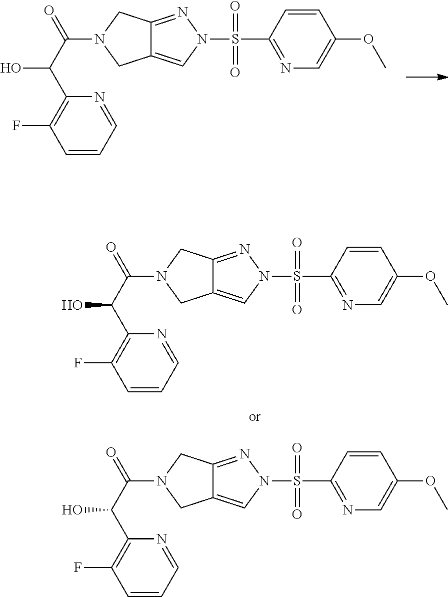 [SMILES: COc1ccc(S(=O)(=O)n2cc3c(n2)CN(C(=O)C(O)c2ncccc2F)C3)nc1 ; COc1ccc(S(=O)(=O)n2cc3c(n2)CN(C(=O)[C@H](O)c2ncccc2F)C3)nc1 ; COc1ccc(S(=O)(=O)n2cc3c(n2)CN(C(=O)[C@@H](O)c2ncccc2F)C3)nc1]

Racemic 2-(3-fluoropyridin-2-yl)-2-hydroxy-1-(2-((4-methoxyphenyl)sulfonyl)-2,6-dihydropyrrolo[3,4-c]pyrazol-5(4H)-yl)ethan-1-one (330 mg) was purified by Prep-SFC with the following conditions: Column, CHIRAL ART Cellulose-SJ, 3*25 cm, 5 um; mobile phase, CO₂ (65%) and MeOH (0.1% 2M NH₃-MeOH) (35%); Detector, 254. This resulted in 116 mg (70.30%%) of (2R)-2-(3-fluoropyridin-2-yl)-2-hydroxy-1-{2-[(5-methoxypyridin-2-yl)sulfonyl]-2H,4H,5H,6H-pyrrolo[3,4-c]pyrazol-5-yl}ethan-1-one or (2S)-2-(3-fluoropyridin-2-yl)-2-hydroxy-1-{2-[(5-methoxypyridin-2-yl)sulfonyl]-2H,4H,5H,6H-pyrrolo[3,4-c]pyrazol-5-yl}ethan-1-one as a white solid. ¹H NMR (300 MHz, DMSO-d₆, ppm): δ 8.40-8.32 (m, 2H), 8.21 (d, J=16.2 Hz, 1H), 8.28-8.17 (m, 1H), 7.80-7.63 (m, 2H), 7.52-7.41 (m, 1H), 5.78 (br, 1H), 5.63-5.54 (m, 1H), 4.86-4.78 (m, 1H), 4.58-4.39 (m, 2H), 4.19-4.06 (m, 1H), 3.93 (s, 3H). LCMS (ES, m/z): [M+H]⁺: 433.

### Example 1.118

### Synthesis of (2R)-2-(3-fluoropyridin-2-yl)-2-hydroxy-1-{2-[(5-methoxypyridin-2-yl)sulfonyl]-2H,4H,5H,6H-pyrrolo[3,4-c]pyrazol-5-yl}ethan-1-one or (2S)-2-(3-fluoropyridin-2-yl)-2-hydroxy-1-{2-[(5-methoxypyridin-2-yl)sulfonyl]-2H,4H,5H,6H-pyrrolo[3,4-c]pyrazol-5-yl}ethan-1-one (Compound 105)

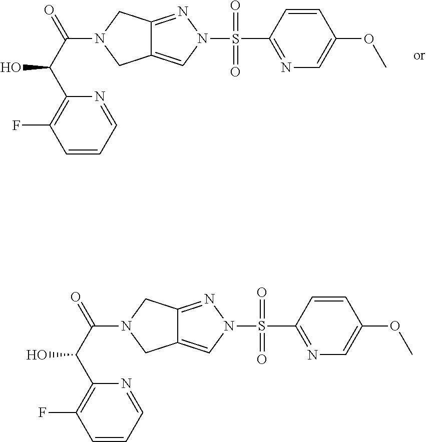 [SMILES: COc1ccc(S(=O)(=O)n2cc3c(n2)CN(C(=O)[C@H](O)c2ncccc2F)C3)nc1 ; COc1ccc(S(=O)(=O)n2cc3c(n2)CN(C(=O)[C@@H](O)c2ncccc2F)C3)nc1]

The title compound was synthesized by the procedure described for Compound 104. Racemic 2-(3-fluoropyridin-2-yl)-2-hydroxy-1-(2-((5-methoxypyridin-2-yl)sulfonyl)-2,6-dihydropyrrolo[3,4-c]pyrazol-5(4H)-yl)ethan-1-one (180 mg) was purified by Prep-SFC with the following conditions: Column, CHIRAL ART Cellulose-SJ, 3*25 cm, 5 um; mobile phase, CO₂ (65%) and MeOH (0.1% 2M NH₃-MeOH) (35%); Detector, 254. This resulted in 114 mg (69.09%) of (2R)-2-(3-fluoropyridin-2-yl)-2-hydroxy-1-{2-[(5-methoxypyridin-2-yl)sulfonyl]-2H,4H,5H,6H-pyrrolo[3,4-c]pyrazol-5-yl}ethan-1-one or (2S)-2-(3-fluoropyridin-2-yl)-2-hydroxy-1-{2-[(5-methoxypyridin-2-yl)sulfonyl]-2H,4H,5H,6H-pyrrolo[3,4-c]pyrazol-5-yl}ethan-1-one as a white solid. ¹ NMR (300 MHz, DMSO-d₆, ppm): δ 8.40-8.32 (m, 2H), 8.21 (d, J=16.2 Hz, 1H), 8.28-8.17 (m, 1H), 7.80-7.63 (m, 2H), 7.52-7.41 (m, 1H), 5.78 (br, 1H), 5.63-5.54 (m, 1H), 4.86-4.78 (m, 1H), 4.58-4.39 (m, 2H), 4.19-4.06 (m, 1H), 3.93 (s, 3H). LCMS (ES, m/z): [M+H]⁺: 433.

Compound 62 can be synthesized in a similar manner to the above compounds using intermediates I-6 and II-1/2/3/4.

### II. BIOLOGICAL EXAMPLES

### Example 2.1

### Pyruvate Kinase Activation Methods

Protein Production and Purification

Human pyruvate kinase R (PKR), residues 14-574, and human pyruvate kinase M2, residues 1-531, were cloned into expression plasmids and obtained from ATUM Bio (Newark, Calif.). Proteins were translated as fusions with 6×-His, 8×-Arg, and SUMO at the N-terminus. A third construct of human PKR was truncated, residues 50-574, and similarly cloned for use in crystallography experiments. All proteins were cloned, expressed in E. coli, and purified using similar protocols. Cells were grown at 30° C. in Luria-Bertani broth supplemented with 0.4% glucose to OD600=0.8 and induced with 0.4 mM IPTG at 16° C. for 18 hours. Cells were harvested by centrifugation, resuspended in 50 mM potassium phosphate pH 8.0, 500 mM NaCl, 25 mM imidazole pH 8.0, and 3 mM β-mercaptoethanol. Cells were lysed using a LM20 microfluidizer from Microfluidics (Westwood, Mass.). The crude lysate was immediately supplemented with 0.2 mM phenylmethylsulfonyl fluoride (PMSF) and centrifuged at 20,000 g for 20 minutes. The soluble fraction was subsequently incubated with 2 mL Ni-NTA (GE Healthcare) per 1,000 ODs for 1 hour at 4° C. Following incubation with the Ni-NTA resin, lysate was removed by pelleting resin at 2,500 g for 3 minutes and washed 3 times with 9 bed volumes of 50 mM potassium phosphate pH 8.0, 500 mM NaCl, 25 mM imidazole, and 3 mM β-mercaptoethanol. Following the batch wash Ni-NTA resin was loaded onto a gravity column and His-tagged protein was eluted with 6 bed volumes of 10 mM Tris/HCl pH 8.0, 200 mM NaCl, 500 mM imidazole, and 3 mM β-mercaptoethanol. Eluted protein was dialyzed overnight against 10 mM Tris/HCl pH 8.0, 200 mM NaCl, and 1 mM DTT and the 6×-His-8×-Arg-SUMO-tag was cleaved using a 20:1 molar ratio of protein:3C protease. The protein was purified by anion exchange chromatography on a HiTrapQ or MonoQ 10/100 GL column (GE Healthcare) via a linear NaCl gradient and twice by size exclusion chromatography using a Superdex S200 26/60 column (GE Healthcare) run in 10 mM Tris/HCl pH 8.0, 200 mM NaCl, 10 mM MgCl₂, 1 mM DTT. Proteins were concentrated to ˜12 mg/mL for crystallization and flash frozen for storage.

### Example 2.2

Biochemical Assay for Activation of Pyruvate Kinase R

Activation of pyruvate kinase R (PKR) and pyruvate kinase M2 (PKM2) were measured using a luminescence based measurement of ATP generation and the Kinase-Glo luminescent kinase reagent kit. 0.1 nM of PKR or PKM2 was incubated for 1 hour with 0.3 mM ADP, 2 mM FBP, 1% DMSO, compound and Assay Buffer mix (500 mM Tris/HCl pH 7.5, 500 mM NaCl, 50 mM MgCl₂, BSA, and 2 mM DTT, in a total reaction volume of 30 uL. After one hour PEP was added to the reaction at a final concentration of 0.1 mM and incubated for another hour at room temperature. 30 uL of KinaseGlo reagent was added and the reaction was incubated for 15 minutes. Endpoint luminescence data was measured using an EnVision plate reader (PerkinElmer). The assay results are provided in Table 2.

TABLE 2

| Cmpd No. | PKR^{a} | PKM₂^{b} |
|---|---|---|
| from | AC₅₀(nM)/% | AC₅₀(nM)/% |
| Table 1 | activation^{c} | activation^{c} |
| 1 | 28/82 | 33/133 |
| 2 | 556/127 | |
| 3 | 44/104 | 45/101 |
| 4 | 1812/100 | |
| 5 | 82/59 | 38/100 |
| 6 | 2527/150 | |
| 7 | 76/137 | 22/116 |
| 8 | 1977/152 | |
| 9 | 147/131 | |
| 10 | 455/165 | |
| 11 | 7273/164 | |
| 12 | 41/129 | 23/90 |
| 13 | 167/62 | |
| 14 | 110/77 | 53/63 |
| 15 | 45/76 | 26/83 |
| 16 | 156/40 | |
| 17 | 24/112 | 23/135 |
| 18 | 83/116 | |
| 19 | 699/59 | |
| 20 | 44/113 | 14/60 |
| 21 | 209/123 | |
| 22 | 50/99 | 33/79 |
| 23 | 56/95 | 61/116 |
| 24 | 78/65 | 59/63 |
| 25 | 19/44 | 31/42 |
| 26 | 72/69 | 147/47 |
| 27 | 39/41 | |
| 28 | 13/45 | |
| 29 | 92/104 | 25/77 |
| 30 | 371/130 | |
| 31 | 173/76 | |
| 32 | 38/72 | 91/86 |
| 33 | 130/147 | |
| 34 | 210/55 | |
| 35 | 62/115 | 93/89 |
| 36 | 591/38 | |
| 37 | 21/86 | 17/83 |
| 38 | 270/102 | |
| 39 | 41/80 | 53/67 |
| 40 | 60/88 | 31/99 |
| 42 | 17/80 | 16/71 |
| 43 | 75/82 | |
| 44 | 550/86 | |
| 45 | 15/94 | |
| 46 | 129/45 | |
| 47 | 64/40 | |
| 48 | 223/46 | |
| 49 | 17/88 | |
| 50 | 71/99 | |
| 51 | 23/51 | |
| 52 | 13/129 | |
| 53 | 36/56 | |
| 54 | 4000/36 | |
| 55 | >10000/0 | |
| 56 | 30/152 | |
| 57 | 17/96 | |
| 58 | 25/81 | |
| 59 | 48/110 | |
| 60 | 77/90 | |
| 61 | 452/48 | |
| 62 | 47/115 | |
| 63 | 8.3/132 | |
| 64 | 3.4/137 | |
| 65 | 6.2/128 | |
| 66 | 13/142 | |
| 67 | 29/144 | |
| 68 | 10/179 | |
| 69 | 4.7/136 | |
| 70 | 15/80 | |
| 71 | 8.9/79 | |
| 72 | 6/129 | |
| 73 | 13/150 | |
| 74 | 8.5/100 | |
| 75 | 4.4/149 | |
| 76 | 6.9/19 | |
| 77 | 8.3/72 | |
| 78 | 4.7/174 | |
| 79 | 4.9/118 | |
| 80 | 2.1/40 | |
| 81 | 3.8/133 | |
| 82 | 5/96 | |
| 83 | 7.9/77 | |
| 84 | 4.5/90 | |
| 85 | 6.8/121 | |
| 86 | 1.8/103 | |
| 87 | 25/156 | |
| 88 | 3/133 | |
| 89 | 7.9/132 | |
| 90 | 430/13 | |
| 91 | 14/113 | |
| 92 | 800/120 | |
| 93 | 5550/84 | |
| 94 | 17/51 | |
| 95 | 7080/8 | |
| 96 | 53/13 | |
| 97 | >10000/142 | |
| 98 | 210/139 | |
| 99 | 190/92 | |
| 100 | >10000/0 | |
| 101 | 41/25 | |
| 102 | 12/81 | |
| 103 | 220/41 | |
| 104 | 240/103 | |
| 105 | 9400/151 | |
| 106 | 44/66 | |
| 107 | >10000/0 | |
| 108 | >10000/0 | |

^{a}Activity data was measured in PKR biochemical assay.
^{b}Activity data was measured in PKM2 cellular assay.
^{c}The maximal activation level achieved with each compound relative to the activation level achieved by the literature compound AG-348 @ 10 μM.

### Example 2.3

Cell Based Assay for Activation of Pyruvate Kinase M2 in Lung Carcinoma Cell Line

H1299 cells were seeded in 96-well plates at 2,000 cells/well (100 uL). Treated plates were incubated overnight at 37° C. with 5% CO₂. Compounds were diluted in complete media and added to cells in the presence of 1% DMSO. Cells were incubated with compound for 90 minutes at 37° C. and 5% CO₂ before washing three times with PBS to remove residual compound and then lysed in lysis buffer (Cell Signaling). Cell lysate was analyzed using the NADH coupled assay described below with a reaction mixture of 180 uM NADH, 2 mM ADP, 0.5 units of LDH and 0.5 mM of PEP. PKM2 activity was measured at steady state using a coupled enzyme activation system based on NADH consumption. PKM2 produces pyruvate and the coupled system uses lactate dehydrogenase (LDH) to reduce pyruvate to lactate with the concomitant oxidation of NADH to NAD⁺. Conversion of NADH to NAD⁺ was monitored using a SPECTROstar Nano plate reader (BMG Labtech) at a wavelength of 340 nm and subtraction of background absorbance measured at 750 nm. The change in NADH absorbance after PEP addition was monitored and slope obtained by subtracting baseline at 750 nm followed by least squares fitting to a simple linear regression model. A 10-point curve was generated to calculate the AC₅₀ values by fitting the rates of NADH consumption against increasing concentration of compound.

Efforts have been made to ensure accuracy with respect to numbers used (e.g., amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, N.Y.; and Janeway, C., Travers, P., Walport, M., Shlomchik (2001) Immunobiology, 5th Ed., Garland Publishing, New York.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A compound of Formula I: 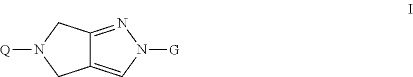
or a pharmaceutically acceptable salt thereof;
wherein,
G is 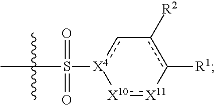
wherein, [symbol]
represents a single or double bond;
X⁴ is selected from the group consisting of N, C and CH;
X¹⁰ and X¹¹ are each independently selected from the group consisting of N, CH₂, and CH, provided that only one of X¹⁰ and X¹¹ can be N;
R¹ is selected from the group consisting of hydrogen, C₁-C₃ alkyl, C₁-C₃ alkoxy, halogen, C₁-C₃ haloalkoxy, amino, —O—R^{aa}, —(C₁-C₃ alkoxy)-R^{aa}, C₃-C₇ cycloalkyl, and hydroxy;
R^{aa} is 4- to 7-membered heterocyclyl or C₃-C₇ cycloalkyl;
R² is amino, C₁-C₃ alkoxy, halogen, C₁-C₃ haloalkoxy, or hydrogen, or, together with R¹ and the carbon to which each of R² and R¹ is attached, forms a 5- or 6-membered heteroaryl or 5- to 7-membered heterocyclyl containing one or two heteroatoms;
wherein said heteroaryl or heterocyclyl formed by R¹ and R² is optionally substituted with one or two substituents, each independently selected from the group consisting of C₁-C₃ alkyl, C₁-C₃ haloalkyl, C₁-C₃ alkoxy, halogen, and C₁-C₃ haloalkoxy;
provided that R¹ and R² cannot both be amino or hydrogen;
Q is:
i. —C(O)—C(J¹)(J²)(J³)
wherein,
J¹ is hydrogen or optionally substituted C₁-C₃ alkyl; and
J² and J³ are each independently selected from the group consisting of C₁-C₃ alkyl, hydroxy, C₁-C₃ alkoxy, hydroxy-C₁-C₃ alkyl, NR^{1a}R^{1b}, —(C₁-C₃ alkyl)-NR^{1a}R^{1b}, C₆-C₁₀ aryl, 5- to 10-membered heteroaryl, C₃-C₁₀ cycloalkyl, and 4- to 10-membered heterocyclyl;
R^{1a} and R^{1b} are each independently hydrogen, hydroxy-C₁-C₃ alkyl, —C(O)H, or C₁-C₃ alkyl; and
wherein said aryl, heteroaryl, or heterocyclyl of J² and J³ is optionally substituted one or two times, in each instance independently with C₁-C₃ alkyl, C₁-C₃ haloalkyl, hydroxy, NR^{2a}R^{2b}, halogen, C₁-C₃ alkoxy, or C₁-C₃ haloalkoxy;
wherein R^{2a} and R^{2b} are each independently hydrogen or C₁-C₃ alkyl; or,
J¹ and J² together with the carbon atom to which they are each attached form a 4- to 7-membered heterocyclyl or C₃-C₇ cycloalkyl;
ii. —C(O)O—R, wherein R is hydrogen or C₁-C₆ alkyl; or,
iii. —S(O)₂-M
wherein M is selected from the group consisting of C₆-C₁₀ aryl, 5- to 10-membered heteroaryl, and 5- to 10-membered heterocyclyl, wherein said aryl, heteroaryl, or heterocyclyl is optionally substituted one or two times, in each instance independently with C₁-C₃ alkyl, C₁-C₃ haloalkyl, NR^{3a}R^{3b}, —C(O)R^{2c}, hydroxy, halogen, C₁-C₃ alkoxy, or C₁-C₃ haloalkoxy;
wherein R^{3a} and R^{3b} are each independently hydrogen or C₁-C₃ alkyl; and R^{2C} is selected from the group consisting of hydrogen, hydroxy, and C₁-C₃ alkyl;
provided that the compound is not: 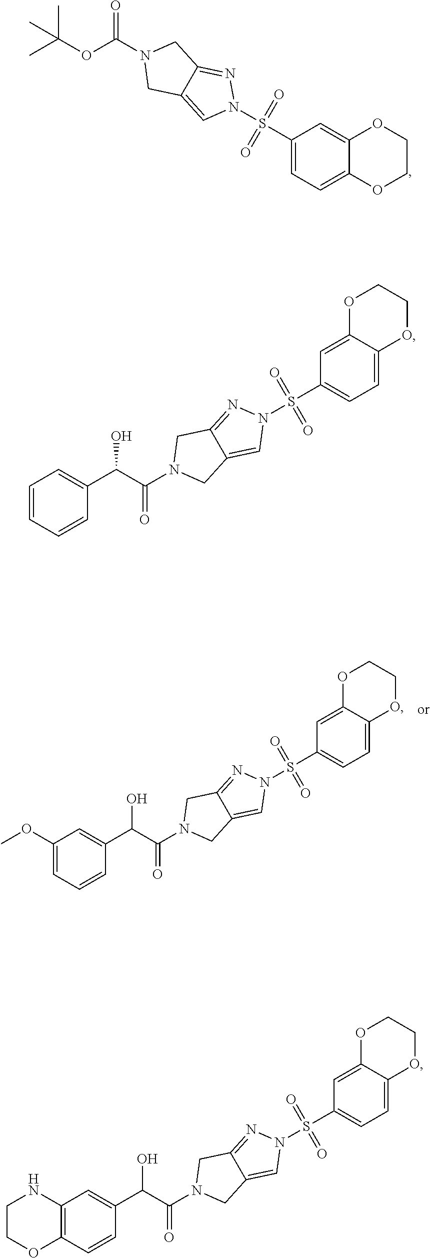 [SMILES: CC(C)(C)OC(=O)N1Cc2cn(S(=O)(=O)c3ccc4c(c3)OCCO4)nc2C1 ; O=C([C@@H](O)c1ccccc1)N1Cc2cn(S(=O)(=O)c3ccc4c(c3)OCCO4)nc2C1 ; COc1cccc(C(O)C(=O)N2Cc3cn(S(=O)(=O)c4ccc5c(c4)OCCO5)nc3C2)c1 ; O=C(C(O)c1ccc2c(c1)NCCO2)N1Cc2cn(S(=O)(=O)c3ccc4c(c3)OCCO4)nc2C1]
or salts thereof.

2. The compound of claim 1, wherein G is: 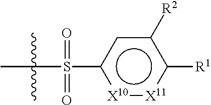
wherein X¹⁰ and X¹¹ are each independently CH or N, provided that only one of X¹⁰ and X¹¹ can be N.

3. The compound of claim 1 or 2, wherein,
Q is —C(O)—C(J¹)(J²)(J³), wherein:
J¹ is hydrogen or C₁-C₃ alkyl;
J² is selected from the group consisting of hydroxy, C₁-C₃ alkyl, —NHR^{1b}, —(C₁-C₃ alkyl)-NHR^{1b}, C₁-C₃ alkoxy, 4- to 6-membered heterocyclyl, and hydroxy-C₁-C₃ alkyl;
R^{1b} is hydrogen, hydroxy-C₁-C₃ alkyl, —C(O)H, or C₁-C₃ alkyl; and
J³ is optionally substituted phenyl or 6- to 10-membered heteroaryl; or,
J¹ and J², together with the carbon atom to which they are each attached, form a 4- to 6-membered heterocyclyl.

4. The compound of claim 3, wherein J³ is phenyl or pyridinyl optionally substituted with halo, methyl, C₁-C₃ haloalkyl, or C₁-C₃ haloalkoxy.

5. The compound of claim 4, wherein J³ is phenyl or pyridinyl optionally substituted with chloro, fluoro, methyl, —CH₂F, —CHF₂, —CF₃, —OCH₂F, —OCHF₂, or —OCF₃.

6. The compound of any one of the preceding claims, wherein Q is 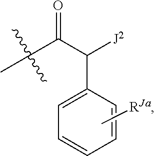
wherein J² is selected from the group consisting of —NH₂, —CH₂NH₂, —NHCH₂CH₂OH, —NHC(O)H, hydroxy, —C(CH₃)₂OH, —CH₂OH, —OCH₃, 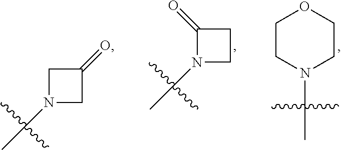
and —CH(OH)CH₃; and
R^{Ja} is hydrogen, chloro, fluoro, —CF₃, —OCF₃, or methyl.

7. The compound of any one of claims 1-5, wherein Q is 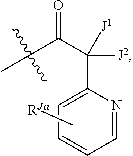
wherein J¹ is methyl or hydrogen and J² is —CH₂OH, or hydroxy; and
R^{Ja} is hydrogen, methyl, fluoro, or chloro.

8. The compound of claim 7, wherein J¹ is hydrogen.

9. The compound of claim 6 or 7, wherein R^{Ja} is ortho on the phenyl or pyridinyl ring.

10. The compound of any one of claims 1-5, wherein J¹ and J², together with the carbon atom to which they are each attached, form a 4- to 7-membered heterocyclyl.

11. The compound of claim 10, wherein J¹ and J², together with the carbon atom to which they are each attached, form a tetrahydrofuranyl, oxetanyl, pyrrolidinyl, or morpholinyl ring.

12. The compound of claim 1 or 2, wherein Q is —S(O)₂-M, wherein M is optionally substituted 5- to 10-membered heterocyclyl or 5- to 10-membered heteroaryl.

13. The compound of claim 12, wherein M is 9- or 10-membered bicyclic heterocyclyl or 9- or 10-membered bicyclic heteroaryl, optionally substituted once with NR^{3a}R^{3b}, C₁-C₃ haloalkoxy, or —C(O)R^{2c};
wherein R^{3a} and R^{3b} are each hydrogen; and R^{2c} is C₁₋C₃ alkyl.

14. The compound of claim 13, wherein M is:

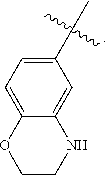 [SMILES: *c1ccc2c(c1)NCCO2]

15. The compound of claim 1, wherein X⁴ is C.

16. The compound of any one of the preceding claims, wherein X¹⁰ is CH.

17. The compound of any one of claims 1-15, wherein X¹⁰ is N.

18. The compound of any one of claims 1-16, wherein X¹¹ is N.

19. The compound of any one of claims 1-17, wherein X¹¹ is CH.

20. The compound of any one of the preceding claims, wherein R¹ is selected from the group consisting of hydrogen, C₁-C₃ alkyl, C₁-C₃ alkoxy, halogen, C₁-C₃ haloalkoxy, amino, —O-R^{aa}, —(C₁-C₃ alkoxy)-R^{aa}, C₃-C₇ cycloalkyl, and hydroxy;
R^{aa} is 4- to 7-membered heterocyclyl or C₃-C₇ cycloalkyl; and
R² is amino, C₁-C₃ alkoxy, halogen, C₁-C₃ haloalkoxy, or hydrogen, provided that R¹ and R² cannot both be amino or hydrogen.

21. The compound of any one of the preceding claims, wherein R¹ is C₁-C₃ haloalkoxy, C₁-C₃ alkoxy, C₃-C₅ cycloalkyl, or —O—R^{aa}; wherein,
R^{aa} is 4- to 6-membered heterocyclyl or C₃-C₅ cycloalkyl; and
R² is hydrogen.

22. The compound of claim 21, wherein R¹ is —OCH₃, —OCH₂F, —OCHF₂, —OCHF₃, cyclopropyl,

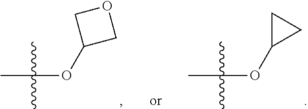 [SMILES: *OC1COC1 ; *OC1CC1]

23. The compound of claim 21 or 22, wherein R¹ is —OCHF₂.

24. The compound of any one of claims 15-23, wherein G is:

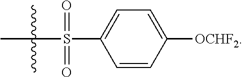 [SMILES: *S(=O)(=O)c1ccc(OC(F)F)cc1]

25. The compound of any one of claims 1-20, wherein R² is C₁-C₃ haloalkoxy and R¹ is hydrogen.

26. The compound of claim 25, wherein R² is —OCHF₂.

27. The compound of claim 1 or 2, wherein R² together with R¹ and the carbon to which each of R² and R¹ is attached form a 5- or 6-membered heteroaryl or 5- or 6-membered heterocyclyl, wherein said heteroaryl or heterocyclyl is optionally substituted with one substituent selected from the group consisting of C₁-C₃ alkyl and C₁-C₃ haloalkoxy.

28. The compound of claim 27, wherein R² together with R¹ and the carbon to which each of R² and R¹ is attached form a thiazolyl ring.

29. The compound of claim 28, wherein G is

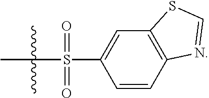 [SMILES: *S(=O)(=O)c1ccc2ncsc2c1]

30. The compound of claim 27, wherein R² together with R¹ and the carbon to which each of R² and R¹ is attached form a morpholinyl, dioxanyl, pyridinyl, pyrimidinyl, or dioxolyl ring.

31. The compound of claim 30, wherein G is selected from the group consisting of

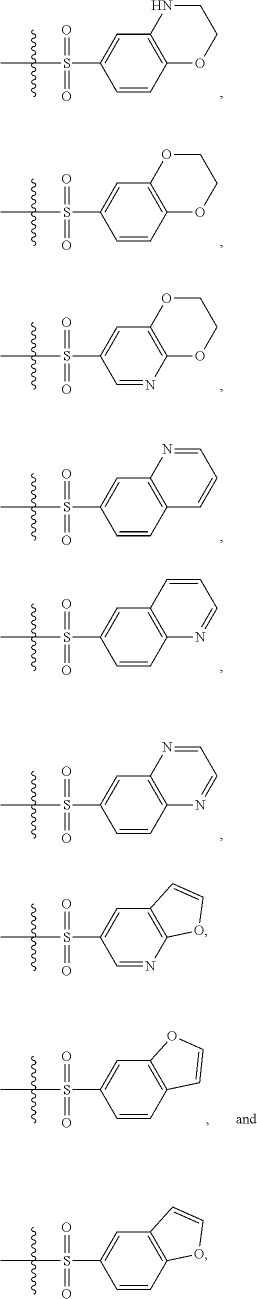

32. The compound of claim 27, wherein R² together with R¹ and the carbon to which each of R² and R¹ is attached form a pyrazolyl ring, wherein said pyrazolyl is optionally substituted with one substituent selected from the group consisting of methyl, —CH₂F, —CHF₂, and —CF₃.

33. The compound of claim 32, wherein G is selected from the group consisting of

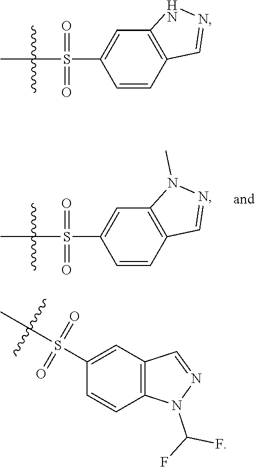 [SMILES: *S(=O)(=O)c1ccc2cn[nH]c2c1 ; *S(=O)(=O)c1ccc2cnn(C)c2c1 ; *S(=O)(=O)c1ccc2c(cnn2C(F)F)c1]

34. The compound of claim 1, selected from Table 1, or a pharmaceutically acceptable salt thereof.

35. A pharmaceutical composition comprising a compound of any one of claims 1-34 or a pharmaceutically acceptable salt thereof, and a pharmaceutically acceptable excipient.

36. A method of treating a disease or disorder associated with modulation of pyruvate kinase (PKR) and/or PKM2 in a subject, comprising administering to the subject an effective amount of a compound of any one of claims 1-34 or the pharmaceutical composition of claim 35.

37. A method of activating PKR and/or PKM2 in a subject, comprising administering to the subject an effective amount of a compound of any one of claims 1-34 or the pharmaceutical composition of claim 35.

38. A method of treating a subject afflicted with a disease associated with decreased activity of PKR and/or PKM2, comprising administering to the subject an effective amount of a compound of any one of claims 1-34 or the pharmaceutical composition of claim 35.

39. The method of claim 38, wherein the disease is selected from the group consisting of sickle cell disease, sickle cell anemia, thalassemia, hereditary non-spherocytic hemolytic anemia, hemolytic anemia, hereditary spherocytosis, hereditary elliptocytosis, abetalipoproteinemia, paroxysmal nocturnal hemoglobinuria, acquired hemolytic, and anemia of chronic diseases.
